(19) United States
(12) Patent Application Publication
(10) Pub. No.: US 2024/0408094 A1
(43) Pub. Date: Dec. 12, 2024

(54) INHIBITION OF USP7 INDUCES P53-INDEPENDENT TUMOR GROWTH SUPPRESSION IN TRIPLE-NEGATIVE BREAST CANCERS BY DESTABILIZING FOXM1

(71) Applicant: The Trustees of Columbia University in the City of New York, New York, NY (US)

(72) Inventors: Wei GU, Paramus, NJ (US); Jingjie YI, SHANG HAI (CN)

(21) Appl. No.: 18/739,284

(22) Filed: Jun. 10, 2024

Related U.S. Application Data
(60) Provisional application No. 63/471,968, filed on Jun. 8, 2023.

(51) Int. Cl. A61K 31/517; A61P 35/00; C07D 401/14

(52) U.S. Cl. CPC A61K 31/517; A61P 35/00; C07D 401/14

(57) ABSTRACT

The invention discloses PU7-1, pharmaceutical compositions comprising PU7-1, and methods of treating cancer in a subject in need thereof comprising administering to the subject a therapeutically effective amount of PU7-1.

This application claims the benefit of and priority to U.S. Provisional Patent Application No. 63/471,968, filed on Jun. 8, 2023, the content of which is hereby incorporated by reference in its entirety.

All patents, patent applications and publications cited herein are hereby incorporated by reference in their entirety. The disclosures of these publications in their entireties are hereby incorporated by reference into this application.

### STATEMENT REGARDING FEDERALLY SPONSORED RESEARCH OR DEVELOPMENT

This invention was made with government support under Grant CA254970 awarded by the National Institutes of Health. The government has certain rights in the invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Numerous studies indicate that inhibition of USP7 suppresses tumor growth by activating p53. However, p53 is frequently mutated in most triple-negative breast cancers (TNBC), characterized as the very aggressive form of breast cancer with limited treatment options and poor patient outcomes. There is currently no mechanistic understanding of USP7 contribution to tumor growth through the p53-independent manner. p53-independent tumor growth suppression will be a critical goal for the treatment of triple-negative breast cancers.

### SUMMARY OF THE INVENTION

In certain aspects, the subject matter disclosed herein is directed to a compound:

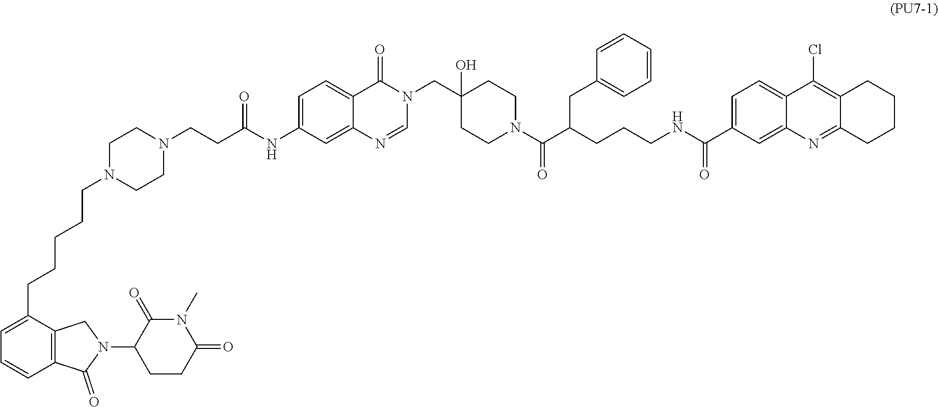 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]

or a pharmaceutically acceptable salt thereof.

In certain aspects, the subject matter disclosed herein is directed to a compound:

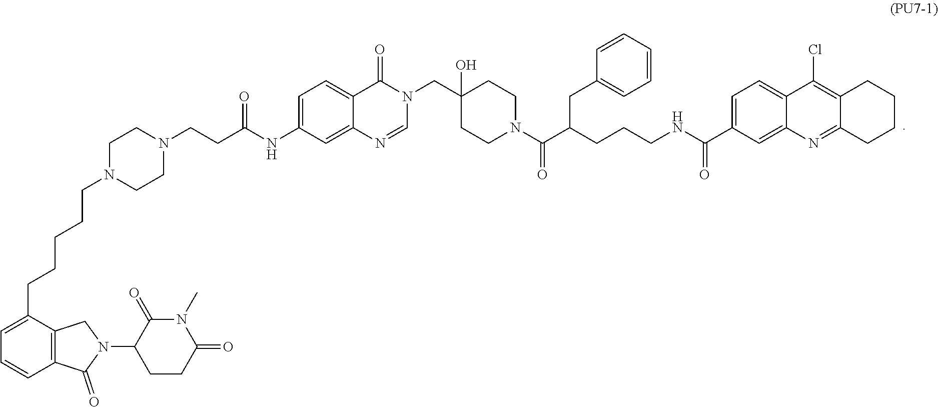 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]

In some embodiments, the subject matter disclosed herein is directed to a pharmaceutical composition comprising a pharmaceutically acceptable excipient and the compound PU7-1, or a pharmaceutically acceptable salt thereof. In some embodiments, the subject matter disclosed herein is directed to a pharmaceutical composition comprising a pharmaceutically acceptable excipient and the compound PU7-1.

In certain aspects, the subject matter disclosed herein is directed to a method of treating cancer in a subject in need thereof comprising administering to the subject a therapeutically effective amount of compound:

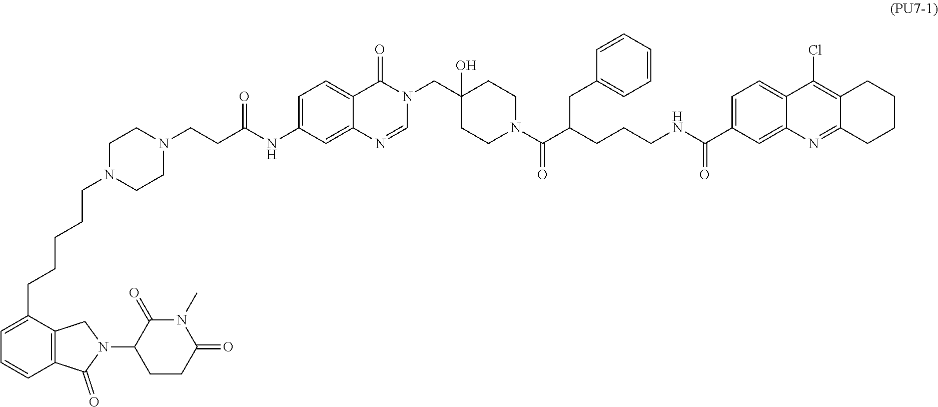 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]

or a pharmaceutically acceptable salt thereof.

In certain aspects, the subject matter disclosed herein is directed to a method of treating cancer in a subject in need thereof comprising administering to the subject a therapeutically effective amount of compound:

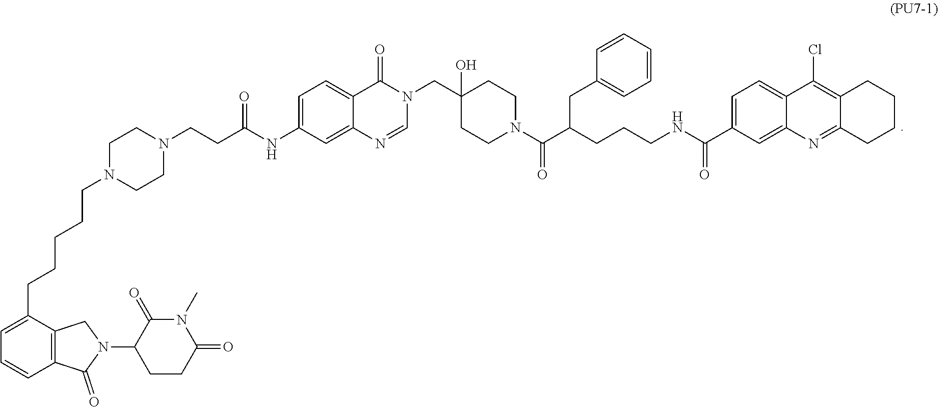 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]

In some embodiments, the compound (PU7-1) or the pharmaceutically acceptable salt thereof is administered as a pharmaceutical composition comprising a pharmaceutically acceptable excipient. In some embodiments, the compound (PU7-1) or the pharmaceutically acceptable salt thereof is administered as a pharmaceutical composition comprising a pharmaceutically acceptable excipient. In some embodiments, the cancer is a p53-independent cancer. In some embodiments, the cancer is triple-negative breast cancer. In some embodiments, the subject is a human.

### BRIEF DESCRIPTION OF FIGURES

This application contains one or more drawings executed in color. Copies of this patent or patent application publication with color drawing(s) will be provided by the United States Patent and Trademark Office upon request and payment of the necessary fec.

FIGS. 1A-H show that FOXM1 directly interacts with USP7 deubiquitinase. FIG. 1A shows FOXM1 expression in TCGA TNBC tumors versus normal tissues. FIG. 1B shows that Western blot analysis was used to detect the expression of FOXM1 in human breast cancer cells MDA-MB-468 (p53 R273H), MDA-MB-231 (p53 R280K), BT549 (p53 R249S), BT474 (p53 E285K), T47D (p53 L194F), SKBR3 (p53 R175H), and MCF7 (p53 wildtype). FIG. 1C shows a schematic representation of the SFB-FOXM1 constructs and the purification of SFB-FOMI complex. FIG. 1D shows a number of unique peptides for USP7 and known FOXM1-binding partners, identified by Mass spectrometry. FIG. 1E shows immunoblots for USP7 after immunoprecipitation of F-FOXM1, with anti-Flag beads, from H1299 cells transfected with constructs as indicated. FIG. 1F shows immunoblots for FOXM1 after immunoprecipitation of HA-USP7, with anti-HA beads, from H1299 cells transfected with constructs as indicated. FIG. 1G shows immunoblots for endogenous USP7 after immunoprecipitation of endogenous FOXM1 from MDA-MB-468 and MDA-MB-231 cells. FIG. 1H shows immunoblots for pulldown of purified F-FOXM1 with purified GST or GST-USP7. These data represent two independent experiments.

FIGS. 2A-F show that USP7 deubiquitinates and stabilizes FOXM1 protein. FIG. 2A shows immunoblots of F-FOXM1 in CAL33 cells that were transfected with indicated constructs for 24 h. FIG. 2B shows MDA-MB-468, MDA-MB-231 and BT549 cells transfected with non-targeting control siRNA or USP7 specific siRNA for 72 h. The expression of FOXM1 protein was detected by immunoblotting. FIG. 2C shows immunoblots for FOXM1 of H1299 cells after transfection with F-FOXM1 alone, and with wildtype USP7 (WT) or USP7 C223S (CS) for 24 h. FIG. 2D shows H1299 cells transfected with indicated constructs for 36 h and treated with 10 μM of MG132 for 4 h before harvest. After anti-Ni-NTA immunoprecipitation under denaturing condition, the ubiquitination of FOXM1 was detected by immunoblots. FIG. 2E shows immunoblots of FOXM1 in H1299 cells transfected with constructs as indicated for 24 h, then incubated with 25 μg/ml of cycloheximide for additional 0, 2.5, 5 and 10 h. FIG. 2F shows quantification of FOXM1 protein levels in (FIG. 2E) by ImageJ. These data represent two independent experiments.

FIG. 3A-J show that PU7-1 is a selective PROTAC degrader for USP7 protein. FIG. 3A shows the chemical structure of USP7 PROTAC degrader PU7-1. FIG. 3B shows CAL33 cells treated with indicated dosages of PU7-1 for 72 h. USP7 protein levels were detected by immunoblots. FIG. 3C shows CAL33 cells treated with 10 μM of PU7-1 for indicated times. USP7 protein levels were measured by immunoblots. FIG. 3D shows quantitative real-time PCR used to determine the USP7 mRNA levels in CAL33 treated with or without PU7-1 for 72 h. ns, p>0.05. FIG. 3E shows immunoblots of USP7 in CAL33 cells treated with or without 10 μM of PU7-1 48 h, then incubated with 25 μg/ml of cycloheximide for additional 0, 8, 12 and 24 h. FIG. 3F shows quantification of USP7 protein levels in (FIG. 3E) by ImageJ. FIG. 3G shows CAL33 cells treated with indicated concentrations of PU7-1 for 24 h. USP7 protein levels were detected by immunoblots, then quantified by ImageJ after normalized to internal control β-actin. Half maximal degradation concentration (DC₅₀) was calculated by Graphpad Prism. FIG. 3H shows immunoblots of CRBN and USP7 in control and CRBN crispr knockout (KO) CAL33 cells that were treated with or without 2.5 μM of PU7-1 for 72 h. FIG. 3I shows immunoblots of USP7 in CAL33 cells that were treated with DMSO or 5 μM of PU7-1 for 48 h, and 5M of MLN4924, 25 μM of Chloroquine (CLQ) or 5 μM of MG132 were added at 24 h before harvest. FIG. 3J shows heatmap of the deubiquitinases in quantitative proteomics analysis of A549 cells that were treated with 2.5M PU7-1 versus DMSO for 72 h. These data represent two independent experiments.

FIGS. 4A-H show that PU7-1 treatment leads to the FOXM1 degradation and the suppression of its transcriptional targets. FIG. 4A shows immunoblots of FOXM1 protein in CAL33 cells that were treated with indicated concentrations of PU7-1 for 96 h. FIG. 4B shows immunoblots of FOXM1 protein in CAL33 cells that were treated with 10 μM of PU7-1 for indicated times. FIG. 4C shows immunoblots of FOXM1 in CAL33 cells treated with or without 10 μM of PU7-1 for 48 h, then incubated with 50 μg/ml of cycloheximide for additional 0, 2, 4 and 6 h. FIG. 4D shows quantification of FOXM1 protein levels in (FIG. 4C) by ImageJ. FIG. 4E shows immunoblots of FOXM1 in CAL33 cells that were treated with 10 μM of PU7-1 for 72 h and treated with 1 μM of MLN4924 and BafA1 for 24 h and 10 μM of MG132 for 6 h before harvest. FIGS. 4F and 4G show CAL33 cells treated with or without 10 μM of PU7-1 for 96 h. Total RNA was extracted and subjected to RNAseq analysis. Differentially expressed genes were further analyzed. Top 10 downregulated KEGG enriched pathways are shown in FIG. 4F. Heatmap in FIG. 4G represents the downregulation of known FOXM1 target genes. FIG. 4H shows QPCR analysis of FOXM1 target genes in CAL33 treated with 10 μM of PU7-1 for 96 h. These data represent two independent experiments.

FIGS. 5A-G show that PU7-1 mediates FOXM1 degradation, cell proliferation inhibition and tumor suppression in TNBC. FIG. 5A shows immunoblots of FOXM1 in TNBC cell lines MDA-MB-468, MDA-MB-231 and BT549 treated with or without 10 μM of PU7-1 for 96 h. FIGS. 5B and 5C show representative images (FIG. 5B) and normalized absorbance at 600 nm (FIG. 5C) of colony-formation assays in MDA-MB468 and BT549 treated with or without 10 μM of PU7-1 for 2 weeks. FIG. 5D shows representative dose-response curves of CellTiter-Glo cell viability assay in MDA-MB-468 and BT549. FIG. 5E shows a treatment schedule for xenograft mouse models. FIGS. 5F and 5G show representative images of MDA-MB-468 tumors (FIG. 5F) and quantification of tumor weight (G) in nude mice xenograft model. **p<0.01. These data represent two independent experiments.

FIGS. 6A-F show that FOXM1 is required for the PU7-1-mediated growth inhibition and tumor suppression in TNBC. FIG. 6A shows immunoblots of FOXM1 in MDA-MB-468 FOXM1 cells treated with or without 1 μg/ml of doxycycline for 72 h. FIG. 6B shows MDA-MB-468 FOXM1 inducible cells induced by 1 μg/ml of doxycycline overnight, then DMSO or 10 μM of PU7-1 were added for 72 h. FOXM1 protein levels were determined by immunoblotting.

FIGS. 6C and 6D show representative images of BrdU staining (FIG. 6C) and quantification of BrdU positive (BrdU+) cells (FIG. 6D) in MDA-MB468 FOXM1 inducible cells that were induced with 1 μg/ml Doxycycline overnight followed by the treatment of 10 μM of PU7-1 for 72 h. **p<0.01. FIGS. 6E and 6F show representative images of MDA-MB-468 FOXM1 tumors (FIG. 6E) and quantification of tumor weight (FIG. 6F) in nude mice xenograft model. **p<0.01. These data represent two independent experiments.

FIGS. 7A-D show that FOXM1 is highly expressed in TNBC tumors. FIG. 7A shows a Kaplan-Meier analysis of overall survival of BRCA patients with different expressions of FOXM1. FIG. 7B shows a violin plot of FOXM1 expression levels in TCGA pan-cancers with different p53 status. FIG. 7C shows the percentage of tumor samples with mutant p53 in BRCA (all subtypes) and TNBC. FIG. 7D shows FOXM1 expression in different subtypes of BRCA in TCGA.

FIGS. 8A-E show that USP7 interacts with and regulates FOXM1 protein. FIG. 8A shows Gelcode blue staining of proteins in SFB-FOXM1 immunoprecipitation complex. FIG. 8B shows the protein sequence of human USP7 (highlighted in red and underlined) with peptides identified in FOXM1 immunoprecipitation complex by Mass spectrometry. FIG. 8C shows immunoblots for endogenous USP7 after immunoprecipitation of endogenous FOXM1 from CAL33 cells. FIG. 8D shows immunoblots for endogenous FOXM1 after immunoprecipitation of endogenous USP7 from H1299 cells. FIG. 8E shows CAL33 and H460 cells transfected with control or USP7 siRNA for 96 h. FOXM1 protein levels were detected by immunoblotting.

FIGS. 9A-B show a Pearson Correlation analysis of USP7 expression and FOXM1 signature in BRCA. FIG. 9A shows a list of FOXM1 signature used for FIG. 9B. FIG. 9B shows a Pearson Correlation analysis of USP7 expression and FOXM1 signature in breast cancer (BRCA) from TCGA dataset.

FIGS. 10A-B show screening of 66 USP7 PROTAC compounds. FIG. 10A shows CAL33 cells treated with 10 μM of USP7 PROTAC compounds for 96 h. The USP7 protein levels were determined by Western blot analysis. FIG. 10B shows CAL33 cells treated with 5 μM of USP7 PROTAC compounds for 72 h. The USP7 protein levels were determined by Western blot analysis.

FIGS. 11A-E show that PU7-1 is a selective and potent PROTAC degrader for USP7. FIG. 11A show human cancer cells harboring wildtype p53 treated with or without 10 μM of PU7-1 for 72 h. The protein levels of p53 and its target p21 were detected by immunoblotting.

FIG. 11B shows immunoblots of USP7 in CAL33 cells treated with indicated concentration of PU7-1 for 24 h. FIG. 11C shows the chemical structure of the negative control PU7-1N (upper panel) and immunoblots of USP7 in CAL33 and A549 cells treated with 2.5 μM of PU7-1N or PU7-1 for 48 h. FIG. 11D shows CAL33 cells treated with 10 μM of PU7-1 for 96 h. The expression of USP proteins was detected by immunoblotting. FIG. 11E shows A549 cells treated with 2.5 μM of PU7-1 for 72 h. Quantitative proteomics analysis were then performed. The volcano plot represents all deferentially expressed genes in DMSO control versus PU7-1.

FIGS. 12A-D show that PU7-1 leads to inhibition of proliferation of human cancer cells. FIGS. 12A and 12B show representative images of BrdU staining (FIG. 12A) and quantification of BrdU positive (BrdU+) cells (FIG. 12B) in CAL33 cells treated with or without 10 μM of PU7-1 for 72 h. ****p<0.0001. FIGS. 12C and 12D show representative images (FIG. 12C) and normalized absorbance at 600 nm (FIG. 12D) of colony-formation assays in BT474, CAL33, DU145, SK-NDZ treated with or without 10 μM of PU7-1 for 2 weeks.

FIGS. 13A-C show that PU7-1 leads to proliferation inhibition in human cancer cells. FIGS. 13A and 13B show representative images of BrdU staining (FIG. 13A) and quantification of BrdU positive (BrdU+) cells (FIG. 13B) in BT549 cells treated with or without 10 μM of PU7-1 for 72 h. ***p<0.001. FIG. 13C show representative immunohistochemistry images of Ki67, USP7 and FOXM1 in MDA-MB-468 tumors.

FIG. 14 shows ¹H NMR spectrum of PU7-1.

FIG. 15 shows ¹³C NMR spectrum of PU7-1.

FIGS. 16A-C show LC-MS spectrum of PU7-1. FIG. 16A shows Total Ion Chromatogram spectral data of PU7-1. FIG. 16B shows Time of Flight spectral data of PU7-1. FIG. 16C shows LC-MS total wave chromatogram spectral data of PU7-1.

FIG. 17 shows shows ¹H NMR spectrum of PU7-1N.

FIG. 18 shows ¹³C NMR spectrum of PU7-1N.

FIGS. 19A-C show LC-MS spectrum of PU7-1N. FIG. 19A shows Total Ion Chromatogram spectral data of PU7-1N. FIG. 19B shows Time of Flight spectral data of PU7-1N. FIG. 19C shows LC-MS total wave chromatogram spectral data of PU7-1N.

FIG. 20 shows MS data of SFB-FOXM1 complexes.

FIG. 21 shows peptide counts of deubiquitinases identified by proteomics analysis in A549 cells treated with or without PU7-1 for 72 h.

FIG. 22 shows RNAseq data, p<0.01.

### DETAILED DESCRIPTION OF THE INVENTION

In certain aspects, the subject matter disclosed herein is directed to a compound:

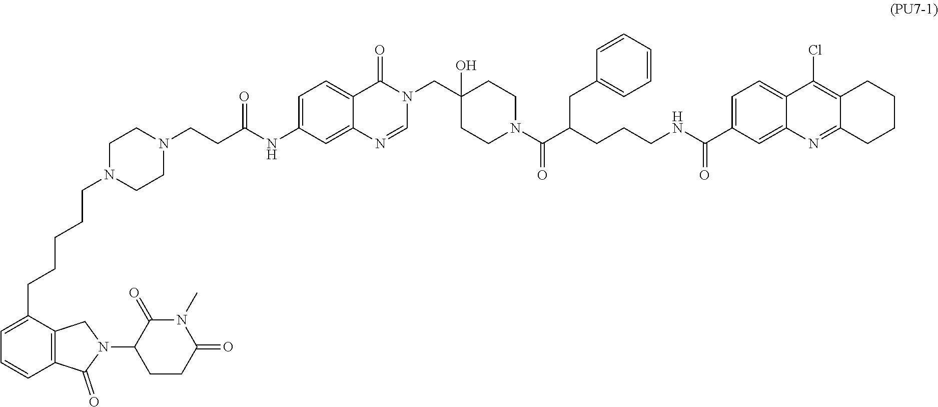 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]

or a pharmaceutically acceptable salt thereof.

In certain aspects, the subject matter disclosed herein is directed to a compound:

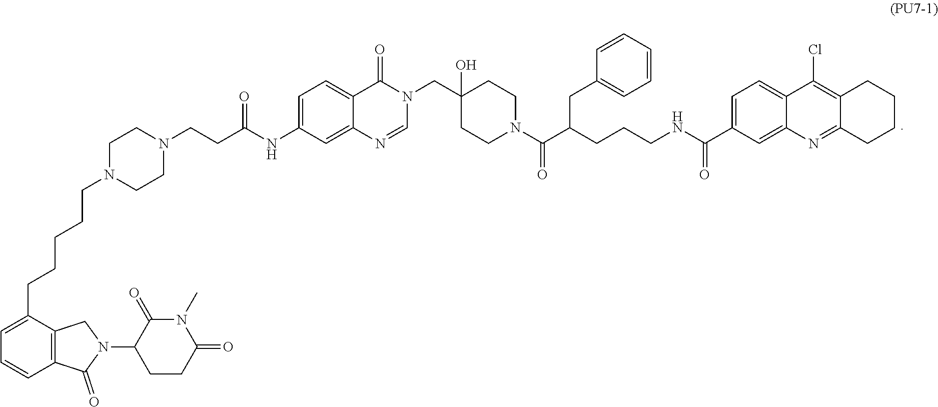 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]

In some embodiments, the subject matter disclosed herein is directed to a pharmaceutical composition comprising a pharmaceutically acceptable excipient and the compound PU7-1, or a pharmaceutically acceptable salt thereof. In some embodiments, the subject matter disclosed herein is directed to a pharmaceutical composition consisting essentially of a pharmaceutically acceptable excipient and the compound PU7-1, or a pharmaceutically acceptable salt thereof. In some embodiments, the subject matter disclosed herein is directed to a pharmaceutical composition consisting of a pharmaceutically acceptable excipient and the compound PU7-1, or a pharmaceutically acceptable salt thereof.

In some embodiments, the subject matter disclosed herein is directed to a pharmaceutical composition comprising a pharmaceutically acceptable excipient and the compound PU7-1. In some embodiments, the subject matter disclosed herein is directed to a pharmaceutical composition consisting essentially of a pharmaceutically acceptable excipient and the compound PU7-1. In some embodiments, the subject matter disclosed herein is directed to a pharmaceutical composition consisting of a pharmaceutically acceptable excipient and the compound PU7-1.

In certain aspects, the subject matter disclosed herein is directed to a method of treating cancer in a subject in need thereof comprising administering to the subject compound:

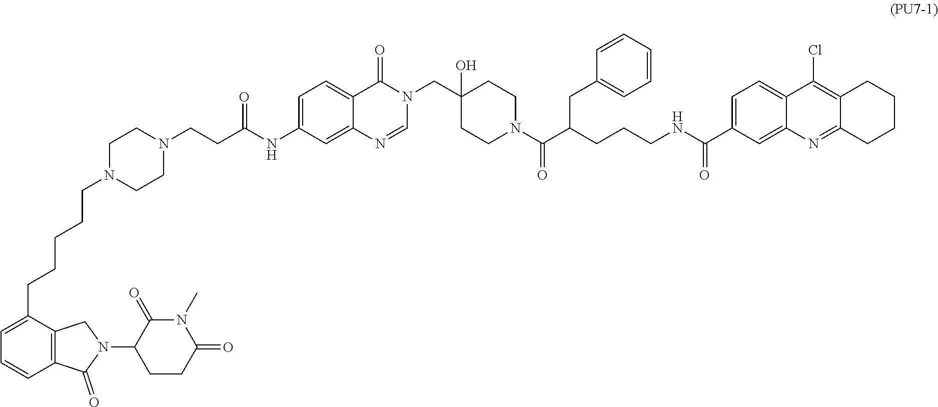 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]

or a pharmaceutically acceptable salt thereof. In some embodiments, the subject is administered a therapeutically effective amount of PU7-1 or a pharmaceutically acceptable salt thereof. In some embodiments, the subject matter disclosed herein is directed to a method of treating cancer in a subject in need thereof comprising administering to the subject a compound:

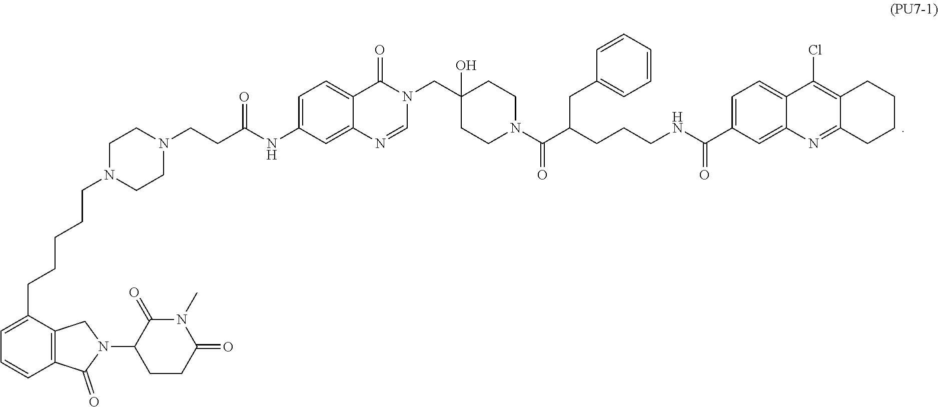 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]

In some embodiments, the subject is administered a therapeutically effective amount of PU7-1.

In some embodiments, the compound (PU7-1) or pharmaceutically acceptable salt thereof is administered as a pharmaceutical composition comprising a pharmaceutically acceptable excipient and the compound PU7-1, or pharmaceutically acceptable salt thereof. In some embodiments, the compound (PU7-1) is administered as a pharmaceutical composition comprising a pharmaceutically acceptable excipient and the compound PU7-1. In some embodiments, the cancer is a p53-independent cancer. In some embodiments, the cancer is triple-negative breast cancer. In some embodiments, the subject is a human.

### Triple-Negative Breast Cancer (TNBC)

According to the American Cancer Society, triple-negative breast cancer (TNBC) accounts for about 10-15% of all breast cancers. This type of cancer is called triple negative because the cancer cells do not express estrogen (ER) or progesterone (PR) receptors and also do not produce HER2 protein. Thus, these cancer cells are negative for the three markers. Unlike other types of breast cancer, TNBC generally grows and spreads rapidly, responds to fewer treatments, and is associated with a poor prognosis. Once a patient has been diagnosed with breast cancer using imaging techniques or a biopsy, cancer cells are tested for the expression of ER, PR, and HER2 in order for TNBC to be diagnosed. The most common treatment options for TNBC include, but are not limited to, chemotherapy, radiation, immunotherapy, surgery, or any combinations thereof.

P53 is a tumor suppressor protein that functions through binding DNA and ultimately regulates cell division and cell death. p53 is frequently mutated in most triple-negative breast cancers (TNBC), which, as mentioned above, is characterized as the very aggressive form of breast cancers with limited treatment options and poor patient outcomes. In the context of cancer, p53 negatively regulates expression of the oncogenic transcription factor Forkhead Box M1 (FOXM1). p53 loss-of-function can lead to uncontrolled division of cells and tumor development.

In some embodiments, the subject matter described herein relates to the finding that the oncoprotein Forkhead Box M1 (FOXM1) acts as a potential driver for tumor growth in TNBC. Additionally, surprisingly, through a proteomic screen, USP7 was identified as a major regulator of FOXM1 in TNBC cells. USP7 can interact with FOXM1 both in vitro and in vivo. USP7 can stabilize FOXM1 through deubiquitination. Conversely, RNAi-mediated USP7 knockdown in TNBC cells, can dramatically reduce the levels of FOXM1. Moreover, based upon the proteolysis targeting chimera (PROTAC) technology, the compound PU7-1 was generated as a USP7 specific degrader.

PU7-1 can induce rapid USP7 degradation at low nanomolar concentrations in cells but shows no obvious effect on other USP family proteins. Strikingly, the treatment of TNBC cells with PU7-1 significantly abrogates FOXM1 functions and effectively suppresses cell growth in vitro. By using xenograft mouse models, it is shown herein that PU7-1 can markedly repress tumor growth in vivo. Notably, ectopic overexpression of FOXM1 can reverse the tumor growth suppressive effects induced by PU7-1. In some embodiments, described herein is the finding that FOXM1 is a major target of USP7 in modulating tumor growth in a p53-independent manner. In some embodiments, described herein is the use of PU7-1 as a potential therapeutic tool for the treatment of triple-negative breast cancers (e.g., breast cancer that is ER-negative, PR-negative and HER2-negative).

### Methods of Treating Cancer

In certain aspects, the subject matter disclosed herein provides a method of treating or preventing cancer in a subject in need thereof, the method comprising administering to the subject PU7-1 or a pharmaceutically acceptable salt thereof. In some embodiments, the subject is administered a therapeutically effective amount of PU7-1, or a pharmaceutically acceptable salt thereof. In some embodiments, the method comprises administering to the subject PU7-1. In some embodiments, the subject is administered a therapeutically effective amount of PU7-1. In some embodiments, the compound (PU7-1), or pharmaceutically acceptable salt thereof, is administered as a pharmaceutical composition as described herein.

In some embodiments, the treatment results in improved survival of the subject. In some embodiments, the treatment results in improved overall survival, disease-free survival, objective response rate, time to progression, progression-free survival, time-to-treatment failure, time to progression of cancer symptoms, or a combination thereof. In certain embodiments, the improvement is measured as compared to one or more conventional treatment regimens.

In some embodiments, the cancer is a p53-independent cancer. In some embodiments, the cancer is breast cancer, TNBC, kidney cancer, brain tumor, head and neck cancer, liver cancer, melanoma, non-small cell lung cancer (NSCLC), bladder cancer, cervical cancer, colon cancer, renal cell cancer, skin cancer, stomach cancer, rectal cancer, gastrointestinal, pancreatic cancer, lung cancer, thymic carcinoma, ovarian cancer, prostate cancer, glioma, or endometrial cancer. In some embodiments, the cancer is a liquid cancer, such as leukemia, lymphoma, Hodgkin lymphoma, or myeloma. In some embodiments, the cancer is breast cancer. In some embodiments, the cancer is TNBC.

In some embodiments, the subject matter disclosed herein relates to a preventive medical treatment started after following diagnosis of a disease (e.g., cancer, TNBC) in order to prevent the disease from worsening or curing the disease. In one embodiment, the subject matter disclosed herein relates to prophylaxis of subjects who are believed to be at risk for moderate or severe disease associated with cancer or TNBC or have previously been diagnosed with another disease, such as cancer or TNBC.

The compound disclosed here (i.e., PU7-1), a pharmaceutically acceptable salt thereof, or the pharmaceutical compositions comprising PU7-1 or a pharmaceutically acceptable salt thereof may be administered to a cell, mammal, or human by any suitable means. In some embodiments, the compound or composition is administered to a human. As will be readily apparent to one skilled in the art, the effective in vivo dose to be administered and the particular mode of administration will vary depending upon the age, weight, and species treated, and the specific use for which the compound or pharmaceutically acceptable salt thereof are employed. The determination of effective dose levels, that is the dose levels necessary to achieve the desired result, can be accomplished by one skilled in the art using routine pharmacological methods. Typically, human clinical applications of products are commenced at lower dose levels, with dose level being increased until the desired effect is achieved. Alternatively, acceptable in vitro studies can be used to establish useful doses and routes of administration of the compositions identified by the present methods using established pharmacological methods. Effective animal doses from in vivo studies can be converted to appropriate human doses using conversion methods known in the art.

In certain aspects, the subject matter disclosed herein provides a method of inhibiting the growth of p53-independent cancer or inhibiting the growth of triple negative breast cancer in a subject in need thereof, the method comprising administering to the subject PU7-1, or a pharmaceutically acceptable salt thereof. In certain aspects, the subject matter disclosed herein provides a method of inhibiting the growth of p53-independent cancer or inhibiting the growth of triple negative breast cancer in a subject in need thereof, the method comprising administering to the subject PU7-1. In some embodiments, the compound (PU7-1), or pharmaceutically acceptable salt thereof, is administered as a pharmaceutical composition as described herein.

In some embodiments, the method of treating or prevent cancer in a subject in need thereof further comprises administering a PARP inhibitor. In some embodiments, the PARP inhibitor is Olaparib. In some embodiments, the PARP inhibitor is Talazoparib. In some embodiments Olaparib or Talazoparib are administered according to their FDA label, the content of each of which is hereby incorporated by reference in their entireties (www.accessdata.fda.gov/drugsatfda_docs/label/2023/208558s025lbl.pdf; www.accessdata.fda.gov/drugsatfda_docs/label/2023/211651s010lbl.pdf). Said PARP inhibitor can be administered before, simultaneously, or after PU7-1. In some embodiments, a pharmaceutical composition comprising a PARP inhibitor and PU7-1 is administered. In some embodiments, the PARP inhibitor and PU7-1 are administered as separate pharmaceutical compositions. In some embodiments, administration of PU7-1 enhances sensitivity to PARP inhibitors.

In some embodiments, the method of treating or prevent cancer in a subject in need thereof further comprises administering an anti-PD-1/PD-L1 antibody. In some embodiments, the anti-PD-1/PD-L1 antibody is pembrolizumab, nivolumab, cemiplimab, atezolizumab, duravalumab, retifanlimab, toripalimab, dostarlimab, or avelumab. In some embodiments, the anti-PD-1/PD-L1 antibody is pembrolizumab. In some embodiments pembrolizumab is administered according to its FDA label, the content of which is hereby incorporated by reference in its entirety (www.accessdata.fda.gov/drugsatfda_docs/label/2021/125514s096lbl.pdf). Said anti-PD-1/PD-L1 antibody can be administered before, simultaneously, or after PU7-1. In some embodiments, a pharmaceutical composition comprising anti-PD-1/PD-L1 antibody and PU7-1 is administered. In some embodiments, the anti-PD-1/PD-L1 antibody and PU7-1 are administered as separate pharmaceutical compositions. In some embodiments, administration of PU7-1 enhances sensitivity to an anti-PD-1/PD-L1 antibody.

In some embodiments, in the methods disclosed herein the subject is a human subject.

### Pharmaceutical Compositions

In some embodiments, described herein are pharmaceutical compositions comprising PU7-1, or a pharmaceutically acceptable salt thereof. In some embodiments, the pharmaceutical composition comprises a pharmaceutically acceptable excipient and PU7-1, or a pharmaceutically acceptable salt thereof. In some embodiments, the pharmaceutical composition consists essentially of a pharmaceutically acceptable excipient and PU7-1, or a pharmaceutically acceptable salt thereof. In some embodiments, the pharmaceutical composition consists of a pharmaceutically acceptable excipient and the compound PU7-1, or a pharmaceutically acceptable salt thereof. In some embodiments, the pharmaceutical composition comprises PU7-1.

In some embodiments, described herein are pharmaceutical compositions comprising a therapeutically effective amount of PU7-1, or a pharmaceutically acceptable salt thereof. In some embodiment, said compositions are for treating cancer, such as TNBC. In some embodiments, the compositions comprise, consist essentially of, or consist of PU7-1, or a pharmaceutically acceptable salt thereof and a pharmaceutically acceptable excipient. A “therapeutically effective amount,” or “effective amount,” or “therapeutically effective,” as used herein, refers to that amount which provides a therapeutic effect for a given condition and administration regimen. A therapeutically effective amount can be determined by a skilled person based on patient characteristics, such as age, weight, sex, condition, complications, other diseases, etc., as is well known in the art.

A pharmaceutically acceptable excipient can comprise any and all carriers, diluents, fillers, solvents, dispersion media, coatings, antibacterial and antifungal agents, isotonic and absorption delaying agents, glidants, lubricants, colorants, disintegrants, and the like, compatible with pharmaceutical administration. The use of such media and agents for pharmaceutically active substances is well known in the art. Any conventional media or agent that is compatible with the active compound can be used. Supplementary active compounds can also be incorporated into the compositions.

A pharmaceutical composition as described herein can be formulated to be compatible with its intended route of administration.

In some embodiments, the pharmaceutical compositions described herein can be administered as solid compositions, for example, for oral administration. In some embodiments, the solid compositions comprise excipients including but not limited to lactose, starch, cellulose, milk sugar or high molecular weight polyethylene glycols. In some embodiments, the pharmaceutical compositions described herein can be administered as aqueous suspensions and/or elixirs. In some embodiments, the pharmaceutical compositions described herein may be combined with various sweetening or flavouring agents, coloring matter or dyes, with emulsifying and/or suspending agents and with diluents such as water, ethanol, propylene glycol and glycerin, and combinations thereof.

In some embodiments, the pharmaceutical compositions described herein can be administered parenterally, for example, intravenously, intra-arterially, intraperitoneally, intra-thecally, intraventricularly, intrasternally, intracranially, intra-muscularly or subcutaneously, or they may be administered by infusion techniques. In some embodiments, the pharmaceutical compositions described herein can be administered in the form of a sterile aqueous solution which may contain other substances, for example, enough salts or glucose to make the solution isotonic with blood. The preparation of suitable parenteral formulations under sterile conditions is readily accomplished by standard pharmaceutical techniques well-known to those skilled in the art.

In some embodiments, pharmaceutical compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The pharmaceutical compositions can be presented in unit-dose or multi-dose containers. The pharmaceutical compositions can be sealed ampoules or vials. The pharmaceutical compositions can be stored in a freeze-dried (lyophilised) condition requiring only the addition of the sterile liquid carrier, such as water for injections, immediately prior to use.

The pharmaceutical compositions of the present invention can be employed in combination therapies, that is, the compounds and pharmaceutical compositions can be administered concurrently with, prior to, or subsequent to, one or more other desired therapeutics or medical procedures (e.g., PU7-1 can be used in combination treatment with another cancer treatment e.g., PARP inhibitors such as Olaparib, Talazoparib). The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, PU7-1 may be administered concurrently with another therapeutic or prophylactic for TNBC).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### Pharmaceutical Compositions for Injection

In some embodiments, the subject matter described herein provides a pharmaceutical composition for administration via injection. In some embodiments, the pharmaceutical composition for injection is for treating one or more cancers. In some embodiments, the cancer is TNBC. In some embodiments, the pharmaceutical composition comprises PU7-1 or a pharmaceutically acceptable salt thereof. In some embodiments, the pharmaceutical composition further comprises a pharmaceutically acceptable excipient. In some embodiments, the pharmaceutical composition comprises a therapeutically effective amount of PU7-1 or a pharmaceutically acceptable salt thereof. In some embodiments, the pharmaceutical composition comprises PU7-1.

Pharmaceutical compositions for injection can be formulated with any pharmaceutical excipient known in the field that is suitable for injection. The formulations include but are not limited to an aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. In some embodiments, aqueous solutions in saline are conventionally used for injection. Ethanol, glycerol, propylene glycol and liquid polyethylene glycol (and suitable mixtures thereof), and vegetable oils can be employed with the compositions described herein. Various antibacterial and antifungal agents known in the art can be added to the compositions described herein to prevent the action of micro-organisms, for example, parabens, chlorobutanol, phenol, sorbic acid and thimerosal. Sterile powders for the preparation of sterile injectable solutions can be prepared by vacuum-drying and freeze-drying techniques known in the art which yield a powder of the active ingredient PU7-1 or a pharmaceutically effective salt thereof.

### Pharmaceutical Compositions for Oral Administration

In some embodiments, the subject matter described herein provides a pharmaceutical composition for oral administration. In some embodiments, the oral pharmaceutical composition is for treating one or more cancers. In some embodiments, the cancer is TNBC. In some embodiments, the pharmaceutical composition comprises PU7-1 or a pharmaceutically acceptable salt thereof. In some embodiments, the pharmaceutical composition further comprises a pharmaceutically acceptable excipient. In some embodiments, the pharmaceutical composition comprises a therapeutically effective amount of PU7-1 or a pharmaceutically acceptable salt thereof. In some embodiments, the pharmaceutical composition comprises PU7-1.

Pharmaceutical compositions of the disclosure suitable for oral administration can be presented as discrete dosage forms, such as capsules, gelatin capsules, cachets, or tablets, or liquids or aerosol sprays each containing a predetermined amount of an active ingredient as a powder or in granules, a solution, a syrup, or a suspension in an aqueous or non-aqueous liquid, an oil-in-water emulsion, water-in-oil liquid emulsion, emulsions, or suspensions of microspheres or nanospheres or of lipid or polymeric vesicles for controlled release.

In some embodiments, the disclosure further encompasses anhydrous pharmaceutical compositions and dosage forms. For example, water may be added (e.g., 5%) in the pharmaceutical arts as a means of facilitating long-term storage. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. Anhydrous compositions may be packaged using materials known to prevent exposure to water. Examples of suitable packaging include, but are not limited to, hermetically scaled foils, plastic packages, unit dose containers, blister packs, and strip packs.

The pharmaceutical composition comprising PU7-1 or a pharmaceutically acceptable salt thereof can be combined in an admixture with a pharmaceutical carrier according to conventional pharmaceutical compounding techniques. The carrier can take a wide variety of forms depending on the form of preparation desired for administration. Carriers can be water, glycols, oils, alcohols, flavoring agents, preservatives, coloring agents, and the like in the case of oral liquid preparations (such as suspensions, solutions, and elixirs) or aerosols. Carriers can also be starches, sugars, micro-crystalline cellulose, diluents, granulating agents, lubricants, binders, and disintegrating agents can be used in the case of oral solid preparations.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof. Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. A sufficient amount of disintegrant that is neither too little nor too much to detrimentally alter the release of the PU7-1 or pharmaceutically acceptable salt thereof may be used to form the dosage forms of the compounds disclosed herein. The amount of disintegrant used may vary based upon the type of formulation and mode of administration, and may be readily discernible to those of ordinary skill in the art. Disintegrants that can be used include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof. Lubricants include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof.

Tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

In an embodiment, the composition may include a solubilizer to ensure good solubilization and/or dissolution of the compound of the present disclosure and to minimize precipitation of the compound of the present disclosure. This can be especially important for compositions for non-oral use, such as for compositions for injection. A solubilizer may also be added to increase the solubility of the hydrophilic drug and/or other components, such as surfactants, or to maintain the composition as a stable or homogeneous solution or dispersion. Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, epsilon-caprolactone and isomers thereof, 8-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Examples may include, but are not limited to, sodium, potassium, lithium, magnesium, calcium and ammonium.

### Dosages of PU7-1

The amount of PU7-1 or a pharmaceutically acceptable salt thereof administered will be dependent on the subject being treated, the severity of the disorder or condition, the rate of administration, the formulation of PU7-1, and the discretion of the prescribing physician. However, an effective dosage can be in the range of about 0.001 mg to about 100 mg per kg body weight per day, such as about 1 to about 35 mg/kg/day, in single or divided doses. For a 70 kg human, this would amount to about 0.05 g/day to 7 g/day, such as about 0.05 to about 2.5 g/day. In some instances, dosage levels below the lower limit of the aforesaid range may be more than adequate. In other instances doses larger than the upper limit may be administered without causing any harmful side effect—e.g., by dividing such larger doses into several small doses for administration throughout the day.

In some embodiments, PU7-1 or a pharmaceutically acceptable salt thereof is administered in once daily, twice daily, three times daily, or more often. In some embodiments, PU7-1 or a pharmaceutically acceptable salt thereof is administered once every other day, twice a week, once a week, or once a month. In some embodiments, administration of PU7-1 or a pharmaceutically acceptable salt thereof may continue as long as necessary to treat cancer. In some embodiments, administration of PU7-1 or a pharmaceutically acceptable salt thereof may continue as long as necessary to reduce the size of a tumor. In some embodiments, administration of PU7-1 or a pharmaceutically acceptable salt thereof may continue as long as necessary to reduce the growth rate of a tumor. In some embodiments, administration of PU7-1 or a pharmaceutically acceptable salt thereof continues for the duration of the subject's natural life. In some embodiments, the administration is oral. In some embodiments, the administration is via an injection. However, other routes may be used as appropriate. In some embodiments, PU7-1 or a pharmaceutically acceptable salt thereof is administered in one or more doses for treating or preventing cancer. In some embodiments the cancer is breast cancer. In some embodiments, the breast cancer is TNBC.

In some embodiments, PU7-1 or a pharmaceutically acceptable salt thereof is administered for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days. In some embodiments, PU7-1 or a pharmaceutically acceptable salt thereof is administered for less than 28, 14, 7, 6, 5, 4, 3, 2, or 1 day. In some embodiments, the days are consecutive days. In some embodiments, the days are not consecutive days. In some embodiments, the non-consecutive days are equally spread-out (i.e., there is the same amount of time between each day of administration). In some embodiments, the non-consecutive days are not equally spread-out (i.e., there is a varying amount of time between each day of administration).

In some embodiments, an effective dosage of PU7-1 or a pharmaceutically acceptable salt thereof is in the range of about 1 mg to about 1000 mg, about 10 mg to about 500 mg, about 20 mg to about 450 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 202 mg. In some embodiments, an effective dosage of PU7-1 or a pharmaceutically acceptable salt thereof is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 240 mg, about 250 mg, about 260 mg, about 270 mg, about 280 mg, about 290 mg, about 300 mg, about 310 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg about 550 mg, about 600 mg, about 650 mg, about 700 mg, about 750 mg, about 800 mg, about 850 mg, about 900 mg, about 950 mg, or 1000 mg. In some embodiments, an effective dosage of PU7-1 or a pharmaceutically acceptable salt thereof is about 1 g, about 1.5 g, about 2 g, about 2.5 g, or about 3 g.

In some embodiments, an effective dosage of PU7-1 or a pharmaceutically acceptable salt thereof is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg. In some embodiments, an effective dosage of PU7-1 or a pharmaceutically acceptable salt thereof is about 0.01 mg/kg, about 0.05 mg/kg, about 0.10 mg/kg, about 0.15 mg/kg, about 0.20 mg/kg, about 0.25 mg/kg, about 0.35 mg/kg, about 0.40 mg/kg, about 0.45 mg/kg, about 0.50 mg/kg, about 0.55 mg/kg, about 0.60 mg/kg, about 0.65, mg/kg about 0.70 mg/kg, about 0.75 mg/kg, about 0.80 mg/kg, about 0.85 mg/kg, about 0.90 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, or about 4 mg/kg.

In some embodiments, the pharmaceutical composition comprising PU7-1 or a pharmaceutically acceptable salt thereof is administered by injection at a dosage of about 0.001 mg/ml, about 0.005 mg/ml, about 0.01 mg/ml, about 0.02 mg/ml, about 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.1 mg/ml, about 1.2 mg/ml, about 1.3 mg/ml, about 1.4 mg/ml, about 1.5 mg/ml, about 1.6 mg/ml, about 1.7 mg/ml, about 1.8 mg/ml, about 1.9 mg/ml, about 2.0 mg/ml, about 2.1 mg/ml, about 2.2 mg/ml, about 2.3 mg/ml, about 2.4 mg/ml, about 2.5 mg/ml, about 2.6 mg/ml, about 2.7 mg/ml, about 2.8 mg/ml, about 2.9 mg/ml, about 3.0 mg/ml, about 3.1 mg/ml, about 3.2 mg/ml, about 3.3 mg/ml, about 3.4 mg/ml, about 3.5 mg/ml, about 3.6 mg/ml, about 3.7 mg/ml, about 3.8 mg/ml, about 3.9 mg/ml, about 4.0 mg/ml, about 4.1 mg/ml, about 4.2 mg/ml, about 4.3 mg/ml, about 4.4 mg/ml, about 4.5 mg/ml, about 4.6 mg/ml, about 4.7 mg/ml, about 4.8 mg/ml, about 4.9 mg/ml, about 5.0 mg/ml, about 5.1 mg/ml, about 5.2 mg/ml, about 5.3 mg/ml, about 5.4 mg/ml, about 5.5 mg/ml, about 5.6 mg/ml, about 5.7 mg/ml, about 5.8 mg/ml, about 5.9 mg/ml, about 6.0 mg/ml, about 6.1 mg/ml, about 6.2 mg/ml, about 6.3 mg/ml, about 6.4 mg/ml, about 6.5 mg/ml, about 6.6 mg/ml, about 6.7 mg/ml, about 6.8 mg/ml, about 6.9 mg/ml, about 7.0 mg/ml, about 7.1 mg/ml, about 7.2 mg/ml, about 7.3 mg/ml, about 7.4 mg/ml, about 7.5 mg/ml, about 7.6 mg/ml, about 7.7 mg/ml, about 7.8 mg/ml, about 7.9 mg/ml, about 8.0 mg/ml, about 8.1 mg/ml, about 8.2 mg/ml, about 8.3 mg/ml, about 8.4 mg/ml, about 8.5 mg/ml, about 8.6 mg/ml, about 8.7 mg/ml, about 8.8 mg/ml, about 8.9 mg/ml, about 9 mg/ml, about 9.1 mg/ml, about 9.2 mg/ml, about 9.3 mg/ml, about 9.4 mg/ml, about 9.5 mg/ml, about 9.6 mg/ml, about 9.7 mg/ml, about 9.8 mg/ml, about 9.9 mg/ml, about 10 mg/ml, about 10.5 mg/ml, about 11 mg/ml, about 11.5 mg/ml, about 12 mg/ml, about 12.5 mg/ml, about 13 mg/ml, about 13.5 mg/ml, about 14 mg/ml, about 14.5 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, or about 50 mg/ml.

In some embodiments, the pharmaceutical composition comprising PU7-1 or a pharmaceutically acceptable salt thereof is administered by injection at a volume of about 0.01 ml, about 0.02 ml, about 0.03 ml, about 0.04 ml, about 0.05 ml, about 0.06 ml, about 0.07 ml, about 0.08 ml, about 0.09 ml, about 0.1 ml, about 0.15 ml, about 0.2 ml, about 0.25 ml, about 0.30 ml, about 0.35 ml, about 0.40 ml, about 0.45 ml, about 0.5 ml, about 0.55 ml, about 0.60 ml, about 0.65 ml, about 0.70 ml, about 0.75 ml, about 0.80 ml, about 0.85 ml, about 0.90 ml, about 0.95 ml, about 1.0 ml, about 1.1 ml, about 1.2 ml, about 1.3 ml, about 1.4 ml, about 1.5 ml, about 1.6 ml, about 1.7 ml, about 1.8 ml, about 1.9 ml, about 2.0 ml, about 2.5 ml, about 3.0 ml, about 3.5 ml, about 4.0 ml, about 4.5 ml, about 5.0 ml, about 5.5 ml, about 6.0 ml, about 6.5 ml, about 7.0 ml, about 7.5 ml, about 8.0 ml, about 8.5 ml, about 9.0 ml, about 9.5 ml, about 10.0 ml, about 15.0 ml, about 20.0 ml, about 25.0 ml, about 30.0 ml, about 35.0 ml, about 40.0 ml, about 45.0 ml, or about 50.0 ml.

A therapeutically effective amount of PU7-1 or a pharmaceutically acceptable salt thereof may be administered in either single, double, triple, or multiple doses by any of the modes of administration known in the art of agents having similar utilities, including buccal, sublingual, and transdermal routes, by intra-arterial injection, intravenously, parenterally, intramuscularly, subcutaneously or orally.

### EXAMPLES

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

Those skilled in the art will recognize, or be able to ascertain, using no more than routine experimentation, numerous equivalents to the specific substances and procedures described herein. Such equivalents are considered to be within the scope of this invention, and are covered by the following claims.

All publications and other references mentioned herein are incorporated by reference in their entirety, as if each individual publication or reference were specifically and individually indicated to be incorporated by reference. Publications and references cited herein are not admitted to be prior art.

### Example 1—p53-Independent Tumor Growth Suppression in Triple-Negative Breast Cancers by Destabilizing FOXM1

Without being bound by theory, tumor suppressor p53 is a short-life protein and its protein levels are tightly controlled by MDM2-mediated polyubiquitination and Ubiquitin-specific-processing proteases (USPs)-mediated deubiquitination^{1, 2, 3, 4, 5, 6}. USP7, also known as Herpesvirus-associated ubiquitin-specific protease (HAUSP), was considered as a promising therapeutic target for cancer due to its critical regulation of several oncoproteins and tumor suppressors such as MDM2, p53, N-MYC, PTEN and DNMT1^{2, 3, 7, 8, 9, 10}. USP7 can have a dynamic role in the MDM2-p53 network^{2, 3}. On one hand, USP7 can stabilize p53 by deubiquitination; on the other hand, MDM2, a critical E3 ligase of p53, is also a target of USP7. Structure-based study revealed that MDM2 and p53 competitively recognize the same surface groove in USP7, whereas USP7 has higher binding affinity for MDM2 than p5311. Therefore, ablation of USP7 can predominantly lead to MDM2 degradation and ultimately p53 activation. These observations indicated that USP7 inhibition is potentially beneficial for therapeutic purposes in human cancers harboring wild-type p53. p53 inactivation was unable to completely rescue the embryonic lethality in USP7 knockout mice^{12, 13}, implicating the existence of p53-independent substrates of USP7. Without being bound by theory, USP7 can regulate tumor growth through both p53-dependent and p53-independent networks and inhibition of USP7 can lead to significant tumor suppression in many types of human cancer^{2, 3, 7, 8, 9, 10}. In the past decades, intensive efforts result in the development of several small-molecule inhibitors of USP7^{3, 14, 15}. However, due to issues in selectivity, potency, solubility, and metabolism properties in vivo, none of the current USP7 inhibitors have entered clinical trials.

FOXM1 belongs to the forkhead box transcription factor family, in which members share an evolutionary conserved fork-head or winged-helix DNA-binding domain¹⁶. FOXM1 is a master regulator of cell cycle¹⁶. FOXM1 can mediate the transcription of SKP2 and CKS1, which are key subunits of the SKP1-Cullin1-F-Box (SCF) ubiquitin ligase complex that targets the cyclin-dependent kinase inhibitors p21 and p27 for degradation during G1-S transition¹⁷. In addition, FOXM1 is essential for the transcription of the mitotic regulatory genes CCNB1/2, Aurora A/B, CDC25A/B, centromere proteins CENPA/B/F, PLK1 etc., modulating G2-M transition^{17, 18}. Unlike other FOX family proteins, FOXM1 is be predominantly expressed in proliferating cells and overexpression of FOXM1 is a hallmark of various human cancers^{16, 19}. High expression of FOXM1 is associated with a poor clinical prognosis of many malignancies, suggesting that it has an important role in cancer progression. Numerous studies demonstrate that FOXM1 plays a critical role in tumorigenesis, angiogenesis, invasion, and metastasis, as well as drug resistance^{16, 19, 20, 21, 22, 23, 24}.

Breast cancer is the second leading cause of cancer death in women. The American Cancer Society estimates that about 43700 women will die from breast cancer in the United States in 2023. Triple-negative breast cancer (TNBC) represents the most invasive subtype of breast cancers that are characterized by the lack of expression of the estrogen receptor (ER), progesterone receptor (PR) and absence of epidermal growth factor receptor 2 (ERBB2, also known as HER2) amplification²⁵. TNBC accounts for about 10%-15% of all breast cancers and has a high prevalence in women of African descent and women who carry the mutation of BRCA1/2 gene²⁶. It differs from other subtypes of breast cancer in more aggressive and metastatic behavior, fewer treatment options and worse prognosis than other subtypes of breast cancer. Despite the advances of the estrogen therapy and HER2-targeted therapy for breast cancer, chemotherapy has long been the only available therapeutic option for TNBC due to the lack of therapeutic targets²⁷. Given the limited benefit of chemotherapy, persistent efforts were focused on the development of new therapies for TNBC. Recently, PARP inhibitors have been successfully used in the triple-negative breast cancer with BRCA1/2 mutation 28. Although 69% of breast cancers with BRCA1/2 mutations are TNBCs²⁶, the prevalence rates of BRCA1/2 mutation in triple-negative breast cancer is about 20%^{29, 30, 31}. Therefore, the majority of TNBC patients still do not benefit from PARP inhibitors. The molecular heterogeneity of the disease makes the optimal treatment strategy for patients with TNBC still a major unmet need.

In some embodiments, the subject matter described herein relates to the finding that the oncogenic transcription factor FOXM1 is highly expressed in TNBC cell lines and tumors. Mass spectrometry analysis of FOXM1 protein complex identified USP7 as a novel interaction protein of FOXM1. USP7 can directly bind, deubiquitinate and stabilize the FOXM1 protein. Moreover, overexpression of USP7 in breast cancers is associated with poor prognosis and drug resistance^{32, 33, 34}. It suggests that targeting USP7 to downregulate FOXM1 can be an effective strategy for TNBC treatment. Therefore, a set of PROtcolysis Targeting Chimeras (PROTAC) compounds to target USP7 for degradation were synthesized and screened. PU7-1 was identified as a selective USP7 degrader. PU7-1 can significantly decrease FOXM1 protein levels and lead to proliferation inhibition in TNBC cells with the IC50 of 1.8 μM in MDA-MB-468 and 2.8 μM in BT549. Notably, PU7-1 can also markedly repress the growth of MDA-MB-468 xenografts in an in vivo mouse model. Exogenous expression of FOXM1 rescued PU7-1-mediated growth inhibition in MDA-MB-468 cells and xenografts, indicating that targeting USP7 by PU7-1 in TNBC elicits tumor suppression by antagonizing FOXM1 network.

### FOXM1 is Highly Expressed in TNBC

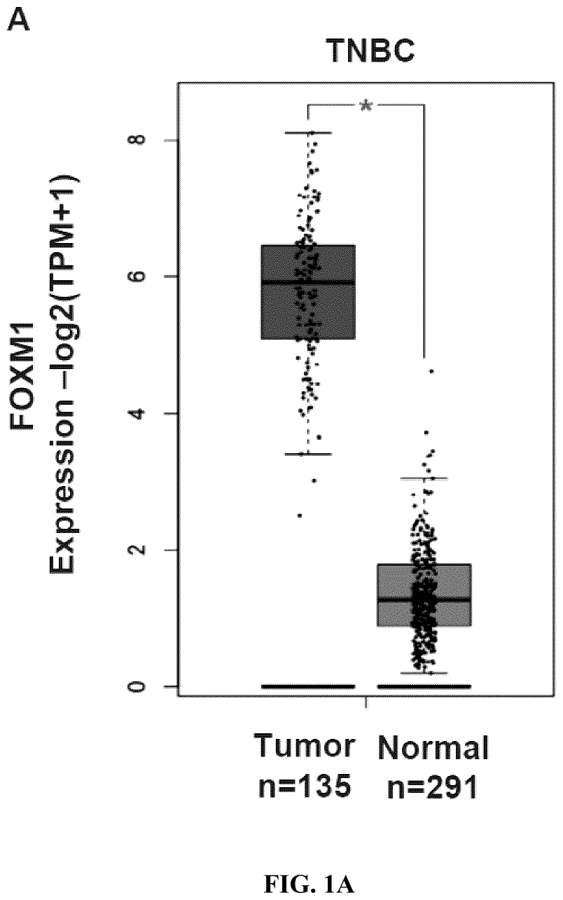

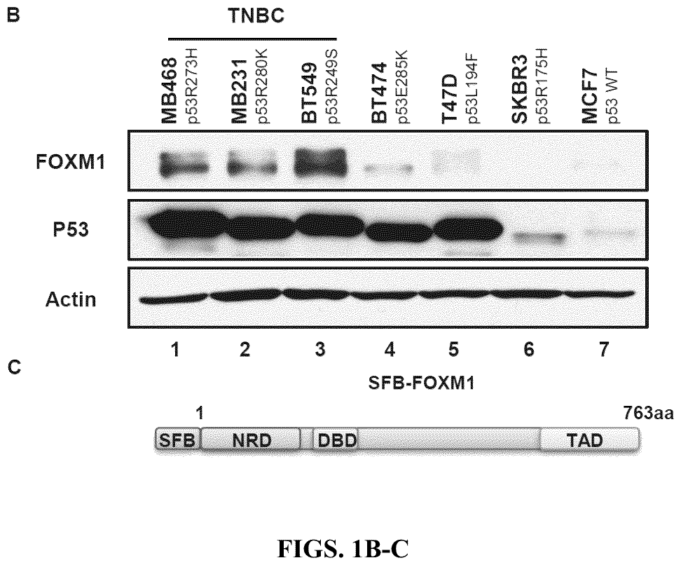

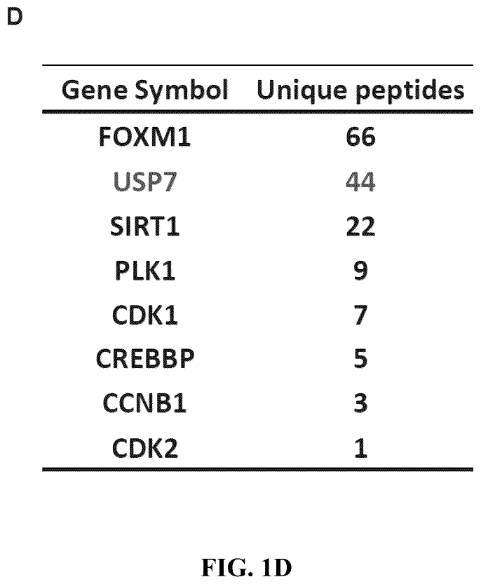

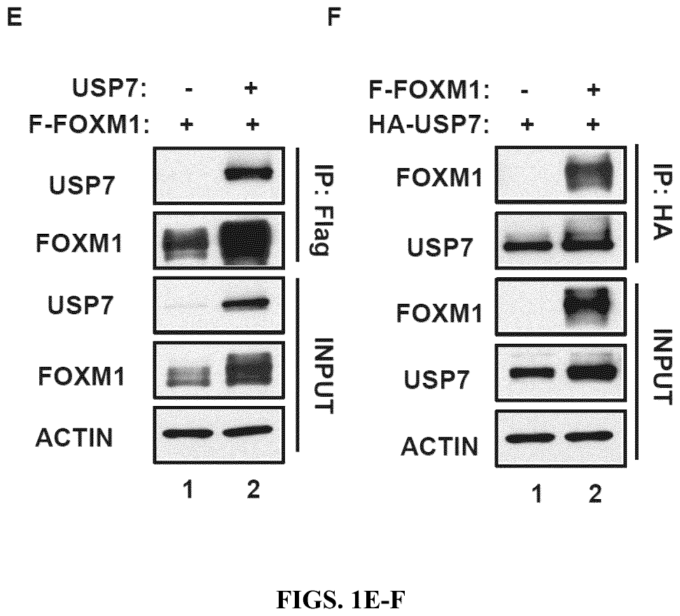

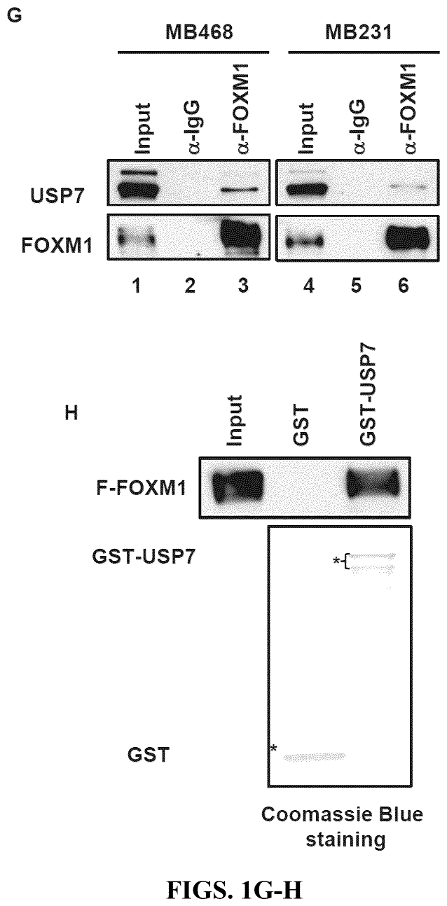

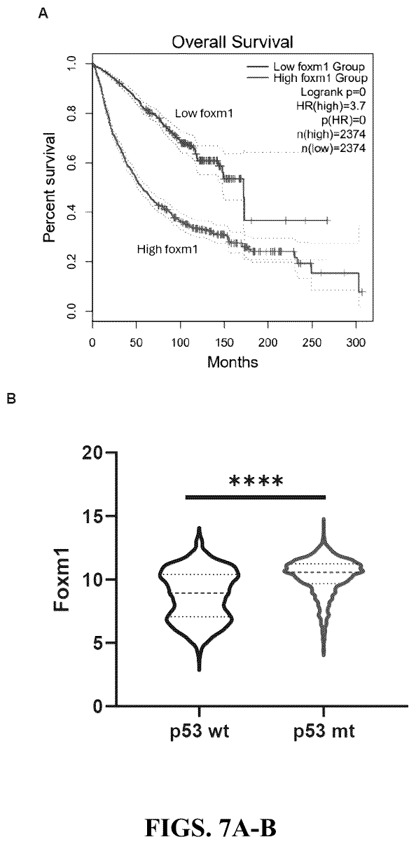

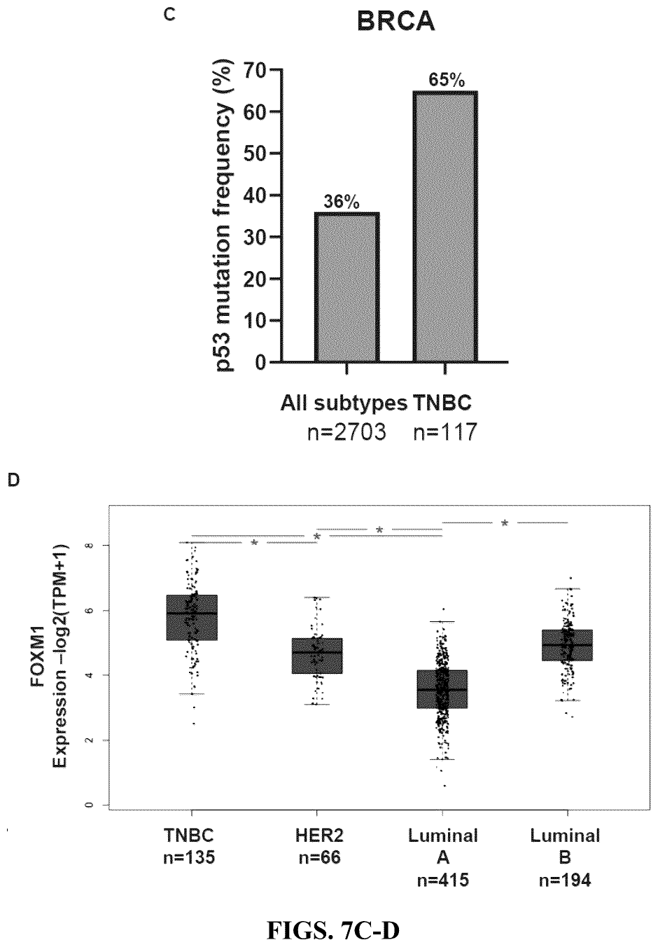

Overall survival analysis in The Cancer Genome Atlas (TCGA) pan-cancers revealed that higher expression of oncogenic transcription factor FOXM1 positively correlates with the shorter survival time of patients (FIG. 7A). Previous studies reported that FOXM1 is negatively regulated by wild-type p53^{35, 36}. Indeed, the TCGA cancer dataset was downloaded and the expression of FOXM1 was compared between the wild-type and mutant p53 cohort. A significant elevated FOXM1 expression was observed in cancers harboring mutant p53 (FIG. 7B). As the most aggressive subtype of breast cancer, TNBCs harbor mutant TP53 gene at much higher frequency than the other subtypes. According to the Catalogue Of Somatic Mutations In Cancer (COSMIC) database, 65% of TNBCs express mutant p53, in contrast, only 36% of all breast cancers have mutant p53 (FIG. 7C). Therefore, without being bound by theory, TNBC is likely to express high levels of FOXM1. The FOXM1 expression profiles in TNBC versus normal tissue and TNBC versus other subtypes of BRCA were explored. As shown in FIG. 1A, the expression of FOXM1 in TNBC is about 3-fold higher than in normal tissue. Moreover, TNBC exhibits the highest FOXM1 expression levels in all four BRCA subtypes (FIG. 7D). To further validate it, several breast cancer cell lines were randomly screened and FOXM1 protein levels were measured in these cells by Western blot analysis. Three of them (MDA-MB-468, MDA-MB-231 and BT549 harboring p53 R273H, R280K and R249S respectively) are triple-negative breast cancer cells. As shown in FIG. 1B, the expression levels of FOXM1 are indeed higher in the breast cancer cell lines with p53 mutants than the one in the MCF7 cell line expressing wild type p53. Interestingly, the levels of FOXM1 are especially higher in three TNBC cell lines (lanes 1-3) than other breast cell lines with mutated p53 (lanes 4-6), suggesting that additional undefined mechanisms may be involved in upregulation of FOXM1 in TNBC cells. Taken together, these results demonstrate that oncogenic transcription factor FOXM1 is highly expressed in TNBC cells, and it is a potential therapeutic target for TNBC.

FOXM1 can Directly Interact with USP7 Deubiquitinase

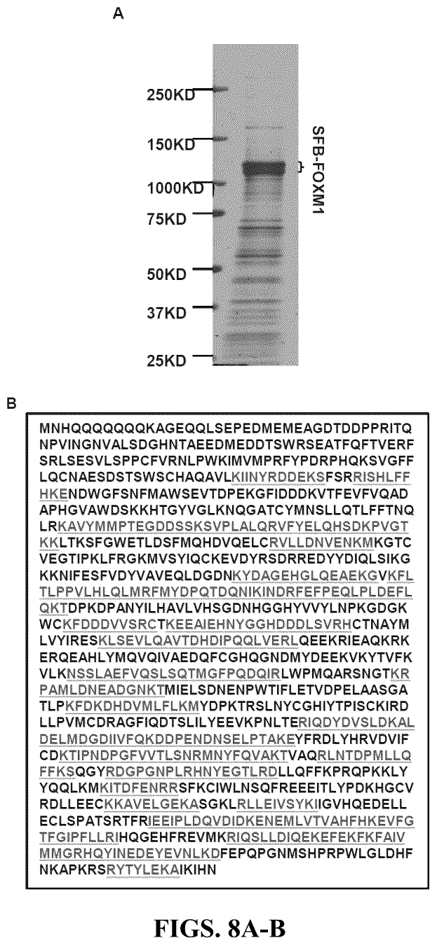

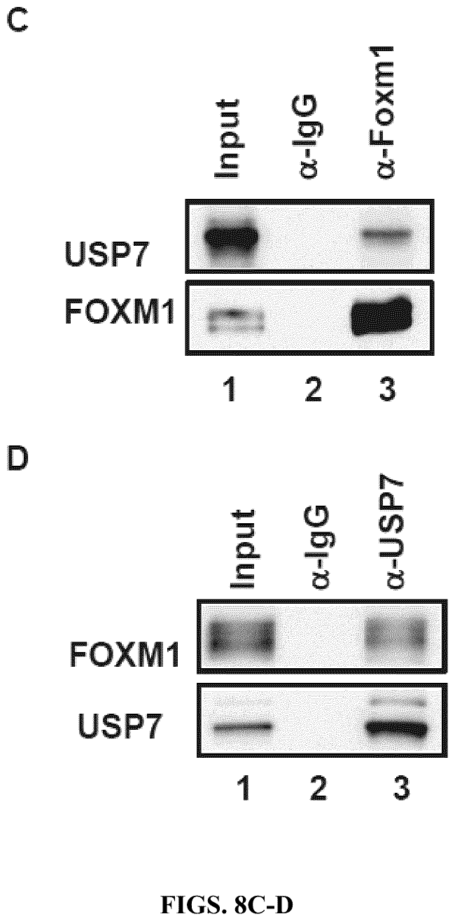

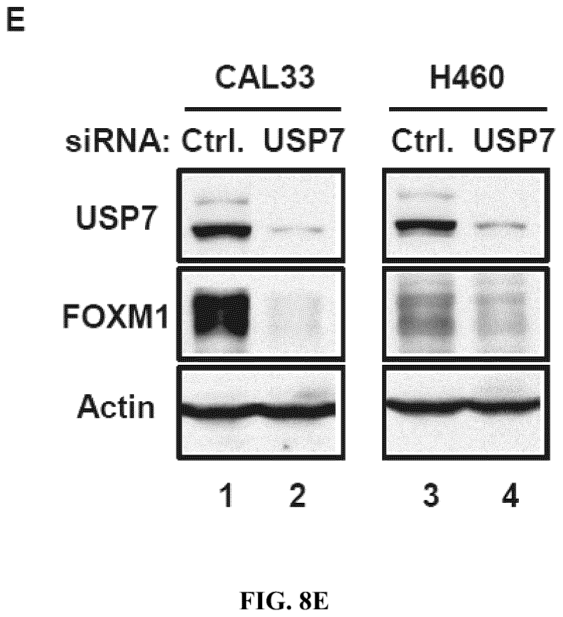

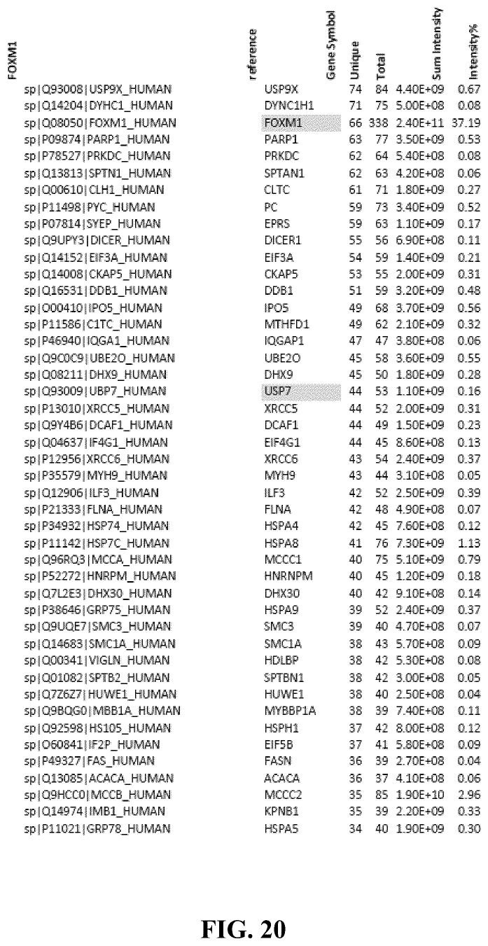

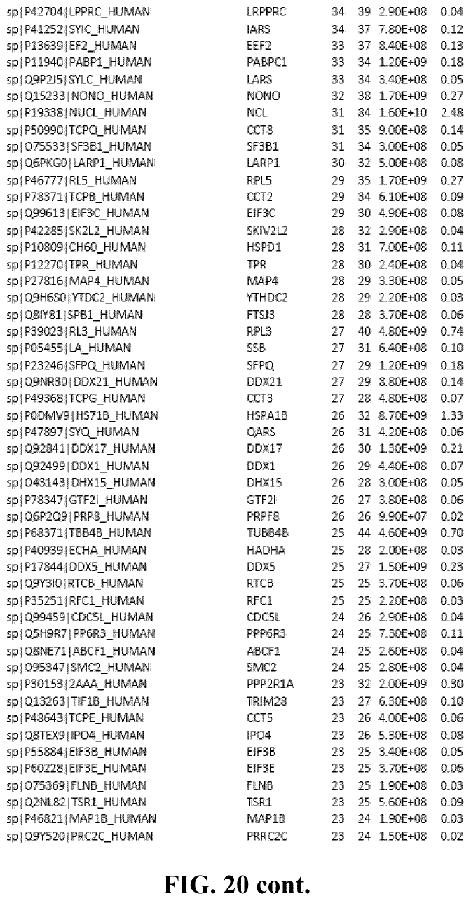

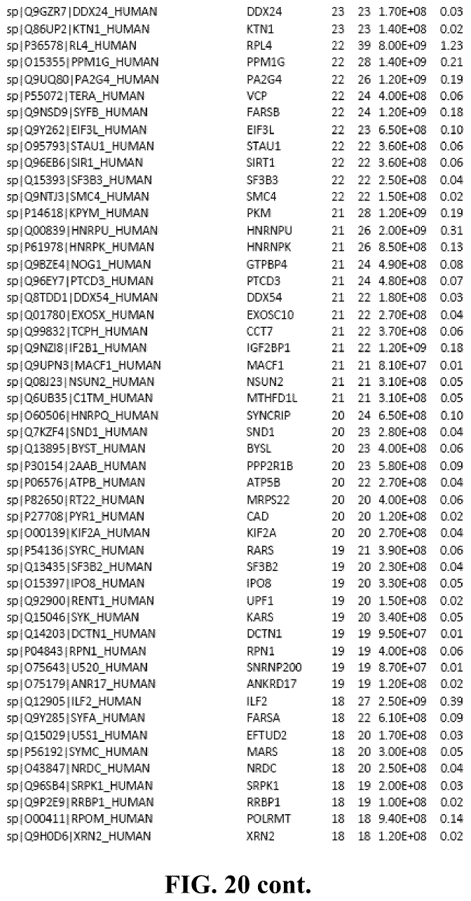

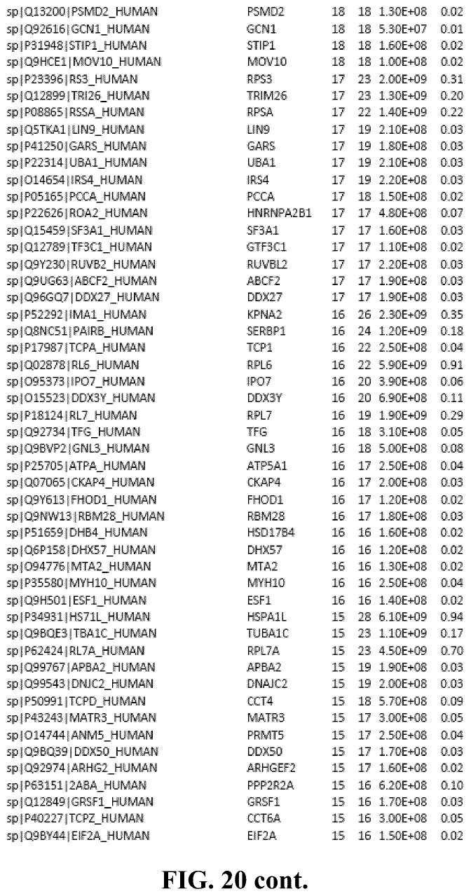

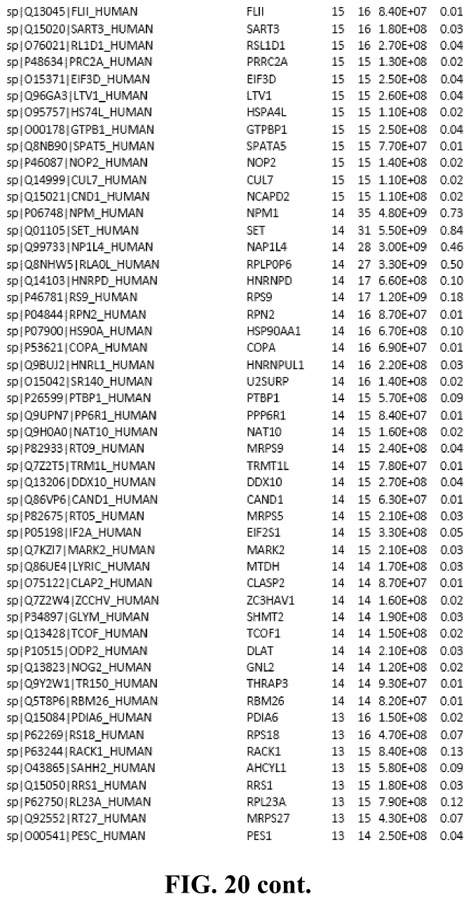

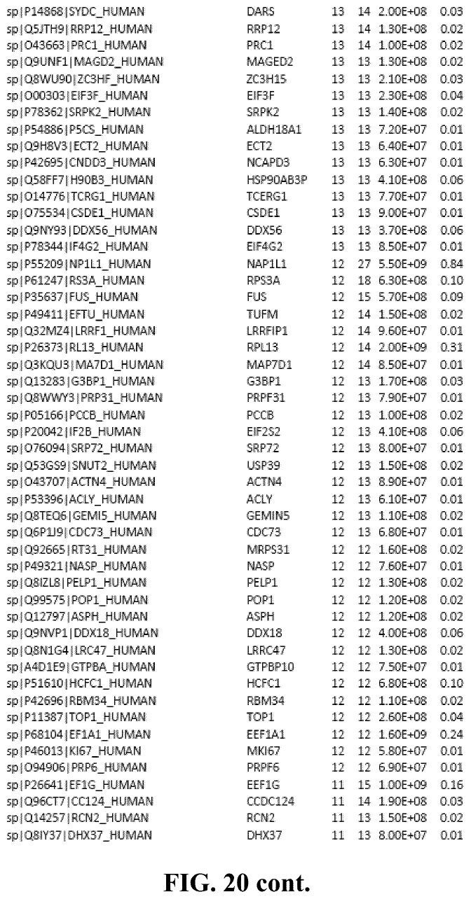

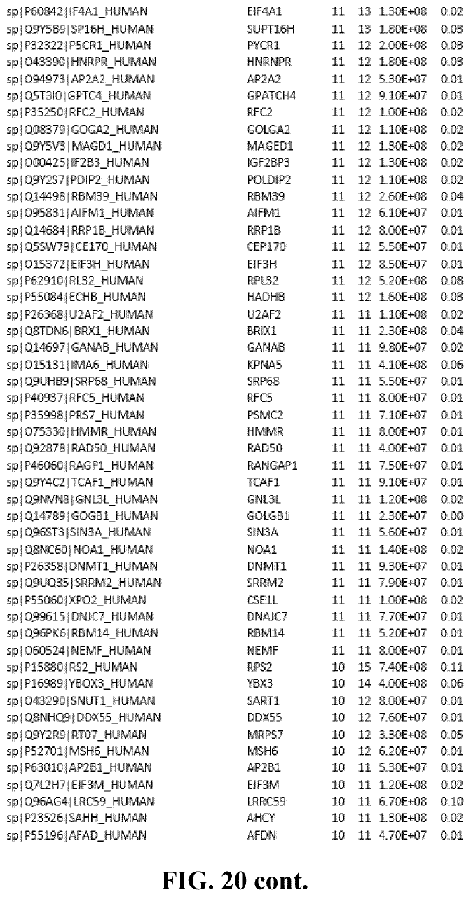

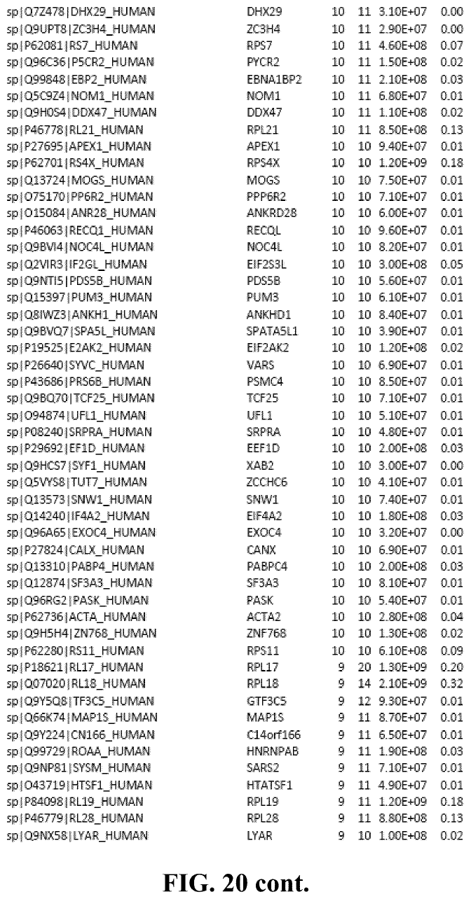

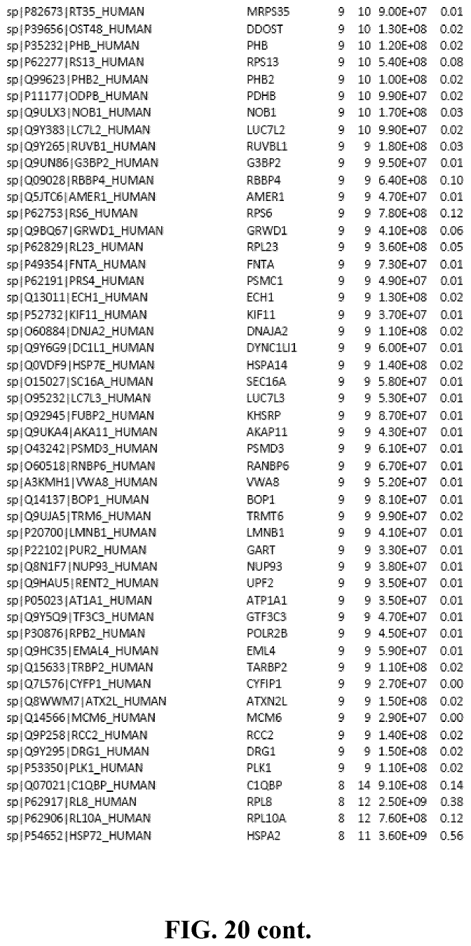

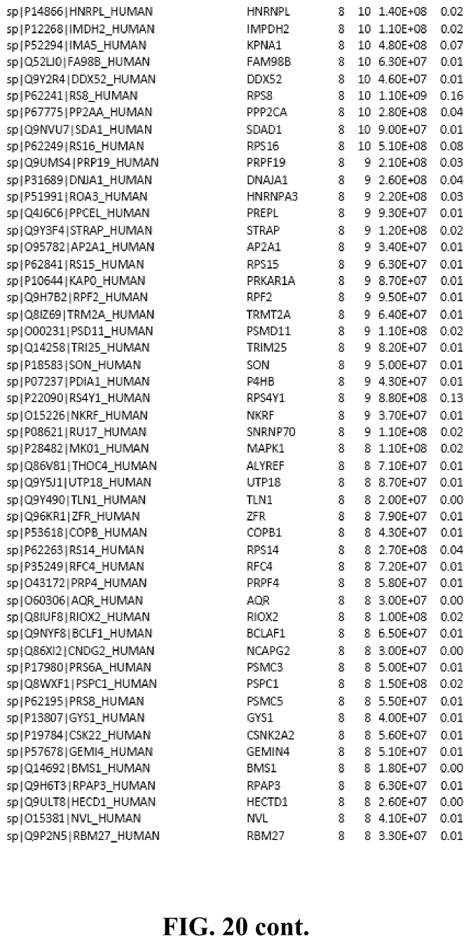

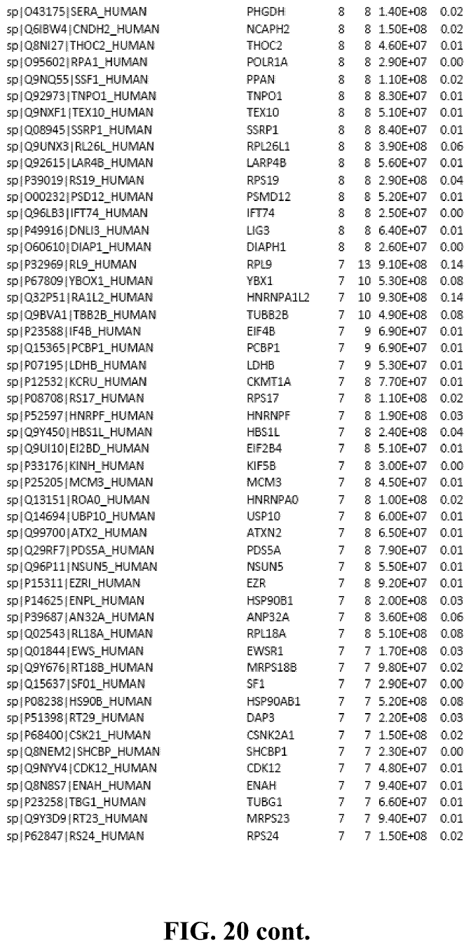

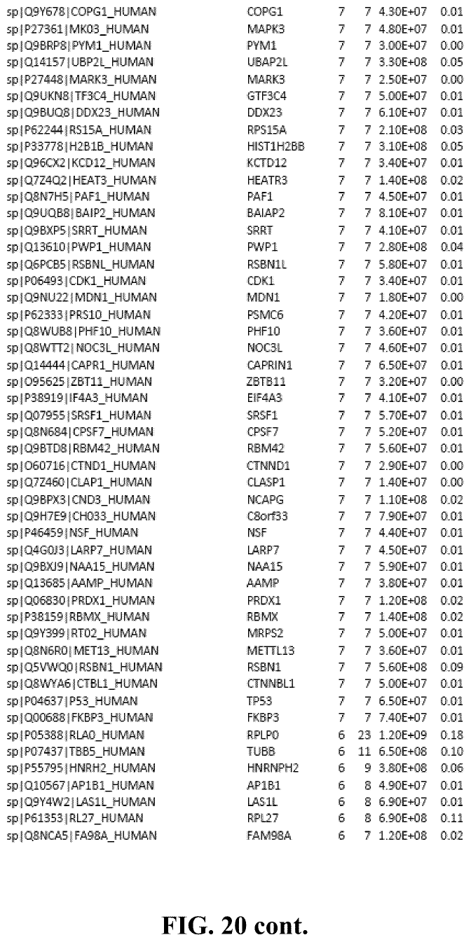

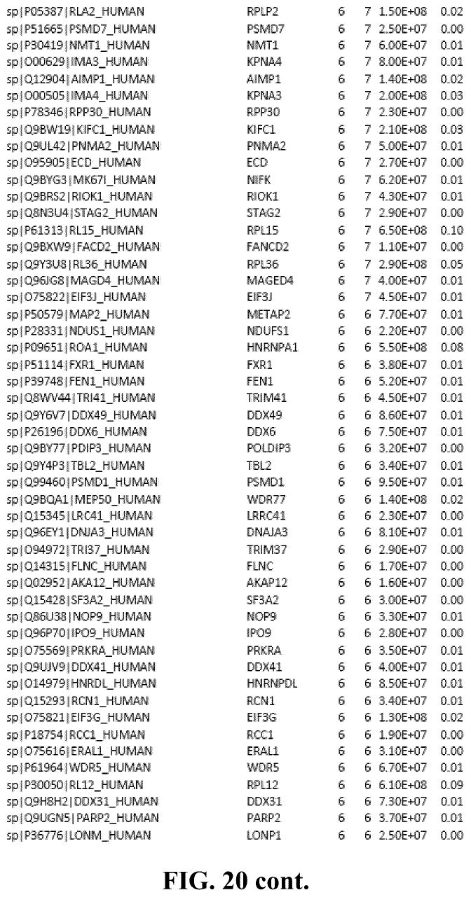

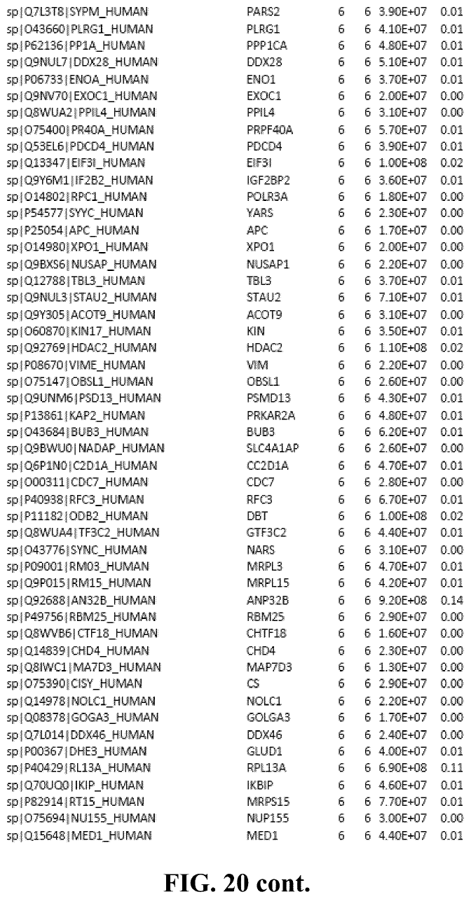

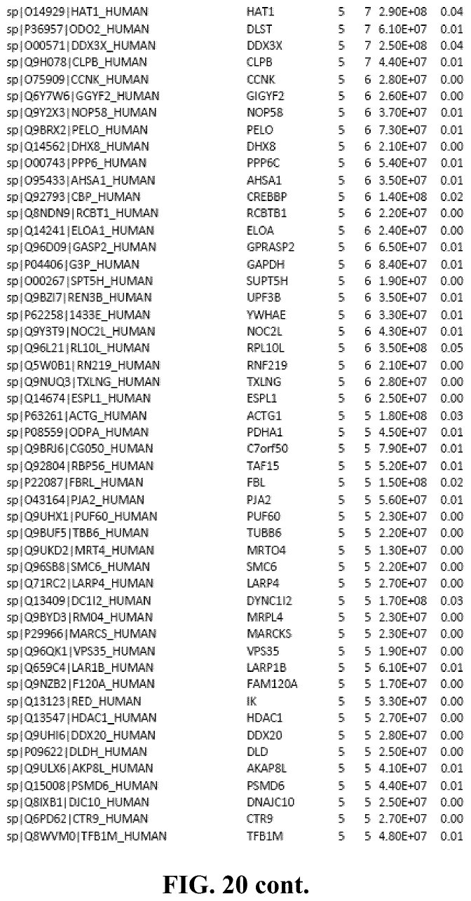

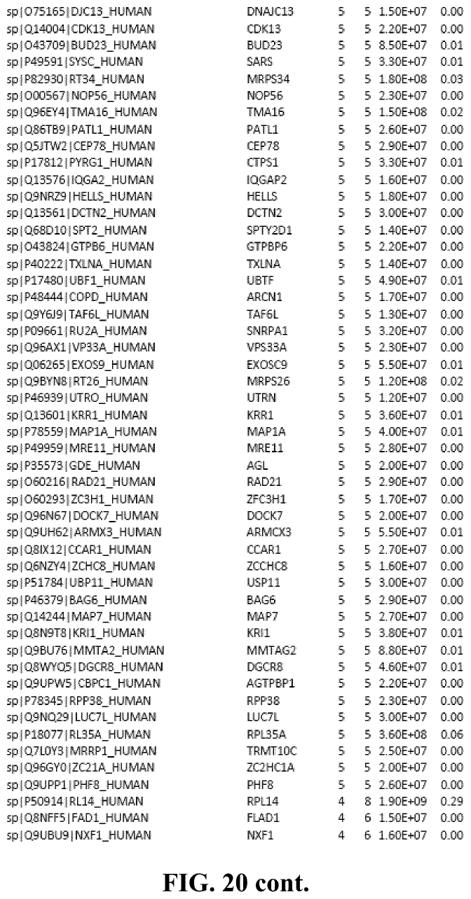

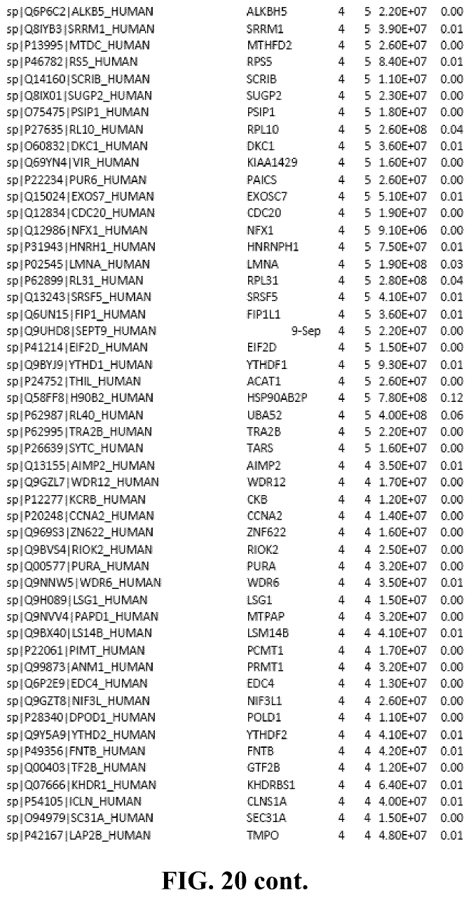

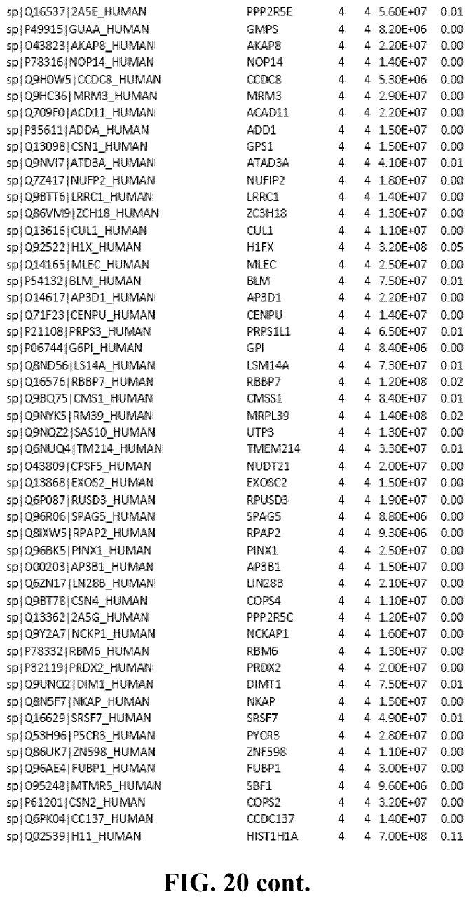

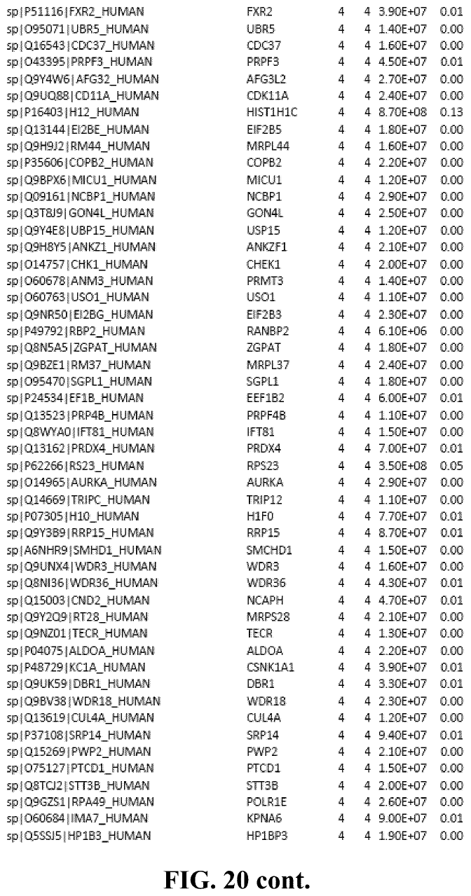

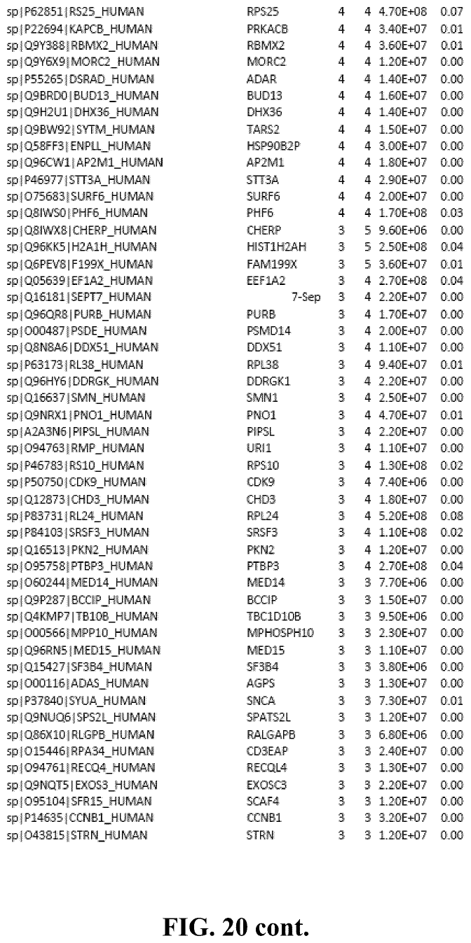

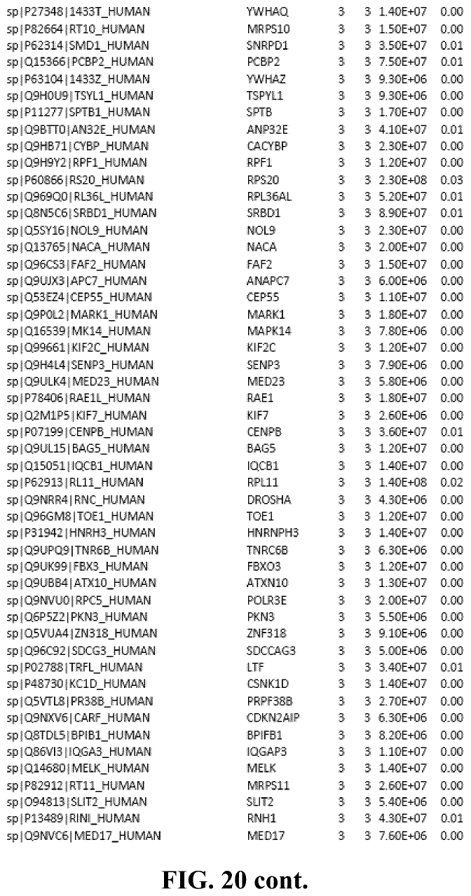

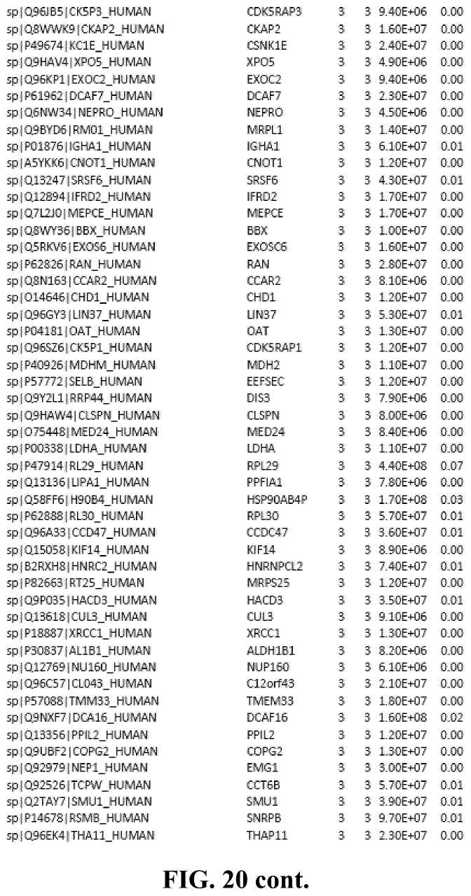

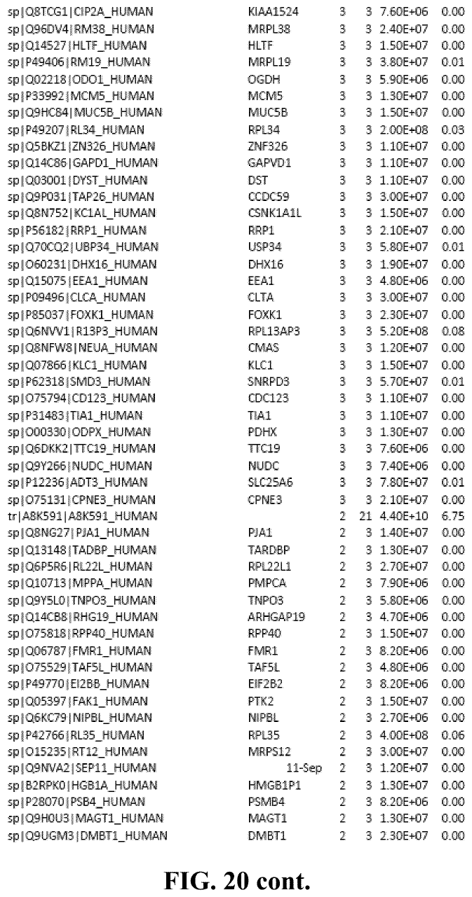

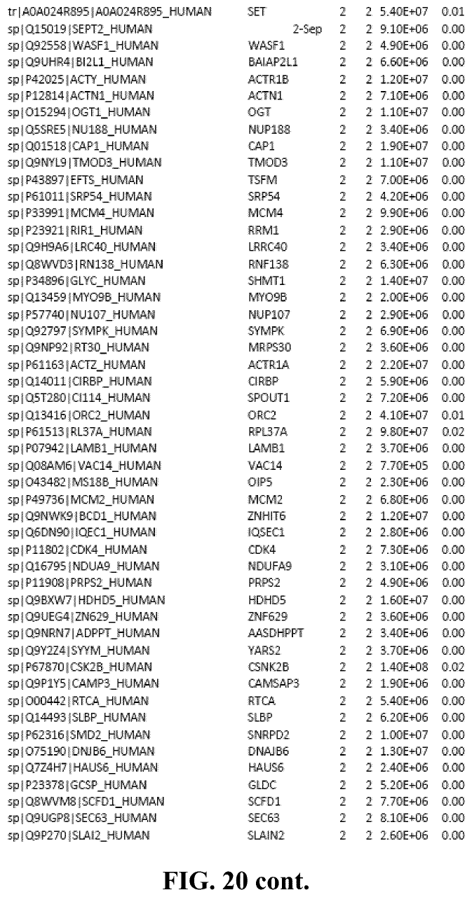

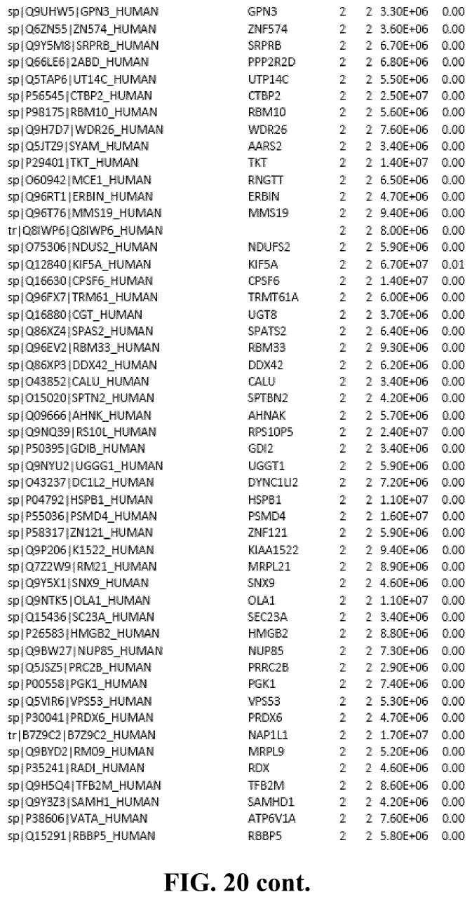

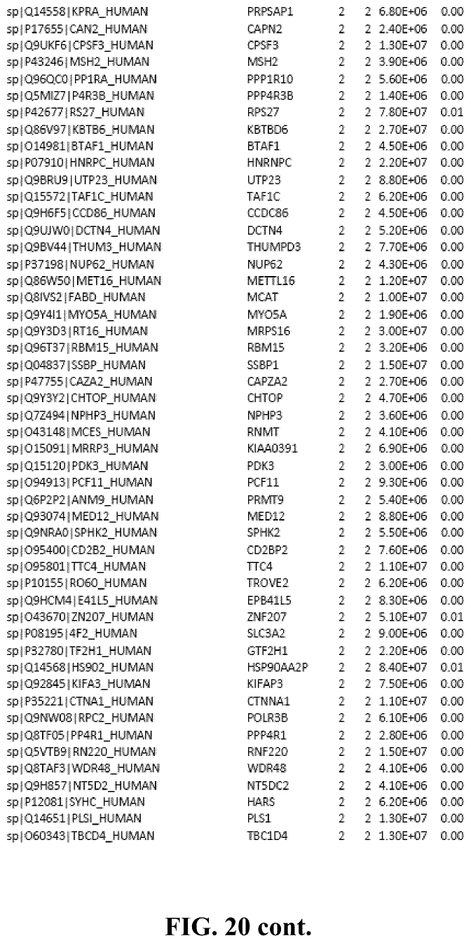

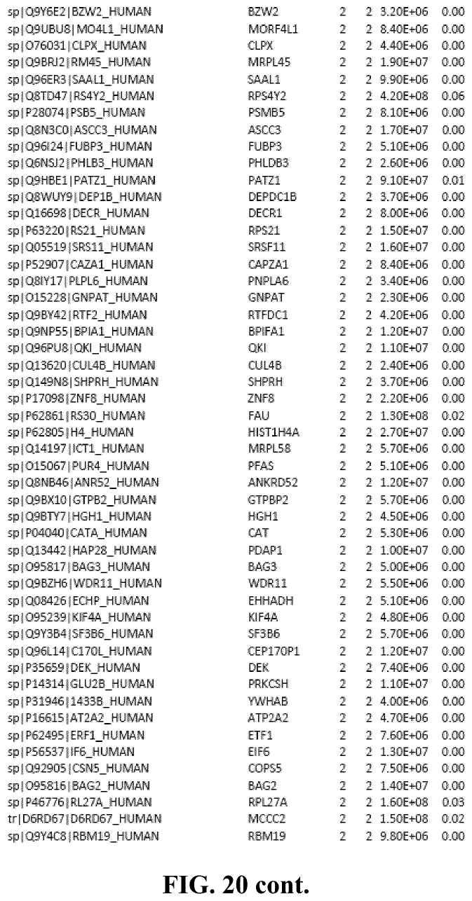

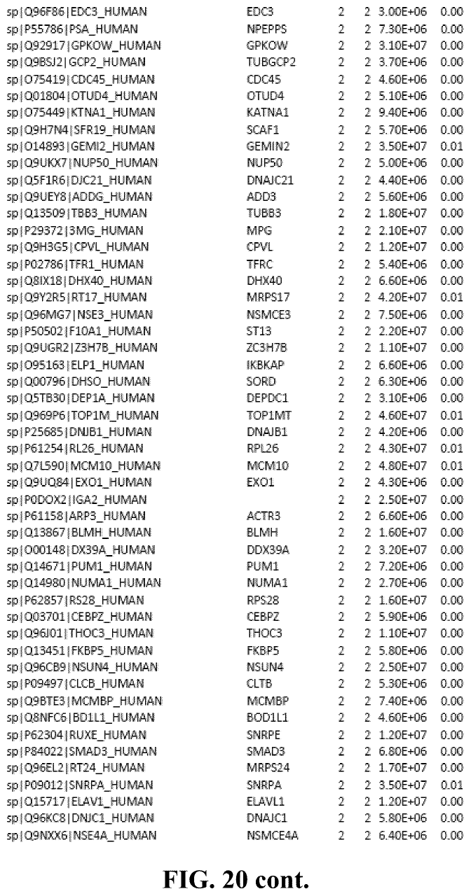

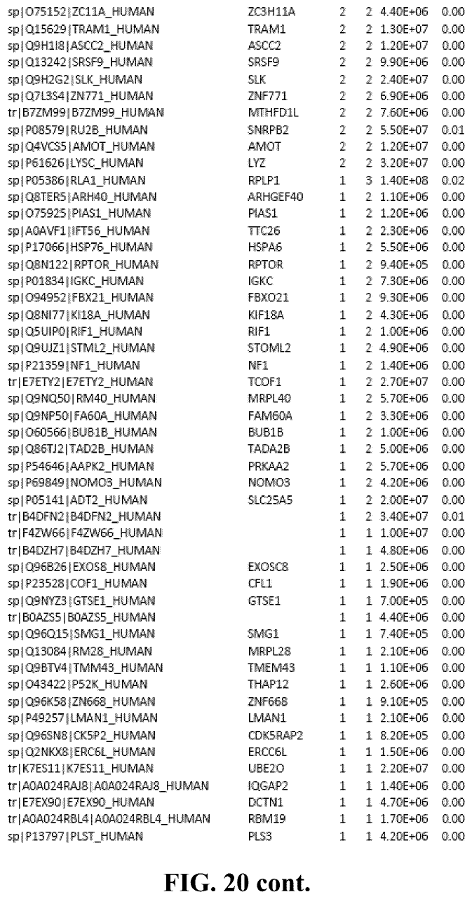

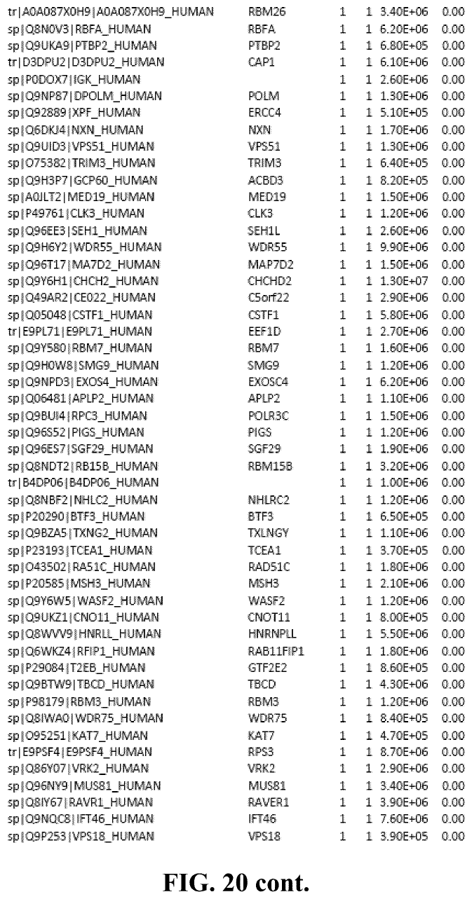

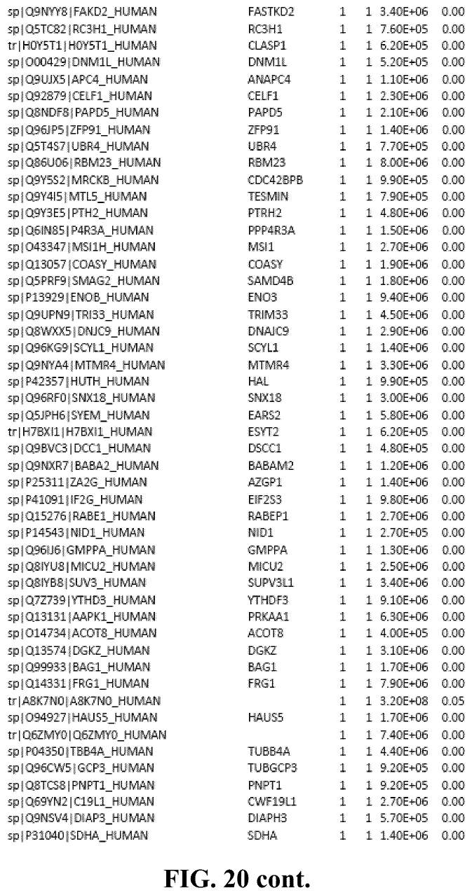

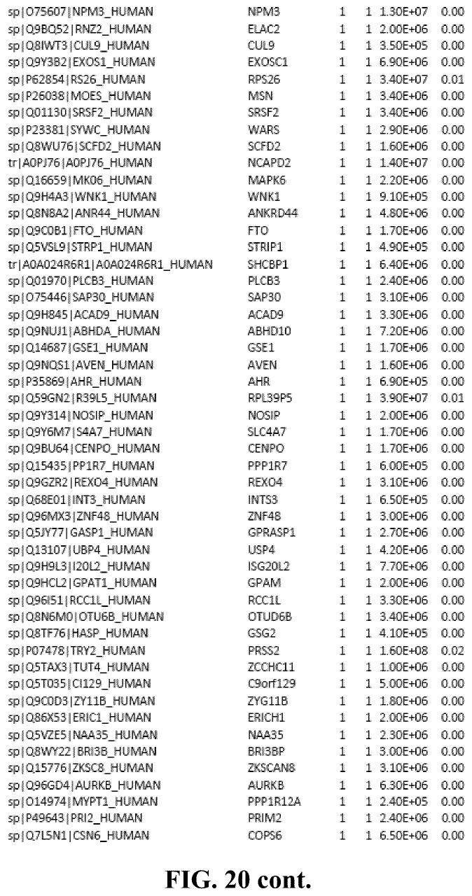

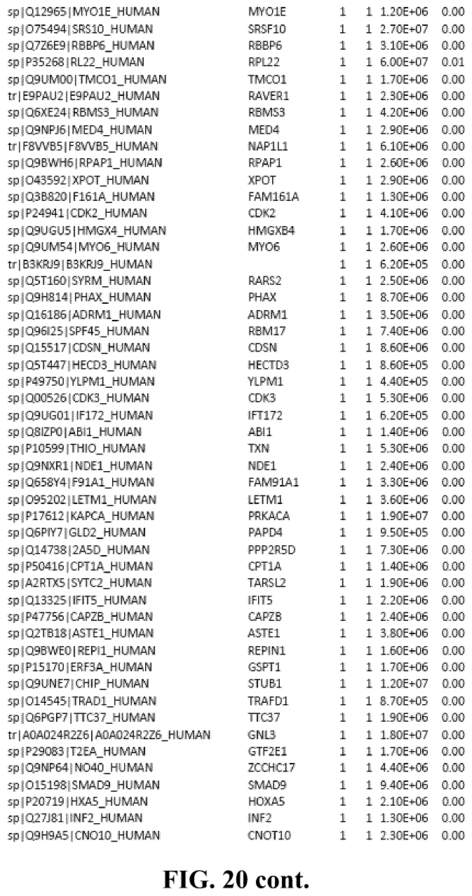

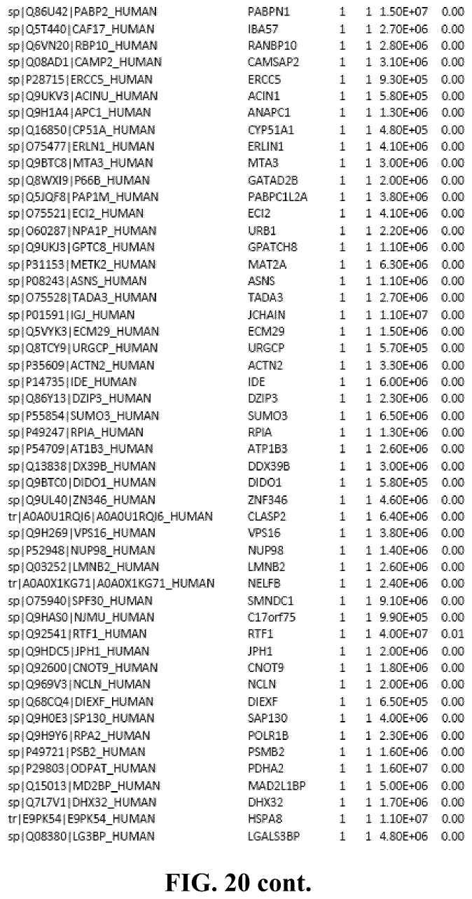

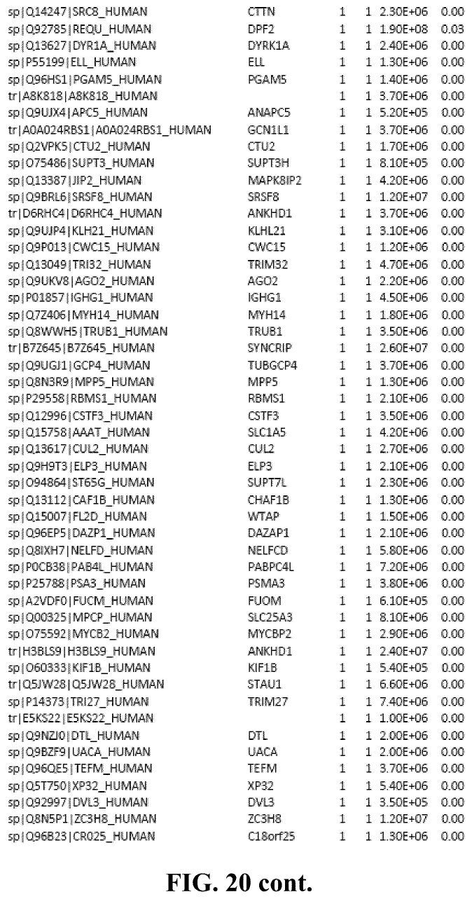

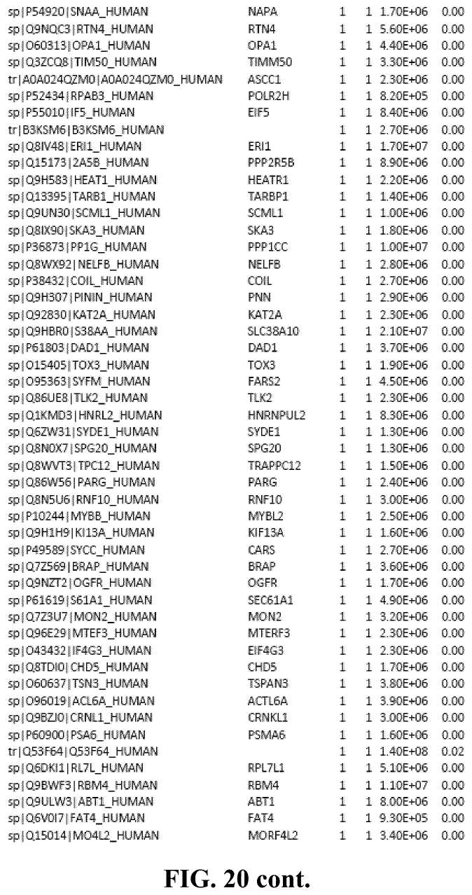

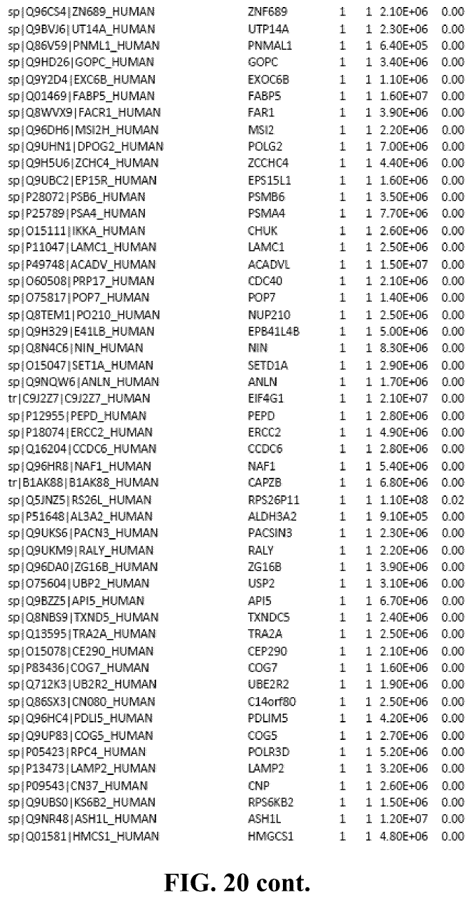

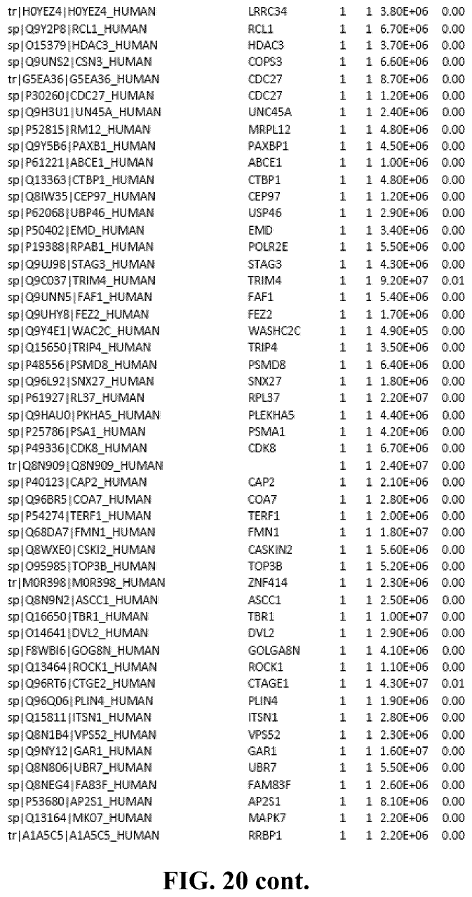

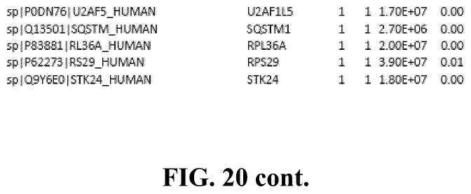

Except nuclear factors, transcription factors are generally “undruggable” by small-molecule ligands due to significant structural disorder and lack of small-molecule binding pockets. Hence, to achieve the repression of FOXM1 by modulating its binding partner, a new interaction partner of FOXM1 was identified herein. a cell line was established by stably expressing SFB-tagged FOXM1 in human lung cancer cell H1299 and then SFB-FOXM1-associated protein complex was isolated from the whole cell extracts using streptavidin beads (FIG. 1C and FIG. 8A). Analysis of this complex by liquid chromatography mass spectrometry/mass spectrometry (LC-MS/MS) identified several known FOXM1-binding partners such as Sirt1³⁷, PLK1³⁸, CDK1/2³⁹, CREBBP⁴⁰ and CCNB1³⁹, as well as a novel interaction protein USP7 (FIG. 20). Of note, as much as 44 of unique USP7 peptides was obtained in FOXM1-associated protein complex (FIG. 1D and FIG. 8B). To validate the interaction between FOXM1 and USP7, H1299 cells were transfected with a vector expressing FLAG-tagged FOXM1 in the presence or absence of a vector expressing untagged USP7. As shown in FIG. 1E, USP7 was readily detected in the immunoprecipitated complex of FOXM1 by flag agarose beads. Conversely, FOXM1 was co-immunoprecipitated with HA-tagged USP7 using HA agarose beads (FIG. 1F). To evaluate this interaction under physiological conditions, we performed co-immunoprecipitation assays with endogenous proteins from human TNBC cells MDA-MB-468 and MDA-MB-231. As shown in FIG. 1G, endogenous USP7 protein was co-precipitated by a FOXM1-specific antibody in both TNBC cells. The interaction of endogenous FOXM1 and USP7 was also validated in H1299 and human head and neck squamous cell cancer CAL33 cells (FIGS. 8C and 8D). To ascertain whether FOXM1 and USP7 interact directly, in vitro GST pull-down assays were performed by incubating purified Flag-tagged FOXM1 with a GST-fusion protein containing full-length USP7. As shown in FIG. 1H, FOXM1 strongly bound the GST-USP7 fusion protein but not GST alone. Thus, USP7 is a bona fide binding partner of FOXM1 both in vivo and in vitro.

### USP7 can Deubiquitinate and Stabilize the FOXM1 Protein

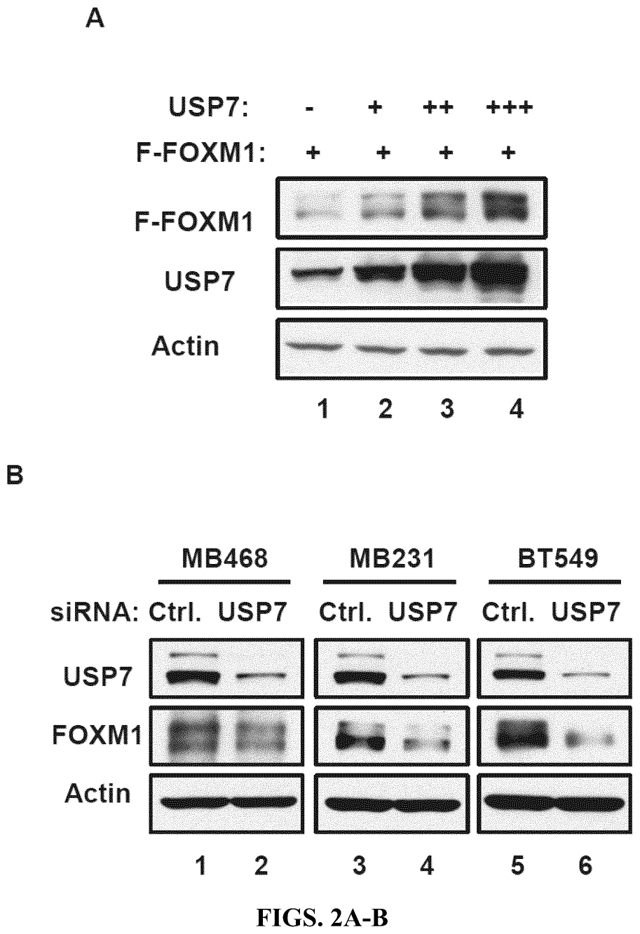

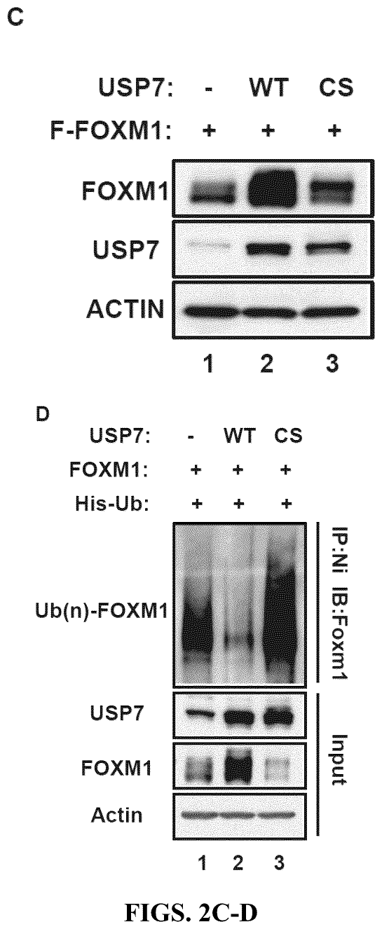

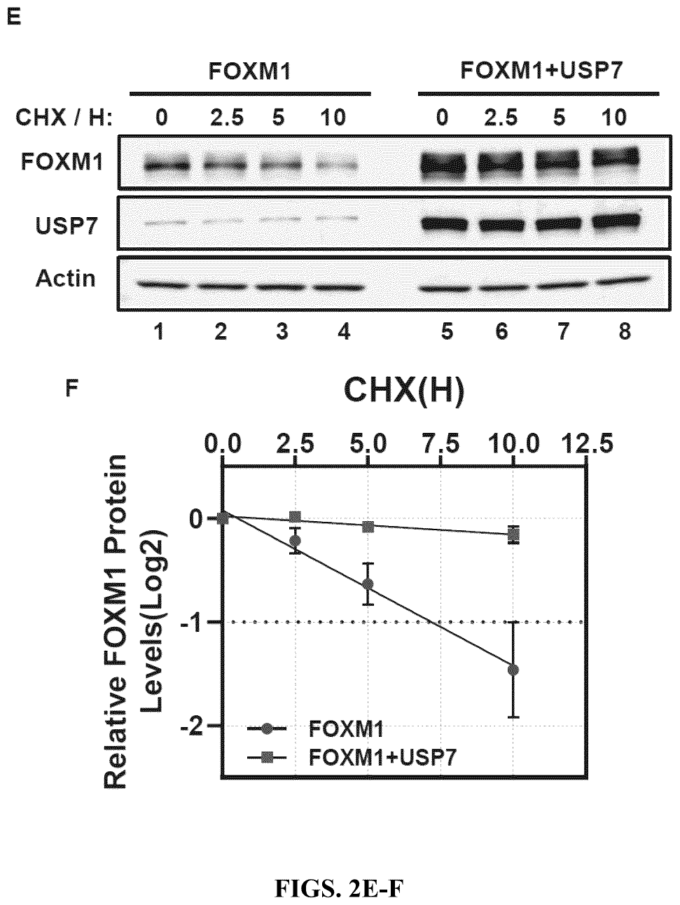

To explore the consequence of the interaction between FOXM1 and USP7, it was tested if the function of USP7 as a deubiquitinase affects the FOXM1 protein stability. H1299 cells were transfected with Flag-tagged FOXM1 and increasing amounts of USP7 expression vector. Western blot showed that FOXM1 protein levels were significantly increased upon USP7 expression in a dose-dependent manner (FIG. 2A). Conversely, USP7 depletion by specific siRNA dramatically reduced FOXM1 protein levels in several human cancer cells including triple-negative breast cancer MDA-MB-468, MDA-MB-231, BT549; head and neck squamous cell cancer CAL33 and lung cancer cell H460 (FIG. 2B and FIG. 8E). These results indicate that the USP7 expression upregulates FOXM1 protein levels.

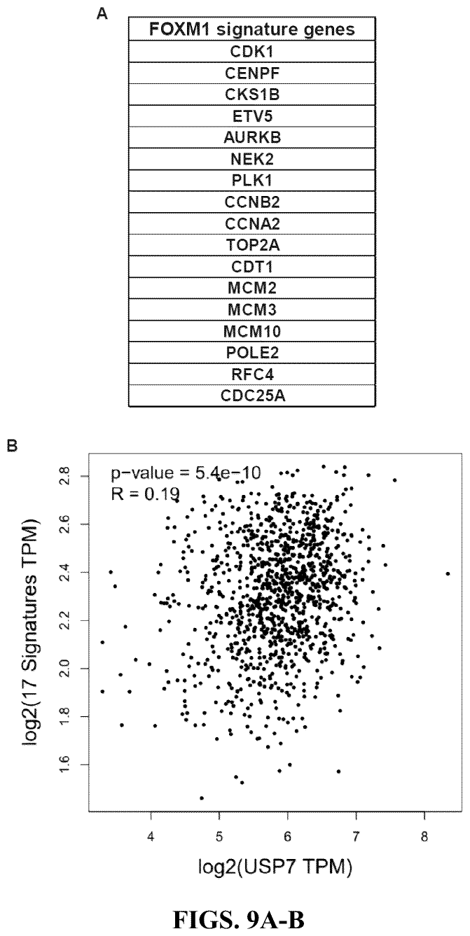

To see if this effect requires the deubiquitinase activity of USP7, a USP7 mutation C223S was utilized that lacks the deubiquitinase activity. H1299 cells were co-transfected with Flag-tagged FOXM1-expressing vector and wild-type or catalytic mutant USP7 C223S expressing vector. Wild-type, but not USP7 C223S mutant, was able to increase the FOXM1 protein levels (FIG. 2C). These results suggest that USP7 regulates FOXM1 through its deubiquitinase activity. Next, the effect of wild-type USP7 and USP7 C223S mutant was investigated on the FOXM1 polyubiquitination. As shown in FIG. 2D, co-transfection of wild-type USP7, but not C223S, dramatically reduced the polyubiquitination levels of FOXM1. Consistently, the FOXM1 protein half-life in H1299 cells was determined and co-expression of USP7 was found to markedly extend the FOXM1 protein half-life from ˜5 h to over 10 h (FIGS. 2E and 2F). In addition, Pearson correlation analysis between the expression of FOXM1 signature and USP7 in BRCA revealed a positive correlation, albeit the correlation coefficient is small (FIGS. 9A and 9B). Taken together, these results demonstrate that FOXM1 is a bona fide substrate of USP7 deubiquitinase.

### PU7-1 is a PROTAC Degrader for USP7 Protein

Recently, PROTAC technology is emerging as a breakthrough in drug discovery and development⁴¹. A PROTAC degrader can contain three moieties: ligand for protein of interest (POI), linker and ligand for cellular E3 ligase. Mechanistically, it has at least three major advantages over traditional small-molecule inhibitors. First, PROTACs can achieve higher selectivity due to the need of recruiting both POI and E3 ligase to form a productive ternary complex^{42,43}. Second, PROTACs can achieve high potency at lower doses. Since PROTAC functions like a catalytic enzyme, it can be recycled after POI is degraded. The off-target toxicity caused by high dosage is often avoided. Third, all functions associated with the POI can be effectively blocked. Small-molecule inhibitors usually can abolish the activity of POI but are unable to block protein-protein interaction. PROTACs completely degrade POI and eliminate all corresponding cellular functions. Therefore, to repress FOXM1 in triple-negative breast cancer, we developed a novel USP7 PROTAC degrader.

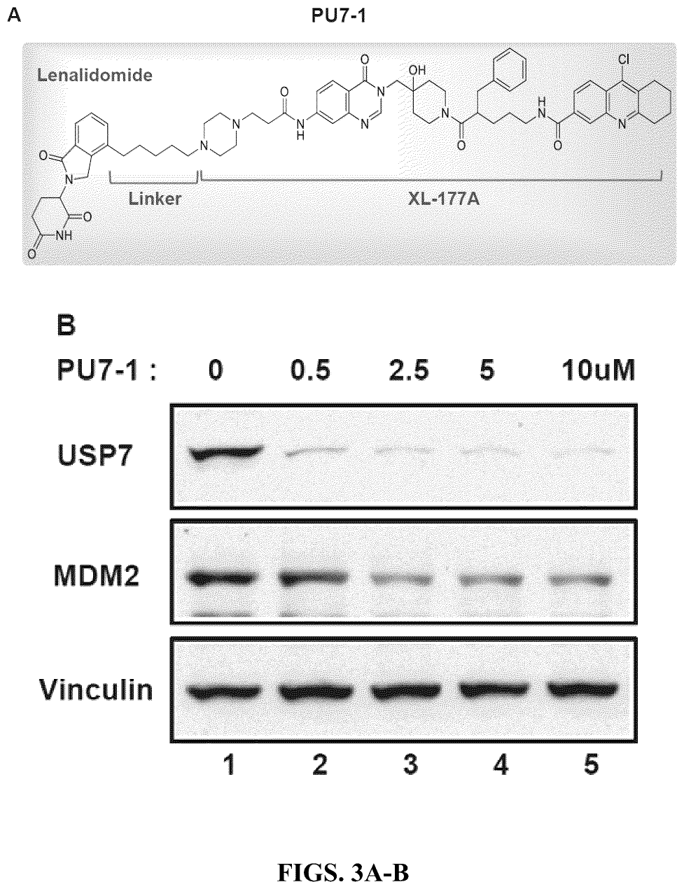

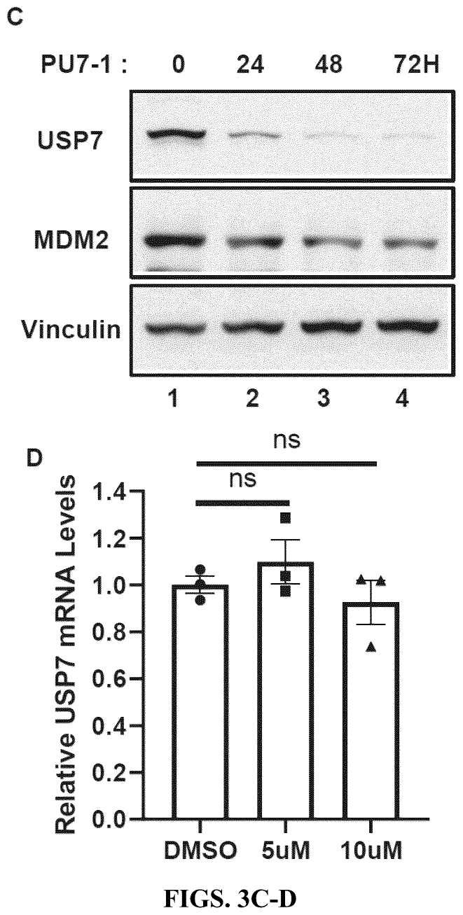

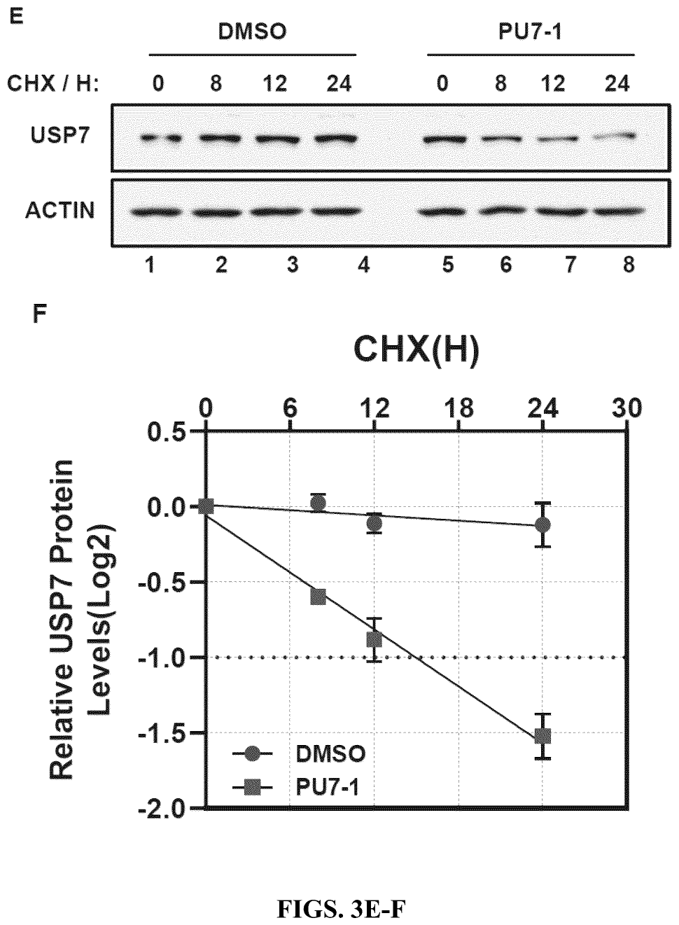

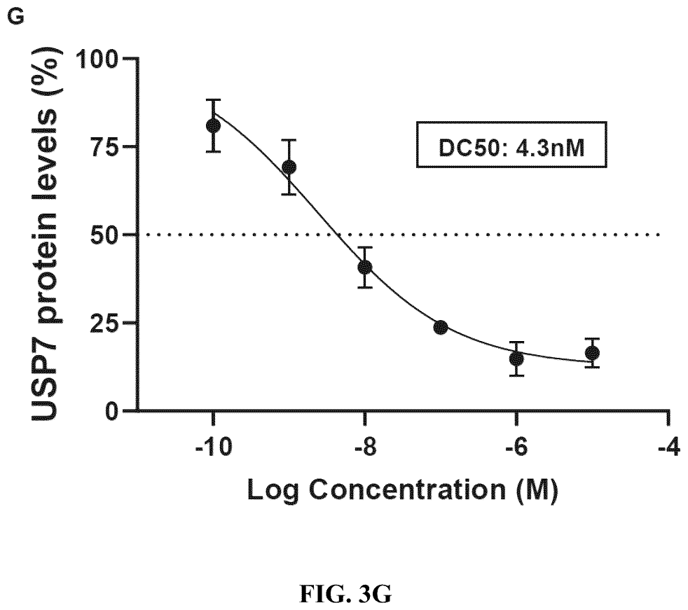

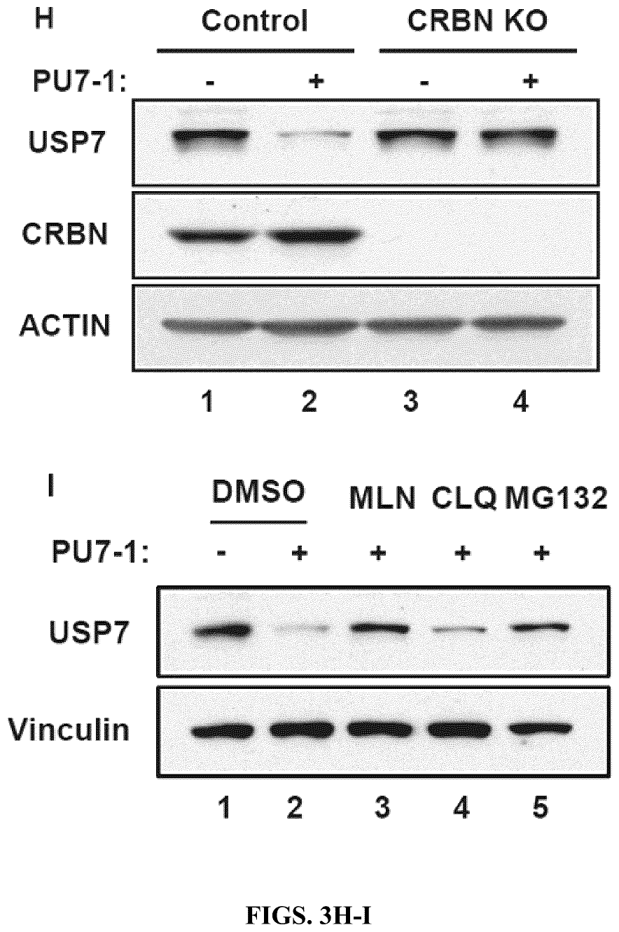

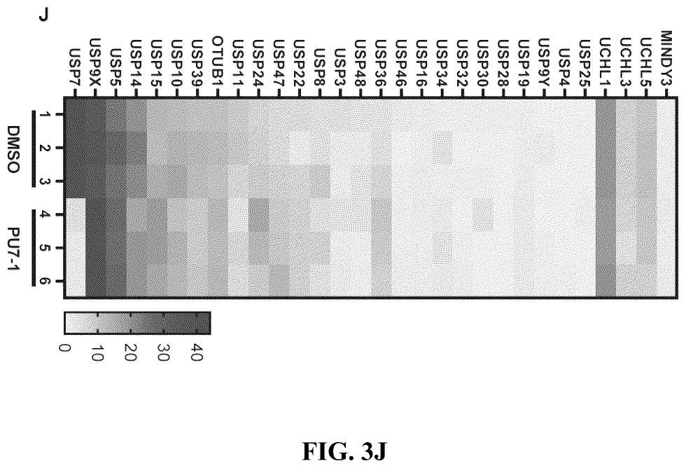

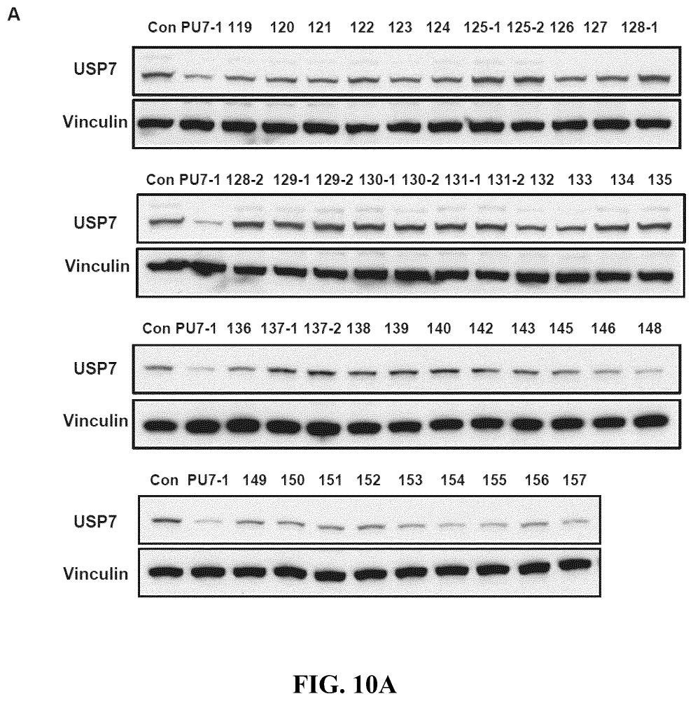

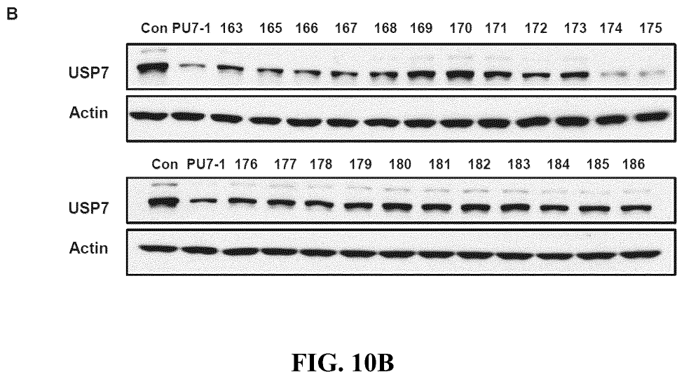

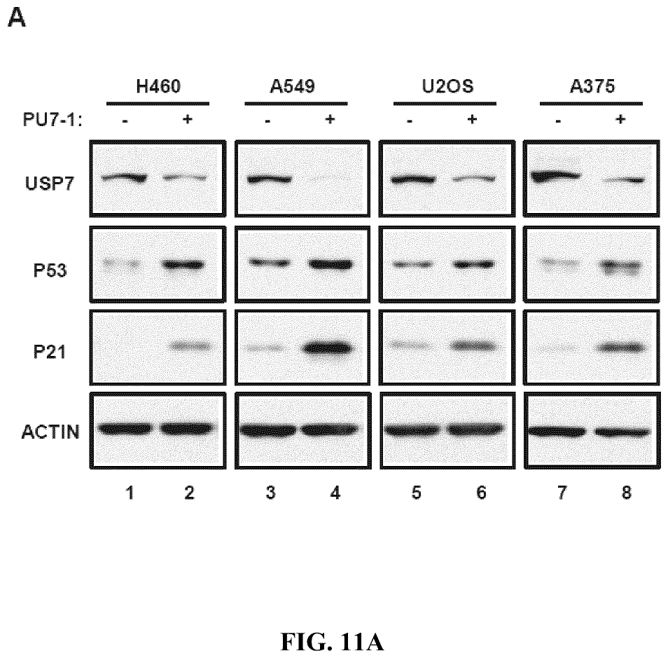

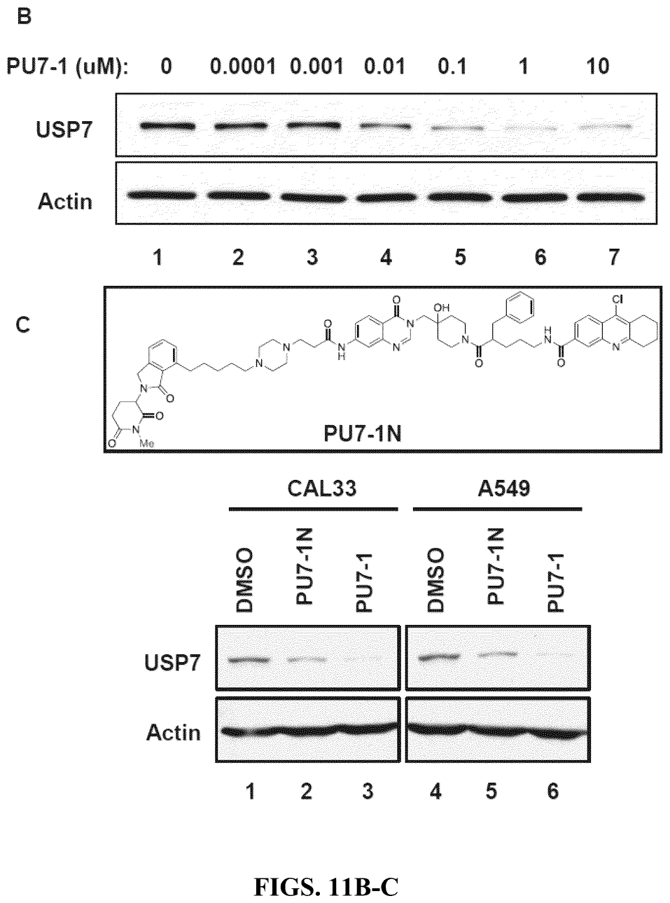

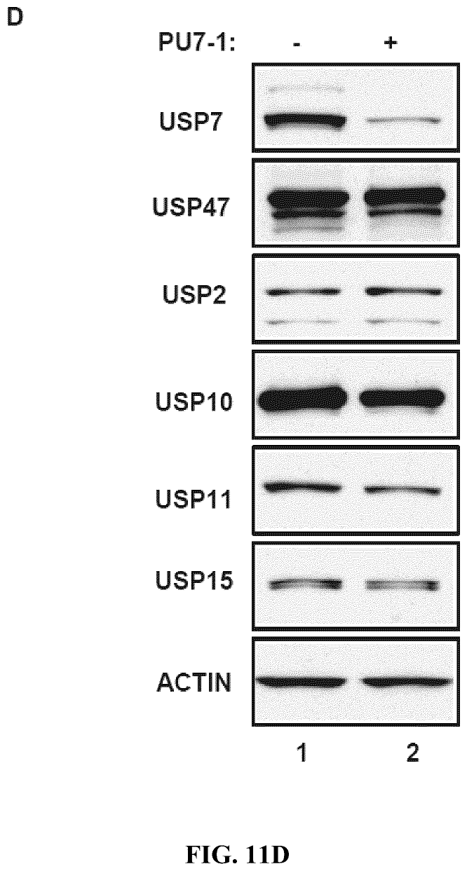

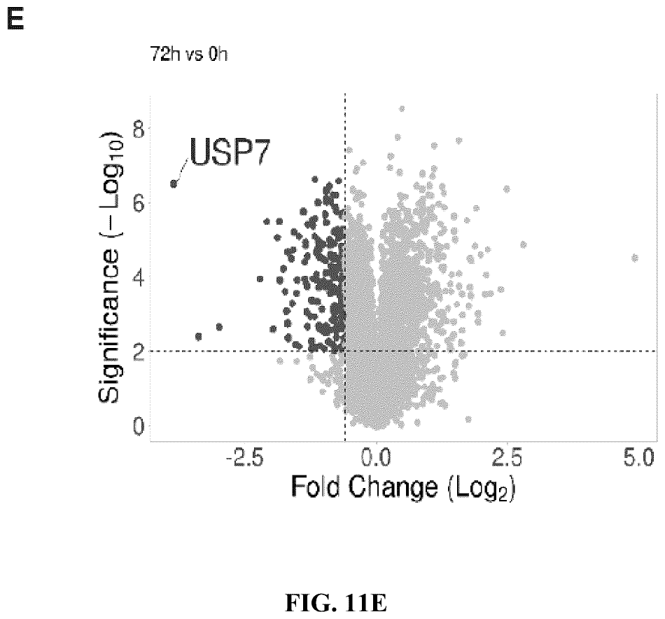

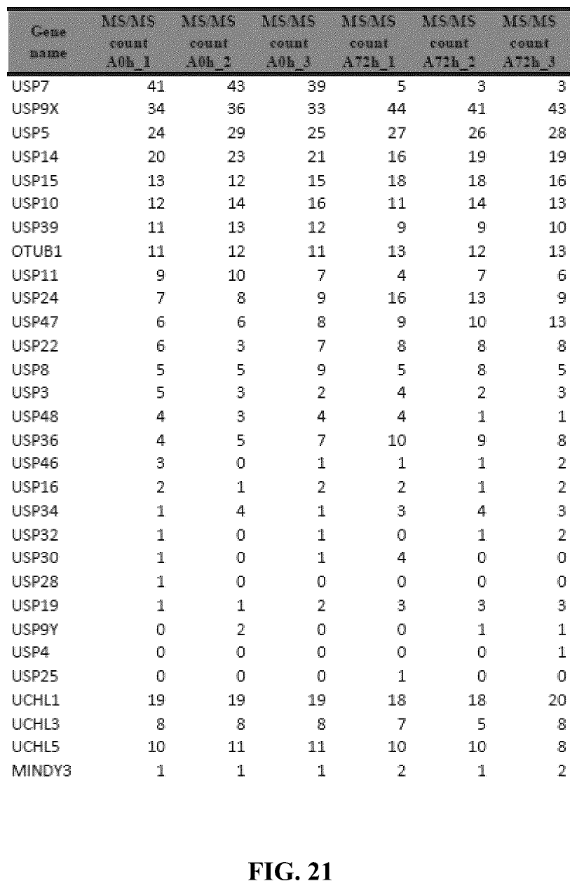

To specifically degrade USP7, a set of 66 PROTAC compounds were designed and synthesized that contain an USP7 specific binding moiety XL177A and an E3 ubiquitin ligase (CRBN or VHL) binding moiety connected by different linkers. All putative USP7 PROTACs were used to treat CAL33 cells that express high levels of USP7 protein, and then the USP7 protein levels were determined by western blot (FIGS. 10A and 10B). Through initial screening, PU7-1 was identified as a potential USP7 degrader (FIG. 3A and FIG. 10). As shown in FIGS. 3B and 3C, USP7 protein levels were dramatically reduced in CAL33 cells upon PU7-1 treatment in a dose- and time-dependent manner. Consistent with this effect, MDM2, a target of USP7 deubiquitinase, decreased significantly in a similar manner. The effect of PU7-1 was also tested in several human cancer cells harboring wild-type p53. The data showed that PU7-1 treatment effectively reduced USP7 protein levels and activated p53 pathway (FIG. 11A). In addition, to exclude the possibility that PU7-1 affects the USP7 protein levels by regulating its transcription, a quantitative real-time PCR was performed in CAL33 treated with PU7-1 for 72 h. These data suggested that the expression levels of USP7 mRNA were not altered by PU7-1 treatment (FIG. 3D). In addition, the half-life of USP7 protein was examined in CAL33 cells in the presence or absence of PU7-1 treatment. As shown in FIGS. 3E and 3F, the PU7-1 treatment remarkably decreased the USP7 half-life from over 24 h to ˜15 h. CAL33 were also treated with the different dosage of PU7-1 ranging from 0.0001 to 10 UM for 24 h. Western blot and densitometry analysis of USP7 protein bands using IMAGE J software revealed that PU7-1 effectively decreased USP7 protein at a DC50 of 4.3 nM (FIG. 11B, FIG. 3G and FIG. 21). These findings imply that PU7-1 can effectively decrease cellular USP7 protein through targeting its protein stability.

To investigate the mechanism of action of PU7-1, CRBN knockout (KO) CAL33 cell line was generated using CRISPR-Cas9 technology. Control and CRBN KO cells were treated with 2.5 μM of PU7-1 for 72 h and then the expression levels of USP7 and CRBN proteins were detected by western blot analysis. CRBN protein was undetectable in the KO cells (FIG. 3H). Notably, CRBN depletion completely abrogated the USP7 degradation mediated by PU7-1 treatment. In addition, PU7-1N, in which a methyl group was installed to impair the binding of CRBN, also exhibited defective capability to degrade USP7 protein (FIG. 11C). CAL33 cells were first treated with 5 μM of PU7-1 for 48 h to degrade the USP7 proteins, and then treated with 5 μM of MLN4924, 25 μM of Chloroquine (CLQ) or 5 μM of MG132 for additional 24 h. Western blot analysis revealed that both Cullin E3 ligase inhibitor MLN4924 and proteosome inhibitor MG132 significantly rescued the USP7 degradation mediated by PU7-1, whereas lysosome inhibitor Chloroquine (CLQ) had no effect on USP7 abundance (FIG. 3I). Taken together, these data demonstrate that the USP7 degradation by PU7-1 requires cellular CRBN/Cullin E3 ligase via ubiquitination-proteosome pathway.

Lastly, because of the high homology in USP protein family, the selectivity of PU7-1 against potential off-target effects was studied. Western blot analysis of several USPs including USP47, the closest homolog of USP7, were performed in CAL33 cells treated with 10 μM of PU7-1 for 72 h. The result indicated that USP7 was the only protein degraded upon treatment, while the protein levels of the other USPs had no change (FIG. 11D). To further investigate the selectivity of PU7-1 proteome-wide, mass spectrometry-based quantitative proteomic analysis was performed in human lung cancer A549 cells treated with or without 2.5 μM of PU7-1 for 72 h. As shown in FIG. 11E, of ˜5500 proteins quantified, USP7 was the most significantly decreased protein upon the treatment. In contrast, there was no measurable decrease in the protein levels of the other 29 deubiquitinases (FIG. 3J). These findings imply that PU7-1 is an effective and selective PROTAC degrader for the USP7 protein.

### PU7-1 Treatment Leads to FOXM1 Degradation and can Suppress its Transcriptional Targets.

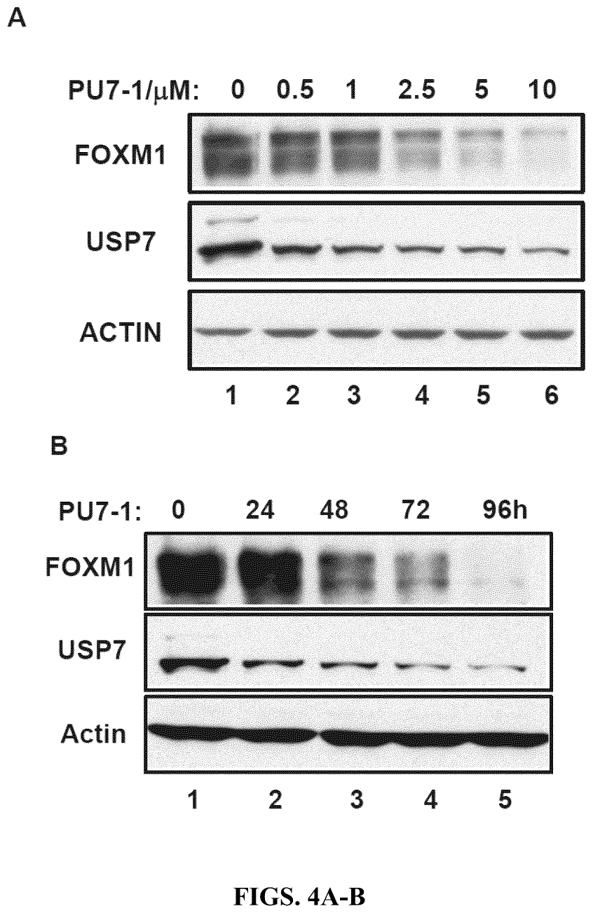

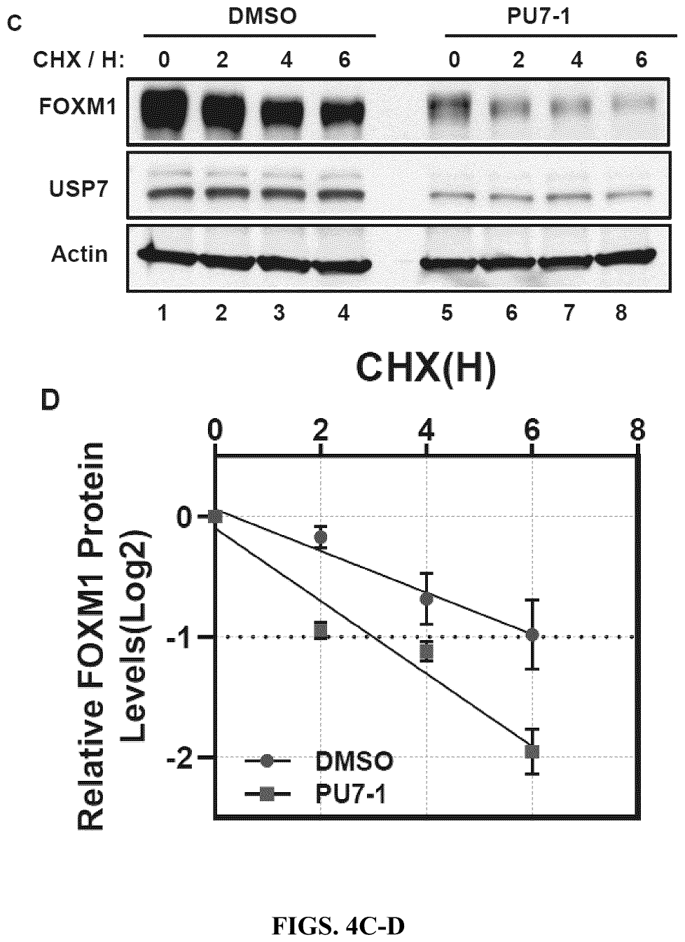

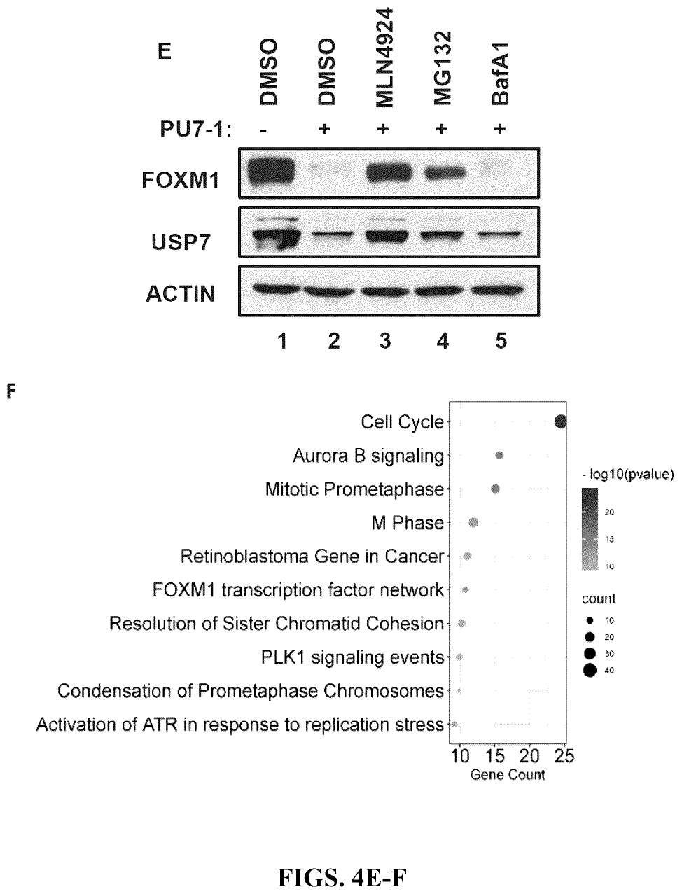

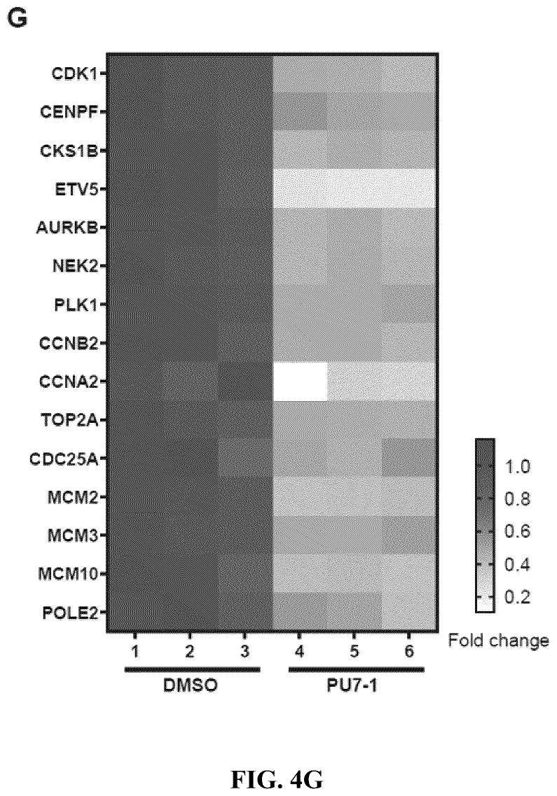

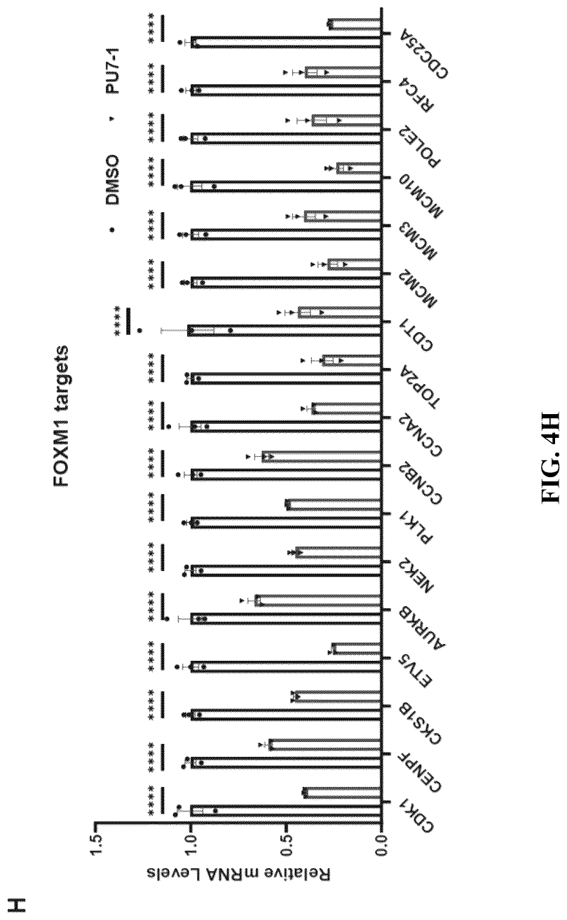

Since the results demonstrate that FOXM1 is a bona fide target of USP7 deubiquitinase, the effect of PU7-1 on FOXM1 was examined next. FOXM1 protein levels significantly decreased in a dose- and time-dependent manner in CAL33 cells upon PU7-1 treatment, well correlated with USP7 protein levels (FIGS. 4A and 4B). Consistently, the FOXM1 half-life was significantly reduced from more than 6 hours to 3 hours upon PU7-1 treatment in CAL33 cells (FIGS. 4C and 4D). These results demonstrate that the treatment with the USP7 degrader PU7-1 results in the destabilization of FOXM1. This effect was readily blocked by Cullin inhibitor MLN4924 and proteosome inhibitor MG132, but not by lysosome inhibitor BafA1 (FIG. 4E), suggesting that PU7-1-induced destabilization of FOXM1 requires the ubiquitination-proteosome pathway.

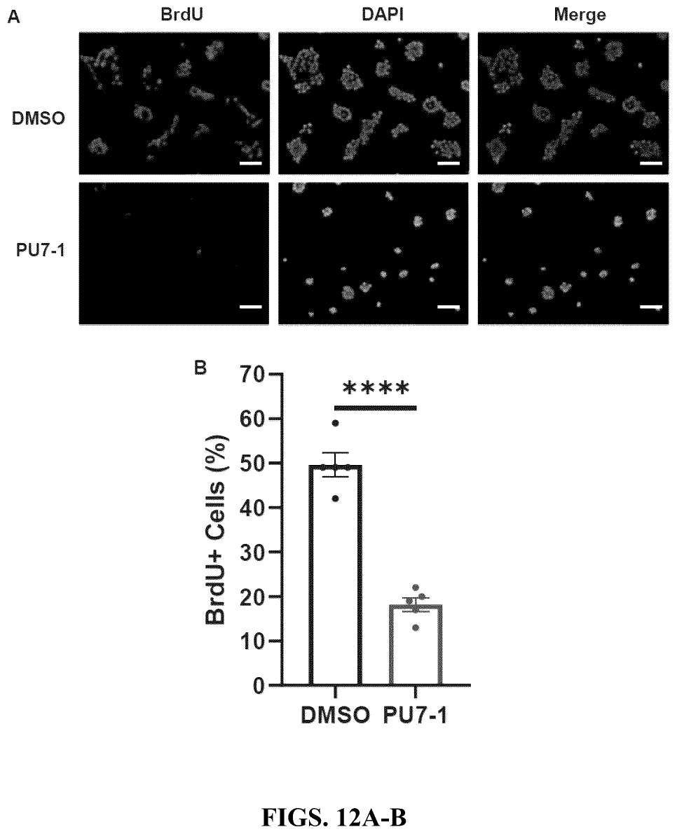

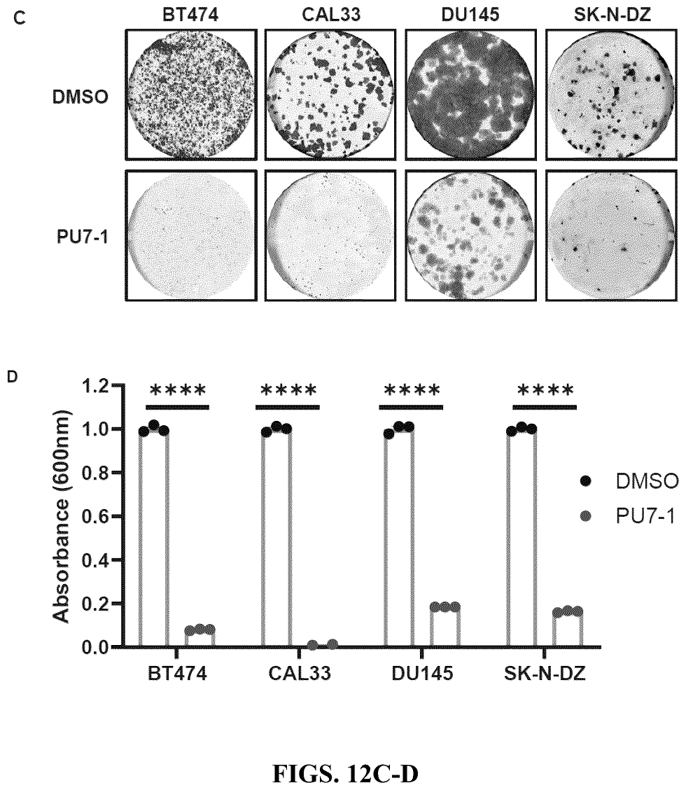

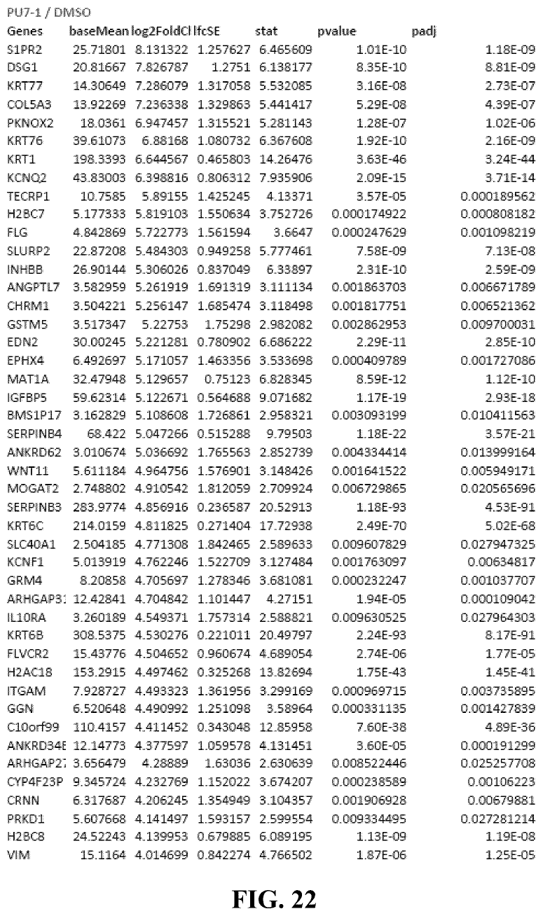

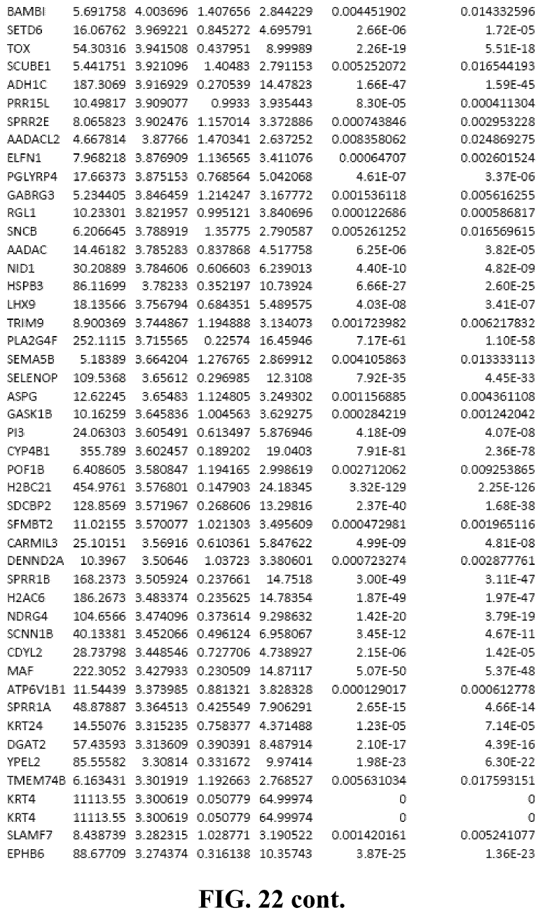

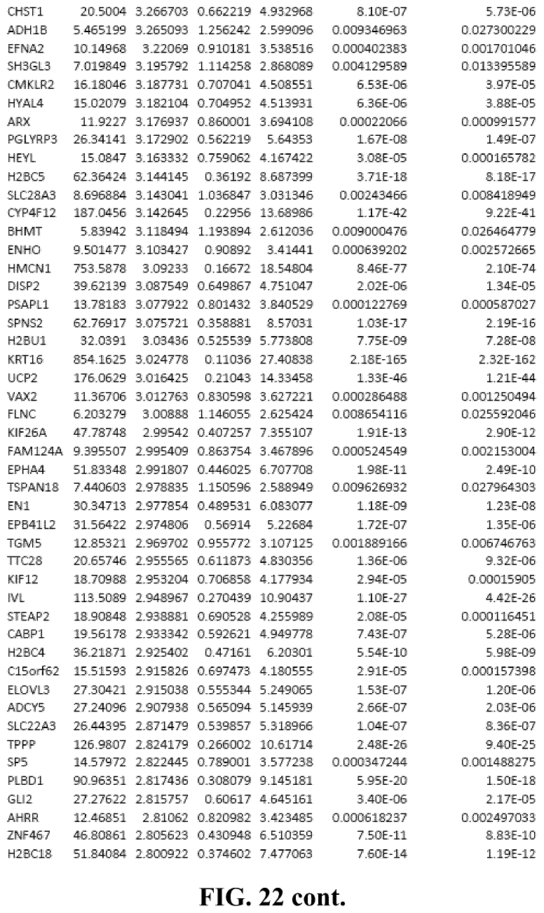

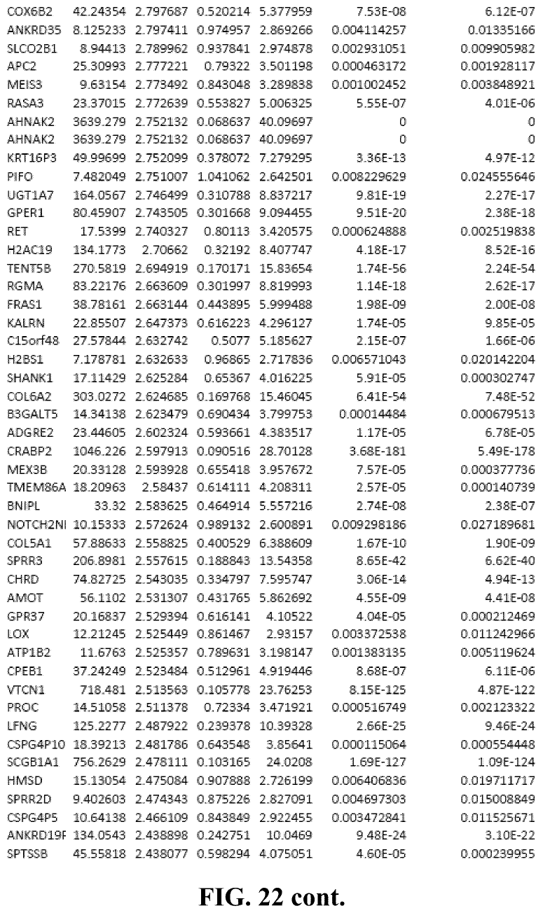

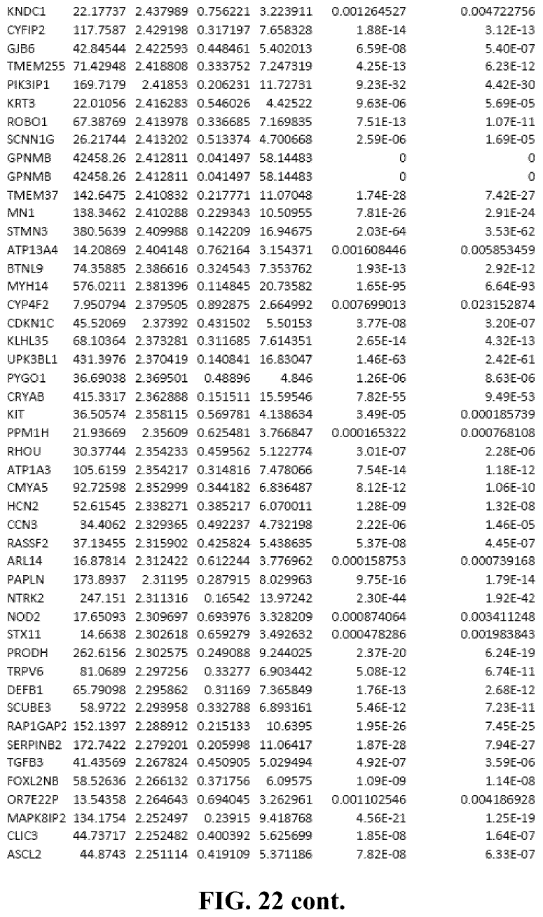

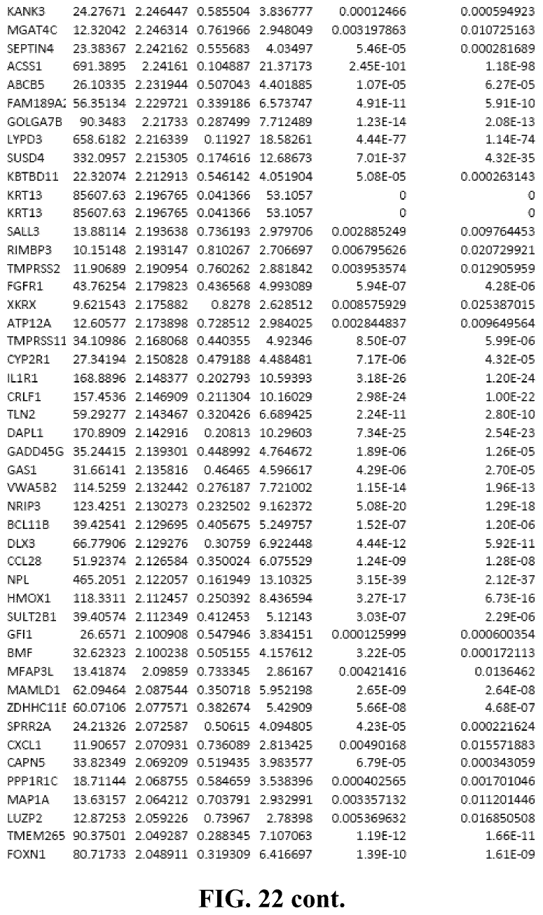

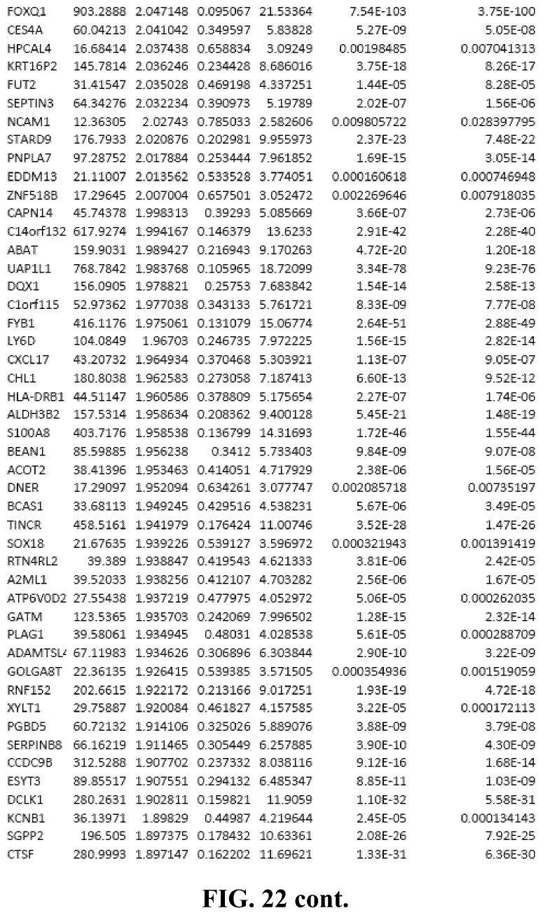

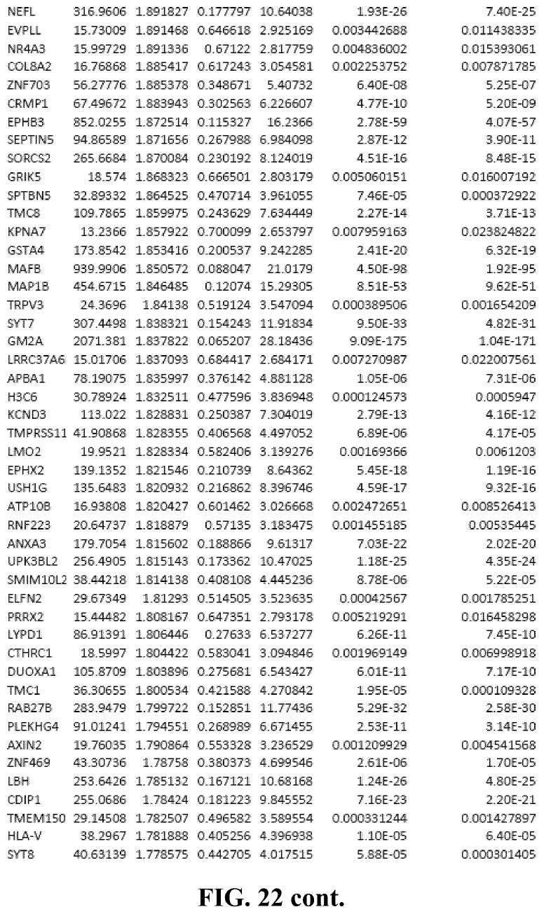

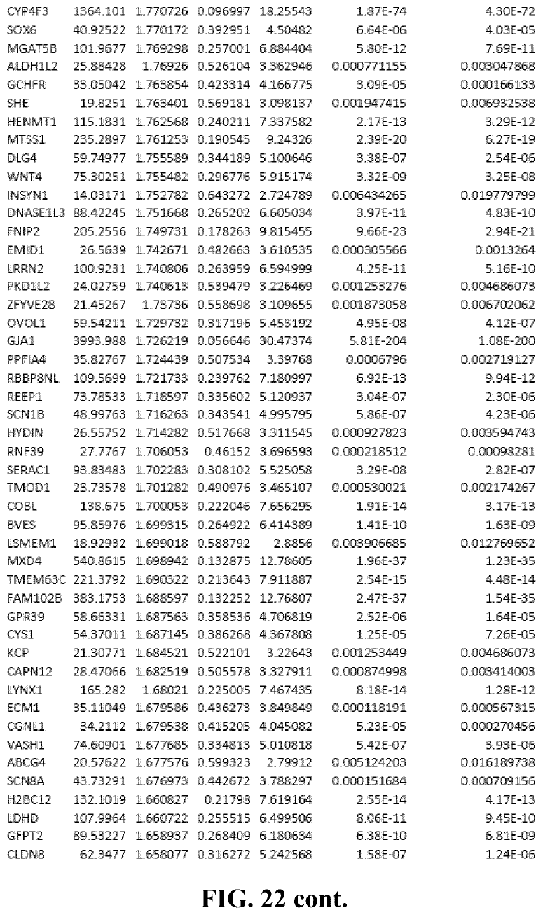

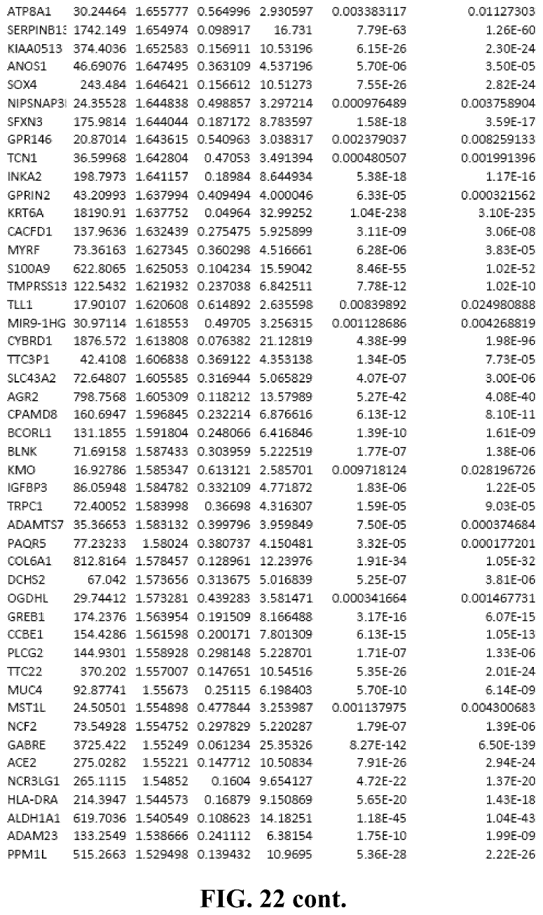

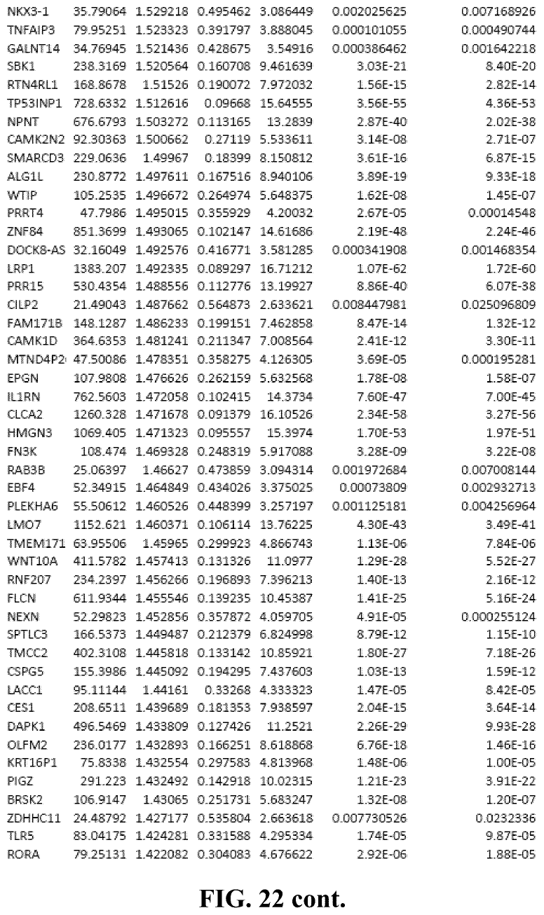

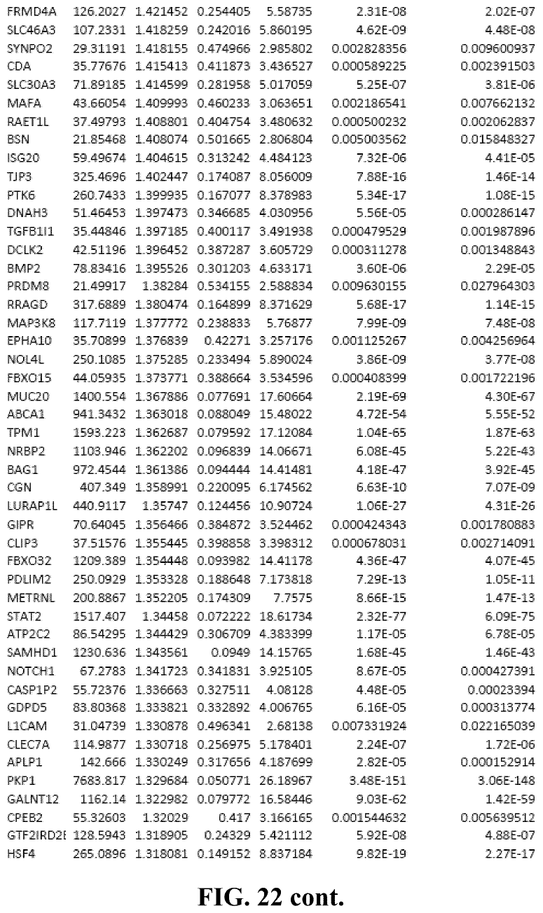

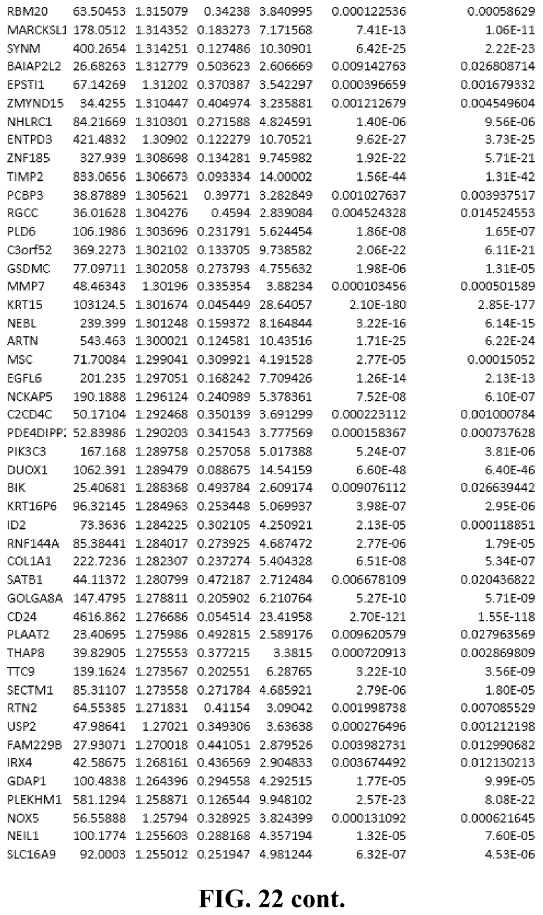

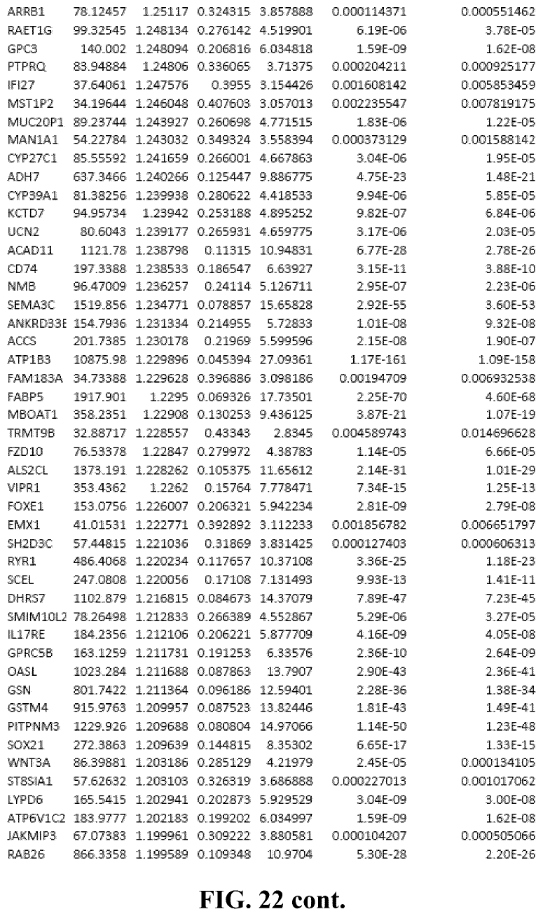

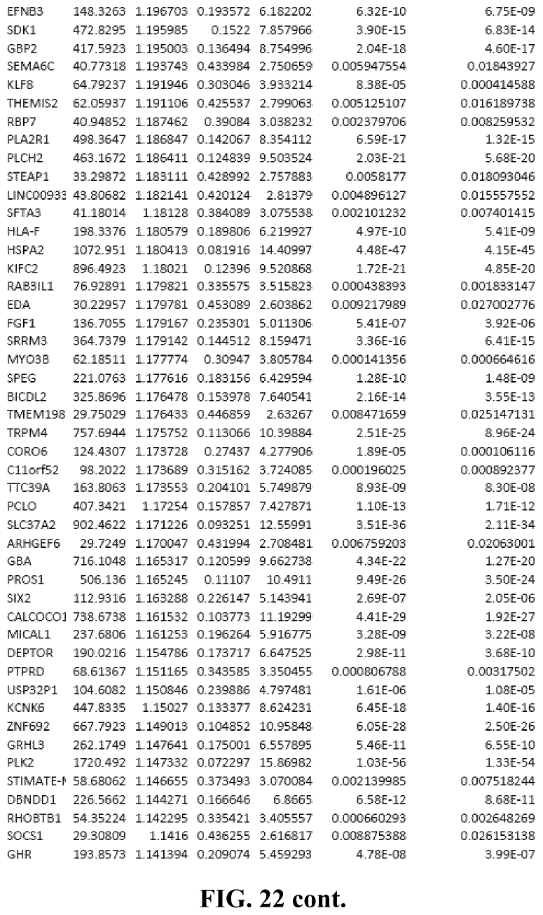

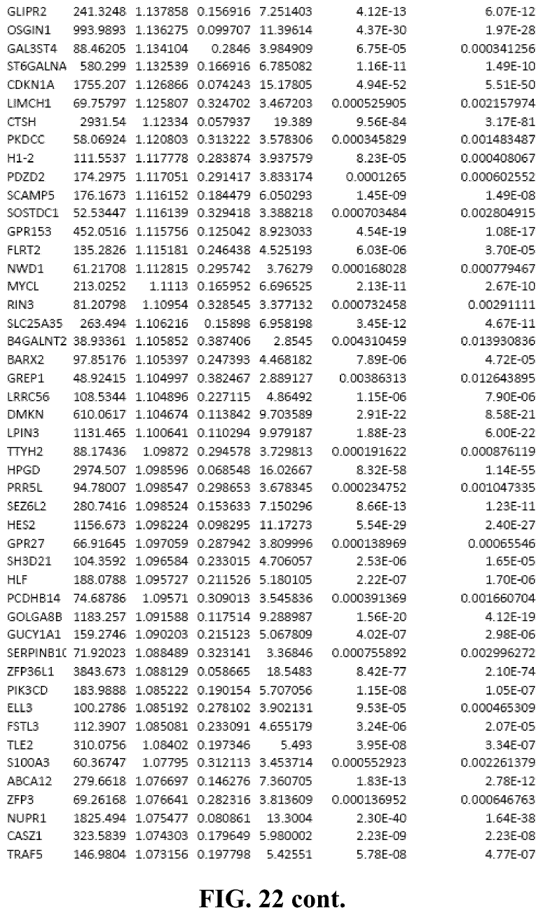

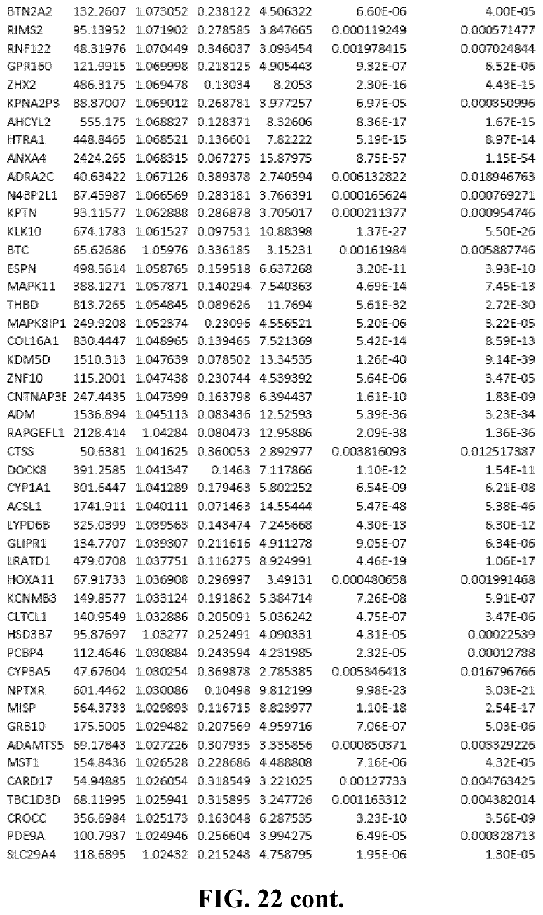

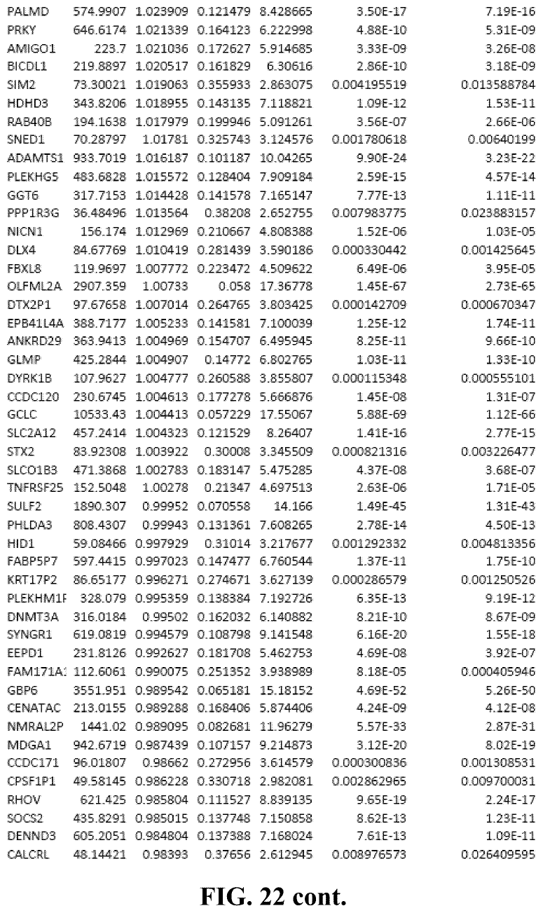

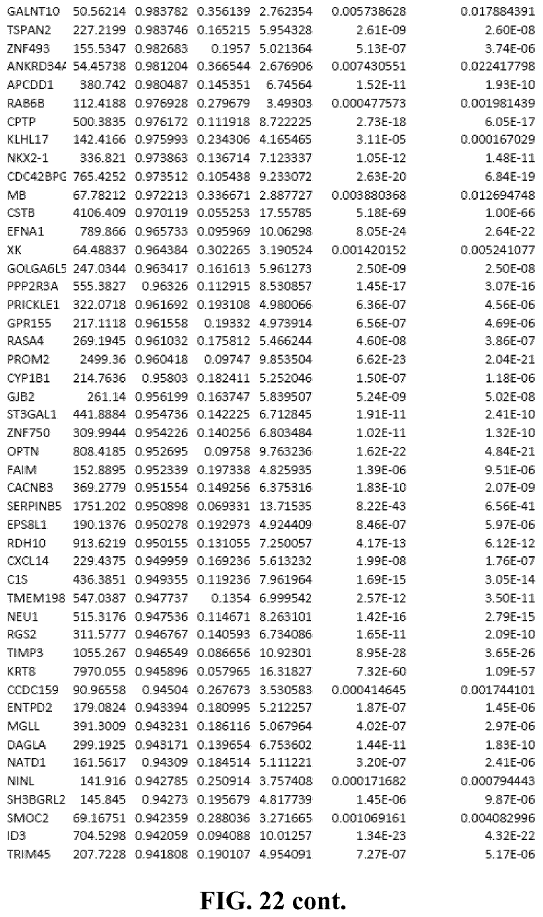

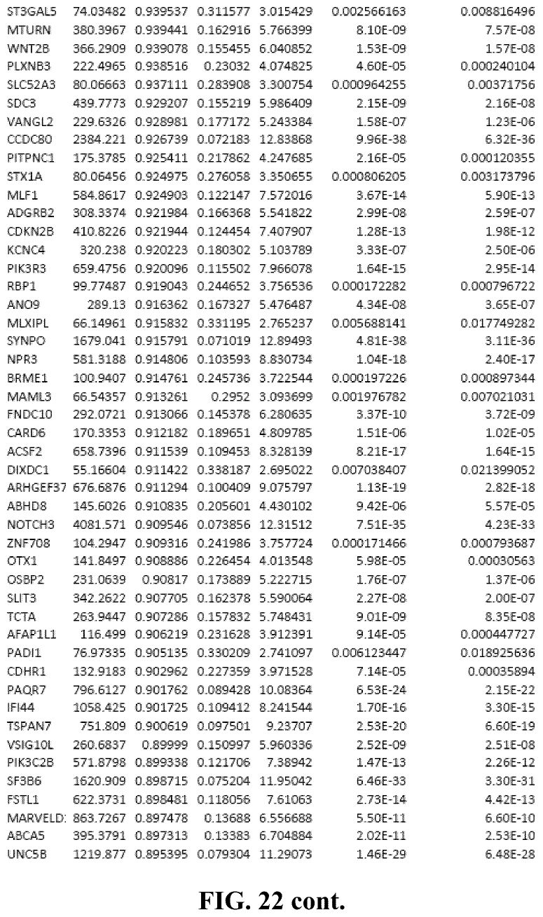

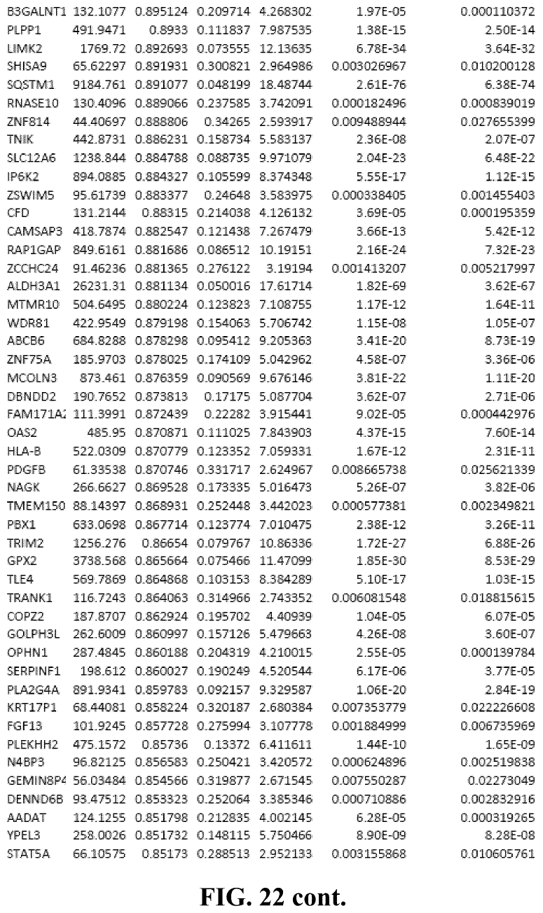

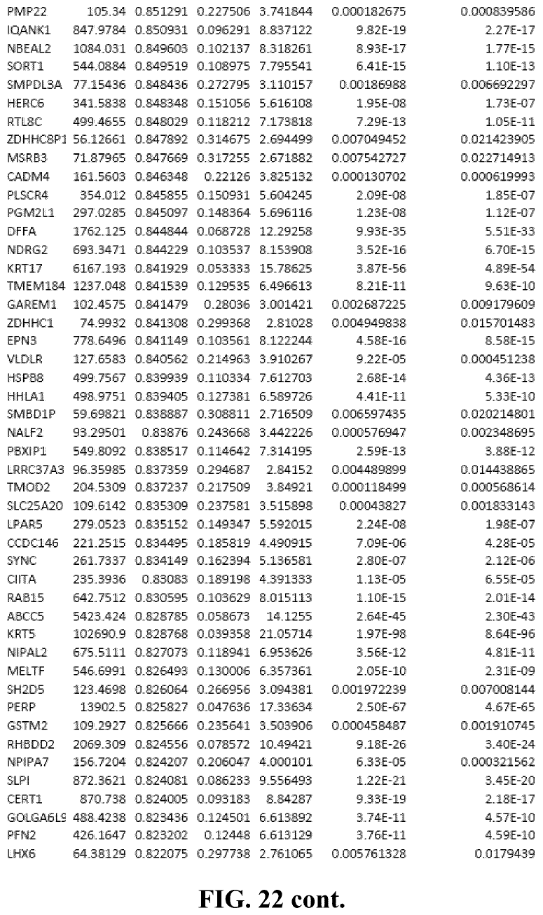

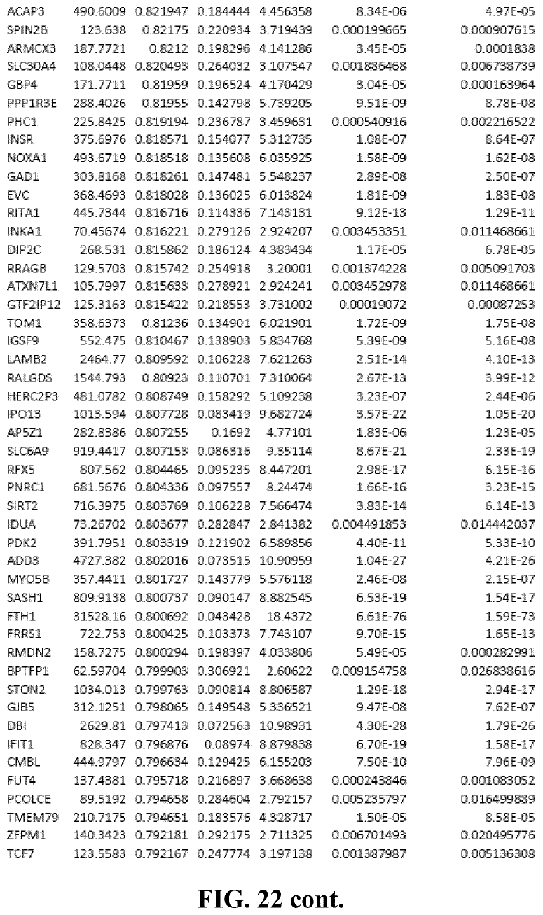

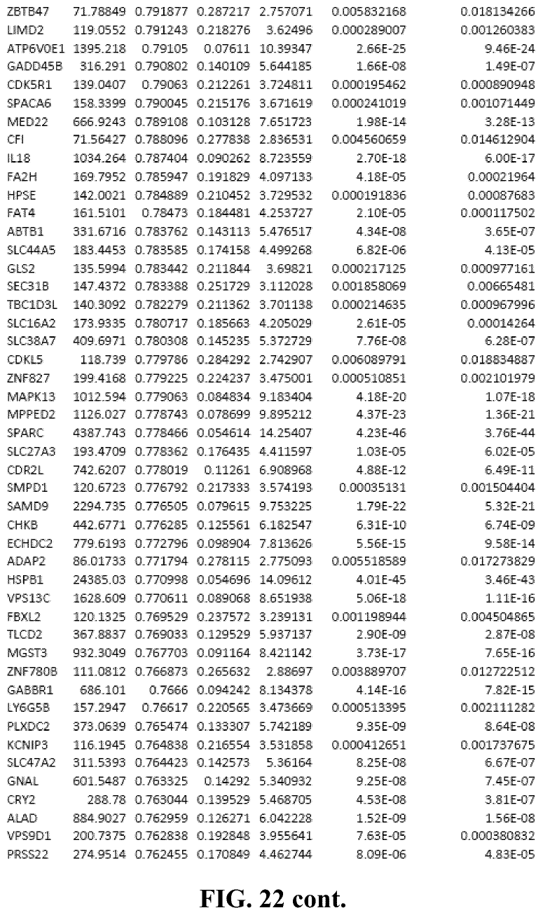

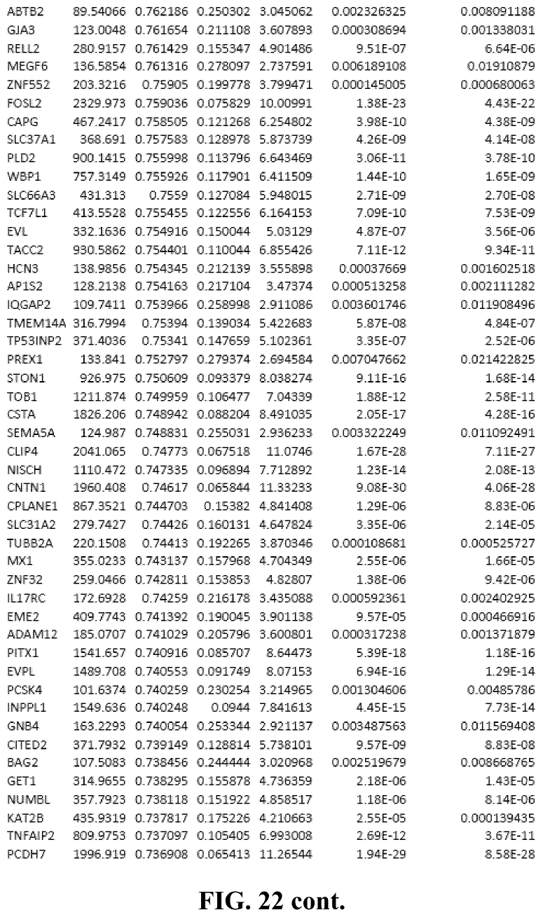

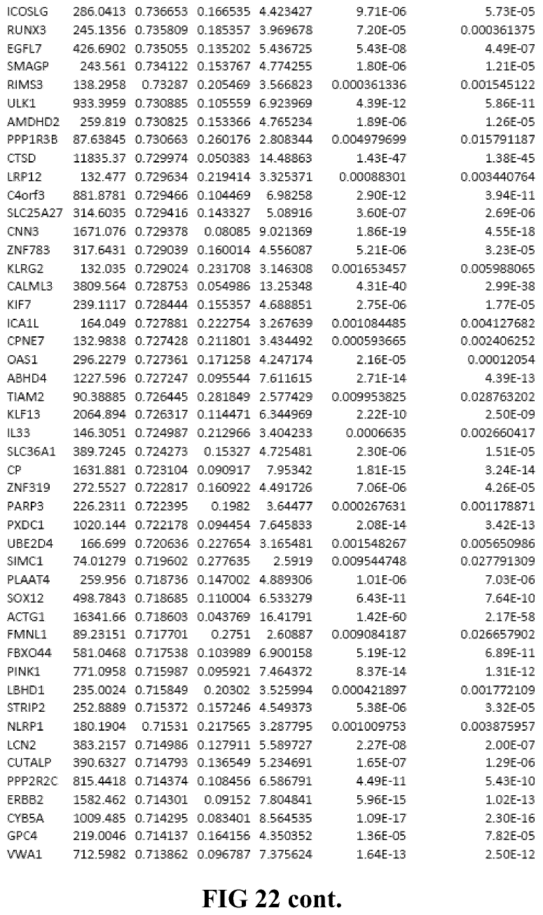

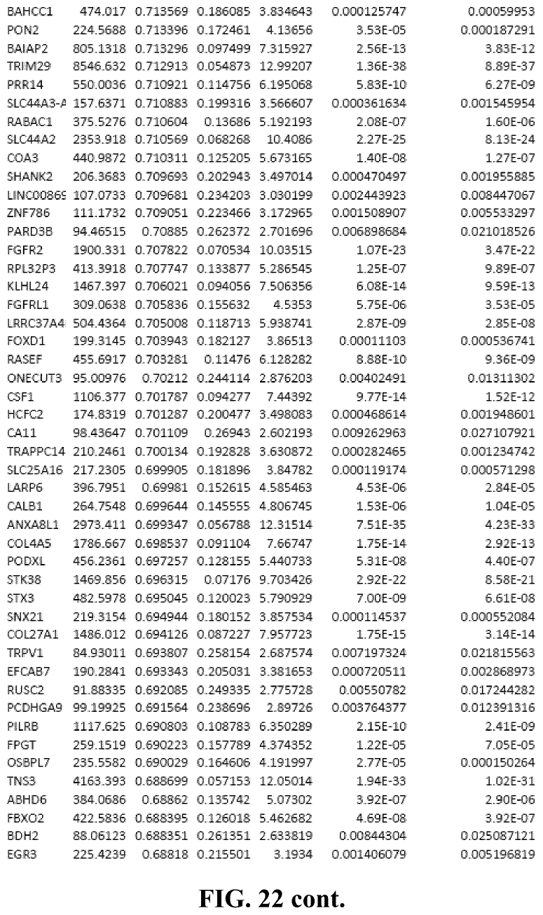

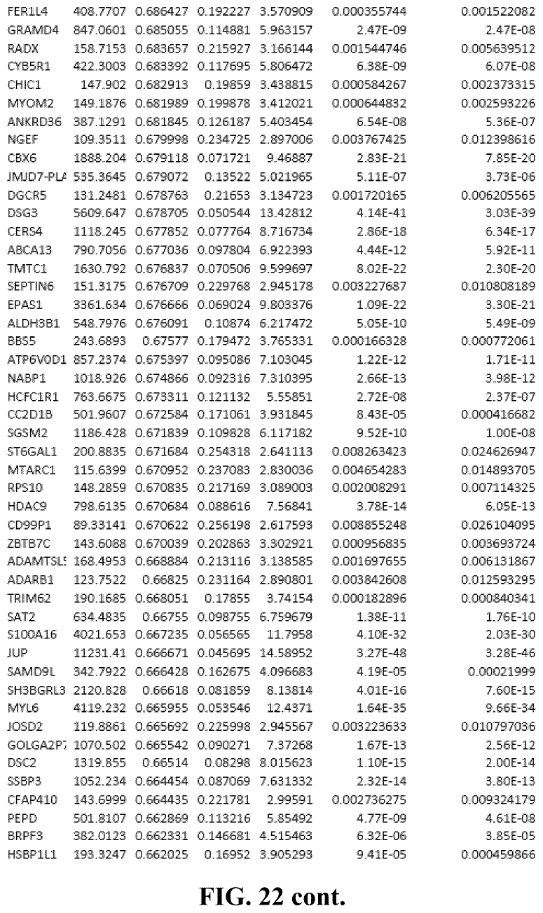

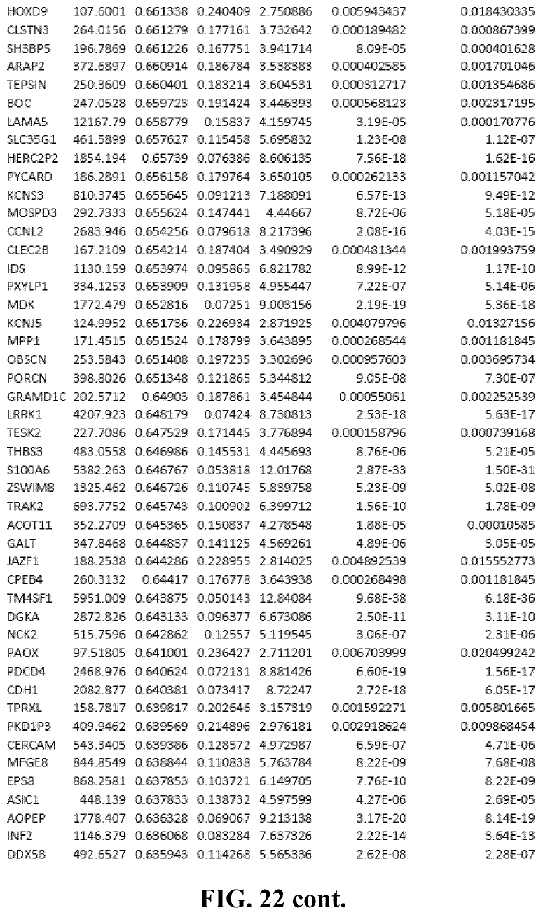

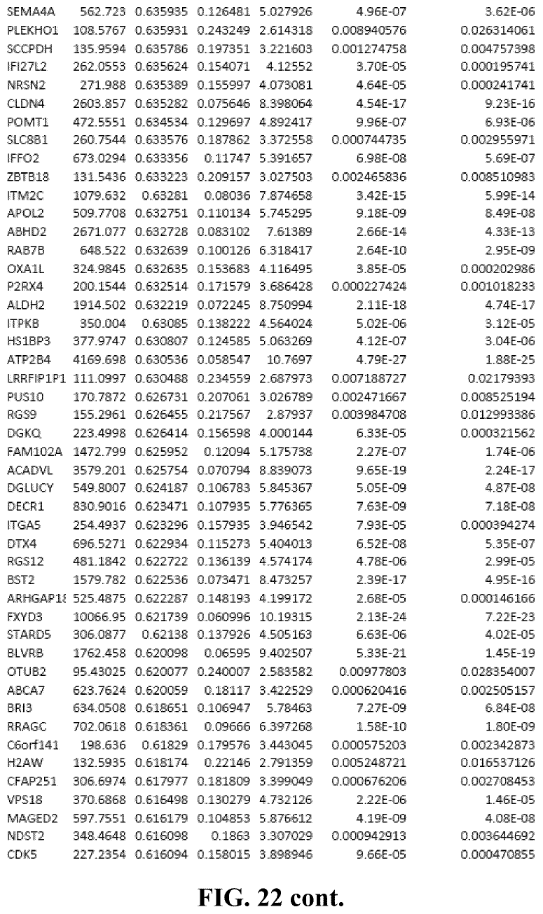

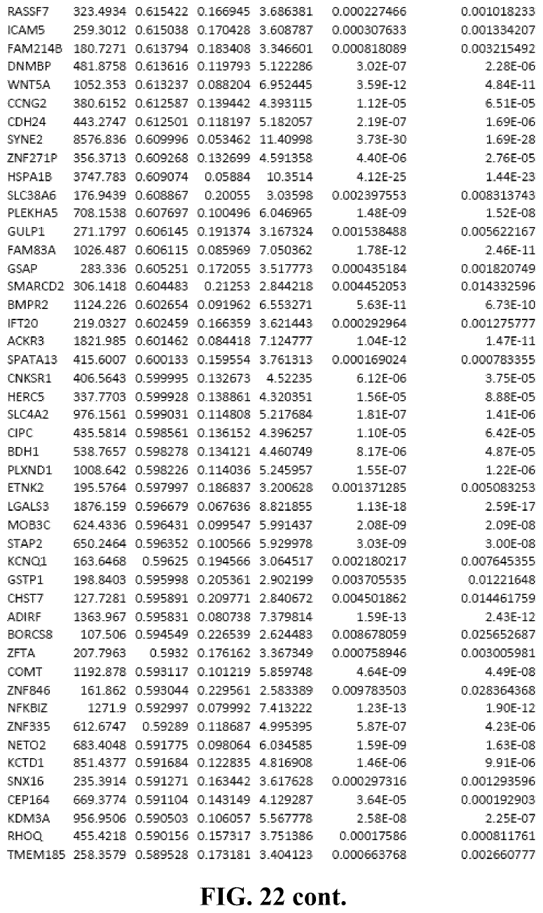

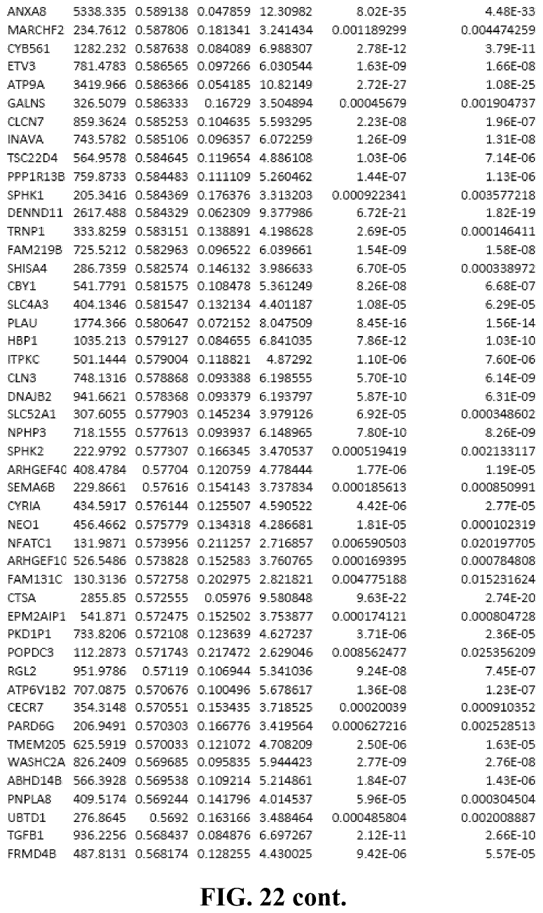

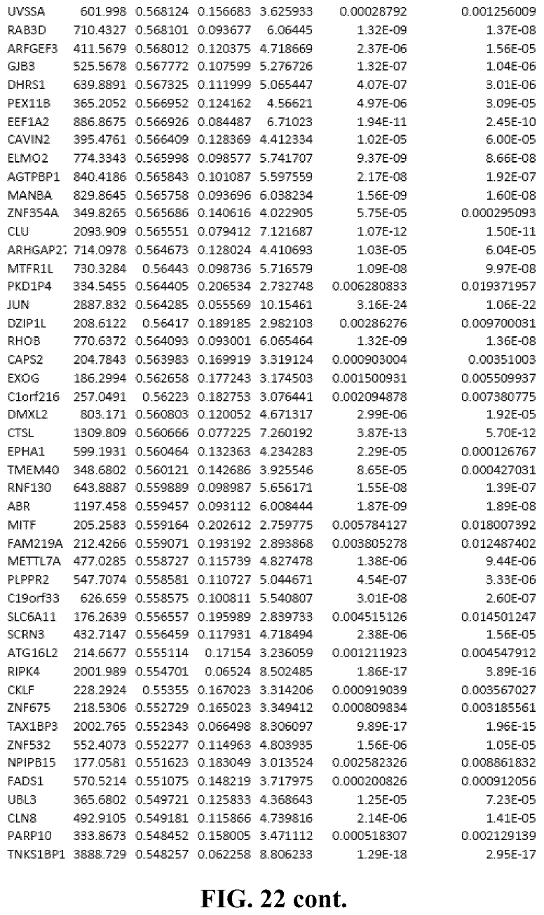

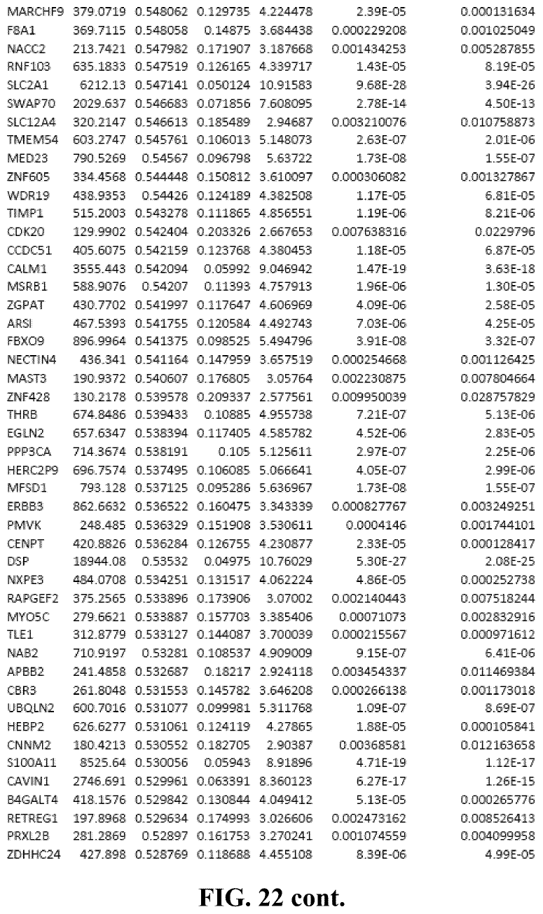

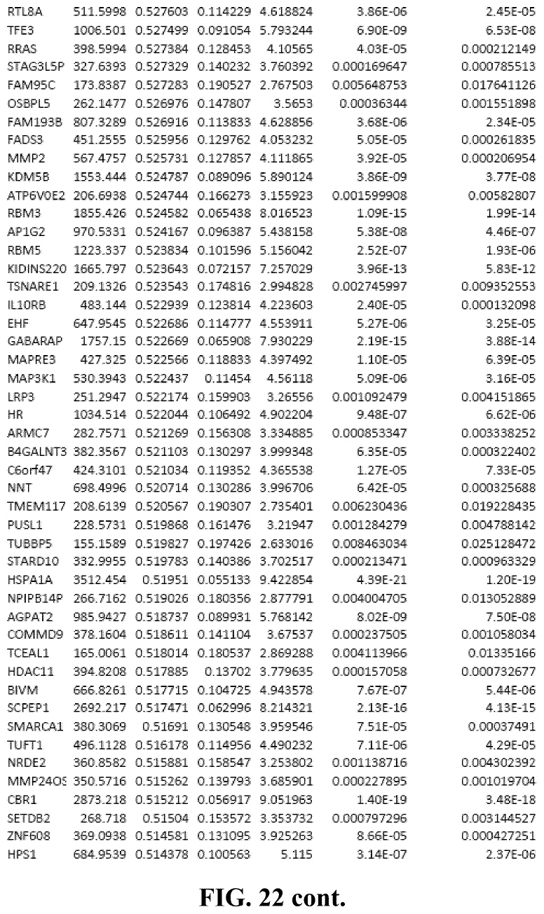

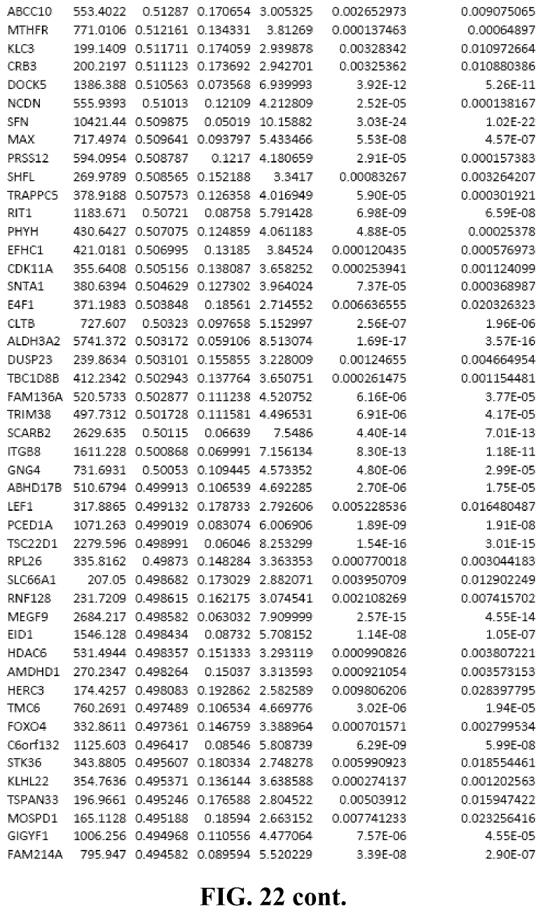

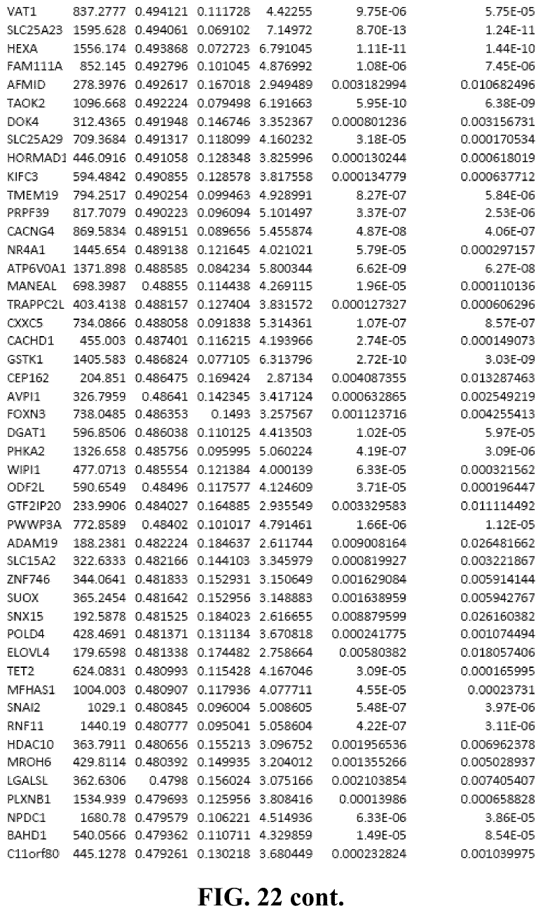

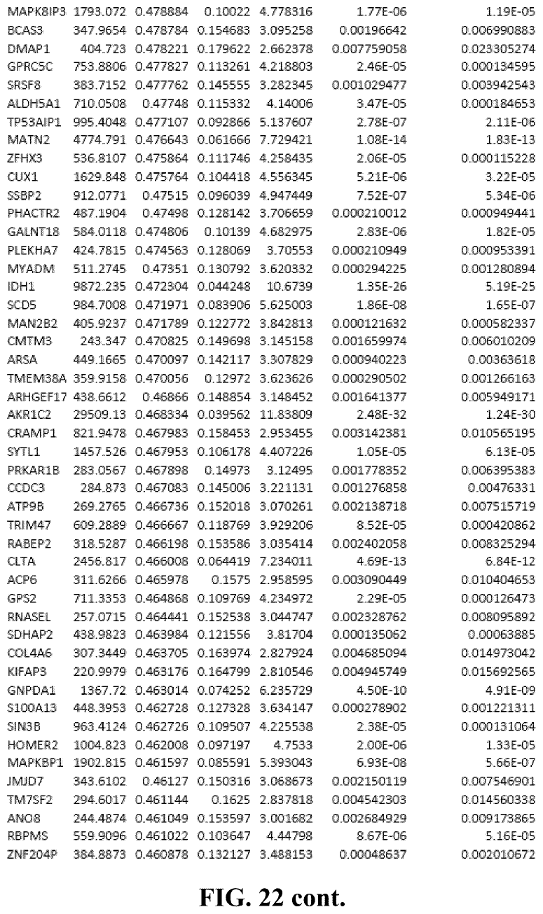

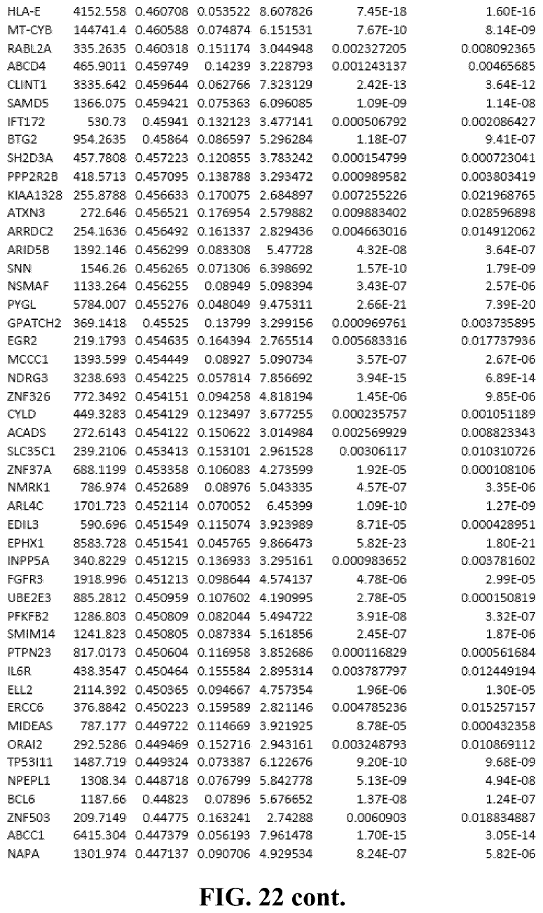

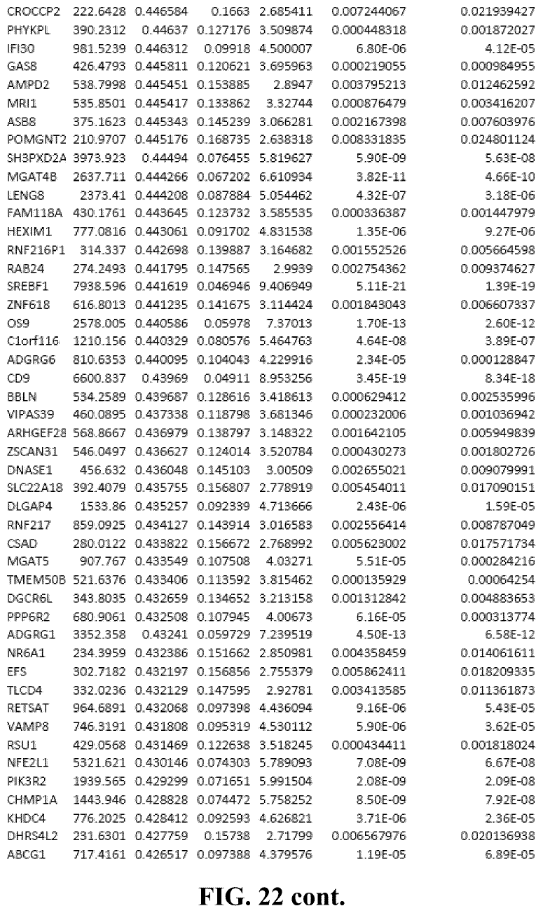

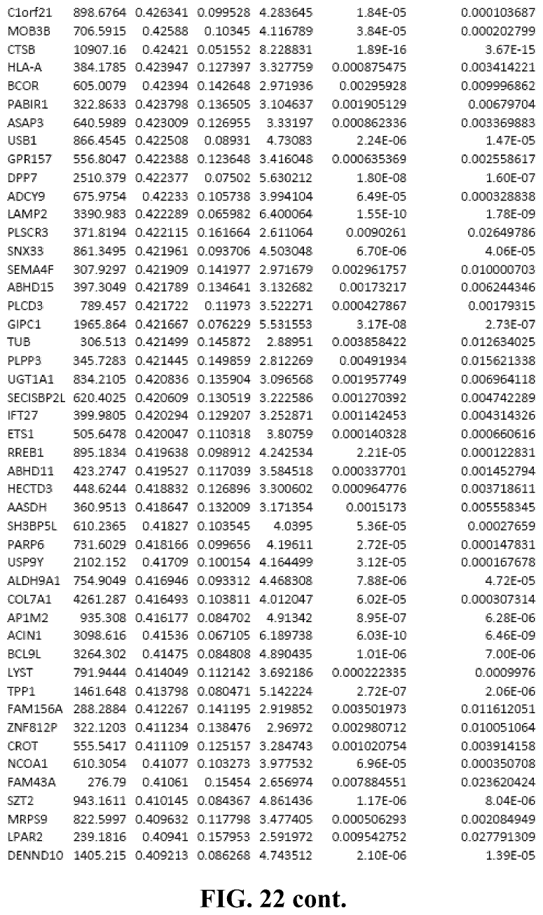

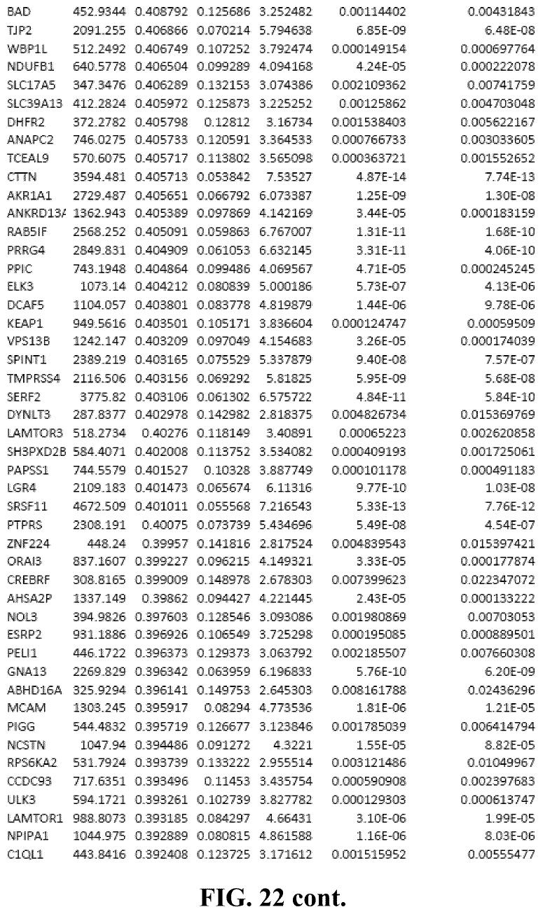

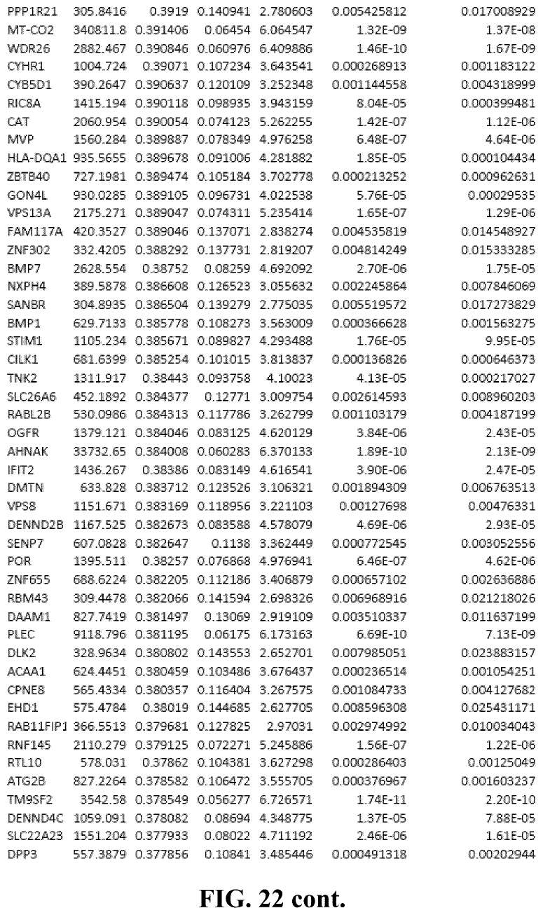

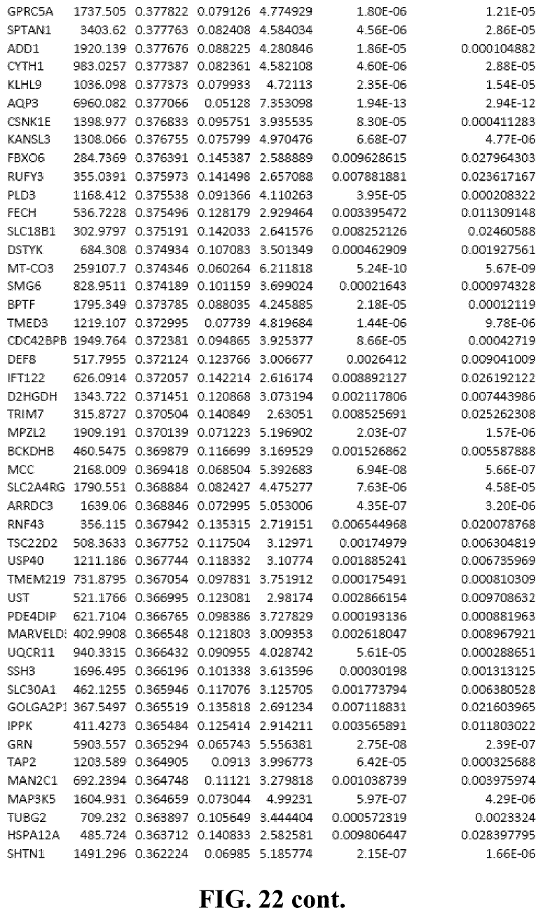

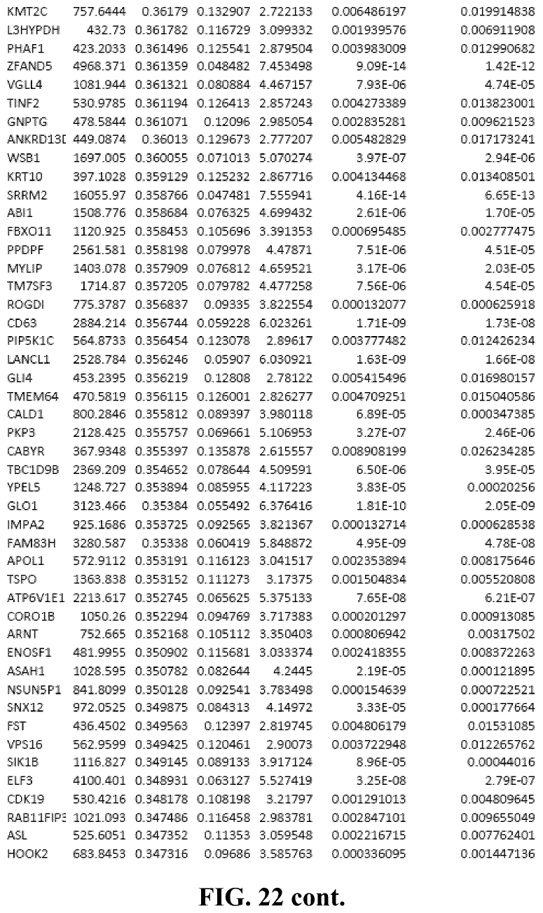

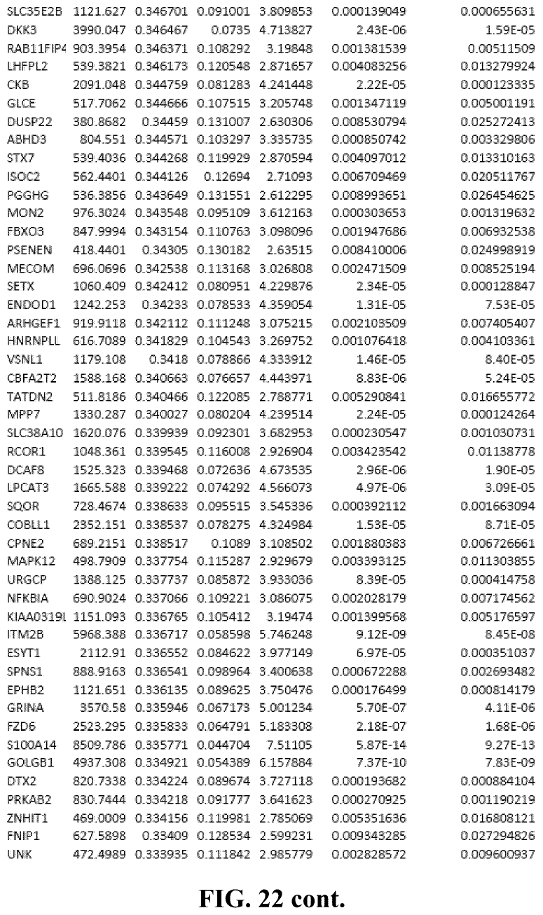

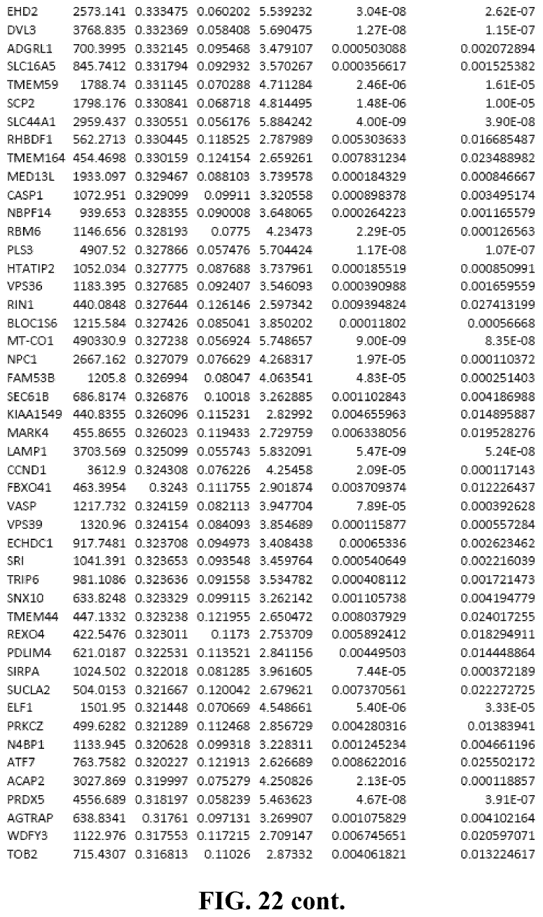

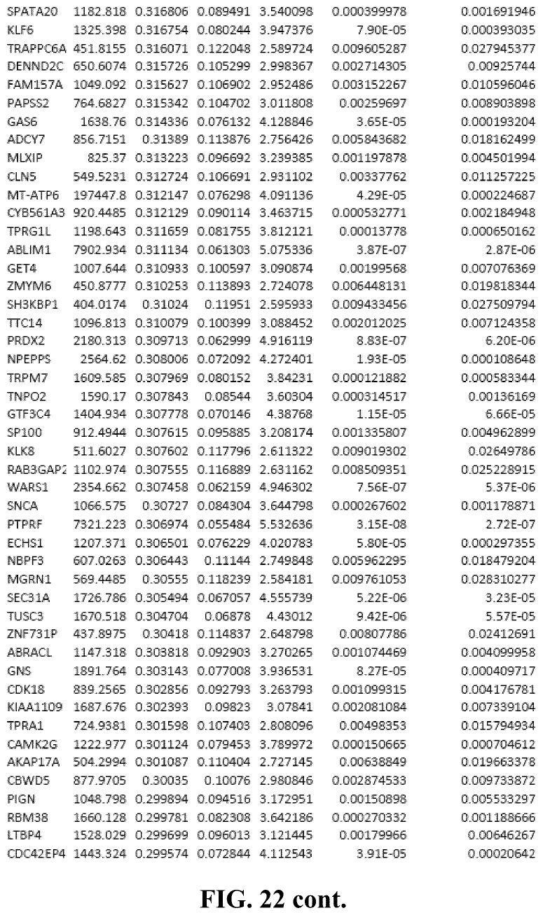

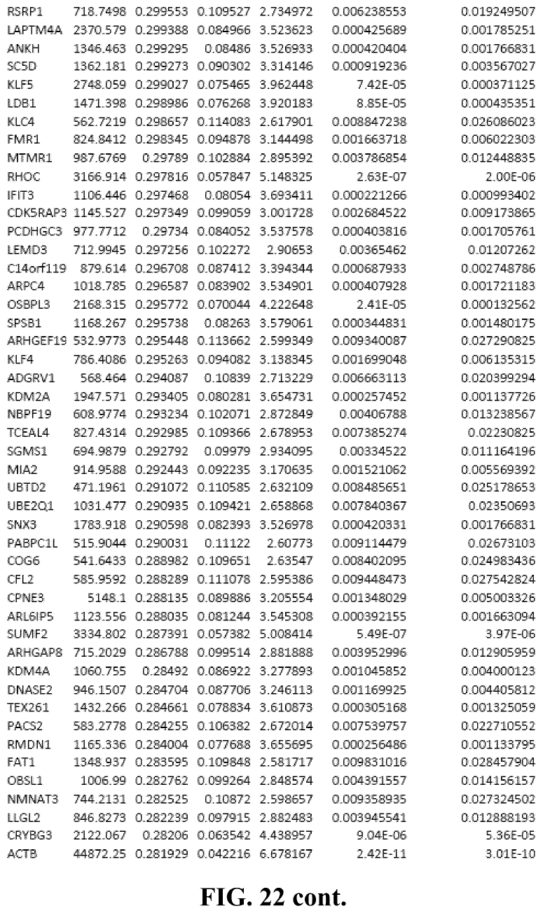

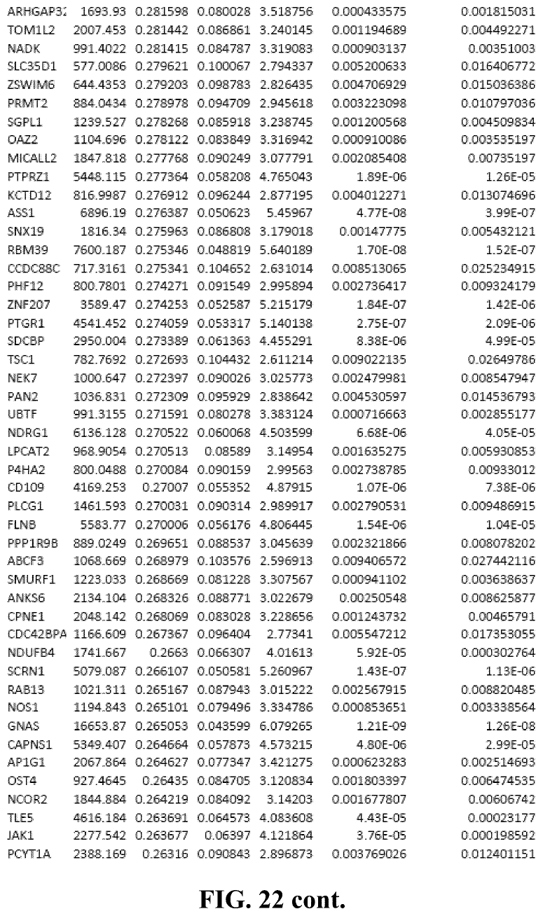

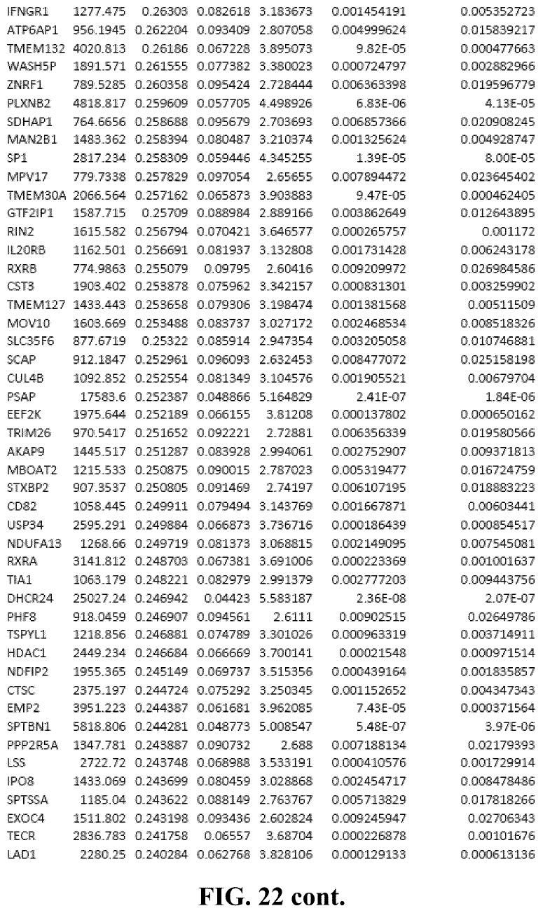

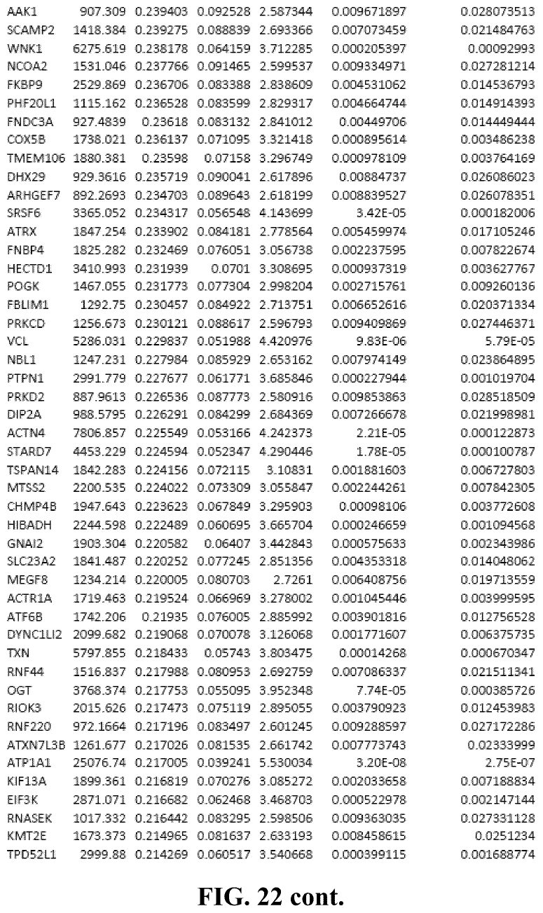

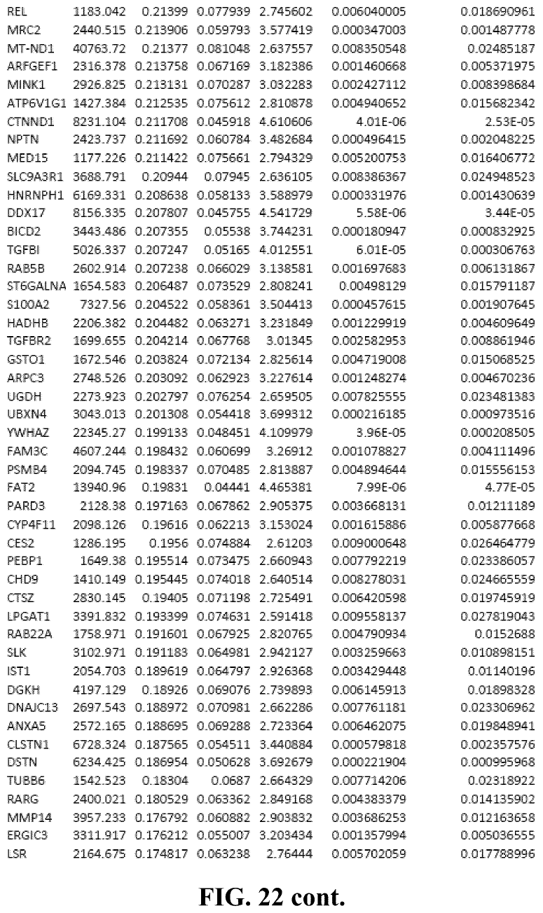

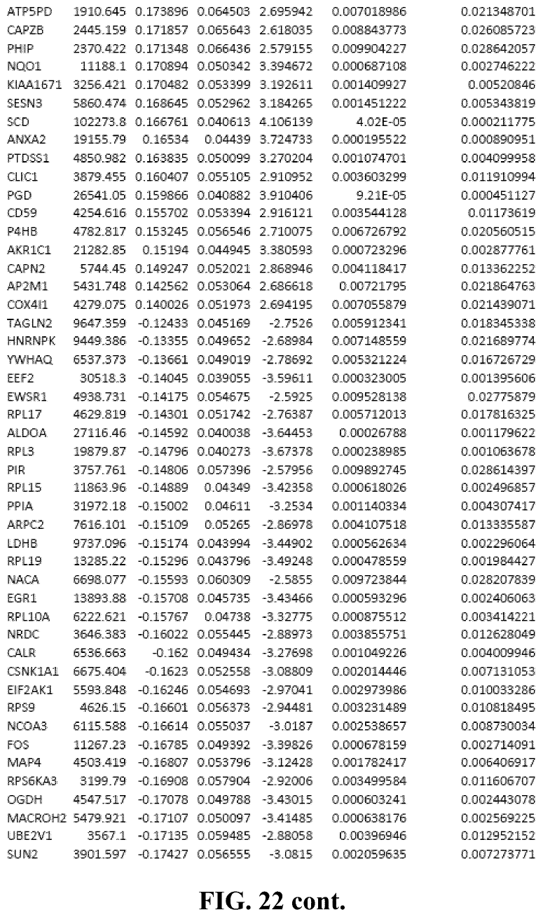

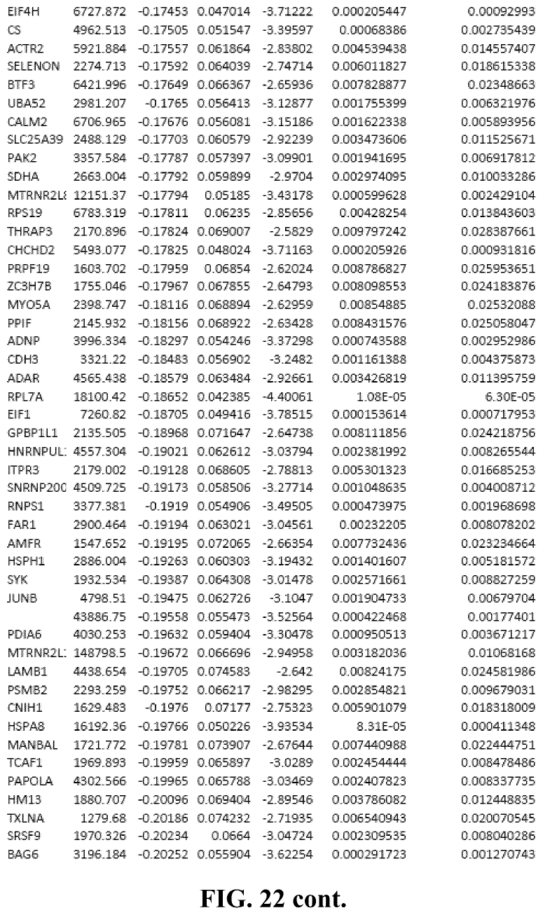

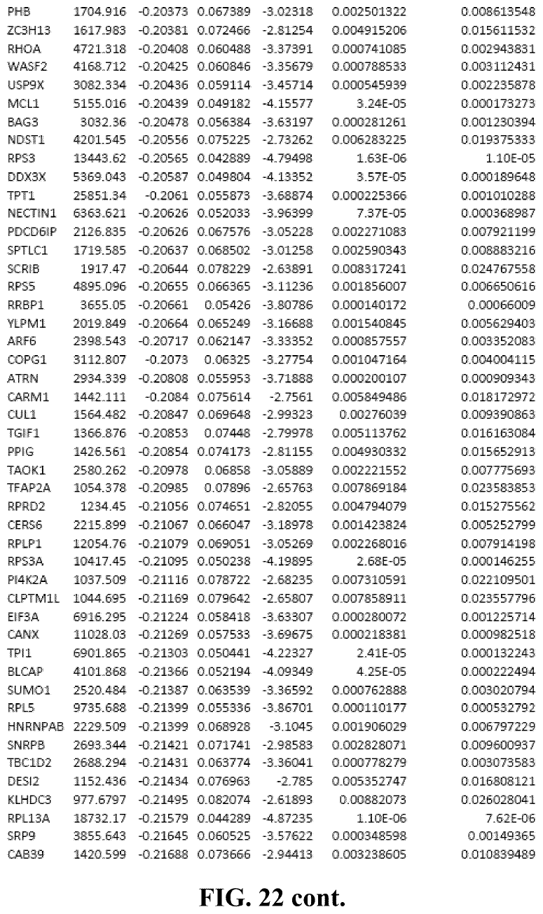

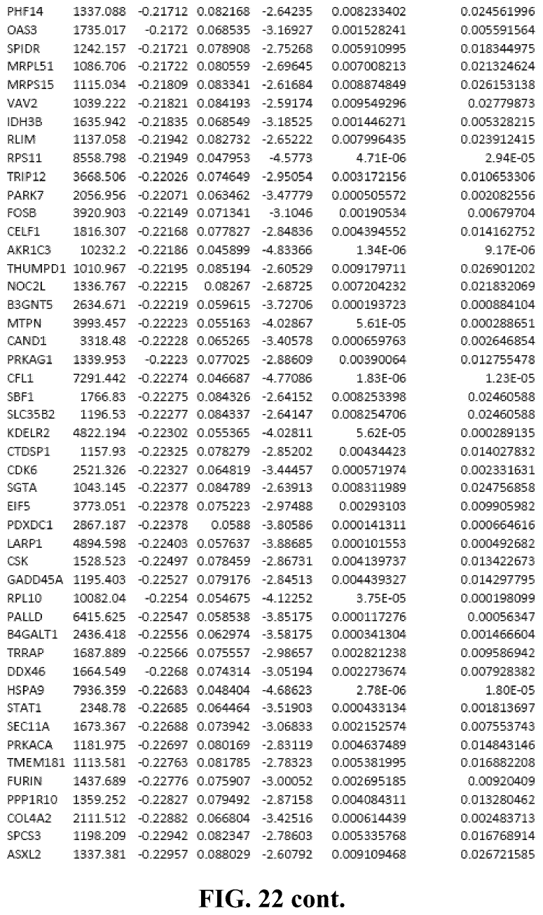

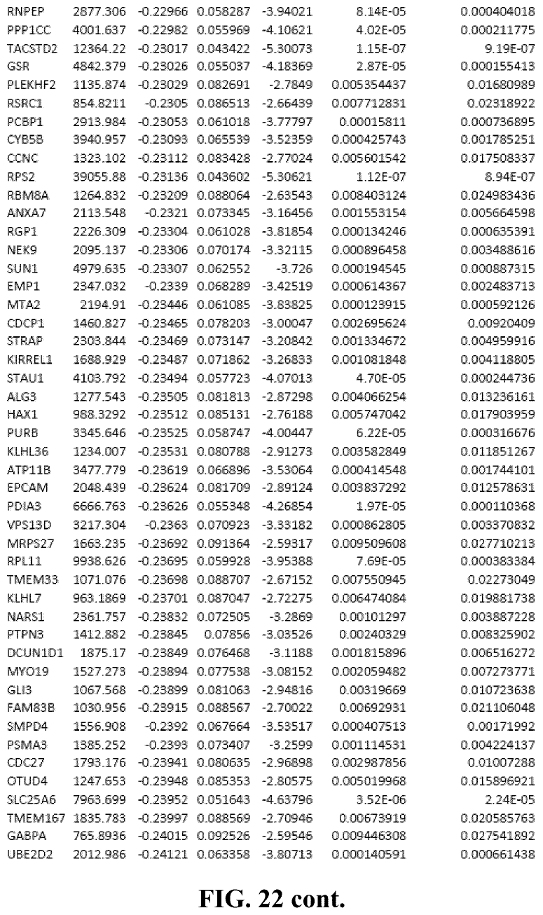

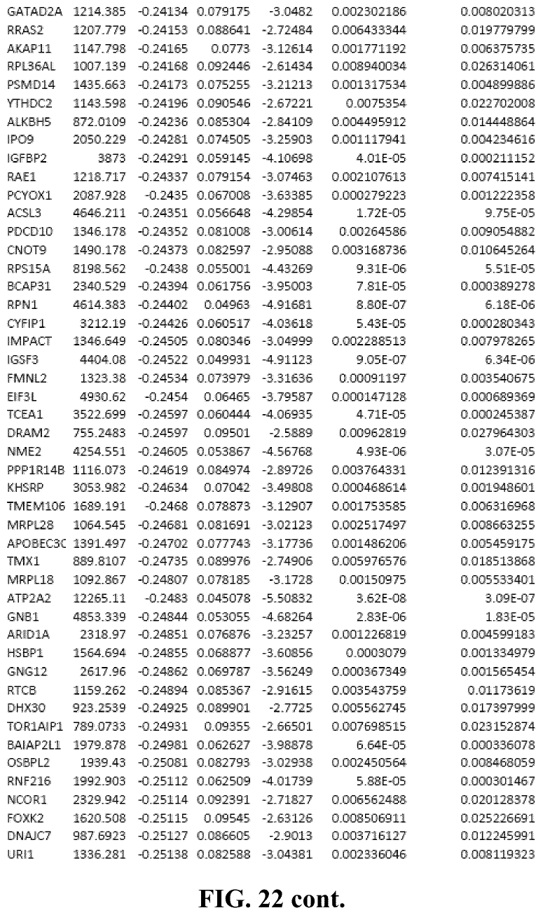

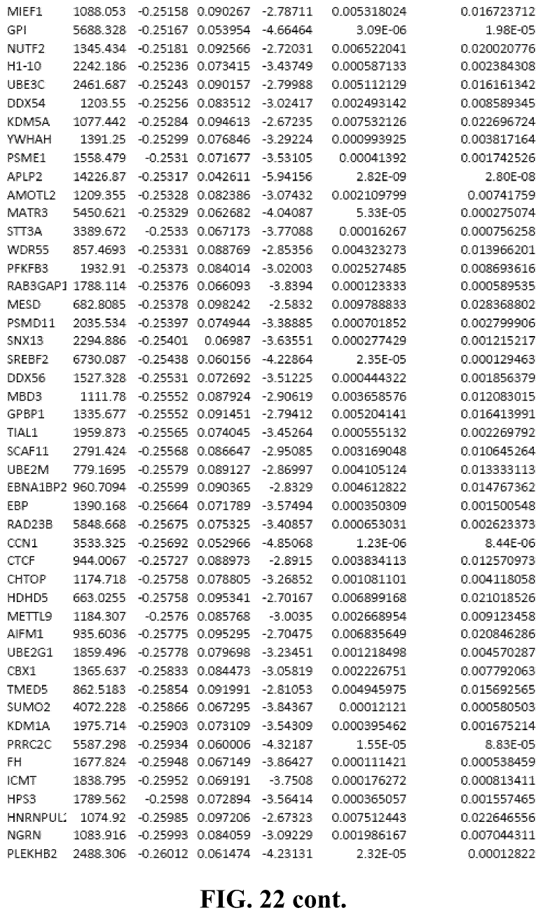

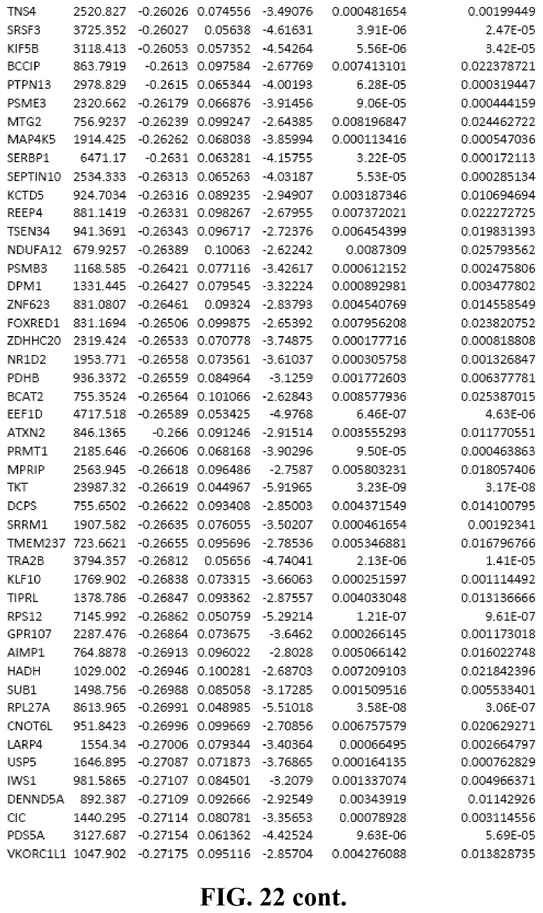

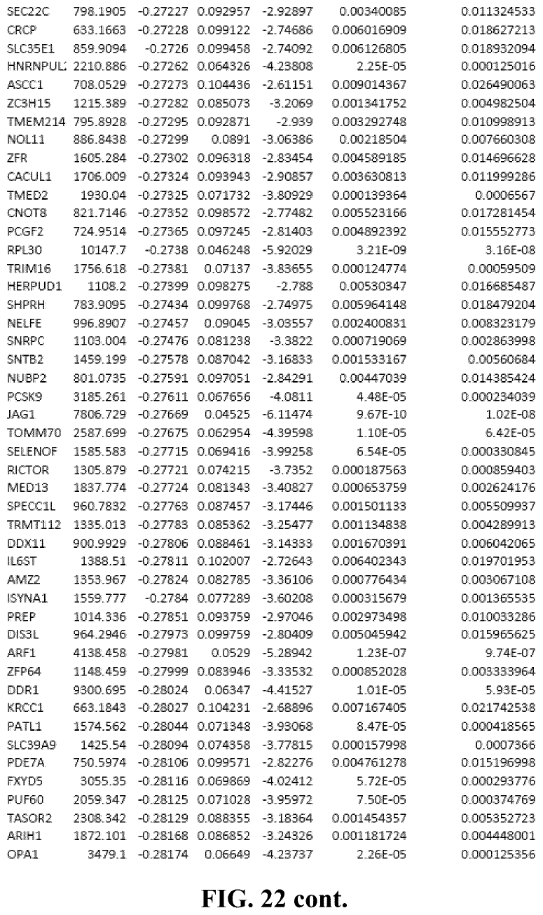

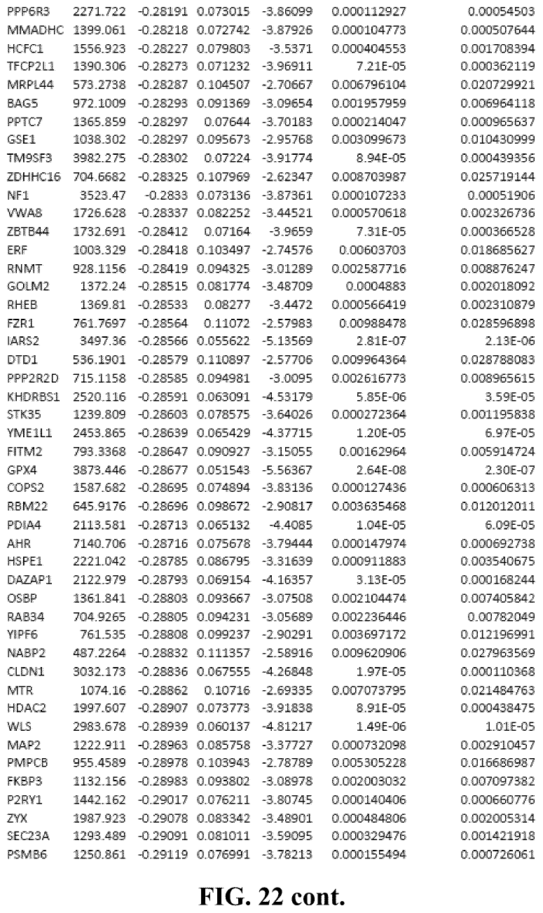

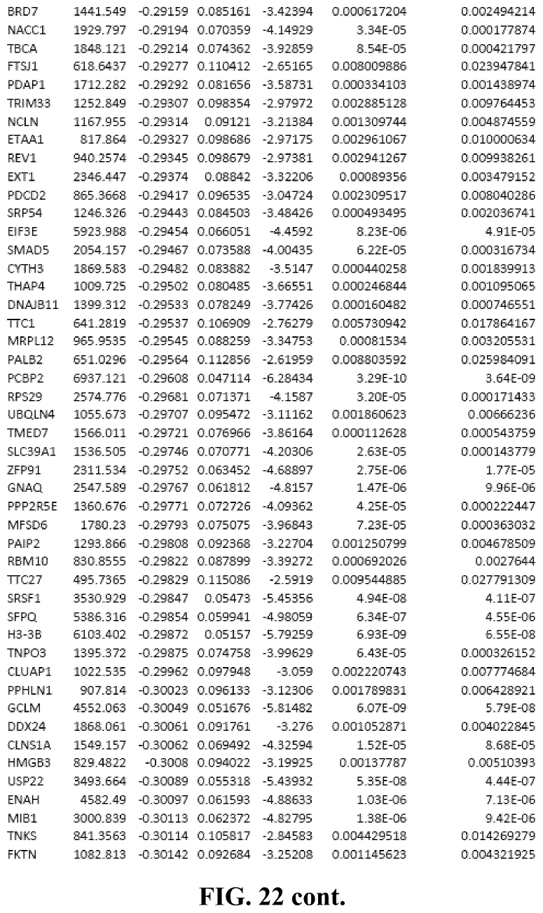

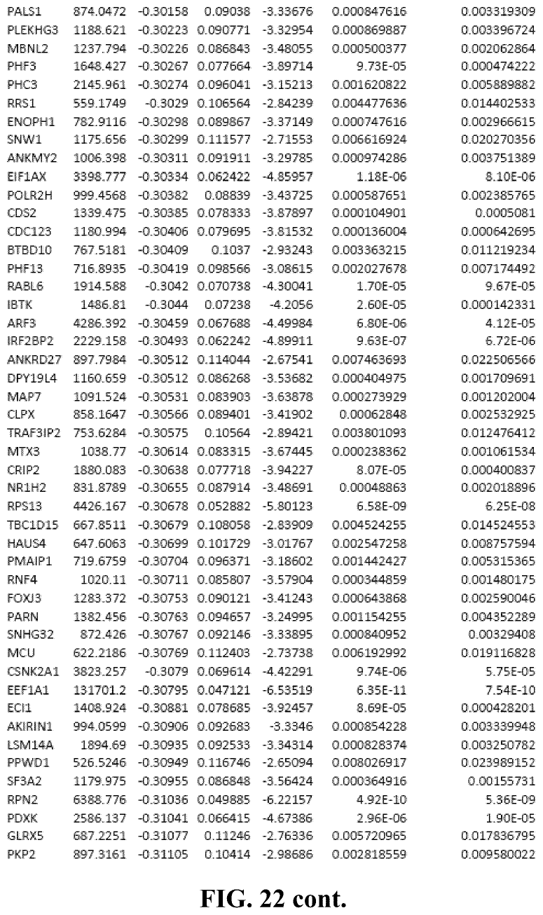

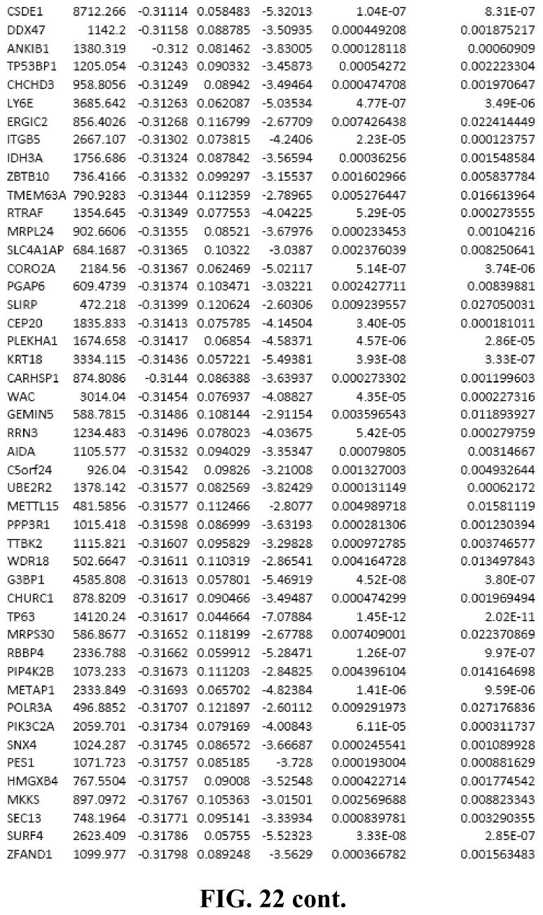

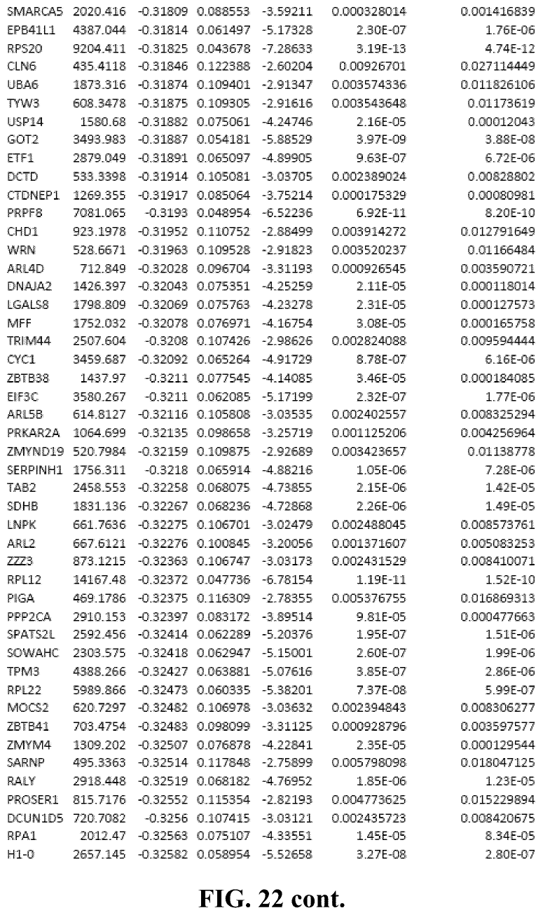

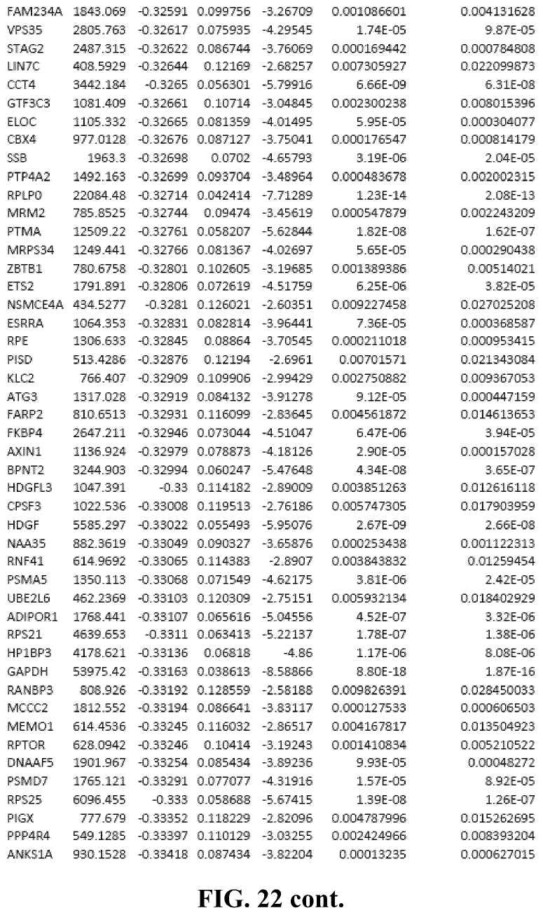

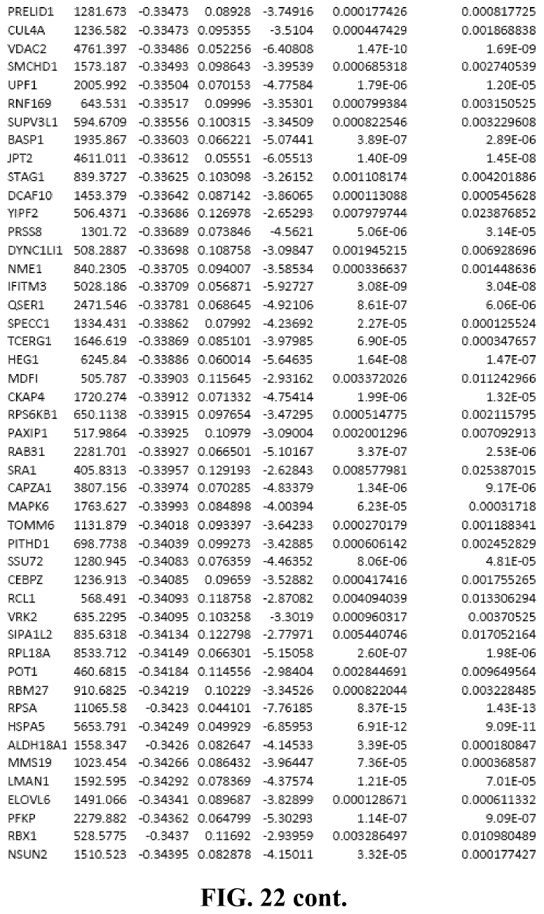

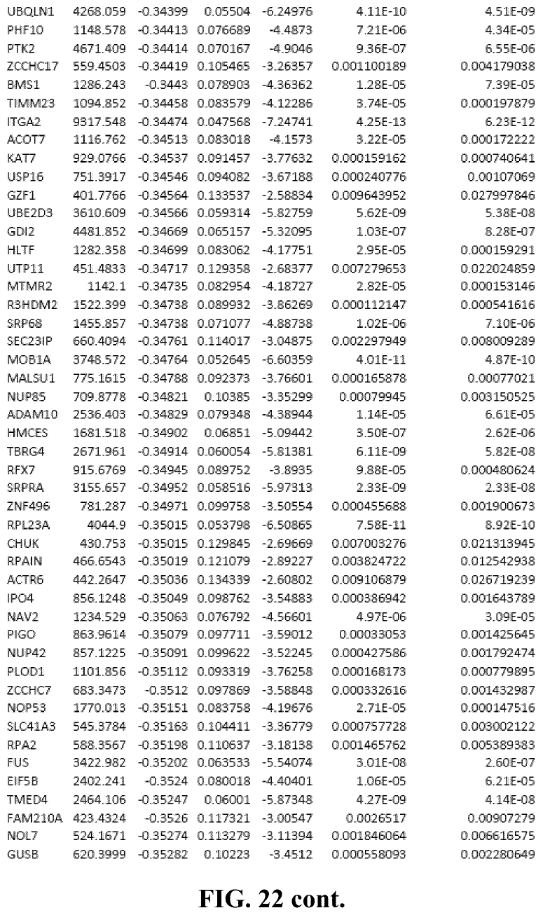

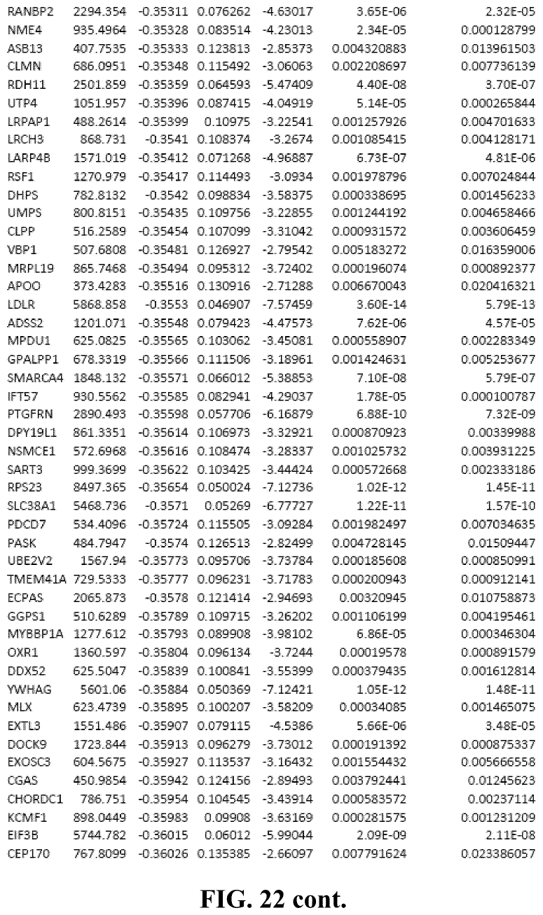

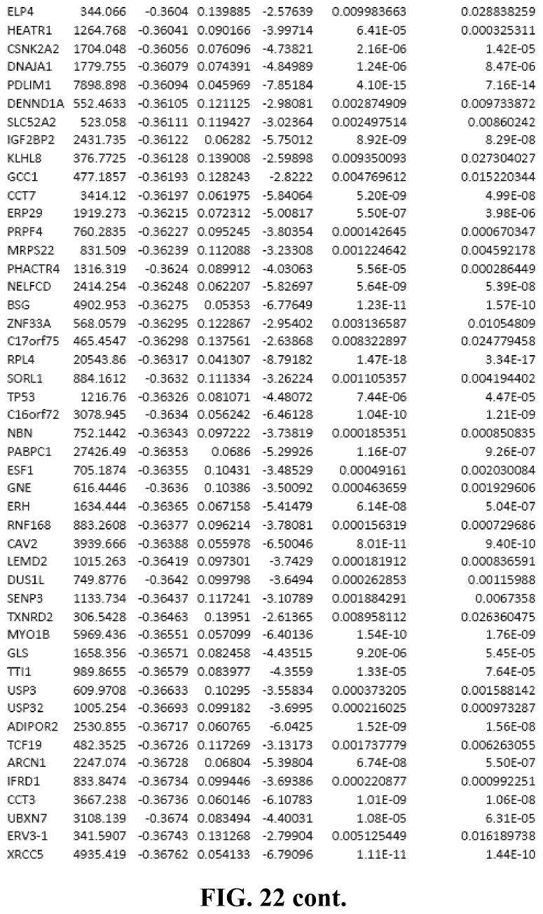

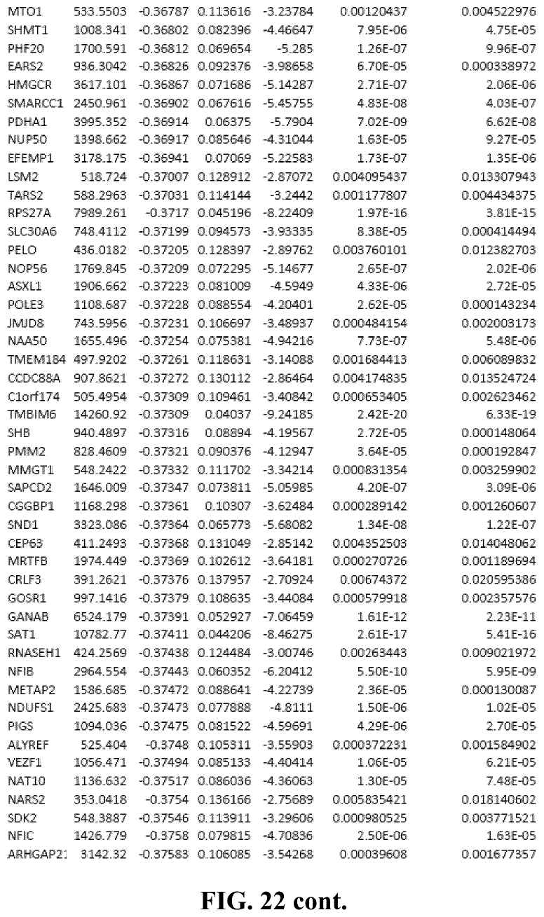

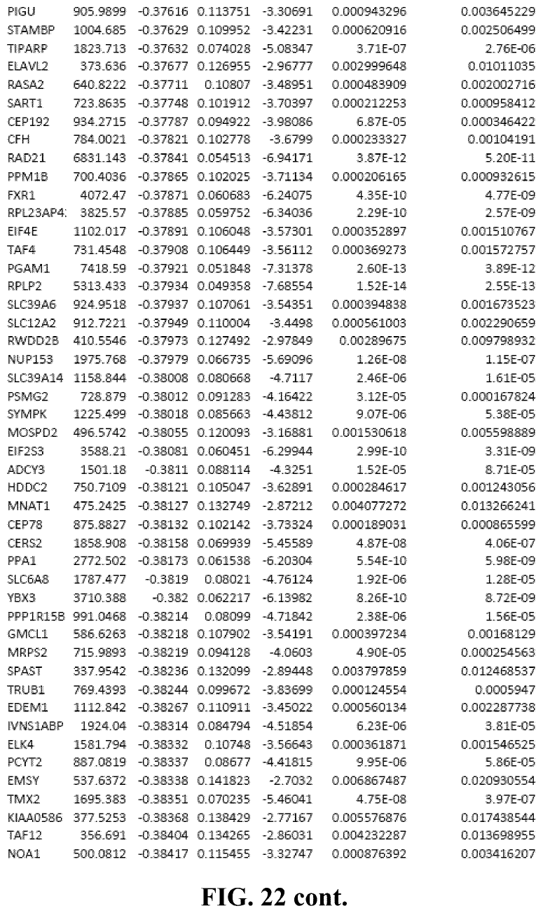

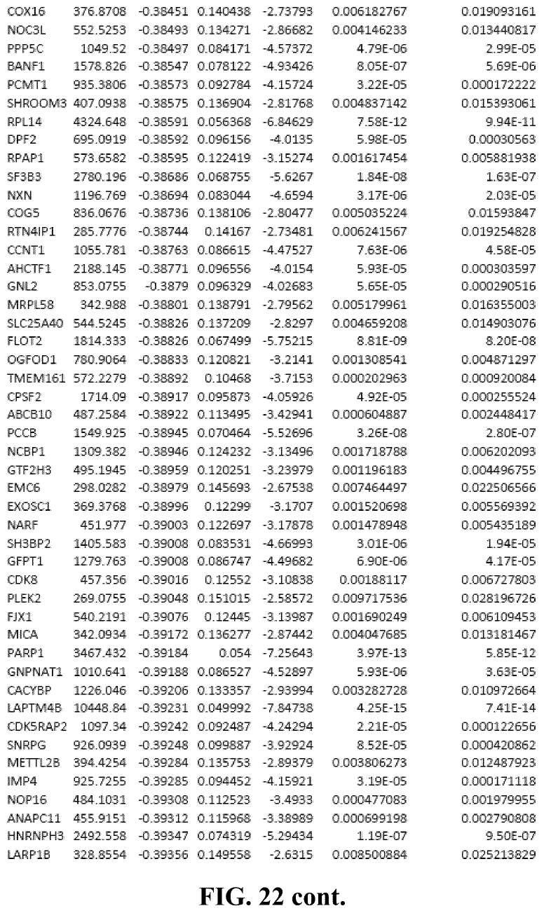

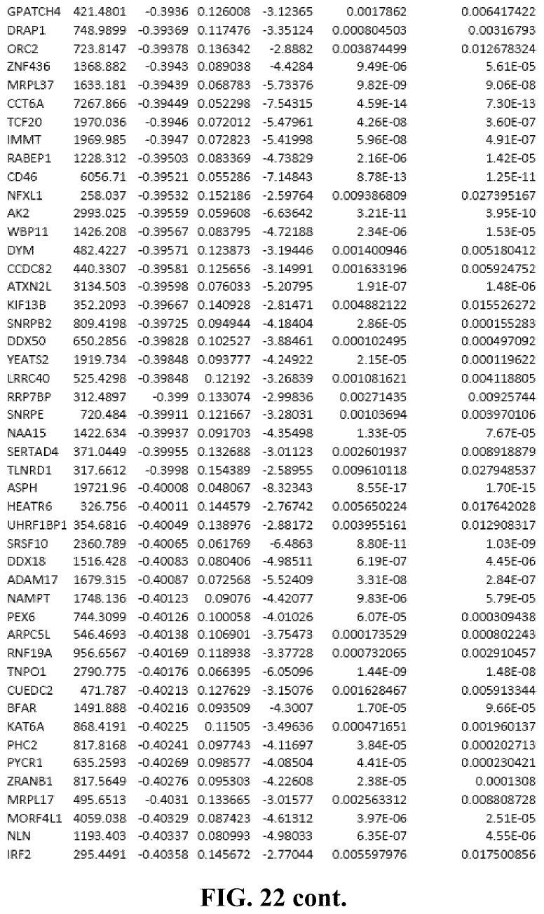

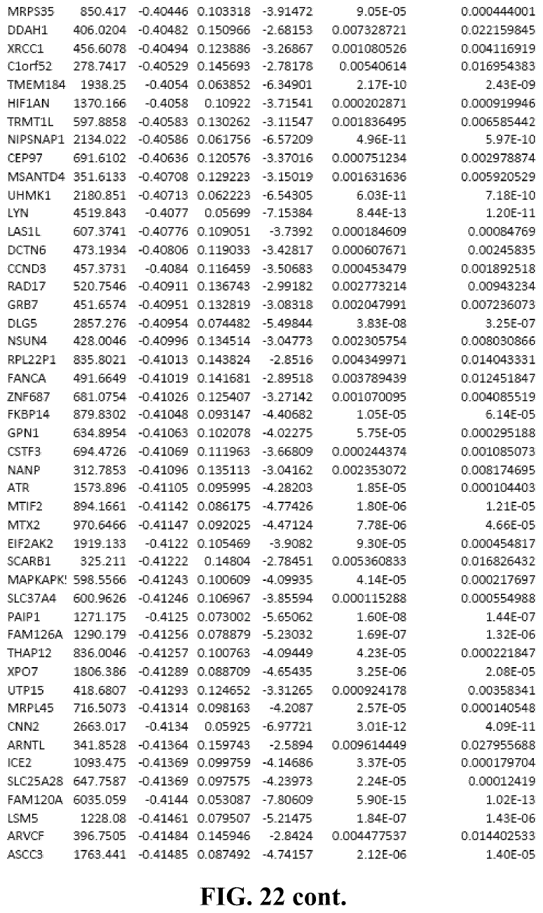

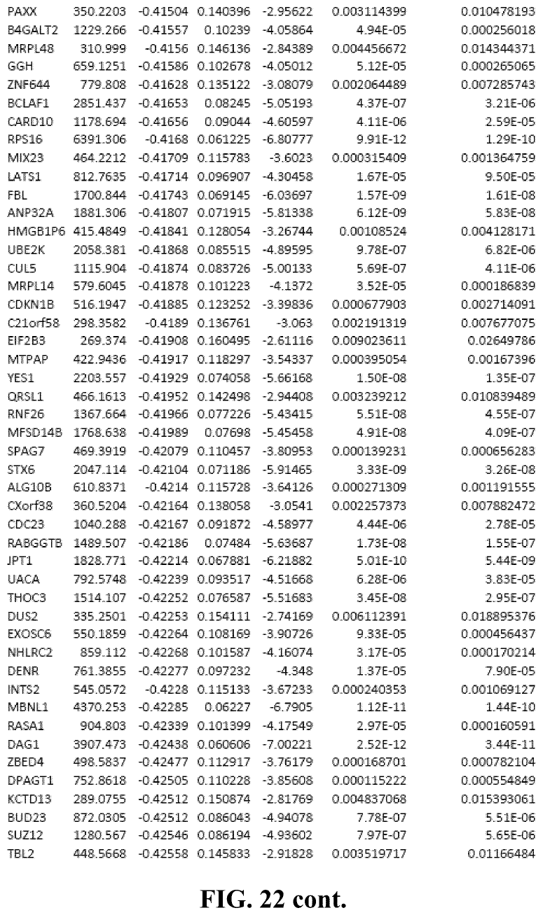

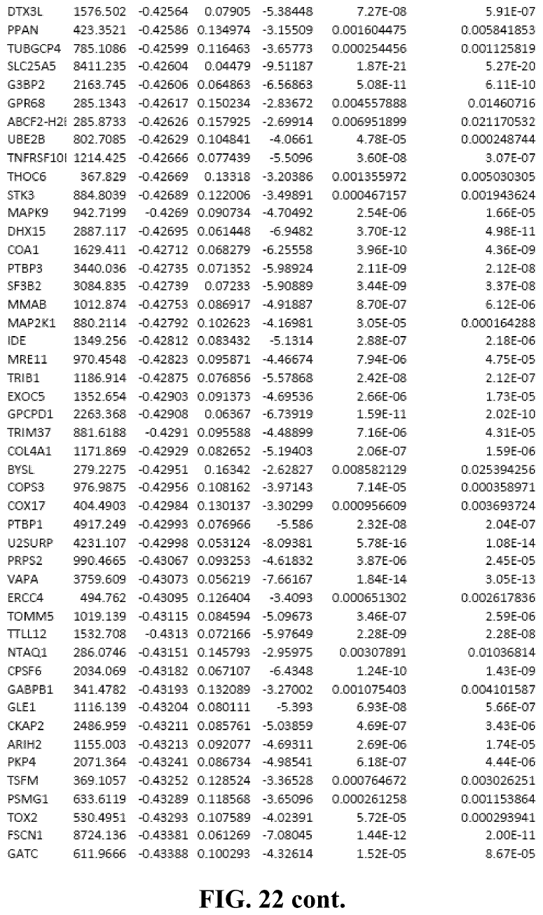

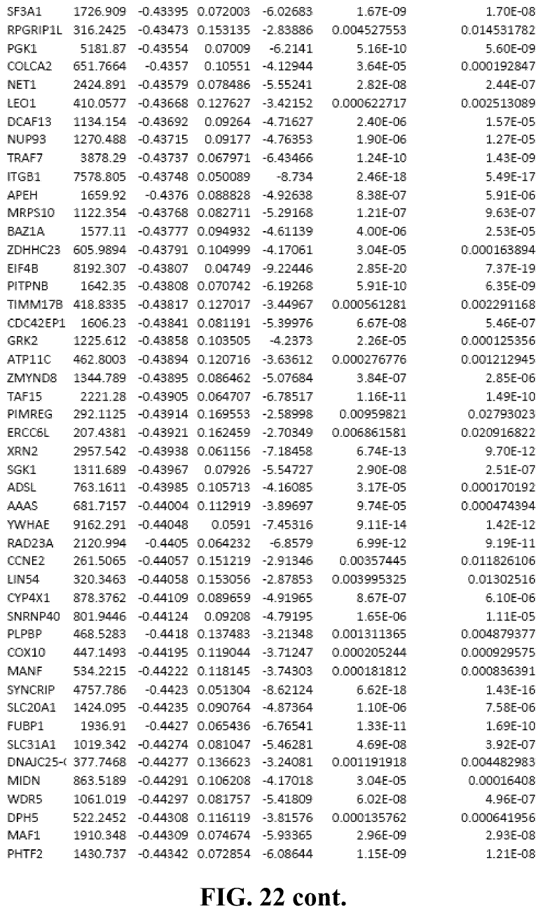

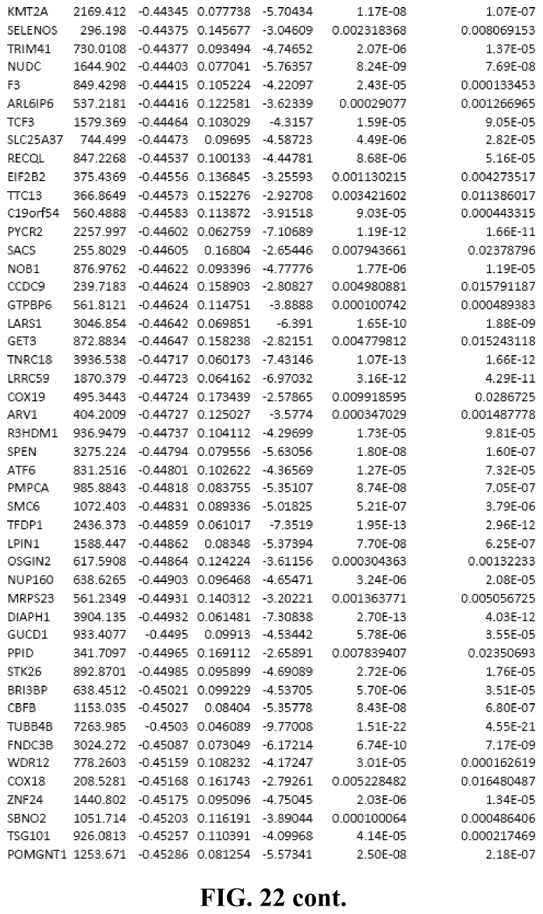

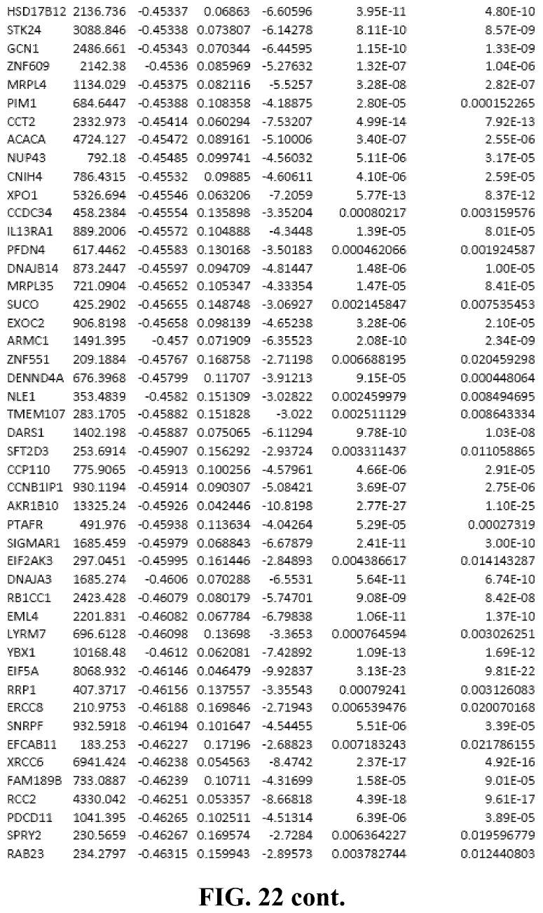

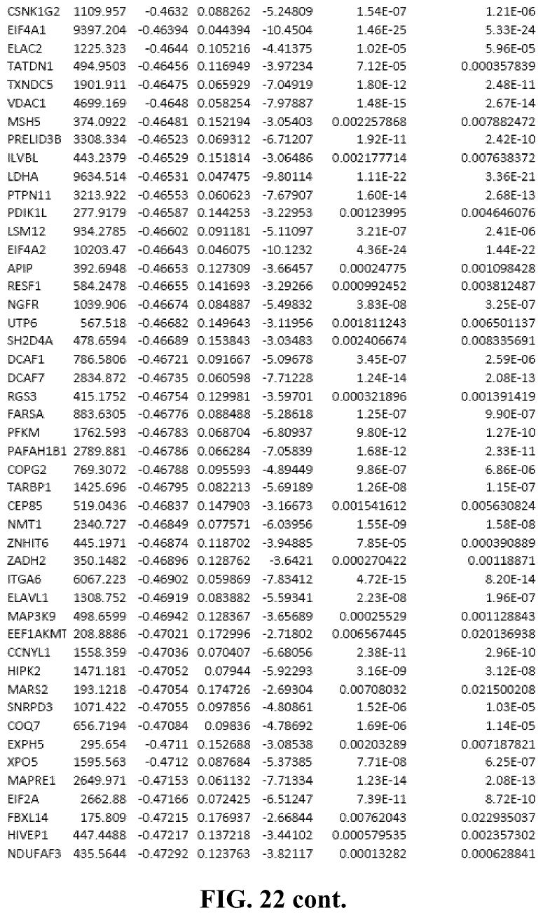

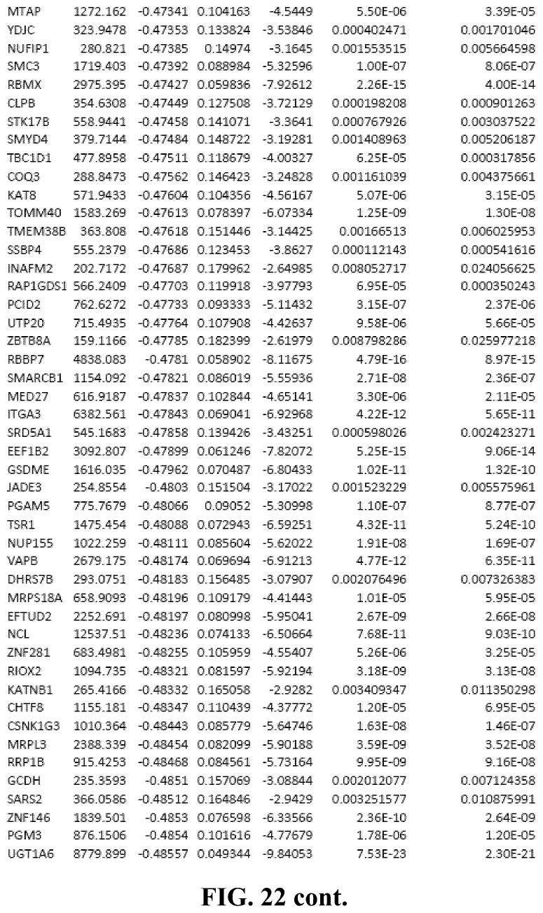

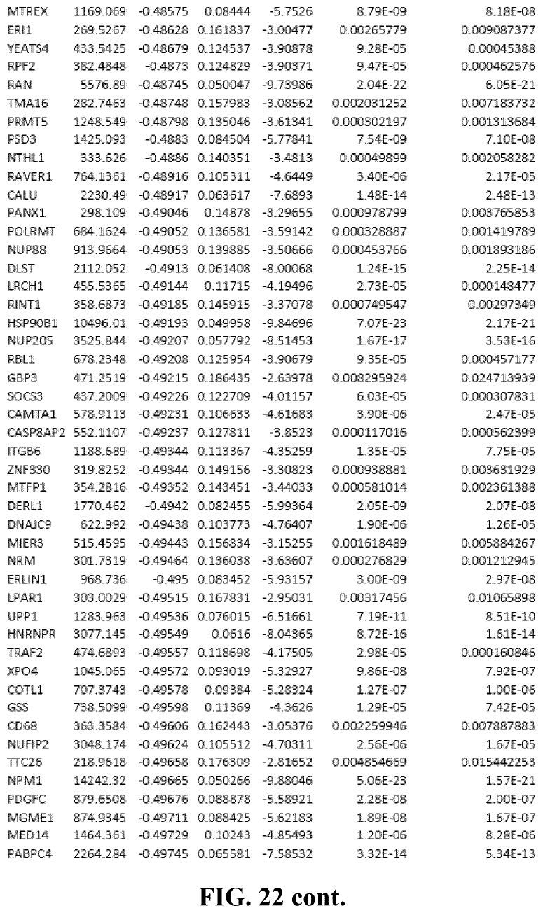

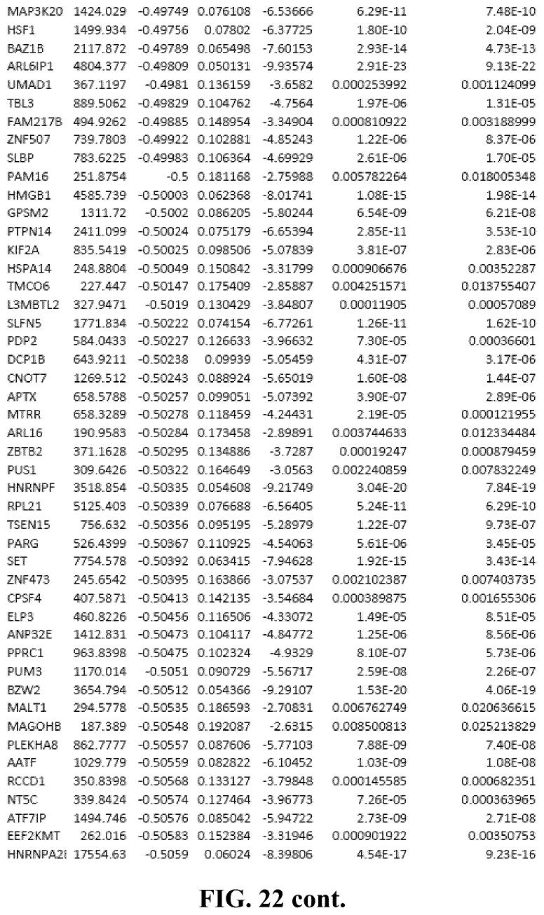

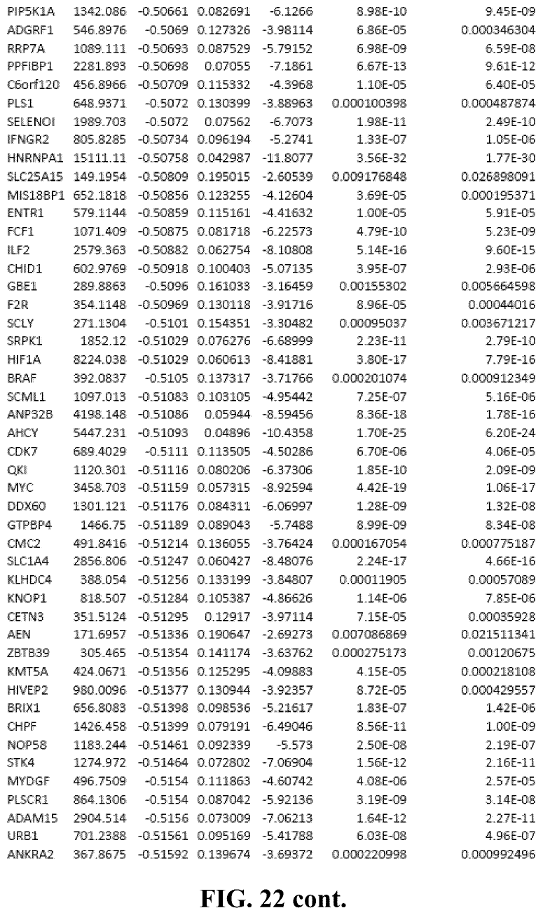

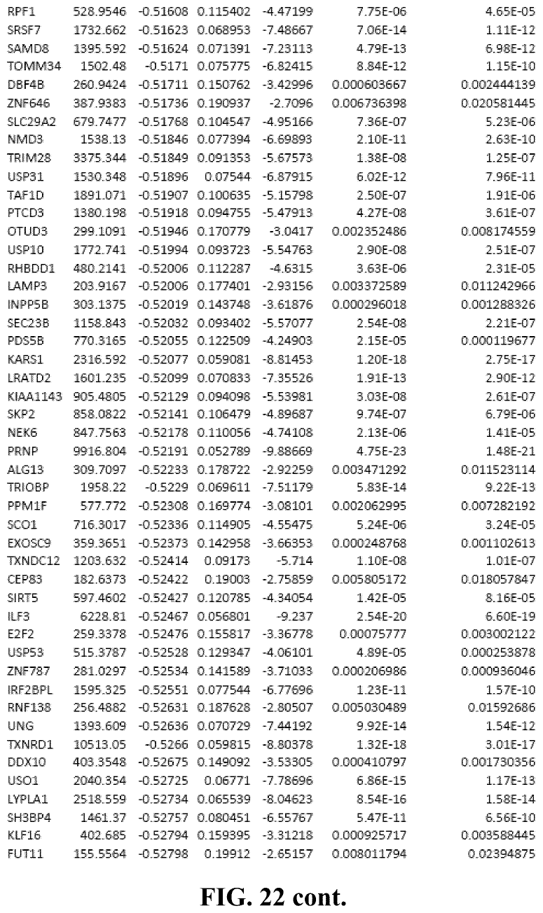

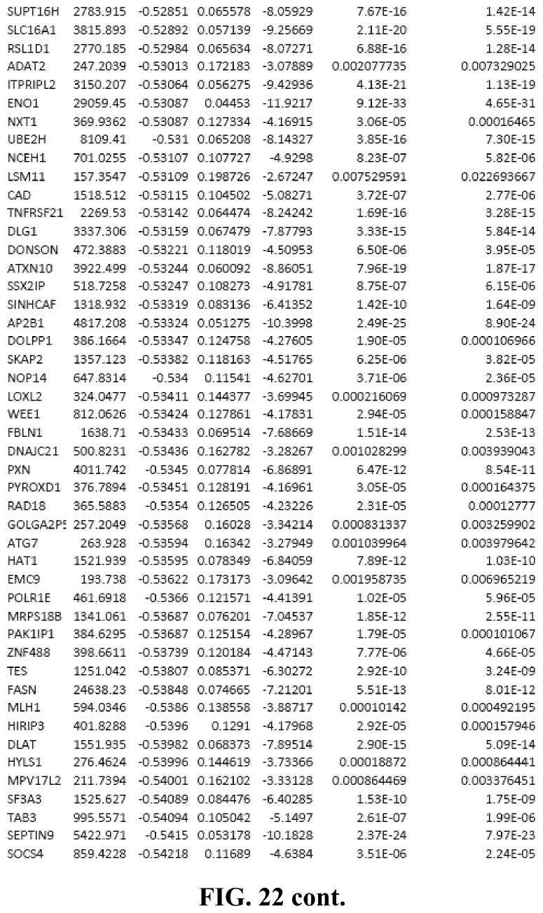

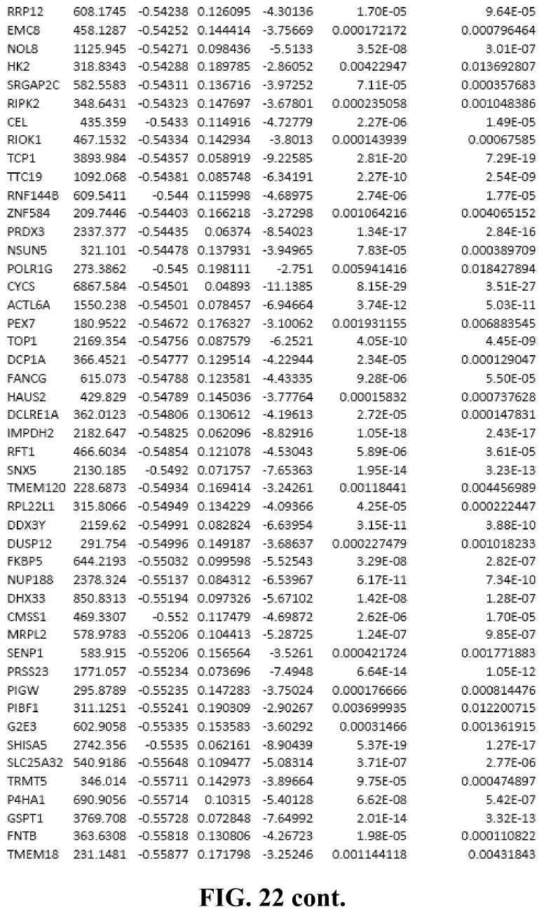

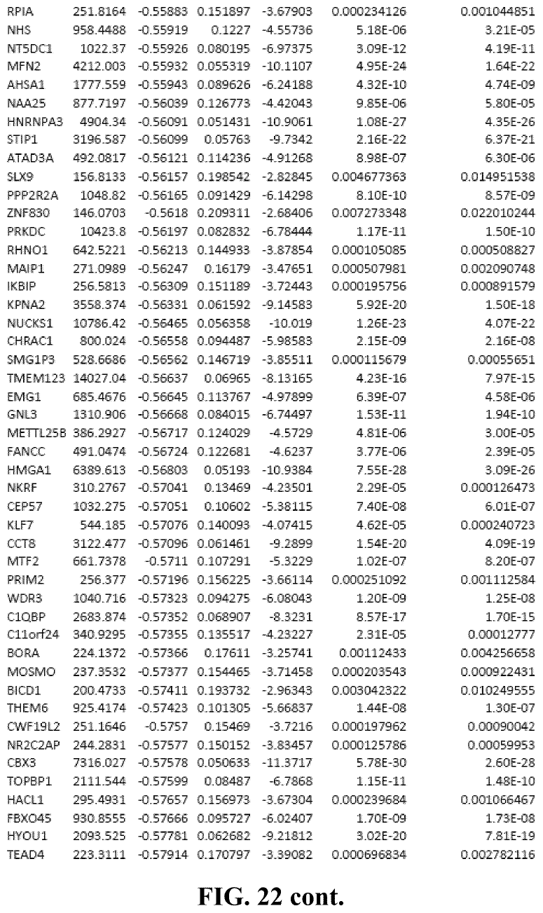

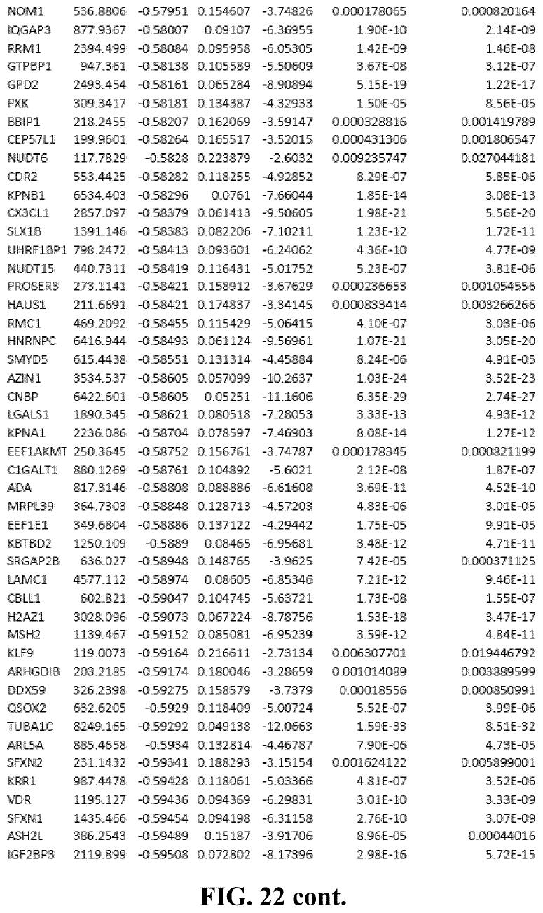

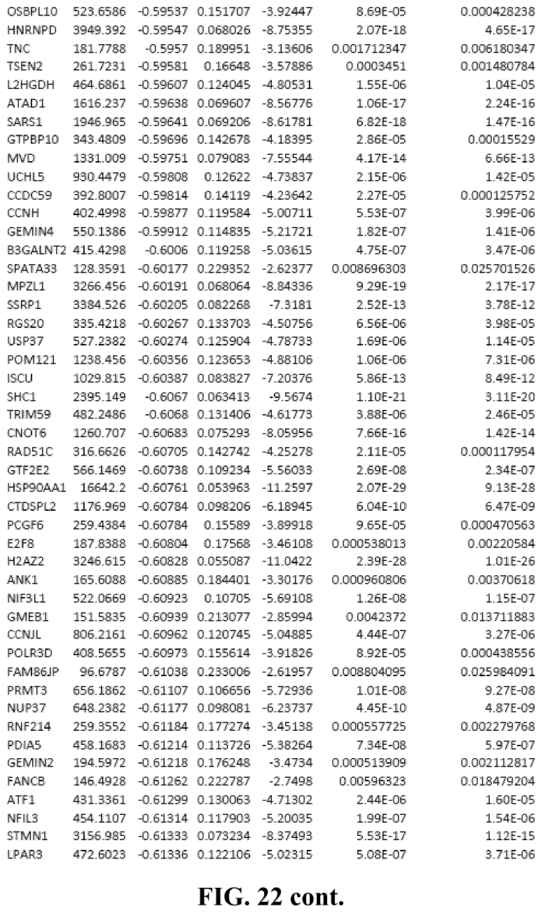

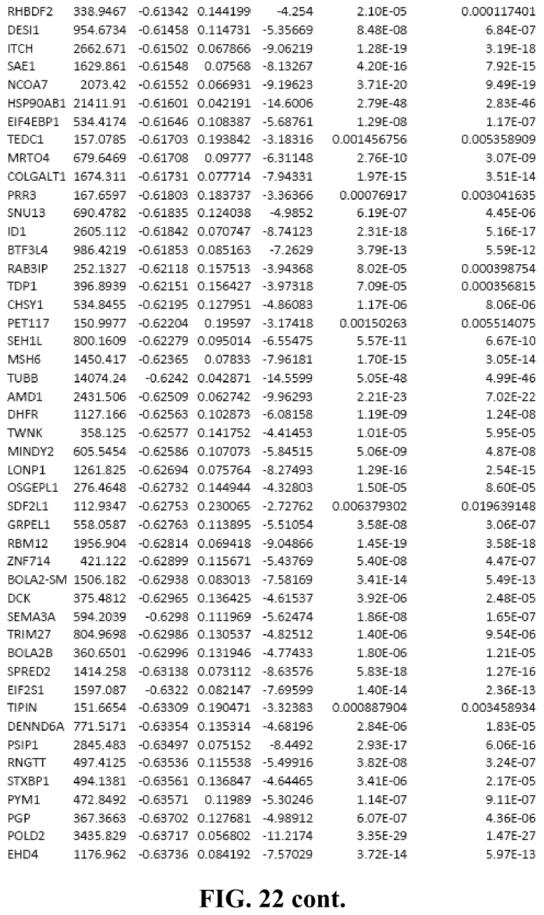

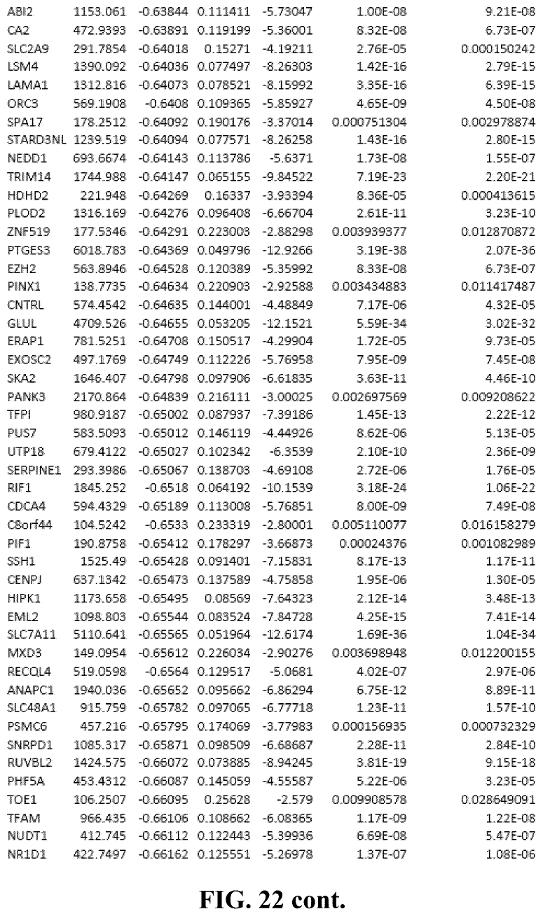

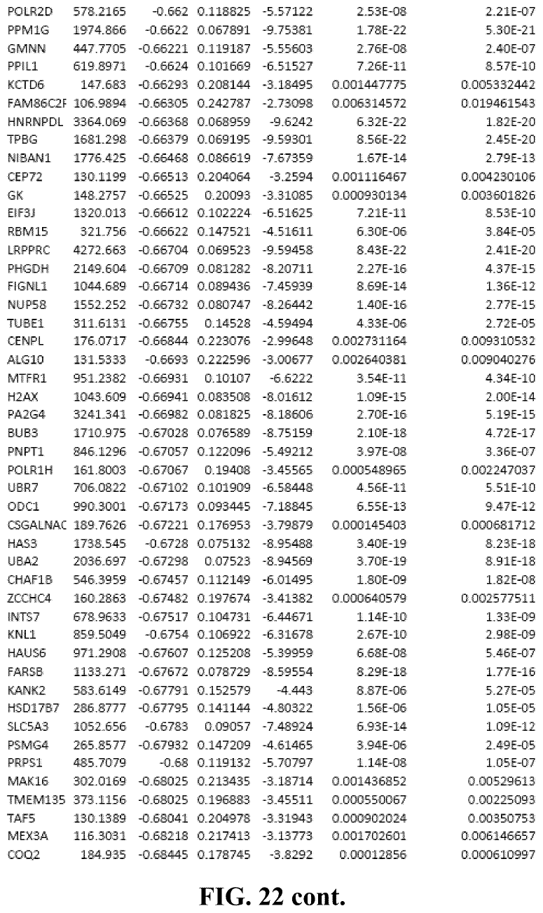

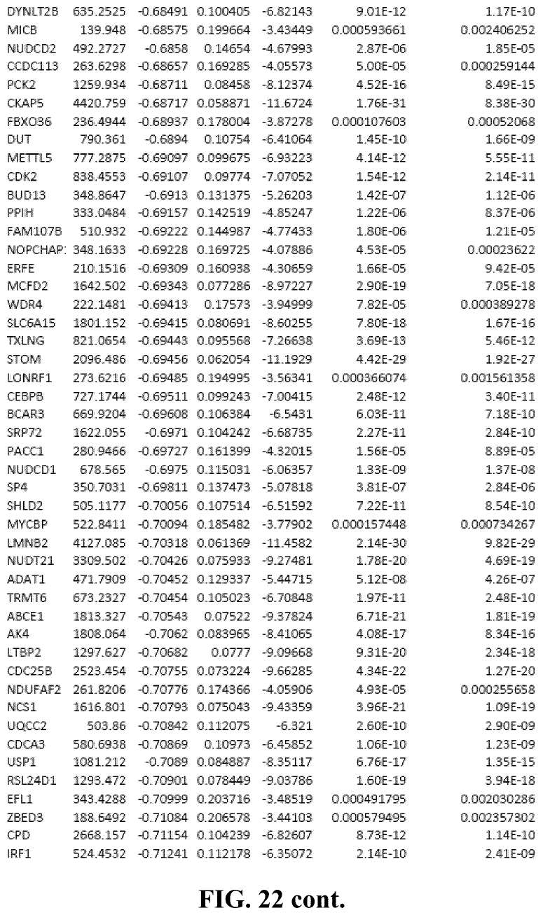

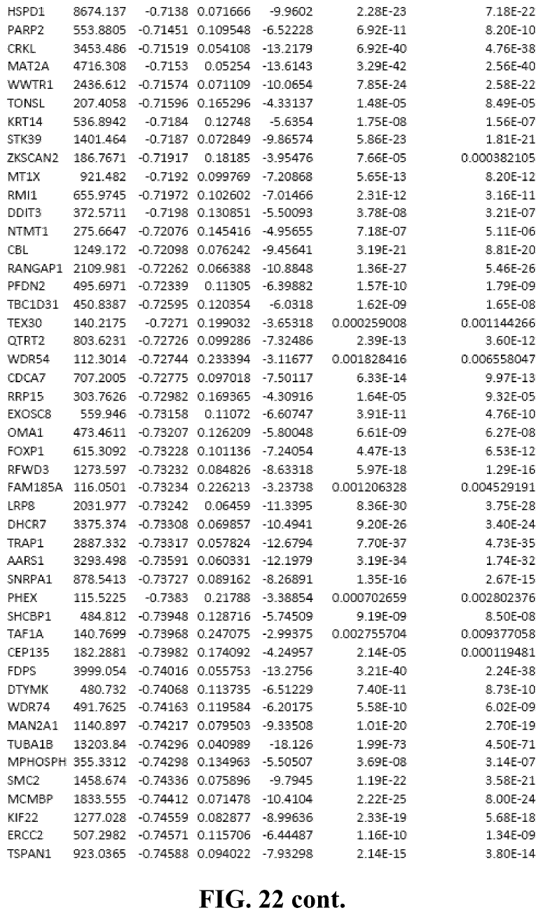

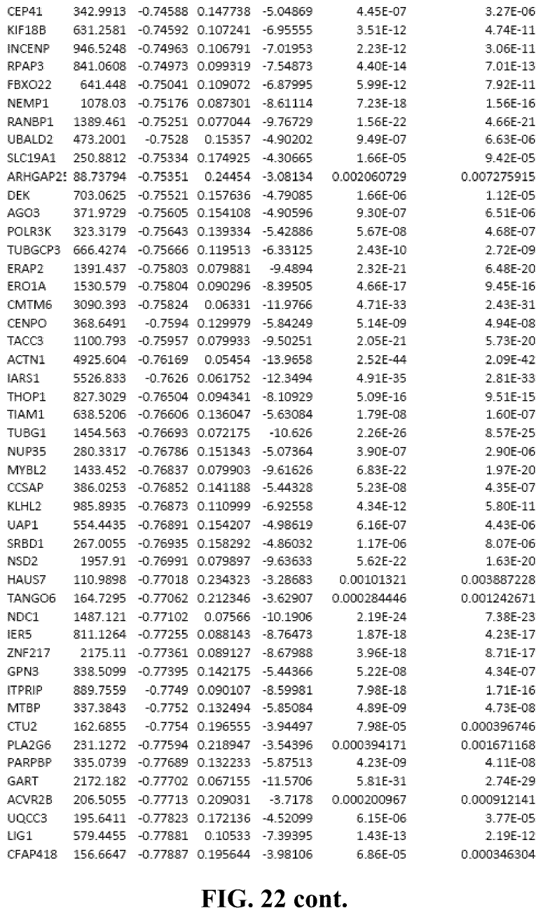

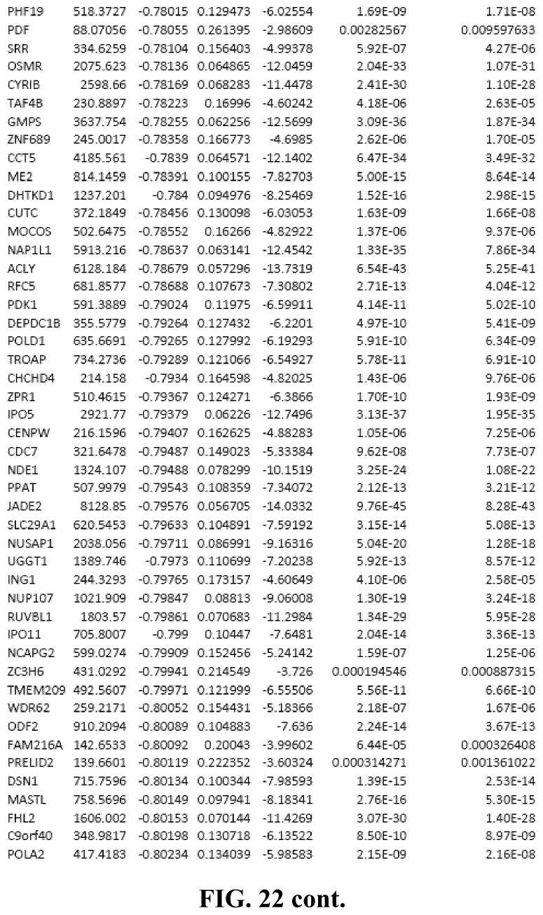

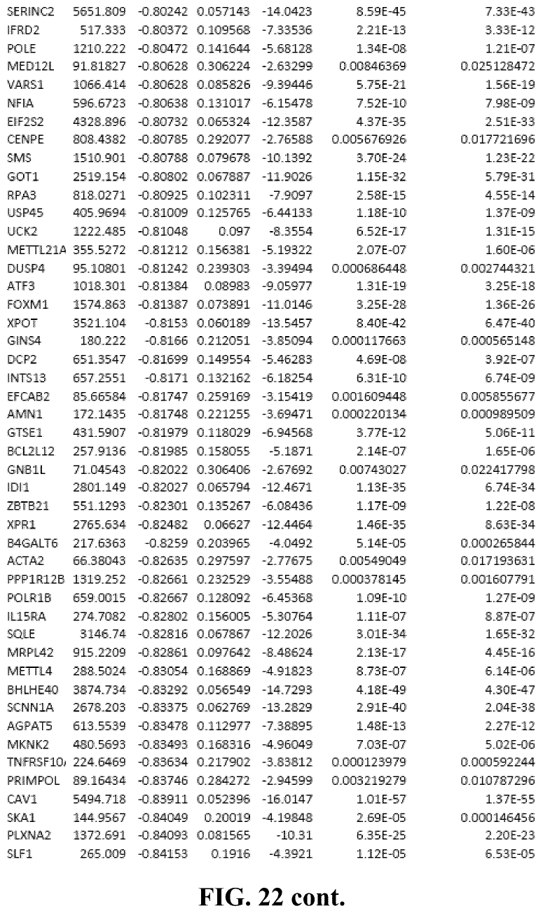

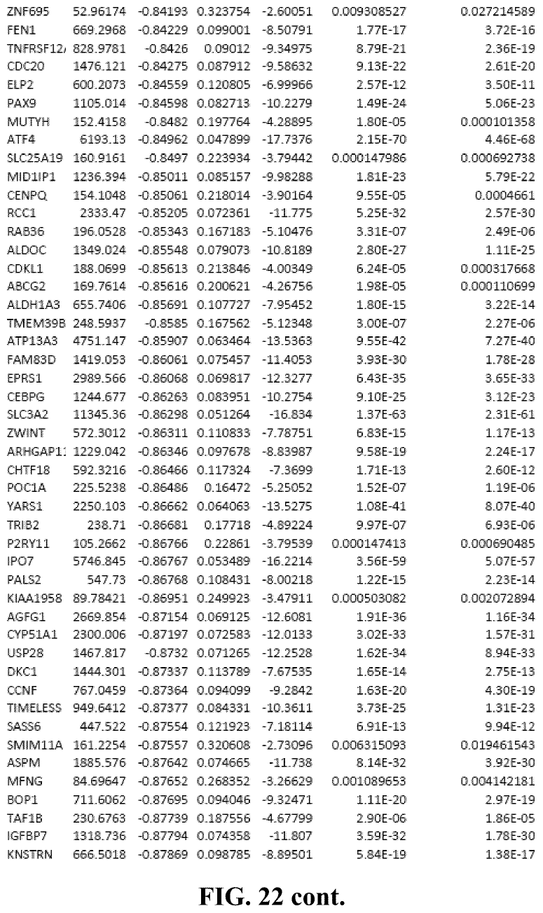

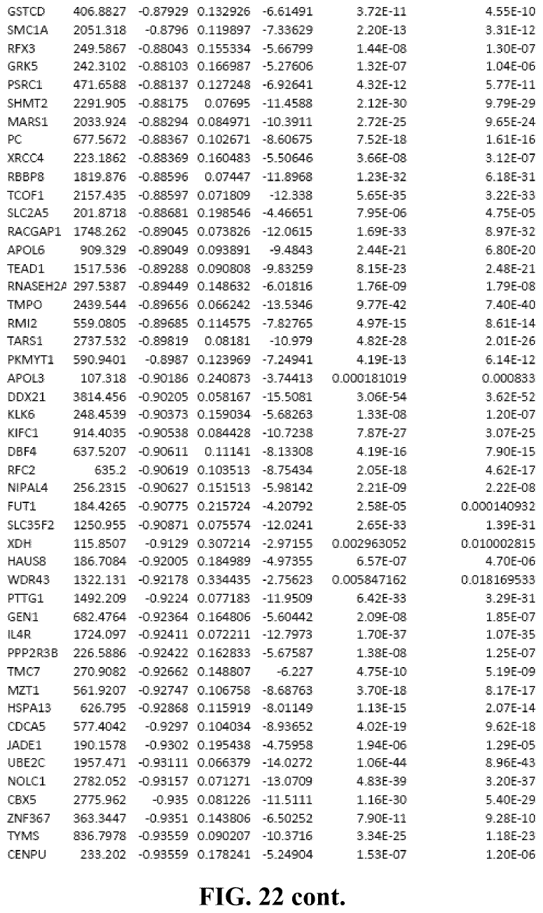

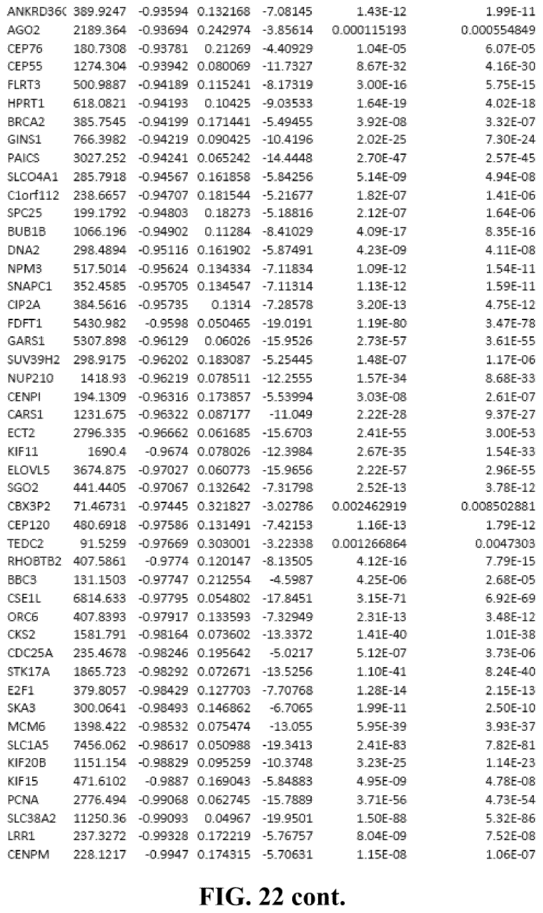

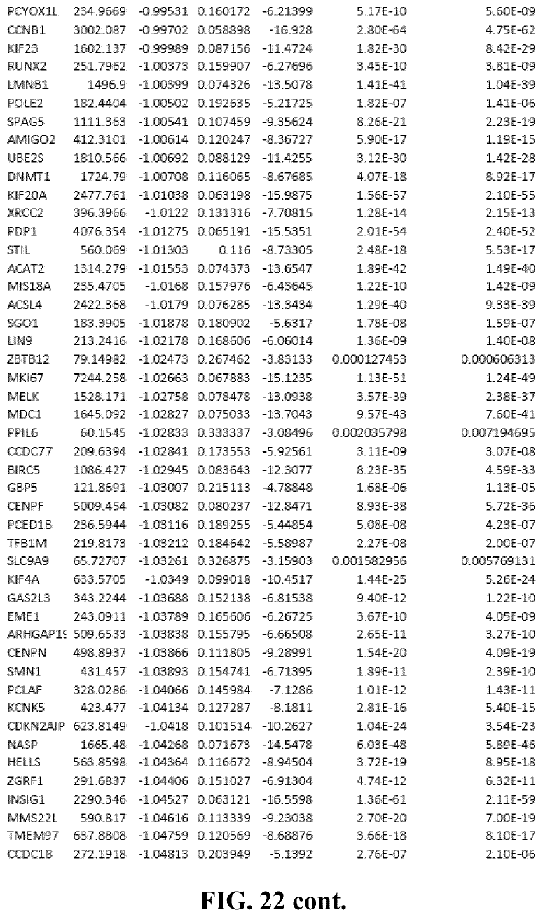

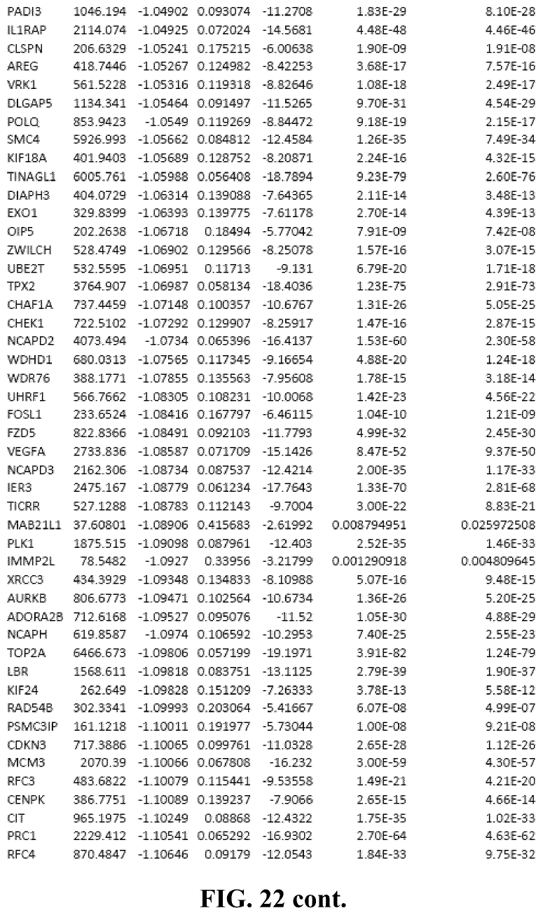

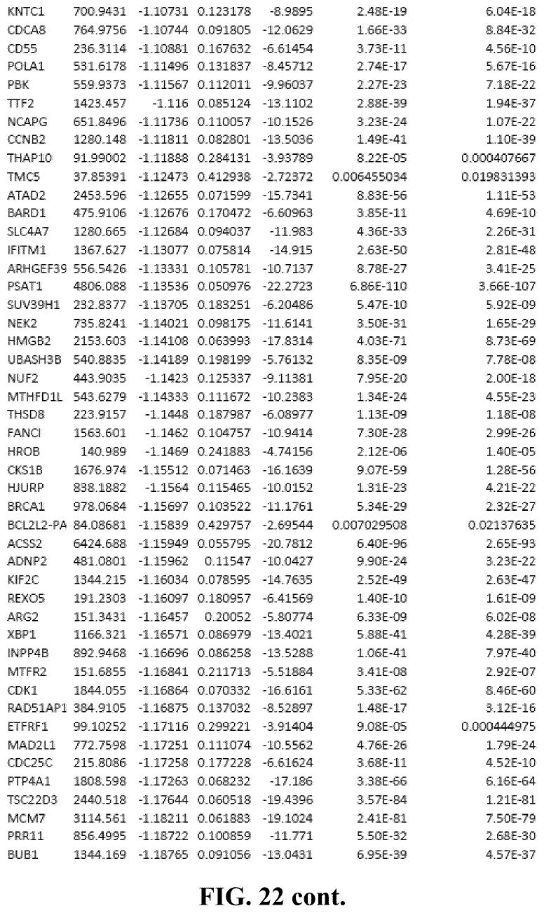

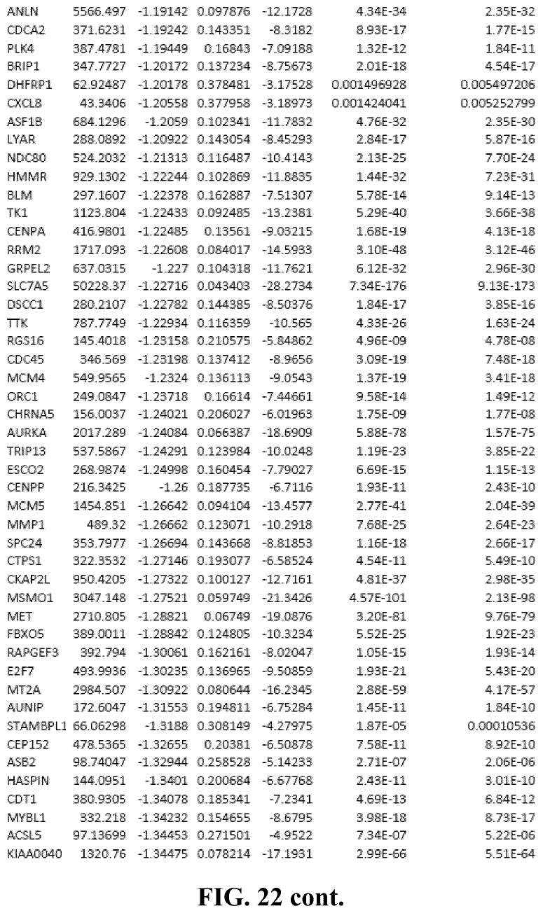

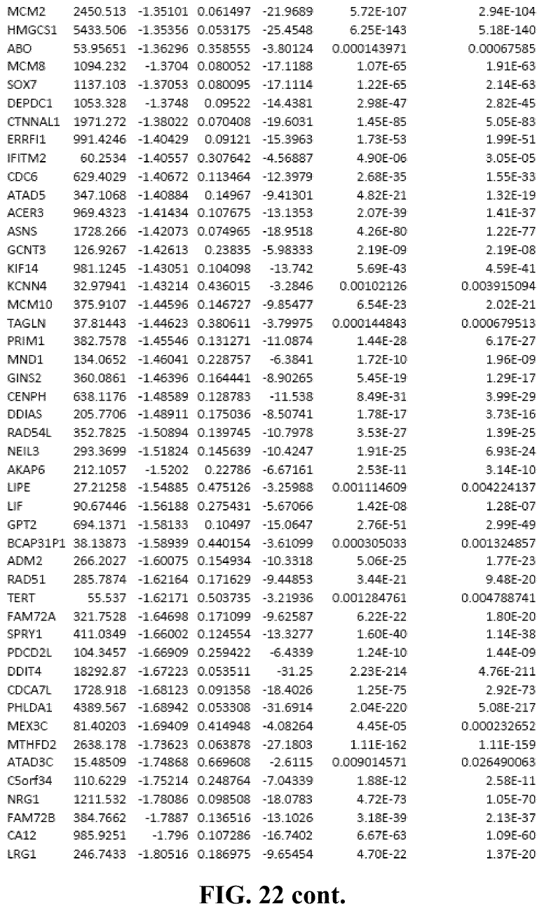

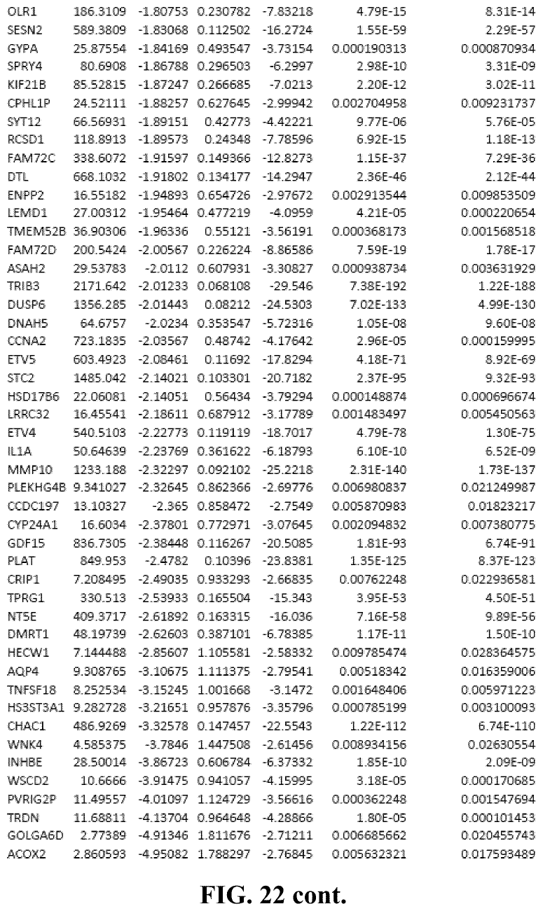

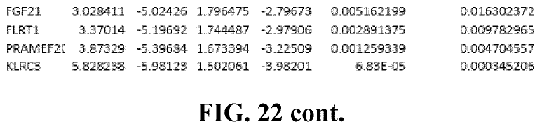

Transcription factor FOXM1 is a master regulator of the cell cycle. It tightly controls the cell cycle progression and maintains sustained cell proliferation via its various downstream target genes crucial for cell cycle regulation 16. To explore the functional consequences of PU7-1-mediated FOXM1 degradation, a transcriptome analysis was performed. CAL33 cells were treated with or without 10 μM of PU7-1 for 96 h and total RNA were extracted for RNAseq analysis. We identified ˜5000 differentially expressed genes (DEGs) in DMSO-treated versus PU7-1-treated cells (FIG. 22). Of note, gene ontology analysis of the DEGs revealed that the FOXM1 pathway was one of the top hits repressed by PU7-1 (FIG. 4F). Gene expression profiles demonstrated that FOXM1 downstream target genes were significantly downregulated upon PU7-1 treatment (FIG. 4G). This effect was further validated in CAL33 cells by quantitative real-time PCR (FIG. 4H). These data suggest that PU7-1 is highly effective in suppressing the transcriptional activities of FOXM1. Consistent with this observation, PU7-1-treated CAL33 cells exhibited a significantly lower percentage of the BrdU incorporation, an indicator of DNA replication and cell proliferation, indicating an impaired cell cycle progression (FIGS. 12A and 12B). Notably, colony formation assay revealed that PU7-1 induced marked growth inhibition in several human cancer cell lines including CAL33, BT474 (breast cancer), DU145 (prostate cancer) and SK-N-DZ (brain cancer) (FIGS. 12C and 12D). Collectively, these data imply that the USP7 degrader PU7-1 can effectively repress FOXM1 transcription network and induce dramatic inhibition of cell proliferation.

### PU7-1 Mediates FOXM1 Degradation, Cell Proliferation Inhibition and Tumor Suppression in TNBC

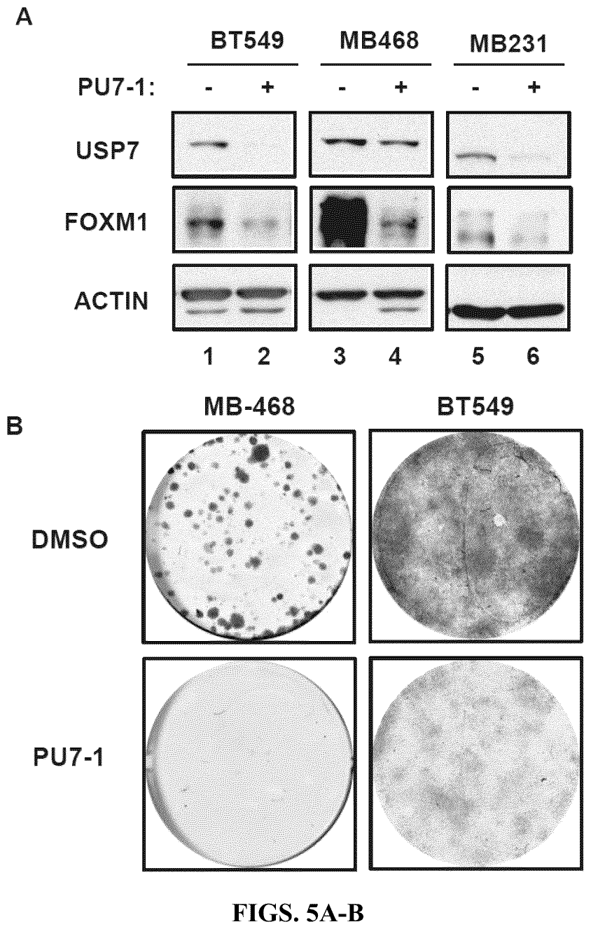

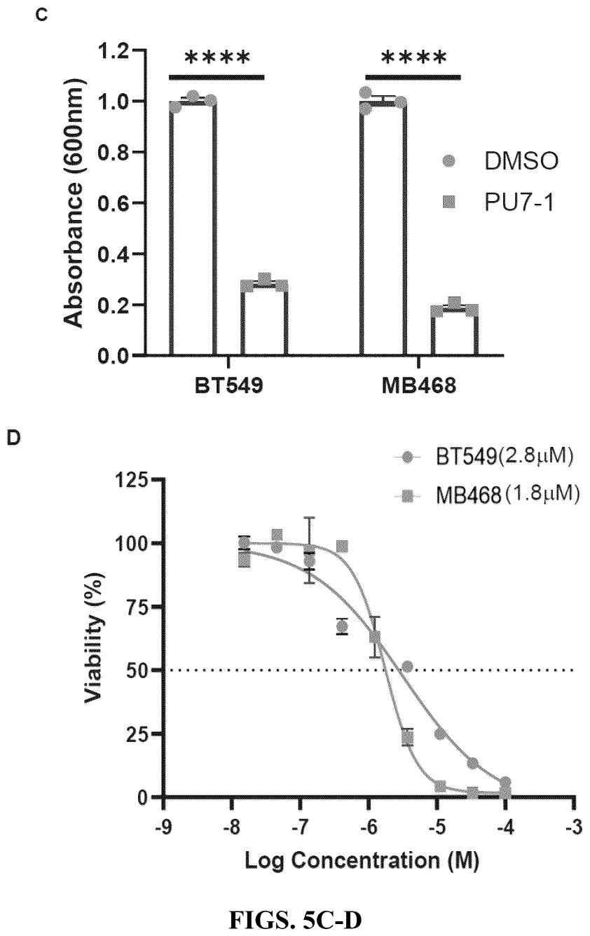

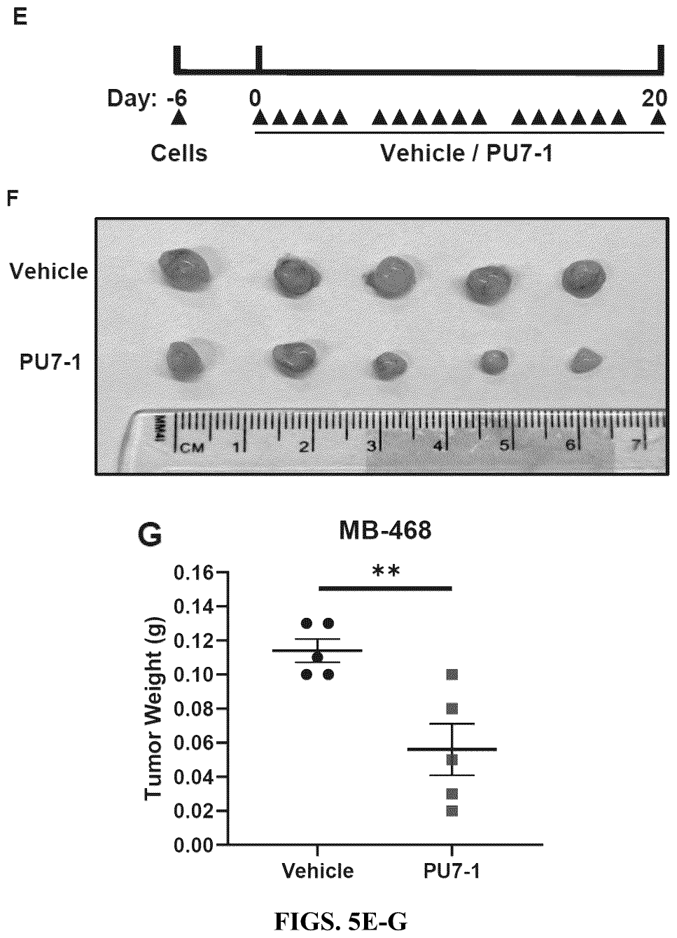

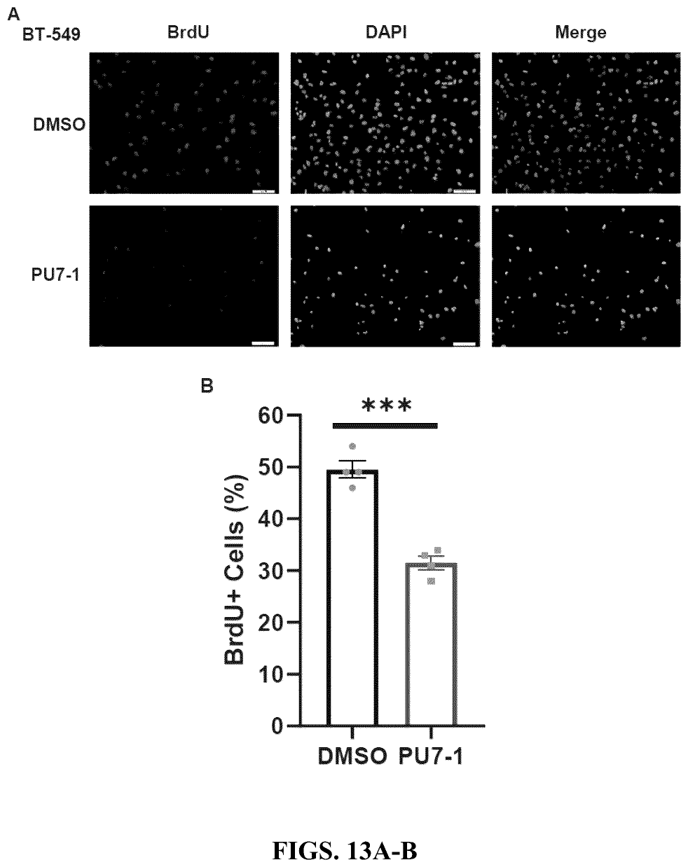

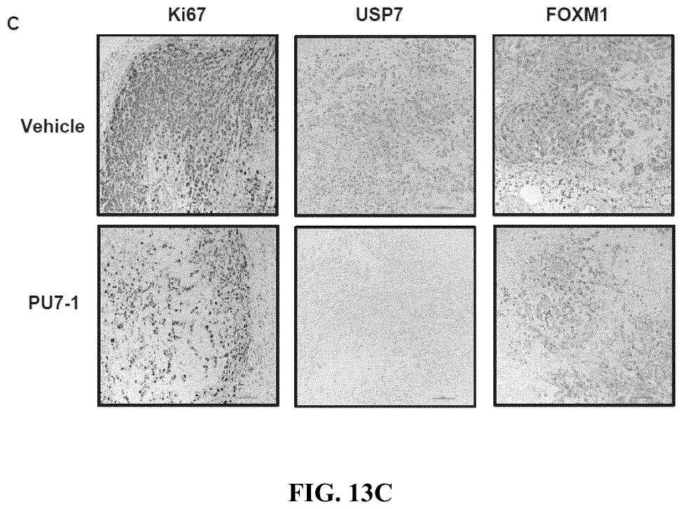

Given the high expression of FOXM1 in triple-negative breast cancer, the effects of the USP7 degrader PU7-1 were next investigated in TNBC cells. First, three TNBC cell lines MDA-MB-468, MDA-MB-231 and BT549 were treated with 10 μM of PU7-1 for 96 h. The protein levels of FOXM1 were determined by Western blot. As shown in FIG. 5A, PU7-1 treatment dramatically decreased the FOXM1 protein levels in all TNBC cells. Next, the effects of PU7-1 were examined on TNBC cell proliferation. MDA-MB468 and BT549 were incubated in the complete growth medium with or without 10 μM of PU7-1 for two weeks. Colonies were stained by crystal violet dye that stains nucleic acids and proteins and is used to reflect the cell number. As shown in FIG. 5B, much less colonies were observed in PU7-1-treated wells. Furthermore, the dye bound to the cells was solubilized with 30% acetic acid and measured by the absorbance at 600 nm. The viability of PU7-1-treated cells is about 20% of the control cells in MDA-MB-468 or 30% of the control cells in BT549 (FIG. 5C). In addition, BrdU incorporation assay in BT549 exhibited similar effects (FIGS. 13A and 13B). The IC50 of PU7-1 in TNBC cells were then determined using a luminescent cell viability assay. As shown in FIG. 5D, PU7-1 treatment led to the proliferation inhibition in TNBC cells with IC50 of 1.8 μM in MDA-MB-468 and 2.8 μM in BT549. These results demonstrate that PU7-1 induces significant cell proliferation inhibition in TNBC cells. Lastly, the xenograft mouse model was utilized to investigate the in vivo efficacy of PU7-1 in TNBC. Two million of MDA-MB-468 cells were subcutaneously inoculated into 6-8-week female nude mice. PU7-1 was intraperitoneally administrated at the dosage of 37.5 mg/kg daily for 3 weeks, 6 days per week (FIG. 5E). At the endpoint, tumors were dissected, weighed, and photographed. The results showed that PU7-1 significantly reduced tumors sizes and weight in xenograft mouse model using MDA-MB-468 cells (FIGS. 5F and 5G), indicating a promising therapeutic potential of PU7-1 in treating triple-negative breast cancer. In addition, the expression of USP7, FOXM1 and the proliferation marker Ki67 in tumors was examined by immunohistochemistry. As shown in FIG. 13C, tumor sections from PU7-1-treated mice displayed dramatically lower levels of USP7, FOXM1 and Ki67. Collectively, these findings demonstrate that the USP7 degrader PU7-1 inhibits cellular proliferation and tumor growth in TNBC.

### FOXM1 is Required for the PU7-1-Mediated Growth Inhibition and Tumor Suppression in TNBC

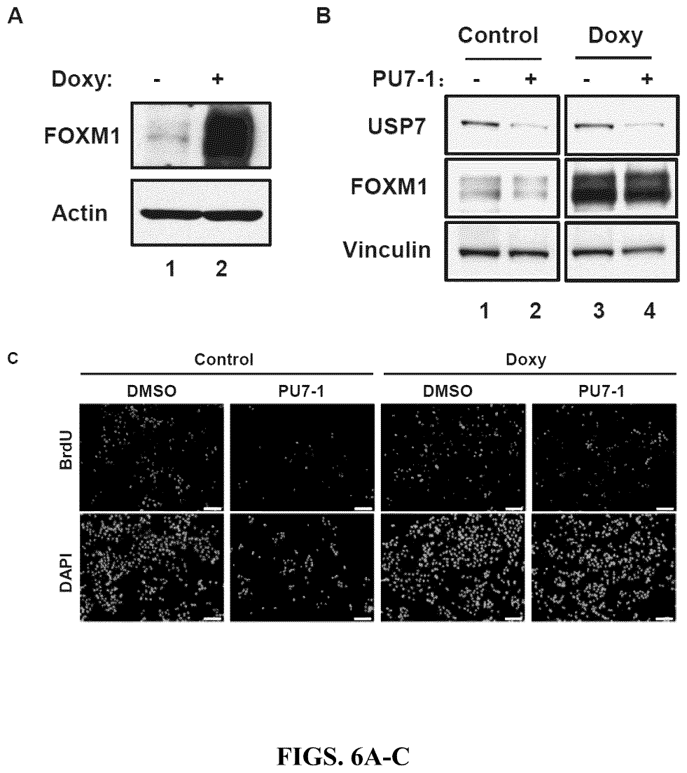

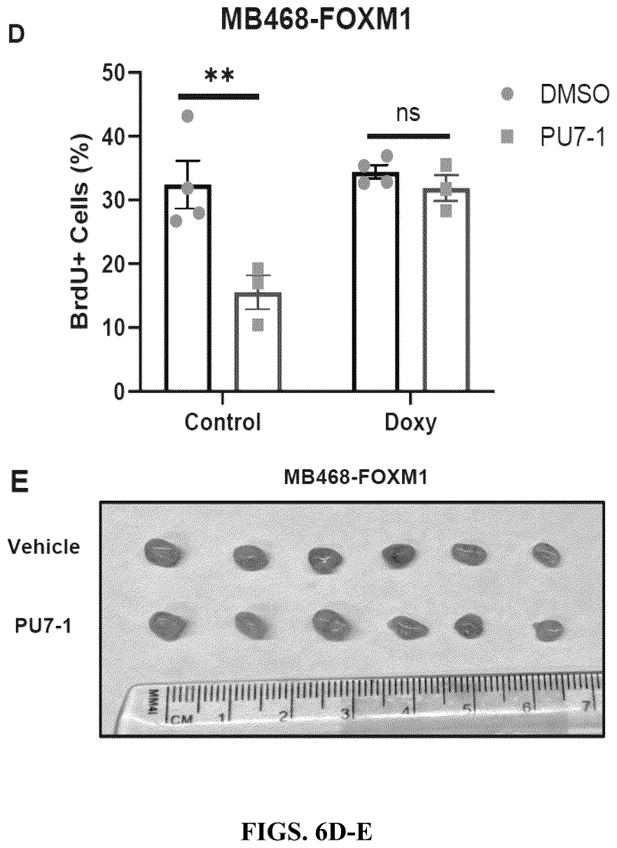

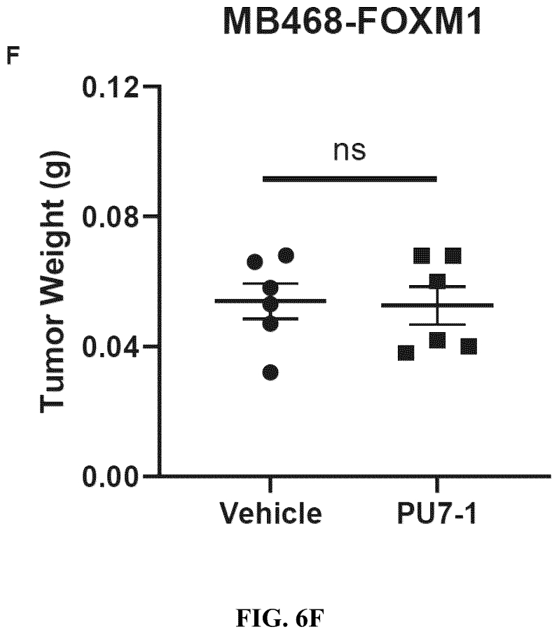

To investigate if PU7-1-mediated effects in TNBC depend on FOXM1, it was first attempted to establish a FOXM1 crispr knockout cell line. Unfortunately, it was not possible to generate the FOXM1 knockout cell line, likely because FOXM1 is an essential gene for cell survival. Previous studies demonstrated that FOXM1 is critical for embryonic development, and FOXM1−/− mice exhibits embryonic lethal phenotype due to multiple abnormalities in the major organs⁴⁴. Alternatively, an inducible FOXM1 overexpression cell line was generated in MDA-MB-468 cells (MDA-MB-468 FOXM1 cells). Doxycycline induces high levels of FOXM1 expression in MDA-MB-468 FOXM1 overexpression cells (FIG. 6A). The effects of PU7-1 on cell proliferation were compared in control and doxycycline-treated MDA-MB-468 FOXM1 cells using BrdU incorporation assay. As shown in FIGS. 6B, 6C and 6D, PU7-1 significantly reduced BrdU positive cells from 32% to 15% in control cells, whereas no reduction of BrdU positive cells was observed after PU7-1 treatment in FOXM1 overexpression cells induced by doxycycline. These results indicate that FOXM1 is required for PU7-1-mediated proliferation inhibition in TNBC cells. Next, the role of FOXM1 in PU7-1-induced tumor suppression was tested. MDA-MB-468 FOXM1 overexpression cells were subcutaneously inoculated into 6-8-week female nude mice fed with doxycycline diet at the dosage of 625 mg/kg to induce exogenous FOXM1 expression. On day 7 after inoculation, mice were randomly separated into two groups. Vehicle or 37.5 mg/kg of PU7-1 were intraperitoneally administrated at the timeline shown in FIG. 5E. At the endpoint, tumors were dissected, weighed, and photographed. As shown in FIGS. 6E and 6F, there is no significant difference in tumor sizes and weights between control and PU7-1 group, suggesting that replenishing of FOXM1 completely abrogates the tumor suppression mediated by PU7-1. These findings demonstrate the critical role of FOXM1 in the PU7-1-mediated proliferation inhibition and tumor suppression in triple-negative breast cancer.

Herein, the overexpression of oncogenic transcription factor FOXM1 was demonstrated in triple negative breast cancer. Further investigation of its interaction partners identified USP7 as the bona fide deubiquitinase of FOXM1. Given the “undruggable” nature of transcription factors, PROTAC technology was utilized and a novel USP7 degrader PU7-1 was discovered to downregulate FOXM1 in TNBC. Notably, PU7-1 significantly elicited cell proliferation inhibition and tumor suppression via a FOXM1-dependent mechanism. These findings confirmed that targeting FOXM1 by USP7 degrader PU7-1 is a feasible strategy. Moreover, Since FOXM1 overexpression in various human malignancies including head and neck cancer, liver cancer, colon cancer, prostate cancer, lung cancer, glioma and ovarian cancer^{45, 46}, it will be very interesting to explore the effect of PU7-1 in other human cancers.

Besides being a monotherapy, inactivation of FOXM1 by PU7-1 also can synergize with current TNBC treatments. In the past few years, FDA have approved two PARP inhibitors (Olaparib and Talazoparib) as monotherapy to treat HER2-negative metastatic breast cancer with germline BRCA1/2 mutations. The mechanism of action of PARP inhibitors in cancer treatment is the synthetic lethality^{47, 48, 49}, in which inhibition of either one of the two genes individually have benign effects, but when inhibition of the two are combined, the cells die. Poly(ADP-ribose) polymerase (PARPs) are a family of related enzymes that contribute to the recognition of DNA single-strand breaks (SSB) and facilitate DNA repair to maintain genomic stability⁵⁰. BRCA1/2 genes encode tumor suppressor proteins that are crucial for the accurate repair of DNA double-strand breaks by homologous recombination (HR). Breast cancer cells with BRCA1/2 deficiency are unable to perform HR. In the absence of HR, alternative DNA repair mechanisms such as PARP-dependent SSB repair are used for cell survival. Both preclinical and clinical studies have demonstrated that Inhibition of PARP has been synthetically lethal in cancer cells with BRCA deficiency^{49, 51, 52, 53}. As mentioned above, about 10%-20% of TNBCs carry BRCA mutations. Therefore, it is not surprising that only a fraction of TNBC patients can benefit from the PARP inhibition. Of note, most patients that initially responded to PARP inhibitors develop resistance to these agents, resulting in disease relapse⁵⁴. Thus, it is still urgent to make sustained effort on broadening the therapeutic options for triple negative breast cancer. Previous studies have reported that FOXM1-deficient cells exhibited increased DNA breaks and reduced expression of BRCA2, implicating an important role of FOXM1 in DNA repair^{55, 56}. A study has reported that PARP inhibitor Olaparib induced the expression and nuclear localization of FOXM1 and targeting FOXM1 disrupted Olaparib-induced adaptive resistance in ovarian cancer⁵⁷. The combination of FOXM1 degradation by PU7-land PARP inhibition can enhance the sensitivity to PARP inhibition in tumor suppression and overcame the adaptive resistance induced by PARP inhibitors in the treatment of TNBCs.

Immune checkpoint inhibitors such as anti-PD-1/PD-L1 antibodies are the major therapeutic advancements in oncology and have rapidly become a standard-of-care treatment for several solid tumor types⁵⁸. Nevertheless, only a small portion (1-10%) of advanced-stage TNBC patients have long-lasting benefit beyond 24 months in the setting of single-agent therapy. A recent study reported that FOXM1 regulates PD-L1 expression by binding directly to its promoter and FOXM1 inhibition enhances the therapeutic outcome of lung cancer immunotherapy by repressing PD-L1 expression⁵⁹. Interestingly, overexpression of PD-L1 was observed in TNBC cells⁶⁰. Hence, targeting FOXM1 by PU7-1 likely suppresses PD-L1 expression, block PD-L1-mediated immune evasion and enhances the efficacy of immune checkpoint inhibitors in triple negative breast cancer.

### Methods

### Statistical Analyses

Data are presented as the means±SD or ±SEM. Statistics were performed using unpaired two-sided student's t test for comparing two sets of data with the software GraphPad Prism. A P value of <0.05 was considered significant.

### Mice

For xenograft mouse model, 2×10⁶ MDA-MB-468 cells were mixed with Matrigel (Corning, #354248) at a 1:1 ratio (Volume) and injected subcutaneous into 6-8-week-old female nude mice (Charles River, #088). When tumors are palpable (˜7 days after injection of tumor cells), mice were treated with either vehicle or PU7-1 at 37.5 mg/kg with daily intraperitoneal injections as indicated schedules. Once mice were euthanized, tumors were collected and weighed for analysis.

### Western Blot

Cells were lysed in FLAG lysis buffer (50 mM Tris-HCl (pH 7.3), 137 mM NaCl, 10 mM NaF, 0.5 mM EDTA, 1% Triton X-100, 0.2% Sarkosyl, 10% glycerol) freshly supplemented with protease inhibitor cocktail. Protein concentration was determined by the Bio-Rad protein assay. Whole cell extracts were then resolved by Novex Tris-Glycine gel (Thermo Scientific) and transferred to a Nitrocellulose membrane (Millipore). Membranes were blocked with 5% (w/v) nonfat milk in Tris-buffered saline with Tween-20 (TBST), incubated with primary antibodies, then secondary HRP-conjugated antibodies (Jackson ImmunoResearch, 1:10000 dilution) and detected on autoradiographic films after incubating with ECL (Thermo Scientific, #32106). Primary antibodies used in this study include anti-USP7 (Cell Signaling, #4833), anti-CRBN (Cell Signaling, #71810); anti-P53 (SantaCruz Biotechnology, sc-126), anti-PUMA (Santa Cruz Biotechnology Cat #sc-28226); anti-p21 (Santa Cruz Biotechnology Cat #sc-53870); anti-FOXM1 (Cell Signaling, #5436); anti-Actin (Sigma-Aldrich Cat #A5441); anti-vinculin (Sigma-Aldrich Cat #V9131).

### Co-Immunoprecipitation

For the co-immunoprecipitation assay of ectopically expressed proteins, cells were lysed in BC100 buffer (50 mM Tris-HCl, pH 8.0, 100 mM NaCl, 0.2% Triton X-100, and 10% glycerol) freshly supplemented with protease inhibitor cocktail. Whole cell extracts were incubated with M2 (Flag) agarose beads (Sigma, A2220) or HA agarose beads (Sigma, A2095) at 4° C. overnight. After four washes with BC100 buffer, the immunoprecipitates were eluted with Flag-peptide (Sigma, F3290) or HA-peptide (Sigma, 12149) for 2 h at 4° C.

For endogenous co-immunoprecipitation assay, cells were lysed in BC100 buffer freshly supplemented with protease inhibitor cocktail. Whole cell extracts were incubated with IgG, anti-USP7 or anti-Foxm1 antibodies as indicated at 4° C. overnight. Next day protein A sepharose (GE healthcare, #17-0780-01) was then added for 4 h at 4° C. After four washes with BC100 buffer, the bound proteins were eluted by boiling with 1×SDS loading buffer.

### Deubiquitination Assay

H1299 cells were transfected with FLAG-FOXM1, USP7, and His-ubiquitin as indicated. At 24 h post-transfection, 10 μM MG132 were added for additional 4 h. 5% of the cells were lysed in FLAG lysis buffer and saved as input. The rest of the cells were lysed in phosphate/guanidine buffer (6 M Guanidine-HCl, 0.1 M Na2HPO4, 0.1M NaH2PO4, 10 mM Tris-HCl, pH 8.0, freshly supplemented with 10 mM β-mercaptoethanol and 5 mM imidazole) with mild sonication. Cell extracts were incubated with Ni-NTA agarose (Qiagen, #30210) at 4° C. overnight. Ni-NTA beads were washed once with lysis buffer, and then four times with urea wash buffer (8M Urea, 0.1 m Na2HPO4, 0.1M NaH2PO4, 10 mM Tris-HCl, pH 6.8, freshly supplemented with 10 mM β-mercaptoethanol and 5 mM imidazole). The bound proteins were eluted with elution buffer (0.5M Imidazole, 10 mM Tris-HCl, pH 6.8, freshly supplemented with 10 mM β-mercaptoethanol).

### GST Pulldown

GST and GST-USP7 proteins were expressed and purified from BL21 E. coli (Milliporesigma, Cat #70954-4). F-FOXM1 proteins were purified by M2 IP from H1299 cells transiently transfected with F-FOXM1 constructs. Purified F-FOXM1 proteins were incubated with GST or GST-USP7-conjugated GST resin at 4° C. overnight. The beads were washed 5 times with BC100 buffer, and then boiled with 1×SDS loading buffer. Precipitates were subjected to western blot analysis and Ponceau S staining.

### Colony Formation Assay

1000 cells (10000 cells for BT474) were seeded in 12-well plates. Next day cells were treated with DMSO or PU7-1 for 2 weeks. Drugs were refreshed every 48 h. After fixation with cold methanol, cells were stained with 0.5% crystal violet solution for 10 min, and then washed with distilled water. For quantification purpose, 1 ml of 30% acetic acid was added to de-stain for 15 min with gentle rotation. The absorbance at 600 nm was measured by using a plate reader in triplicates.

### BrdU Incorporation

Cells were incubated with 10 μg/ml BrdU (BD, 51-2420KC) for 3 h and fixed with cold 70% ethanol. DNA was denatured in 1.5N HCL for 30 min. Cells were blocked with 1% BSA in PBS, and incubated with anti-BrdU antibody (Roche, clone #BMC9318) at 4° C. overnight. After three washes with 1% BSA in PBS, cells were incubated with AlexaFlour 488 conjugated anti-mouse IgG (Thermo scientific, #A32723). Counterstaining with DAPI was performed to visualize the nuclei.

### Cell Viability Assay

Cells were seeded in 96-well white plates (Corning, #3610) and incubated with serially diluted compounds for 6 days. Cell viability was determined using Celltiter glo2.0 cell viability assay (Promega, #G9243) as manufacturer's instruction. IC50 values were determined after fitting curves using Graphpad Prism software.

USP7 siRNA Knockdown Assay

Cells were reversely transfected with non-targeting control or USP7 specific siRNA oligos purchased from Dharmacon for 72 h. The transfection was performed using Lipofectamine3000 (Thermo Scientific, #L3000015) following the manufacturer's instruction.

### Generation of CRNB Knockout Cell Line and Inducible FOXM1 Cell Line

For CRBN knockout cell line, the plenti-px330-CRBN-T1-pGK-Pur construct was purchased from Addgene. HEK293 cells were transiently transfected with plenti-px330-CRBN-T1-pGK-Pur, VSVG and Δ8.9 constructs for lentivirus packaging. Viruses were harvested at 48 h post-transfection. CAL33 cells were transfected with CAS9 constructs and infected with lentiviral particles, and then selected with 1 μg/ml puromycin. CRBN knockout clones were identified by western blot using CRBN specific antibody.

For inducible FOXM1 cell line, the pCW57.1-FOXM1b construct was purchased from Addgene. Lentiviruses were prepared as above. MDA-MB-468 cells were transduced with lentiviruses, and then selected with 1 μg/ml puromycin. The FOXM1 expression was induced with 1 μg/ml doxycycline (Sigma, D9891).

### REFERENCES FOR EXAMPLE 1

1. Yuan J, Luo K, Zhang L, Cheville J C, Lou Z. USP10 regulates p53 localization and stability by deubiquitinating p53. Cell 2010, 140 (3): 384-396.
2. Li M, Chen D, Shiloh A, Luo J, Nikolaev A Y, Qin J, et al. Deubiquitination of p53 by HAUSP is an important pathway for p53 stabilization. Nature 2002, 416 (6881): 648-653.
3. Li M, Brooks C L, Kon N, Gu W. A dynamic role of HAUSP in the p53-Mdm2 pathway. Molecular cell 2004, 13 (6): 879-886.
4. Brooks C L, Gu W. p53 ubiquitination: Mdm2 and beyond. Molecular cell 2006, 21 (3): 307-315.
5. Li M, Brooks C L, Wu-Baer F, Chen D, Baer R, Gu W. Mono-versus polyubiquitination: differential control of p53 fate by Mdm2. Science 2003, 302 (5652): 1972-1975.
6. Kruse J P, Gu W. Modes of p53 regulation. Cell 2009, 137 (4): 609-622.
7. Harakandi C, Nininahazwe L, Xu H, Liu B, He C, Zheng Y C, et al. Recent advances on the intervention sites targeting USP7-MDM2-p53 in cancer therapy. Bioorganic chemistry 2021, 116:105273.
8. Song M S, Salmena L, Carracedo A, Egia A, Lo-Coco F, Teruya-Feldstein J, et al. The deubiquitinylation and localization of PTEN are regulated by a HAUSP-PML network. Nature 2008, 455 (7214): 813-817.
9. Tavana O, Li D, Dai C, Lopez G, Banerjee D, Kon N, et al. HAUSP deubiquitinates and stabilizes N-Myc in neuroblastoma. Nature medicine 2016, 22 (10): 1180-1186.
10. Bronner C. Control of DNMT1 abundance in epigenetic inheritance by acetylation, ubiquitylation, and the histone code. Science signaling 2011, 4 (157): pe3.
11. Hu M, Gu L, Li M, Jeffrey P D, Gu W, Shi Y. Structural basis of competitive recognition of p53 and MDM2 by HAUSP/USP7: implications for the regulation of the p53-MDM2 pathway. PLOS biology 2006, 4 (2): e27.
12. Kon N, Kobayashi Y, Li M, Brooks C L, Ludwig T, Gu W. Inactivation of HAUSP in vivo modulates p53 function. Oncogene 2010, 29 (9): 1270-1279.
13. Kon N, Zhong J, Kobayashi Y, Li M, Szabolcs M, Ludwig T, et al. Roles of HAUSP-mediated p53 regulation in central nervous system development. Cell Death Differ 2011, 18 (8): 1366-1375.
14. Bhattacharya S, Chakraborty D, Basu M, Ghosh M K. Emerging insights into HAUSP (USP7) in physiology, cancer and other diseases. Signal transduction and targeted therapy 2018, 3:17.
15. Valles G J, Bezsonova I, Woodgate R, Ashton N W. USP7 Is a Master Regulator of Genome Stability. Frontiers in cell and developmental biology 2020, 8:717.
16. Lam E W, Brosens J J, Gomes A R, Koo C Y. Forkhead box proteins: tuning forks for transcriptional harmony. Nature reviews Cancer 2013, 13 (7): 482-495.
17. Wang I C, Chen Y J, Hughes D, Petrovic V, Major M L, Park H J, et al. Forkhead box M1 regulates the transcriptional network of genes essential for mitotic progression and genes encoding the SCF (Skp2-Cks1) ubiquitin ligase. Mol Cell Biol 2005, 25 (24): 10875-10894.
18. Chen X, Muller G A, Quaas M, Fischer M, Han N, Stutchbury B, et al. The forkhead transcription factor FOXM1 controls cell cycle-dependent gene expression through an atypical chromatin binding mechanism. Mol Cell Biol 2013, 33 (2): 227-236.
19. Koo C Y, Muir K W, Lam E W. FOXM1: From cancer initiation to progression and treatment. Biochim Biophys Acta 2012, 1819 (1): 28-37.
20. Wang I C, Chen Y J, Hughes D E, Ackerson T, Major M L, Kalinichenko V V, et al. FoxM1 regulates transcription of JNK1 to promote the G1/S transition and tumor cell invasiveness. J Biol Chem 2008, 283 (30): 20770-20778.
21. Huang C, Qiu Z, Wang L, Peng Z, Jia Z, Logsdon C D, et al. A novel FoxMI-caveolin signaling pathway promotes pancreatic cancer invasion and metastasis. Cancer research 2012, 72 (3): 655-665.
22. Zhang N, Wei P, Gong A, Chiu W T, Lee H T, Colman H, et al. FoxMI promotes beta-catenin nuclear localization and controls Wnt target-gene expression and glioma tumorigenesis. Cancer Cell 2011, 20 (4): 427-442.
23. Yao S, Fan L Y, Lam E W. The FOXO3-FOXM1 axis: A key cancer drug target and a modulator of cancer drug resistance. Semin Cancer Biol 2018, 50:77-89.
24. Nilsson M B, Sun H, Robichaux J, Pfeifer M, McDermott U, Travers J, et al. A YAP/FOXM1 axis mediates EMT-associated EGFR inhibitor resistance and increased expression of spindle assembly checkpoint components. Sci Transl Med 2020, 12 (559).
25. Foulkes W D, Smith I E, Reis-Filho J S. Triple-negative breast cancer. N Engl J Med 2010, 363 (20): 1938-1948.
26. Dietze E C, Sistrunk C, Miranda-Carboni G, O'Regan R, Seewaldt V L. Triple-negative breast cancer in African-American women: disparities versus biology. Nature reviews Cancer 2015, 15 (4): 248-254.
27. Bianchini G, Balko J M, Mayer I A, Sanders M E, Gianni L. Triple-negative breast cancer: challenges and opportunities of a heterogeneous disease. Nat Rev Clin Oncol 2016, 13 (11): 674-690.
28. Geenen J J J, Linn S C, Beijnen J H, Schellens J H M. PARP Inhibitors in the Treatment of Triple-Negative Breast Cancer. Clin Pharmacokinet 2018, 57 (4): 427-437.
29. Gonzalez-Angulo A M, Timms K M, Liu S, Chen H, Litton J K, Potter J, et al. Incidence and outcome of BRCA mutations in unselected patients with triple receptor-negative breast cancer. Clin Cancer Res 2011, 17 (5): 1082-1089.
30. Wong-Brown M W, Meldrum C J, Carpenter J E, Clarke C L, Narod S A, Jakubowska A, et al. Prevalence of BRCA1 and BRCA2 germline mutations in patients with triple-negative breast cancer. Breast Cancer Res Treat 2015, 150 (1): 71-80.
31. Pogoda K, Niwinska A, Sarnowska E, Nowakowska D, Jagiello-Gruszfeld A, Siedlecki J, et al. Effects of BRCA Germline Mutations on Triple-Negative Breast Cancer Prognosis. J Oncol 2020, 2020:8545643.
32. Qi S M, Cheng G, Cheng X D, Xu Z, Xu B, Zhang W D, et al. Targeting USP7-Mediated Deubiquitination of MDM2/MDMX-p53 Pathway for Cancer Therapy: Are We There Yet? Frontiers in cell and developmental biology 2020, 8:233.
33. Lin Y T, Lin J, Liu Y E, Chen Y C, Liu S T, Hsu K W, et al. USP7 Induces Chemoresistance in Triple-Negative Breast Cancer via Deubiquitination and Stabilization of ABCB1. Cells 2022, 11 (20).
34. Xia X, Liao Y, Huang C, Liu Y, He J, Shao Z, et al. Deubiquitination and stabilization of estrogen receptor alpha by ubiquitin-specific protease 7 promotes breast tumorigenesis. Cancer Lett 2019, 465:118-128.
35. Pandit B, Halasi M, Gartel A L. p53 negatively regulates expression of FoxM1. Cell cycle 2009, 8 (20): 3425-3427.
36. Barsotti A M, Prives C. Pro-proliferative FoxMI is a target of p53-mediated repression. Oncogene 2009, 28 (48): 4295-4305.
37. Lv C, Zhao G, Sun X, Wang P, Xie N, Luo J, et al. Acetylation of FOXM1 is essential for its transactivation and tumor growth stimulation. Oncotarget 2016, 7 (37): 60366-60382.
38. Fu Z, Malureanu L, Huang J, Wang W, Li H, van Deursen J M, et al. Plk1-dependent phosphorylation of FoxMI regulates a transcriptional programme required for mitotic progression. Nature cell biology 2008, 10 (9): 1076-1082.
39. Major M L, Lepe R, Costa R H. Forkhead box MIB transcriptional activity requires binding of Cdk-cyclin complexes for phosphorylation-dependent recruitment of p300/CBP coactivators. Mol Cell Biol 2004, 24 (7): 2649-2661.
40. Kopanja D, Chand V, O'Brien E, Mukhopadhyay N K, Zappia M P, Islam A, et al. Transcriptional Repression by FoxMI Suppresses Tumor Differentiation and Promotes Metastasis of Breast Cancer. Cancer research 2022, 82 (13): 2458-2471.
41. Dale B, Cheng M, Park K S, Kaniskan H U, Xiong Y, Jin J. Advancing targeted protein degradation for cancer therapy. Nature reviews Cancer 2021, 21 (10): 638-654.
42. Smith B E, Wang S L, Jaime-Figueroa S, Harbin A, Wang J, Hamman B D, et al. Differential PROTAC substrate specificity dictated by orientation of recruited E3 ligase. Nature communications 2019, 10 (1): 131.
43. Zengerle M, Chan K H, Ciulli A. Selective Small Molecule Induced Degradation of the BET Bromodomain Protein BRD4. ACS chemical biology 2015, 10 (8): 1770-1777.
44. Kalin T V, Ustiyan V, Kalinichenko V V. Multiple faces of FoxM1 transcription factor: lessons from transgenic mouse models. Cell cycle 2011, 10 (3): 396-405.
45. Liao G B, Li X Z, Zeng S, Liu C, Yang S M, Yang L, et al. Regulation of the master regulator FOXM1 in cancer. Cell communication and signaling: CCS 2018, 16 (1): 57.
46. Li L, Wu D, Yu Q, Li L, Wu P. Prognostic value of FOXM1 in solid tumors: a systematic review and meta-analysis. Oncotarget 2017, 8 (19): 32298-32308.
47. Lord C J, Ashworth A. Mechanisms of resistance to therapies targeting BRCA-mutant cancers. Nature medicine 2013, 19 (11): 1381-1388.
48. Turner N, Tutt A, Ashworth A. Targeting the DNA repair defect of BRCA tumours. Curr Opin Pharmacol 2005, 5 (4): 388-393.
49. Farmer H, McCabe N, Lord C J, Tutt A N, Johnson D A, Richardson T B, et al. Targeting the DNA repair defect in BRCA mutant cells as a therapeutic strategy. Nature 2005, 434 (7035): 917-921.
50. Jackson S P, Bartek J. The DNA-damage response in human biology and disease. Nature 2009, 461 (7267): 1071-1078.
51. Bryant H E, Schultz N, Thomas H D, Parker K M, Flower D, Lopez E, et al. Specific killing of BRCA2-deficient tumours with inhibitors of poly(ADP-ribose) polymerase. Nature 2005, 434 (7035): 913-917.
52. Tung N M, Robson M E, Ventz S, Santa-Maria C A, Nanda R, Marcom P K, et al. TBCRC 048: Phase II Study of Olaparib for Metastatic Breast Cancer and Mutations in Homologous Recombination-Related Genes. J Clin Oncol 2020, 38 (36): 4274-4282.
53. Fong P C, Boss D S, Yap T A, Tutt A, Wu P, Mergui-Roelvink M, et al. Inhibition of poly(ADP-ribose) polymerase in tumors from BRCA mutation carriers. N Engl J Med 2009, 361 (2): 123-134.
54. Dias M P, Moser S C, Ganesan S, Jonkers J. Understanding and overcoming resistance to PARP inhibitors in cancer therapy. Nat Rev Clin Oncol 2021, 18 (12): 773-791.
55. Zona S, Bella L, Burton M J, Nestal de Moraes G, Lam E W. FOXM1: an emerging master regulator of DNA damage response and genotoxic agent resistance. Biochim Biophys Acta 2014, 1839 (11): 1316-1322.
56. Tan Y, Raychaudhuri P, Costa R H. Chk2 mediates stabilization of the FoxM1 transcription factor to stimulate expression of DNA repair genes. Mol Cell Biol 2007, 27 (3): 1007-1016.
57. Fang P, Madden J A, Neums L, Moulder R K, Forrest M L, Chien J. Olaparib-induced Adaptive Response Is Disrupted by FOXM1 Targeting that Enhances Sensitivity to PARP Inhibition. Mol Cancer Res 2018, 16 (6): 961-973.
58. Bianchini G, De Angelis C, Licata L, Gianni L. Treatment landscape of triple-negative breast cancer-expanded options, evolving needs. Nat Rev Clin Oncol 2022, 19 (2): 91-113.
59. Madhi H, Lec J S, Choi Y E, Li Y, Kim M H, Choi Y, et al. FOXM1 Inhibition Enhances the Therapeutic Outcome of Lung Cancer Immunotherapy by Modulating PD-L1 Expression and Cell Proliferation. Adv Sci (Weinh) 2022, 9 (29): c2202702.
60. Mittendorf E A, Philips A V, Meric-Bernstam F, Qiao N, Wu Y, Harrington S, et al. PD-L1 expression in triple-negative breast cancer. Cancer Immunol Res 2014, 2 (4): 361-370.
61. van Loosdregt J, Fleskens V, Fu J, Brenkman A B, Bekker C P, Pals C E, et al. Stabilization of the transcription factor Foxp3 by the deubiquitinase USP7 increases Treg-cell-suppressive capacity. Immunity 2013, 39 (2): 259-271.

### Example 2—General Chemistry Procedure for PU7-1 Synthesis

All commercial chemical reagents and solvents were used for the reactions without further purification. Flash column chromatography was performed on Teledyne ISCO CombiFlash Rf+ instrument equipped with a 220/254/280 nm wavelength UV detector and a fraction collector. Normal phase column chromatography was conducted on silica gel columns with either hexane/ethyl acetate or dichloromethane/methanol as eluent. Reverse phase column chromatography was conducted on HP C18 RediSep Rf columns, and the gradient was set to 10% of acetonitrile in H2O containing 0.1% TFA progressing to 100% of acetonitrile. All final compounds were purified with preparative high-performance liquid chromatography (HPLC) on an Agilent Prep 1200 series with the UV detector set to 220/254 nm at a flow rate of 40 mL/min. Samples were injected onto a Phenomenex Luna 750×30 mm, 5 μm C18 column, and the gradient was set to 10% of acetonitrile in H2O containing 0.1% TFA progressing to 100% of acetonitrile. All compounds assessed for biological activity have purity>95% as determined by an Agilent 1200 series system with DAD detector and a 2.1 mm×150 mm Zorbax 300SB-C18 5 μm column for chromatography and high-resolution mass spectra (HRMS) that were acquired in positive ion mode using an Agilent G1969A API-TOF with an electrospray ionization (ESI) source. Samples (2 μL) were injected onto a C18 column at room temperature, and the flow rate was set to 0.4 mL/min with water containing 0.1% formic acid as solvent A and acetonitrile containing 0.1% formic acid as solvent B. Nuclear magnetic resonance (NMR) spectra were acquired on Bruker DRX 400 MHz for proton (¹H NMR) and 101 MHz for carbon (¹³C NMR). Chemical shifts for all compounds are reported in parts per million (ppm, δ). The format of chemical shift was reported as follows: chemical shift, multiplicity (s=singlet, d=doublet, t=triplet, q=quartet, m=multiplet), coupling constant (J values in Hz), and integration.

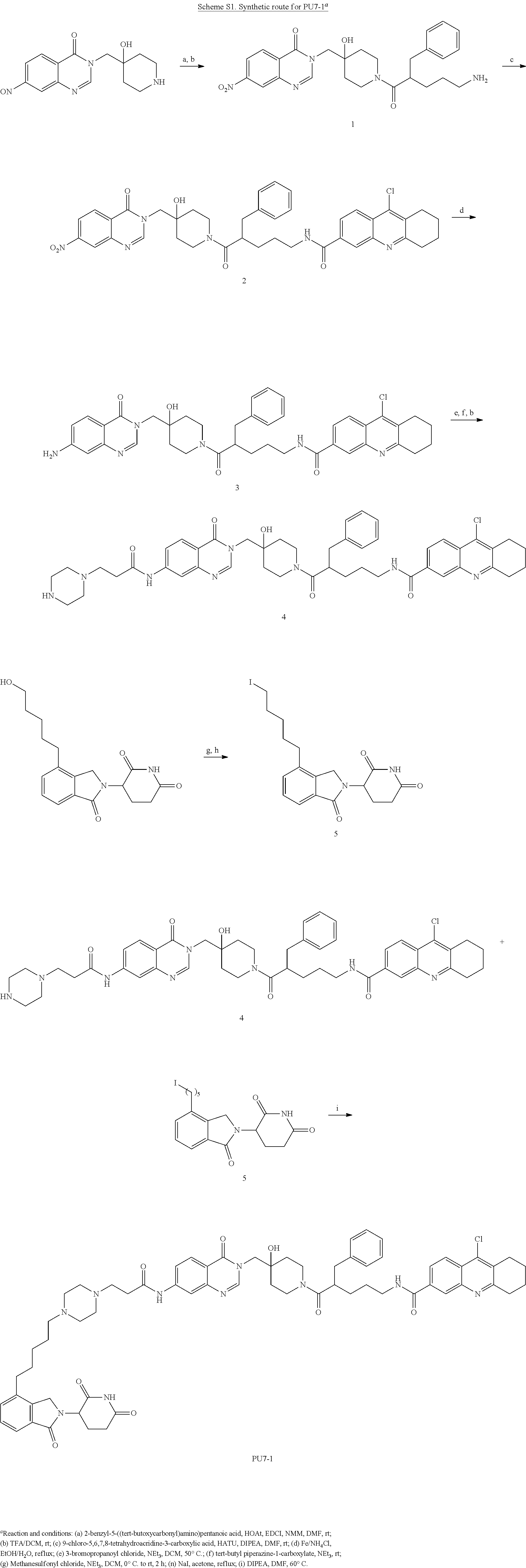

3-((1-(5-amino-2-benzylpentanoyl)-4-hydroxypiperidin-4-yl)methyl)-7-nitroquinazolin-4 (3H)-one (1). To a solution of 3-((4-hydroxypiperidin-4-yl)methyl)-7-nitroquinazolin-4 (3H)-one (commercial available) (1.20 g, 3.8 mmol, 1 equiv) in DMF (6 mL) were added 2-benzyl-5-((tert-butoxycarbonyl)amino)pentanoic acid (commercial available) (1.2 g, 3.8 mmol, 1.0 equiv), HOAt (1-hydroxy-7-azabenzo-triazole) (0.78 g, 5.7 mmol, 1.5 equiv), EDCI (1-ethyl-3-(3-dimethylaminopropyl) carbodiimide) (1.20 g, 5.7 mmol, 1.5 equiv), and NMM (N-methylmorpholine) (1.30 mL, 11.4 mmol, 3 equiv). After being stirred overnight at room temperature, the resulting mixture was purified by reverse phase ISCO to afford the intermediate. To a solution of the obtained intermediate in DCM (2 mL) were added TFA (2 mL). The resulting mixture was stirred at room temperature for 1 h followed by purified by reverse phase ISCO to get compound 1 as a yellow solid (2.01 g, 94% yield). ¹H NMR (400 MHz, CD₃OD) δ 8.48-8.46 (m, 2H), 8.34-8.26 (m, 3H), 7.40-7.36 (m, 1H), 7.25-7.22 (m, 2H), 7.15-7.13 (m, 1H), 4.33 (d, J=9.2 Hz, 1H), 4.15-4.02 (m, 1H), 3.95-3.92 (m, 1H), 3.68-3.65 (m, 1H), 3.22-3.15 (m, 2H), 2.90-2.77 (m, 5H), 1.80-1.72 (m, 1H), 1.66-1.47 (m, 3H), 1.33-1.21 (m, 2H), 1.11 (d, J=13.6, 1H), 0.23-0.15 (m, 1H).

N-(4-benzyl-5-(4-hydroxy-4-((7-nitro-4-oxoquinazolin-3 (4H)-yl)methyl)piperidin-1-yl)-5-oxopentyl)-9-chloro-5,6,7,8-tetrahydroacridine-3-carboxamide (2). To a solution of compound 1 (1.40 g, 2.9 mmol, 1 equiv) in DMF (8 mL) were added 9-chloro-5,6,7,8-tetrahydroacridine-3-carboxylic acid (commercial available) (0.76 g, 2.9 mmol, 1.0 equiv), HATU (1.65 g, 4.35 mmol, 1.5 equiv) and DIPEA (1.51 mL, 8.7 mmol, 3 equiv). After being stirred overnight at room temperature, the resulting mixture was purified by reverse phase ISCO to afford the compound 2 as a yellow solid (1.47 g, 78% yield). ¹H NMR (400 MHZ, CD₃OD) δ 8.51-8.46 (m, 4H), 8.37-8.30 (m, 2H), 8.26-8.18 (m, 2H), 7.38-7.35 (m, 1H), 7.27-7.19 (m, 2H), 7.16-7.13 (m, 1H), 4.31 (d, J=12.8 Hz, 1H), 4.06-4.04 (m, 1H), 3.95-3.91 (m, 1H), 3.71-3.61 (m, 2H), 3.51-3.43 (m, 3H), 3.25-3.12 (m, 5H), 2.83-2.81 (m, 2H), 2.05-2.03 (m, 4H), 1.87-1.79 (m, 1H), 1.69-1.55 (m, 3H), 1.32-1.21 (m, 2H), 1.12 (d, J=13.6 Hz, 1H), 0.25-0.18 (m, 1H).

N-(5-(4-((7-amino-4-oxoquinazolin-3 (4H)-yl)methyl)-4-hydroxypiperidin-1-yl)-4-benzyl-5-oxopentyl)-9-chloro-5,6,7,8-tetrahydroacridine-3-carboxamide (3). To a solution of compound 2 (147 mg, 0.2 mmol, 1 equiv) in EtOH/H2O (3:1, 8 mL) were added Fe powder (45 mg, 0.8 mmol, 4.0 equiv) and NH₄Cl (43 mg, 1.0 mmol, 5 equiv). After being stirred overnight under reflux, the resulting mixture was filtered with celite pad and washed with MeOH (3×5 mL). The filtrate was collected and concentrated, the resulting residue was purified by reverse phase ISCO to afford the compound 3 as a yellow solid (104 mg, 73% yield). ¹H NMR (400 MHz, CD₃OD) δ 8.52-8.50 (m, 1H), 8.44-8.38 (m, 2H), 8.11 (d, J=10.4 Hz, 1H), 7.95 (d, J=8.8 Hz, 1H), 7.35-7.32 (m, 1H), 7.24-7.19 (m, 3H), 7.15-7.13 (m, 1H), 6.90 (dd, J=8.8, 2.4 Hz, 1H), 6.68 (d, J=2.0 Hz, 1H), 4.29 (d, J=12.8 Hz, 1H), 3.99 (d, J=9.6 Hz, 1H), 3.92-3.89 (m, 1H), 3.70-3.59 (m, 2H), 3.47-3.42 (m, 3H), 3.24-3.22 (m, 2H), 3.10-3.09 (m, 3H), 2.83-2.77 (m, 2H), 2.01-2.00 (m, 4H), 1.87-1.78 (m, 1H), 1.70-1.52 (m, 3H), 1.29-1.16 (m, 2H), 1.10 (d, J=12.4 Hz, 1H), 0.23-0.15 (m, 1H).

N-(4-benzyl-5-(4-hydroxy-4-((4-oxo-7-(3-(piperazin-1-yl) propanamido) quinazolin-3 (4H)-yl)methyl)piperidin-1-yl)-5-oxopentyl)-9-chloro-5,6,7,8-tetrahydroacridine-3-carboxamide (4). To a solution of compound 3 (1.07 g, 1.5 mmol, 1 equiv) in DCM (10 mL) were added 3-bromopropanoyl chloride (386 mg, 2.25 mmol, 1.5 equiv) and NEt₃ (0.63 mL, 4.5 mmol, 3 equiv). After being stirred overnight at 50° C., the reaction mixture was cooled down to room temperature and tert-butyl piperazine-1-carboxylate (837 mg, 4.5 mmol, 1.5 equiv) and NEt₃ (0.63 mL, 4.5 mmol, 3 equiv) were added. After being stirred overnight at 50° C., the resulting mixture concentrated and purified by reverse phase ISCO to afford the intermediate. To a solution of the obtained intermediate in DCM (2 mL) were added TFA (2 mL). The resulting mixture was stirred at room temperature for 1 h followed by purified by reverse phase ISCO to get compound 4 as a yellow solid (500 mg, 26% yield). ¹H NMR (400 MHz, CD₃OD) δ 8.51-8.47 (m, 2H), 8.28-8.08 (m, 4H), 7.67 (dd, J=8.8, 2.0 Hz, 1H), 7.35-7.31 (m, 1H), 7.23-7.19 (m, 3H), 7.15-7.13 (m, 1H), 4.30 (d, J=12.9 Hz, 1H), 4.01-3.92 (m, 1H), 3.71-3.60 (m, 2H), 3.49-3.43 (m, 7H), 3.39-3.38 (m, 6H), 3.25-3.13 (m, 4H), 2.95-2.91 (m, 2H), 2.83-2.77 (m, 4H), 2.04-2.03 (m, 4H), 1.87-1.78 (m, 1H), 1.69-1.54 (m, 3H), 1.28-1.22 (m, 2H), 1.12 (d, J=13.6 Hz, 1H), 0.24-0.17 (m, 1H).

3-(4-(5-iodopentyl)-1-oxoisoindolin-2-yl) piperidine-2,6-dione (5). To a solution of 3-(4-(5-hydroxypentyl)-1-oxoisoindolin-2-yl) piperidine-2,6-dione (prepared following previous reported procedure) (Wang; et al., 2017) (1.86 g, 5.6 mmol, 1 equiv) in DCM (30 mL) were added NEt₃ (1.6 mL, 11.2 mmol, 2 equiv), then the reaction mixture was cooled down to 0° C. and methanesulfonyl chloride (0.9 mL, 11.2 mmol, 2 equiv) was added. Then, the reaction mixture was warmed to room temperature and stirred for 2 h, followed by concentrated and purified by reverse phase ISCO to afford the intermediate. To a solution of the obtained intermediate in acetone (15 mL) were added KI (2.8 g, 16.8 mmol, 3 equiv). After being stirred overnight under reflux, the resulting mixture was purified by normal phase ISCO (DCM: EA, 20%-70%) to afford the compound 5 as a white solid (1.0 g, 41% yield with two steps). ¹H NMR (400 MHZ, CD₃OD) δ 7.65-7.63 (m, 1H), 7.49-7.44 (m, 2H), 5.17 (dd, J=13.2, 4.8 Hz, 1H), 4.54-4.43 (m, 2H), 3.23 (t, J=6.8 Hz, 2H), 2.96-2.87 (m, 1H), 2.81-2.70 (m, 3H), 2.59-2.48 (m, 1H), 2.21-2.15 (m, 1H), 1.88-1.81 (m, 2H), 1.74-1.66 (m, 2H), 1.52-1.42 (m, 2H).

N-(4-benzyl-5-(4-((7-(3-(4-(5-(2-(2,6-dioxopiperidin-3-yl)-1-oxoisoindolin-4-yl) pentyl) piperazin-1-yl) propanamido)-4-oxoquinazolin-3 (4H)-yl)methyl)-4-hydroxypiperidin-1-yl)-5-oxopentyl)-9-chloro-5,6,7,8-tetrahydroacridine-3-carboxamide (PU7-1). To a solution of compound 4 (300 mg, 0.31 mmol, 1 equiv) in DMF (3 mL) were added compound 5 (137 mg, 0.31 mmol, 1 equiv) and DIPEA (0.16 mL, 0.93 mmol, 3 equiv). After being stirred overnight at 60° C., the resulting mixture was purified by preparative HPLC to afford the compound PU7-1 as white solid (260 mg, 52% yield). ¹H NMR (400 MHZ, CD₃OD) δ 8.48-8.42 (m, 2H), 8.28-8.03 (m, 4H), 7.65-7.58 (m, 2H), 7.45-7.44 (m, 2H), 7.34-7.30 (m, 1H), 7.21-7.18 (m, 3H), 7.14-7.11 (m, 1H), 5.16 (dd, J=13.2, 4.8 Hz, 1H), 4.49 (d, J=17.2 Hz, 1H), 4.42 (d, J=17.2 Hz, 1H), 4.29 (d, J=13.2 Hz, 1H), 4.00-3.90 (m, 1H), 3.69-3.37 (m, 13H), 3.28-3.10 (m, 8H), 2.96-2.89 (m, 3H), 2.82-2.69 (m, 6H), 2.56-2.45 (m, 1H), 2.19-2.15 (m, 1H), 2.02-2.01 (m, 4H), 1.85-1.53 (m, 9H), 1.46-1.38 (m, 2H), 1.30-1.10 (m, 3H), 0.23-0.16 (m, 1H). ¹³C NMR (101 MHZ, CD₃OD) δ175.50, 174.66, 172.37, 171.77, 171.20, 167.76, 162.07, 161.25, 150.96, 149.38, 148.99, 145.81, 142.01, 141.21, 140.69, 139.80, 138.63, 133.73, 133.29, 132.57, 130.37, 129.81, 129.71, 129.40, 128.78, 128.11, 127.77, 126.35, 123.11, 122.20, 120.40, 118.19, 115.59, 70.93, 70.80, 57.70, 55.71, 55.37, 53.58, 50.83, 50.52, 44.19, 43.88, 42.82, 41.03, 38.79, 36.45, 35.79, 32.99, 32.36, 31.35, 30.28, 30.07, 28.23, 27.08, 24.79, 24.08, 22.49, 22.02. HRMS (ESI) m/z: calcd for C₆₅H₇₆ClN₁₀O₈⁺ [M+H]⁺, 1159.5531; found, 1159.5508.

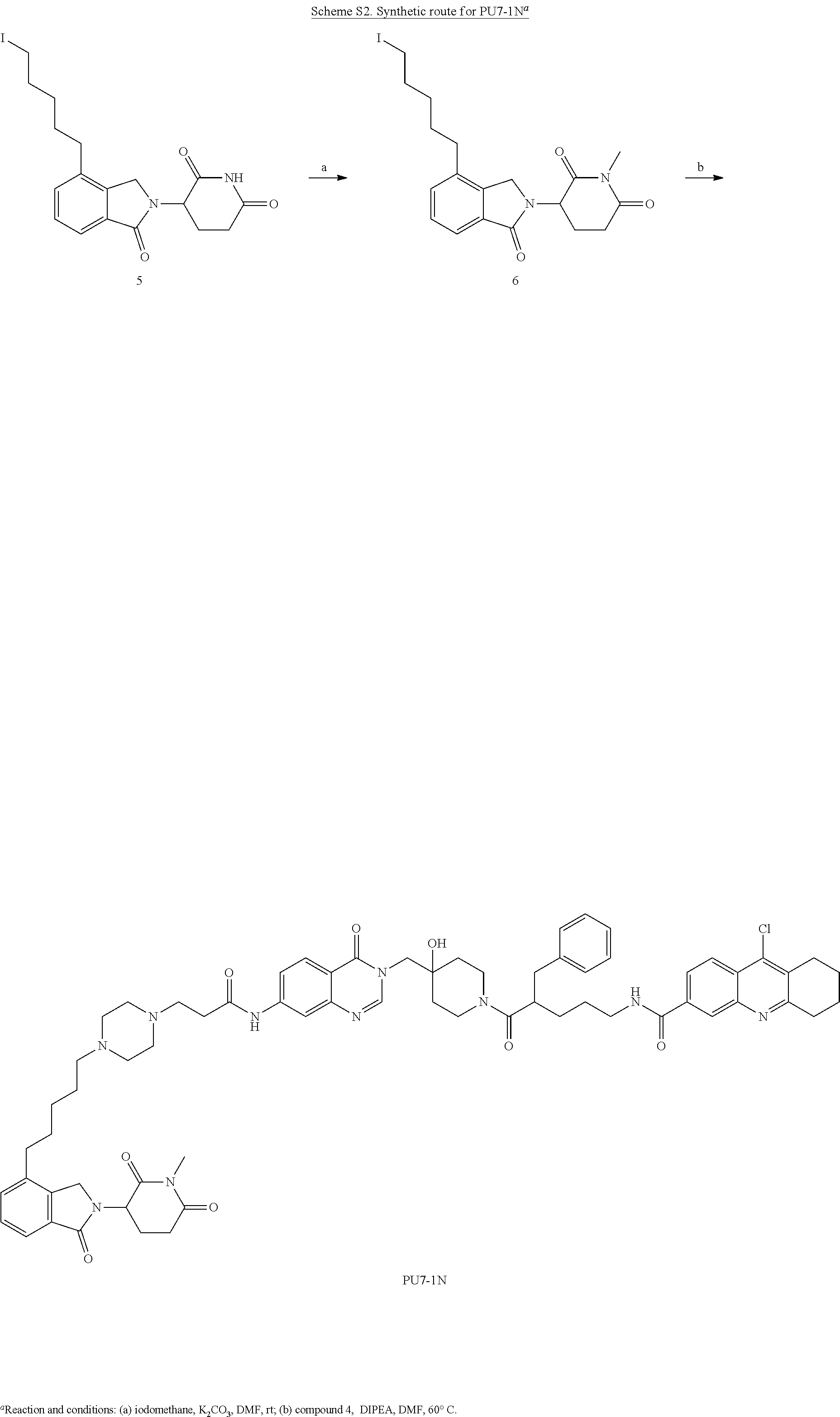 [SMILES: O=C1CCC(N2Cc3c(CCCCCI)cccc3C2=O)C(=O)N1 ; CN1C(=O)CCC(N2Cc3c(CCCCCI)cccc3C2=O)C1=O ; CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O ; "PU7-1N"]

3-(4-(5-iodopentyl)-1-oxoisoindolin-2-yl)-1-methylpiperidine-2,6-dione (6). To a solution of compound 5 (50 mg, 0.11 mmol, 1 equiv) in DMF (1 mL) were added compound K₂CO₃ (30 mg, 0.22 mmol, 2 equiv) and iodomethane (31 mg, 0.22 mmol, 2 equiv). After being stirred 12 h at room temperature, the resulting mixture was purified by preparative HPLC to afford the compound 6 as a white solid (35 mg, 70% yield). ¹H NMR (400 MHZ, CD₃OD) δ 7.65-7.63 (m, 1H), 7.46 (s, 2H), 5.17 (d, J=13.3 Hz, 1H), 4.52-4.40 (m, 2H), 3.24-3.20 (m, 2H), 3.12 (s, 3H), 2.98-2.85 (m, 2H), 2.72-2.67 (m, 2H), 2.55-2.44 (m, 1H), 2.17-2.12 (m, 1H), 1.85-1.79 (m, 2H), 1.70-1.66 (m, 2H), 1.49-1.46 (m, 2H).

N-(4-benzyl-5-(4-hydroxy-4-((7-(3-(4-(5-(2-(1-methyl-2,6-dioxopiperidin-3-yl)-1-oxoisoindolin-4-yl) pentyl) piperazin-1-yl) propanamido)-4-oxoquinazolin-3 (4H)-yl)methyl)piperidin-1-yl)-5-oxopentyl)-9-chloro-5,6,7,8-tetrahydroacridine-3-carboxamide (PU7-1N). To a solution of compound 4 (10 mg, 0.01 mmol, 1 equiv) in DMF (0.5 mL) were added compound 6 (5 mg, 0.01 mmol, 1 equiv) and DIPEA (5 μL, 0.33 mmol, 3 equiv). After being stirred overnight at 60° C., the resulting mixture was purified by preparative HPLC to afford the compound PU7-1N as a white solid (7 mg, 44% yield). ¹H NMR (400 MHZ, CD₃OD) δ 8.49-8.46 (m, 2H), 8.28-8.06 (m, 4H), 7.67-7.60 (m, 2H), 7.48-7.44 (m, 2H), 7.34-7.31 (m, 1H), 7.22-7.17 (m, 3H), 7.15-7.13 (m, 1H), 5.19 (dd, J=13.6, 5.2 Hz, 1H), 4.50 (d, J=17.2 Hz, 1H), 4.42 (d, J=17.2 Hz, 1H), 4.30 (d, J=12.8 Hz, 1H), 4.01-3.92 (m, 1H), 3.71-3.60 (m, 2H), 3.51-3.43 (m, 6H), 3.34-3.31 (m, 3H), 3.24-3.10 (m, 12H), 2.99-2.71 (m, 10H), 2.53-2.42 (m, 1H), 2.17-2.13 (m, 1H), 2.03-2.02 (m, 4H), 1.86-1.54 (m, 9H), 1.47-1.39 (m, 2H), 1.28-1.11 (m, 3H), 0.24-0.16 (m, 1H). ¹³C NMR (101 MHz, CD₃OD) δ 175.52, 173.67, 172.07, 171.77, 171.50, 167.86, 162.16, 161.34, 150.93, 149.21, 145.82, 142.03, 141.23, 140.71, 139.73, 138.64, 133.69, 133.32, 132.63, 130.38, 129.82, 129.73, 129.40, 128.77, 128.15, 127.94, 127.51, 126.34, 123.39, 122.24, 120.38, 118.26, 115.75, 70.94, 70.81, 57.71, 55.69, 54.19, 53.75, 51.17, 50.65, 44.20, 42.83, 41.02, 38.79, 35.81, 35.08, 33.35, 32.67, 32.45, 32.13, 31.35, 30.31, 28.26, 27.32, 27.17, 24.88, 23.32, 22.55, 22.10. HRMS (ESI) m/z: calcd for C₆₆H₇₈ClN₁₀O₈⁺ [M+H]⁺, 1173.5687; found, 1173.5649.

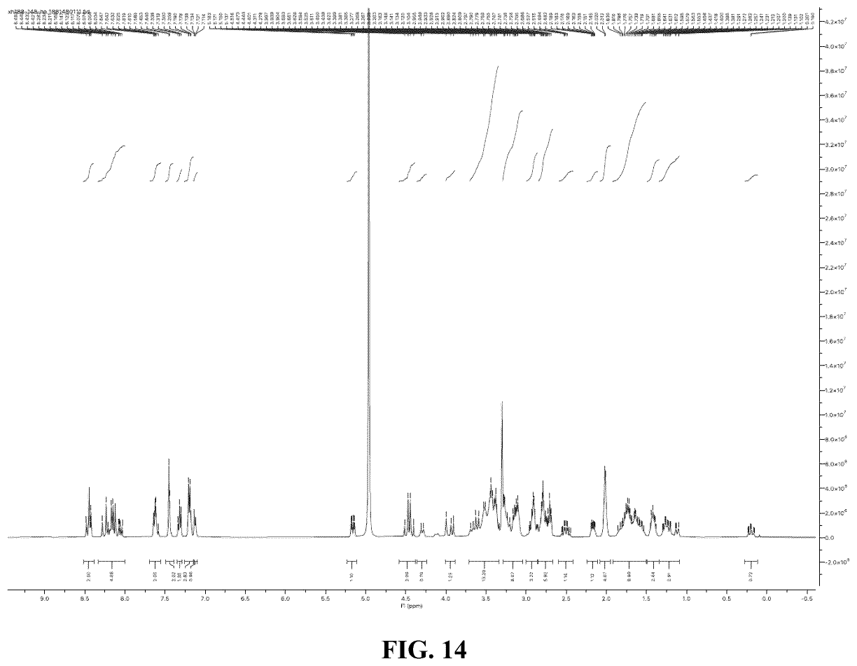

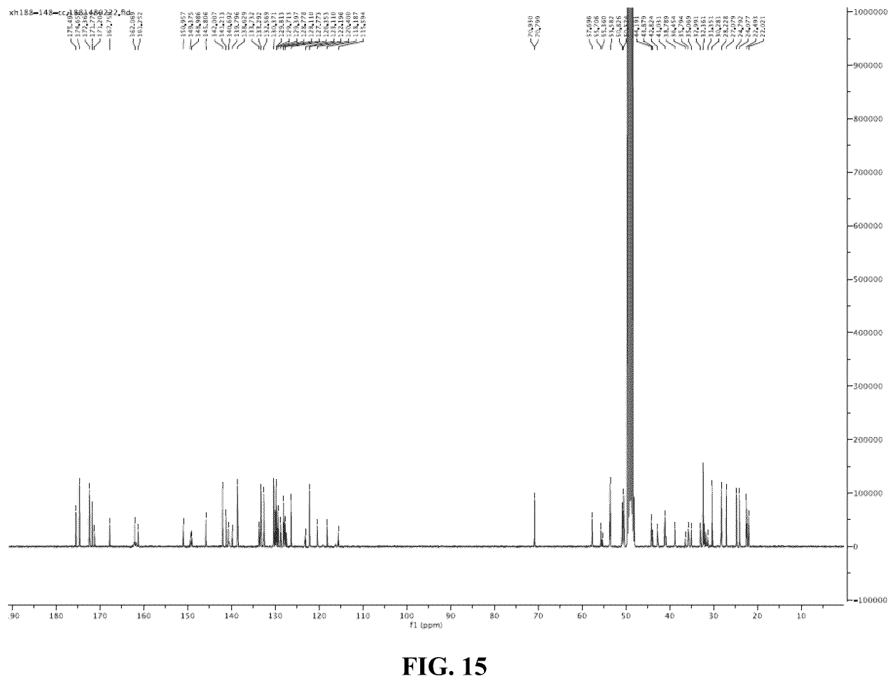

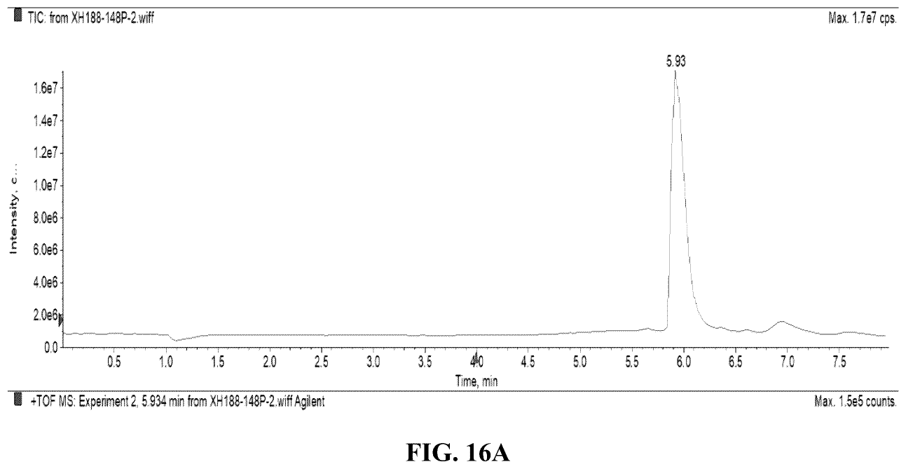

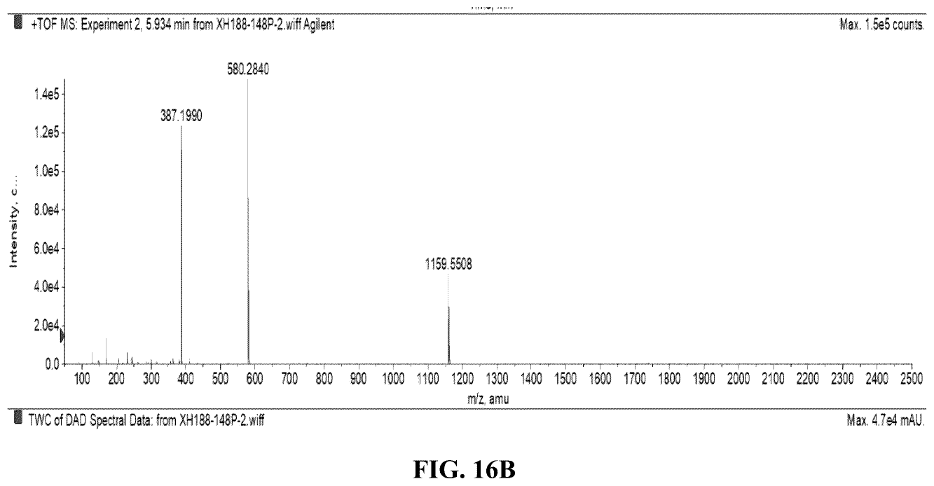

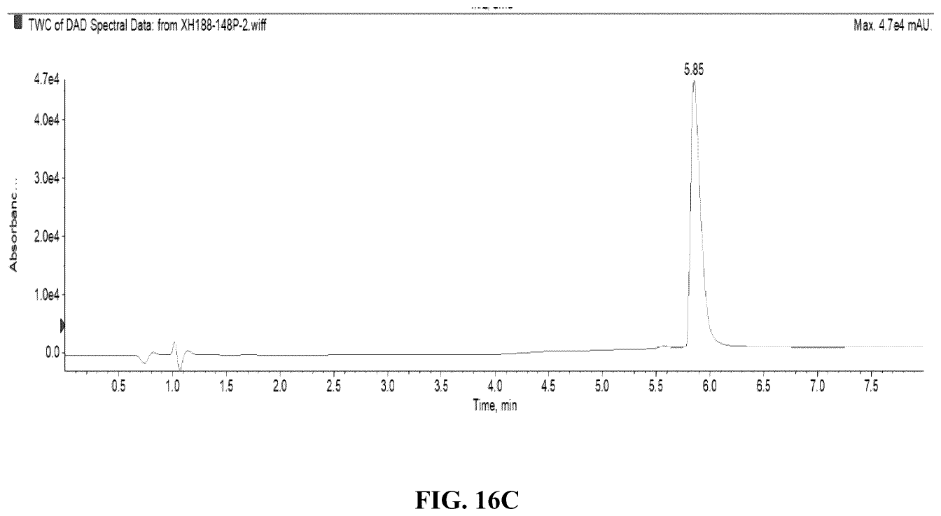

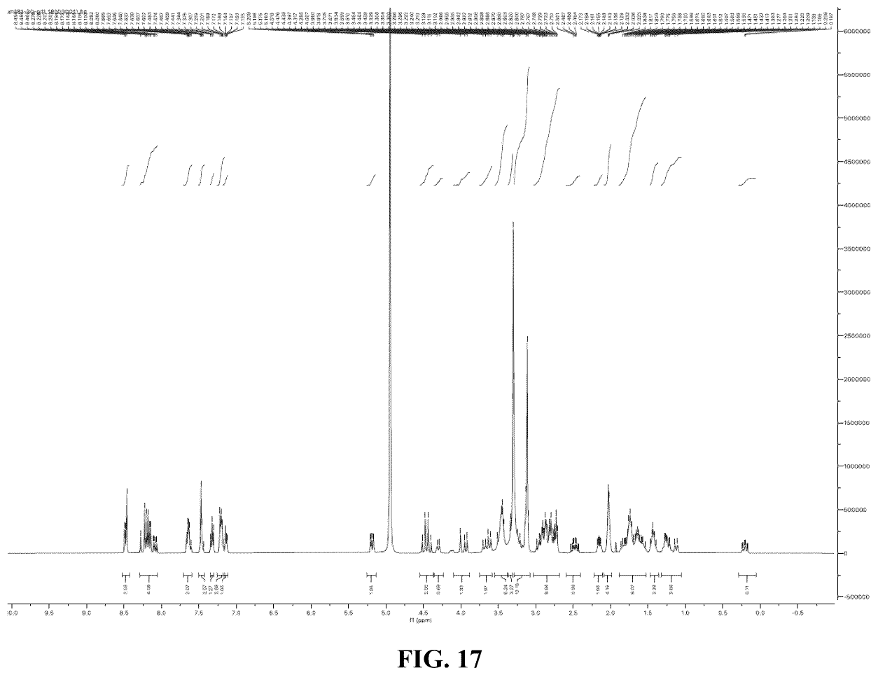

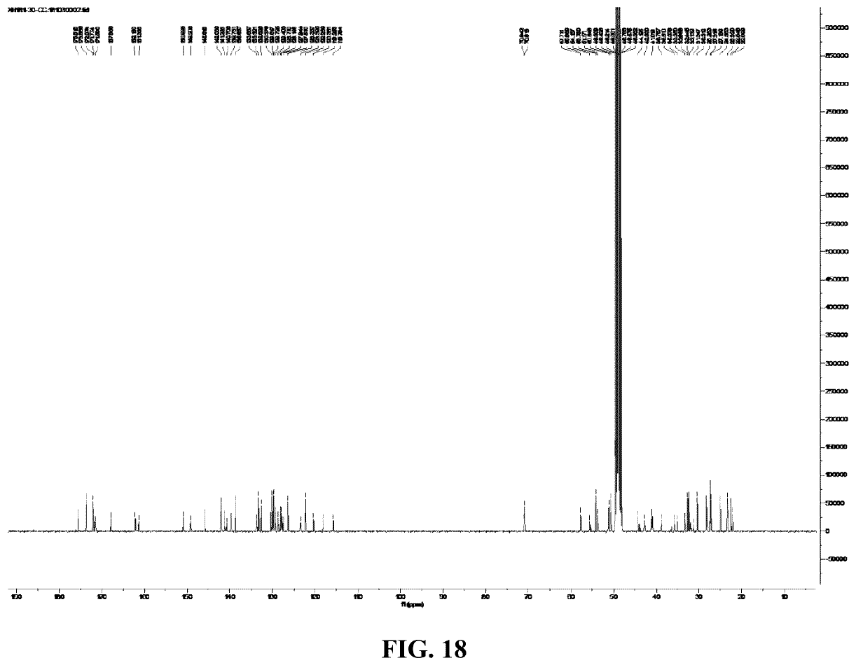

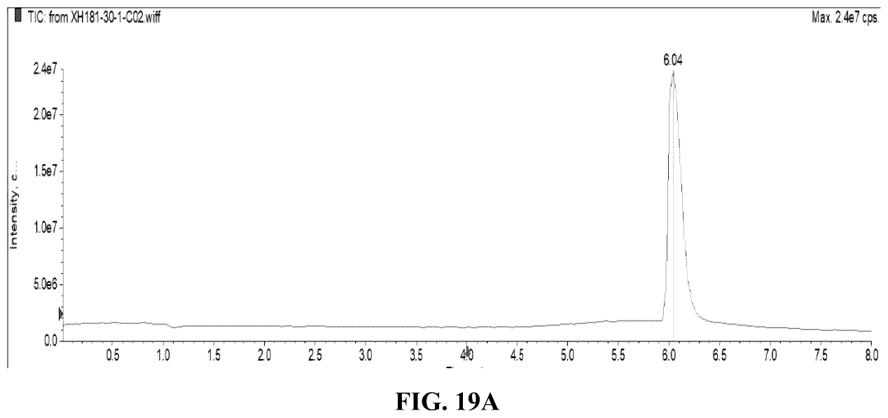

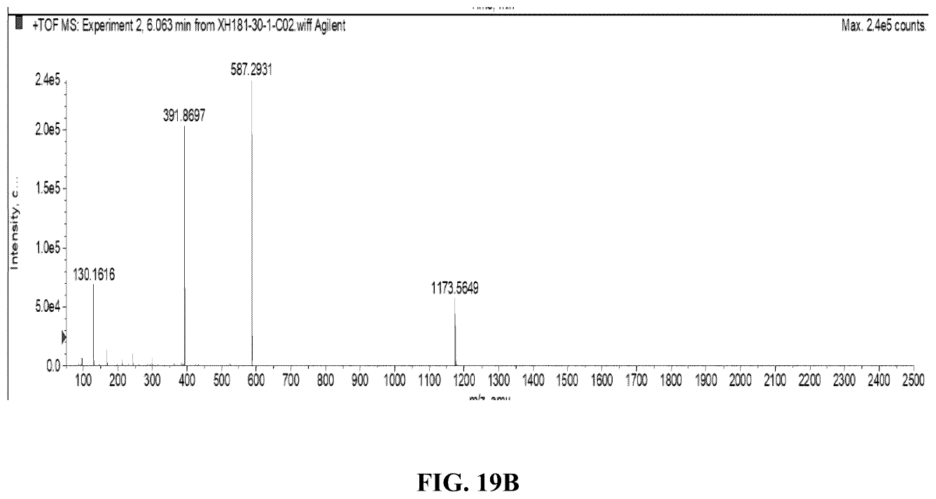

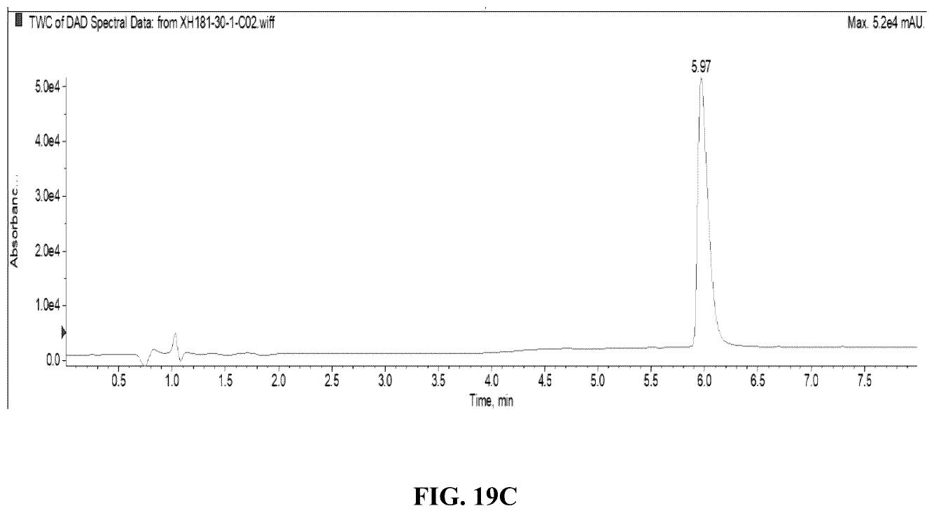

FIG. 14 shows ¹H NMR spectrum of PU7-1. FIG. 15 shows ¹³C NMR spectrum of PU7-1. FIGS. 16A-C show LC-MS spectrum of PU7-1. FIG. 16A shows Total Ion Chromatogram spectral data of PU7-1. FIG. 16B shows Time of Flight spectral data of PU7-1. FIG. 16C shows LC-MS total wave chromatogram spectral data of PU7-1. FIG. 17 shows shows ¹H NMR spectrum of PU7-1N. FIG. 18 shows ¹³C NMR spectrum of PU7-1N. FIGS. 19A-C show LC-MS spectrum of PU7-1N. FIG. 19A shows Total Ion Chromatogram spectral data of PU7-1N. FIG. 19B shows Time of Flight spectral data of PU7-1N. FIG. 19C shows LC-MS total wave chromatogram spectral data of PU7-1N.

### REFERENCES FOR EXAMPLE 2

Wang; S., Li; Y., AGUILAR; A., Zhou; B., HU; J., XU; F., QIN; C., Hu; Y., XIANG; W., REJ; R., et al. (2017). Monofunctional intermediates for ligand-dependent target protein degradation. patent application WO2017176958A1.

What is claimed is:

1. A compound: 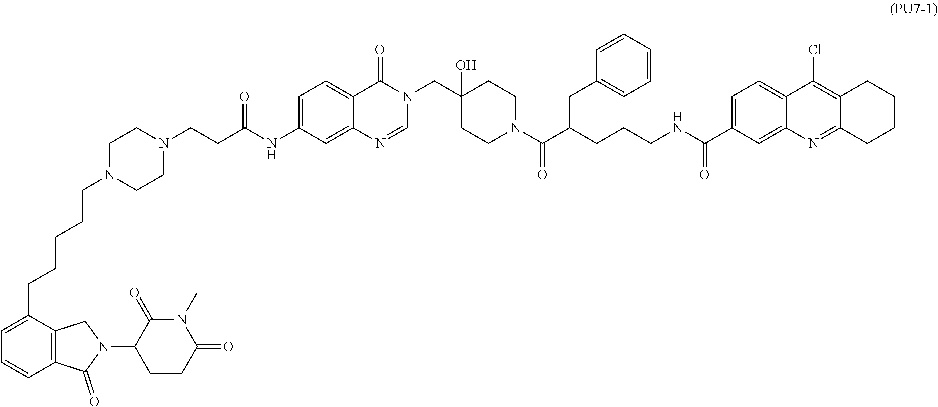 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]
or a pharmaceutically acceptable salt thereof.

2. A compound:

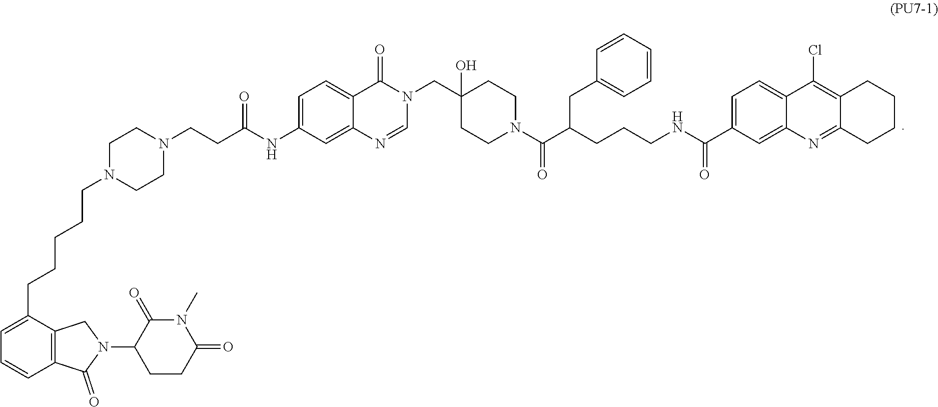 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]

3. A pharmaceutical composition comprising a pharmaceutically acceptable excipient and the compound according to claim 1.

4. A pharmaceutical composition comprising a pharmaceutically acceptable excipient and the compound according to claim 2.

5. The pharmaceutical composition of claim 3, wherein the composition comprises a therapeutically effective amount of the compound according to claim 1.

6. The pharmaceutical composition of claim 4, wherein the composition comprises a therapeutically effective amount of the compound according to claim 2.

7. A method of treating cancer in a subject in need thereof comprising administering to the subject a therapeutically effective amount of compound: 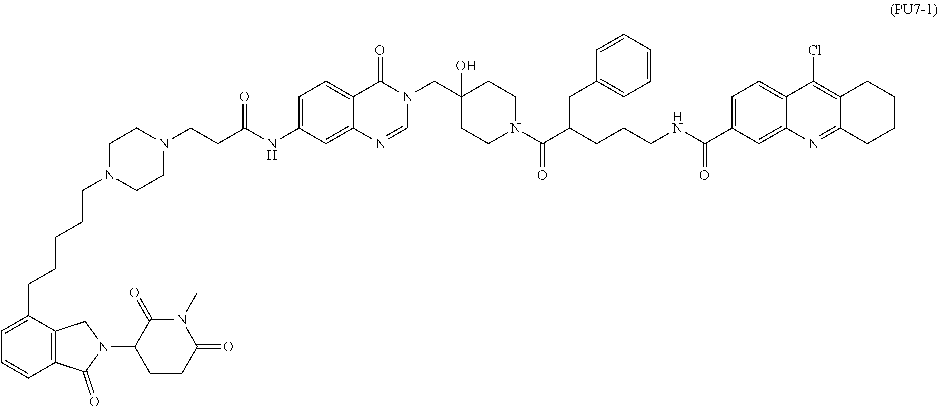 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]
or a pharmaceutically acceptable salt thereof.

8. A method of treating cancer in a subject in need thereof comprising administering to the subject a therapeutically effective amount of compound:

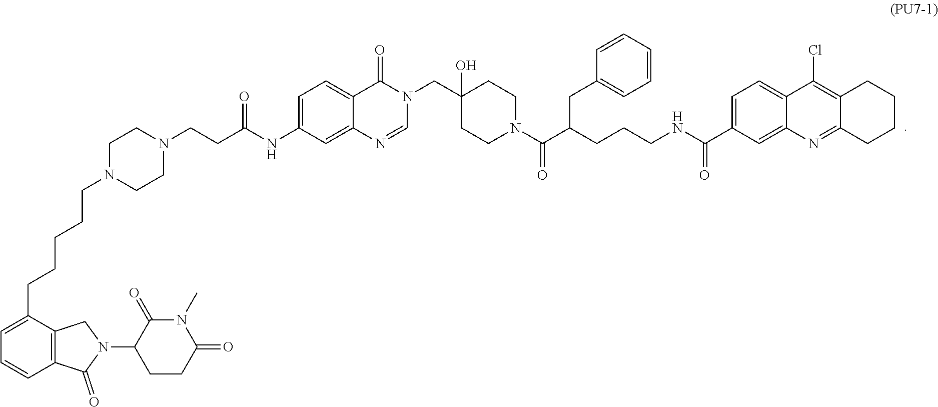 [SMILES: CN1C(=O)CCC(N2Cc3c(CCCCCN4CCN(CCC(=O)Nc5ccc6c(=O)n(CC7(O)CCN(C(=O)C(CCCNC(=O)c8ccc9c(Cl)c%10c(nc9c8)CCCC%10)Cc8ccccc8)CC7)cnc6c5)CC4)cccc3C2=O)C1=O]

9. The method of claim 7, wherein the compound (PU7-1) or the pharmaceutically acceptable salt thereof is administered as a pharmaceutical composition comprising a pharmaceutically acceptable excipient.

10. The method of claim 8, wherein the compound (PU7-1) is administered as a pharmaceutical composition comprising a pharmaceutically acceptable excipient.

11. The method of claim 7, wherein the cancer is a p53-independent cancer.

12. The method of claim 7, wherein the cancer is triple-negative breast cancer.

13. The method of claim 7, wherein the subject is a human.

14. The method of claim 8, wherein the cancer is a p53-independent cancer.

15. The method of claim 8, wherein the cancer is triple-negative breast cancer.

16. The method of claim 8, wherein the subject is a human.

17. The method of claim 9, wherein the cancer is a p53-independent cancer.

18. The method of claim 9, wherein the cancer is triple-negative breast cancer.

19. The method of claim 9, wherein the subject is a human.

20. The method of claim 10, wherein the cancer is a p53-independent cancer.

21. The method of claim 10, wherein the cancer is triple-negative breast cancer.

22. The method of claim 10, wherein the subject is a human.
